(19) United States
(12) Patent Application Publication
(10) Pub. No.: US 2025/0222133 A1
(43) Pub. Date: Jul. 10, 2025

(54) CELL-TYPE SPECIFIC REGULATORY ELEMENTS FOR PHOTORECEPTORS

(71) Applicant: SENTI BIOSCIENCES, INC., South San Francisco, CA (US)

(72) Inventors: Russell Morrison Gordley, South San Francisco, CA (US); Rocky Cheung, South San Francisco, CA (US); Assen Boyanov Roguev, South San Francisco, CA (US); Joseph Draut, South San Francisco, CA (US); Thant Htet Zaw, South San Francisco, CA (US)

(21) Appl. No.: 19/093,913

(22) Filed: Mar. 28, 2025

Related U.S. Application Data
(63) Continuation of application No. PCT/US23/75362, filed on Sep. 28, 2023.
(60) Provisional application No. 63/384,055, filed on Nov. 16, 2022.
(60) Provisional application No. 63/378,046, filed on Sep. 30, 2022.

(51) Int. Cl. A61K 48/00; C12N 5/079; C12N 15/85

(52) U.S. Cl. CPC A61K 48/0041; C12N 5/0621; C12N 15/85; C12N 2830/008

(57) ABSTRACT

The present disclosure provides for, among other things, methods and compositions comprising engineered nucleic acids, such as engineered photoreceptor-specific regulatory elements, that allow for highly selective and efficient transcriptional activity of an operably linked polynucleotide in photoreceptor cells within a human retina over non-photoreceptor cells of the same retina.

### CROSS REFERENCE TO RELATED APPLICATIONS

This application claims the benefit of U.S. Provisional Application Nos. 63/378,046 filed Sep. 30, 2022 and 63/384,055 filed Nov. 16, 2022, each of which is hereby incorporated by reference in their entirety for all purposes.

### SEQUENCE LISTING

The instant application contains a Sequence Listing which has been submitted via EFS-Web and is hereby incorporated by reference in its entirety. Said ASCII copy, created on Month XX, 20XX, is named XXXXXUS_sequencelisting.txt, and is X,XXX,XXX bytes in size.

### BACKGROUND

Gene regulatory elements, such as promoters and enhancers, can possess cell-type specific activities which provide controlled and restricted expression of certain genes in a particular target cell, while minimizing or excluding expression of certain genes in surrounding non-target cells. Such cell-type specific regulatory elements are often adapted for use in therapies, such as gene therapy, to treat diseases that benefit from selective expression of a particular gene. However, problems with transcriptional strength and specificity persist in known cell-specific regulatory systems. Accordingly, there is a need to identify further cell-specific regulatory elements for use in therapy.

### SUMMARY

Use of cell-type specific regulatory elements (e.g., enhancers, promoters, enhancer-promoter combinations, etc.) to restrict expression of therapeutic genes to particular cells is an attractive approach for a variety of therapeutic modalities (e.g., gene therapy, cell therapy, and so forth). Nevertheless, use of such cell-type specific regulatory elements is often hindered by inefficient transcriptional activity, or in some cases, cell-type specificity of a regulatory element is still insufficient for a particular therapeutic application. The present disclosure appreciates that there is a particular need to identify highly active regulatory elements that have specific biological activity in photoreceptor cells over non-photoreceptor cells. For example, genetic mutations that cause retinal degeneration are highly heterogeneous, and accordingly, may result from mutations in genes that have specific regulatory patterns in photoreceptor cells (e.g., rod cells and/or cone cells). Accordingly, identification of transcriptional regulatory elements that allow for sufficient and photoreceptor cell-specific expression of a particular gene (e.g., a therapeutic gene, that offsets any deficiencies within a diseased photoreceptor cell when expressed) would be invaluable in the treatment of ocular diseases or disorders. The present disclosure provides for such regulatory elements, which are photoreceptor-specific, and further show increased activity in photoreceptor cells, as compared to non-photoreceptor cells.

The present disclosure provides for, among other things, methods and compositions comprising engineered nucleic acids, such as engineered photoreceptor-specific regulatory elements, that allow for highly selective and efficient transcriptional activity of an operably linked polynucleotide (e.g., a gene) in photoreceptor cells within a mammalian retina over non-photoreceptor cells with the same retina. The present disclosure sets forth combinations of certain native genomic enhancer regions, when operably linked to a heterologous promoter (e.g., a minimal promoter), exhibit selectivity for activity in photoreceptor cells as compared to non-photoreceptor cells.

The present disclosure also provides engineered photoreceptor-specific regulatory elements that comprise an engineered enhancer region. In some embodiments, an engineered enhancer region includes a portion where at least one nucleotide motif therein is modified, e.g., ablated. In some embodiments, an engineered photoreceptor-specific regulatory element comprises an engineered enhancer region that is operably linked to a promoter (e.g., a minimal promoter).

Also provided by the present disclosure are engineered photoreceptor cells comprising engineered nucleic acids as described herein.

The present disclosure provides for engineered photoreceptor-specific regulatory elements comprising an enhancer region operably linked to a minimal promoter, wherein the enhancer region is heterologous to the minimal promoter, and wherein the engineered photoreceptor-specific regulatory element exhibits greater activity in photoreceptor cells of a mammalian retina as compared to non-photoreceptor cells of the same mammalian retina.

In some embodiments, an enhancer region is about 50 to about 600 base pairs in length, about 100 to about 600 base pairs in length, about 200 to about 600 base pairs in length, about 300 to about 600 base pairs in length, about 400 to about 600 base pairs in length, about 500 to about 600 base pairs in length, about 50 to about 500 base pairs in length, about 50 to about 400 base pairs in length, about 50 to about 300 base pairs in length, about 50 to about 200 base pairs in length, about 50 to about 175 base pairs in length, about 50 to about 150 base pairs in length, about 50 to about 125 base pairs in length, or about 50 to about 100 base pairs in length.

In some embodiments, a minimal promoter is selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, lateADE, minIL2.2, SMP, inducer molecule responsive promoters, CMV, EFS, SFFV, SV40, MND, PGK, UbC, hEFlaV1, hCAGG, hEFlaV2, hACTb, heIF4Al, hGAPDH, hGRP78, hGRP94, hHSP70, hKINb, hUBIb, and tandem repeats thereof.

In some embodiments, an engineered photoreceptor-specific regulatory element as described herein further comprises a spacer sequence, wherein the spacer is operably linked to an enhancer region and a minimal promoter. In some embodiments, a spacer is selected from the group consisting of SEQ ID Nos 107-110. In some embodiments, a spacer comprises the nucleotide sequence: CCTGCAGG (SEQ ID NO: 107).

In some embodiments, an enhancer region comprises the nucleotide sequence: TGTTGAGAGCTCAAGCTCTTTTTAACGCTTTGCCTTGCTGTCTCCAAAGTATTGCCTT CATCCTCATAGTTCAAAGTGTCCACCATCACATTCACGTTTCAGCCAATAGGAAGAA GGAAAGAGAAAAACAGGAAAAGAGTTTACATGCCACTCCTGCTATTGGTCAGAATG TGTCACGTGACCATACTCAGCTGCAAGGGTTGGTGGGAAATGTAGTCTTTTTTTCTG GGAGGCCATGTGTTCGGCTTAAAATTGTCTTTTTTTCTTTTTCTTTATTTCTTTTTTCTT TTCTTTCTTTTTTTTAGACGG (SEQ ID NO: 1). In some embodiments, a minimal promoter is derived from YB TATA. In some embodiments, a minimal promoter comprises the nucleotide sequence: TCTAGAGGGTATATAATGGGGGCCA (SEQ ID NO: 122). In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 138.

In some embodiments, an enhancer region comprises the nucleotide sequence: GGTTACACAACCAGGCGGGGAGGGGCCCCGGGGCGGGGAGGGGGCCGGCCCGCGG GCCGCGCAGCCGGAAGCCGGGACCGCCACCGGCCCCCGGCGAGGGGAGCCCGGCTC CAGGCCCCGCCCCCTGGCGGGCTGGCCCCGCCCCCGCGCCGCGCCGCGCGATCGGC CCGCGCCCATTGGCTCTCCGGCCCGCCGCTCACCGCCCCTCCTCCGCACCGCCCCTA CCCGCAGGCCGCGGCGGGCTGTCGGCGCGGGGCACCCTGGGACTTGTAGTCCAAGC CGCTTGCCACCTGCCGGCTGCAAACGGCGGAGGGACTACGAAGCCCAGAGGTCCCT GCGGCCCTGCCCGCCCACCCGGACACCCCACCCCTTCCCCCTCCTTTCCGAAGCCCC CCTCCCTGTTTTTTCAGCCCCCTC (SEQ ID NO: 2). In some embodiments, a minimal promoter is derived from YB TATA. In some embodiments, a minimal promoter comprises the nucleotide sequence: TCTAGAGGGTATATAATGGGGGCCA (SEQ ID NO: 122). In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 139.

In some embodiments, an enhancer region comprises the nucleotide sequence: CAGGCGGGGAGGGGCCCCGGGGCGGGGAGGGGGCCGGCCCGCGGGCCGCGCAGCC GGAAGCCGGGACCGCCACCGGCCCCCGGCGAGGGGAGCCCGGCTCCAGGCCCCGCC CCCTGGCGGGCTGGCCCCGCCCCCGCGCCGCGCCGCGCGATCGGCCCGCGCCCATTG GCTCTCCGGCCCGCCGCTCACCGCCCCTCCTCCGCACCGCCCCTACCCGCAGGCCGC GGCGGGCTGTCGGCGCGGGGCACCCTGGGACTTGTAGTCCAAGCCGCTTGCCACCT GCCGGCTGCAAACGGCGGAGGGACTACGAAGCCCAGAGGTCCCTGCGGCCCTGCCC GCCCACCCGGACACCCCACCCCTTCCCCCTCCTTTCCGAAGCCCCCCTCCCTGTTTTT TCAGCCCCCTCCCCCCCATCCCCCATGGAGC (SEQ ID NO: 3). In some embodiments, a minimal promoter is derived from YB TATA. In some embodiments, a minimal promoter comprises the nucleotide sequence: TCTAGAGGGTATATAATGGGGGCCA (SEQ ID NO: 122). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 140.

In some embodiments, an enhancer region comprises the nucleotide sequence: TGTTGAGAGCTCAAGCTCTTTTTAACGCTTTGCCTTGCTGTCTCCAAAGTATTGCCTT CATCCTCATAGTTCAAAGTGTCCACCATCACATTCACGTTTCAGCCAATAGGAAGAA GGAAAGAGAAAAACAGGAAAAGAGTTTACATGCCACTCCTGCTATTGGTCAGAATG TGTCACGTGACCATACTCAGCTGCAAGGGTTGGTGGGAAATGTAGTCTTTTTTTCTG GGAGGCCATGTGTTCGGCTTAAAATTGTCTTTTTTTCTTTTTCTTTATTTCTTTTTTCTT TTCTTTCTTTTTTTTAGACGG (SEQ ID NO: 1). In some embodiments, a minimal promoter is derived from minTK. In some embodiments, a minimal promoter comprises the nucleotide sequence: TTCGCATATTAAGGTGACGCGTGTGGCCTCGAACACCGAGCGACCCTGCAGCGACC CGCTTAA (SEQ ID NO: 123). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 141. In some embodiments, a minimal promoter is derived from lateADE. In some embodiments, a minimal promoter comprises the nucleotide sequence: AGACGCTAGCGGGGGGCTATAAAAGGGGGTGGGGGCGTTCGTCCTCACTCT (SEQ ID NO: 126). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 142.

In some embodiments, an enhancer region comprises the nucleotide sequence: AGCACGCGCGAGCTGTCACCAGGGCGCGAGACAACCGAACACGGCCCCGCCCCCTG CCCGTCTATTGGCCCGCCCGCTGGAGCCCCGCCCCTCAGGCCCTGCATTGCGCCAAC GGCGCAGCGCTGGGCCGCGACCCCGGCACCGGCACCCGTTCCGAGGGTTCGCGCCG CAGGCGCAAAGTTTAGGGTCAGCCACAAACGCGCGGCCGCTTTGAGCCTGGCGTAA GGGGCGGCGGCGTAGGGGGACGTTGTGAATAGCTGGTCGAAGTTTGGTTGAAGCAC AGGAGGGTAAATAAGAAGGTTTCCAGACAAAGAGACTCTTAAAGGTACAGACTCTT ATGTCTGTCCCTCCTCCTTAAAGGGCCAGAGACGCTCCCGAGCCCATCTC (SEQ ID NO: 4). In some embodiments, a minimal promoter is derived from SMP. In some embodiments, a minimal promoter comprises the nucleotide sequence: AAAATGTGCGCATGTGCAGCCATTGCCTGGGACGCATGCGTAGGGAGCCGCGCGAC AAACTGAGCCATTGCGGCAAGACTAGCGCAGAGAGGAGAGGGAGCCGGAGATGCC AGACGCTTGGTTCTGAGGAGTGATTTGCAACGCAATGGAGCGAGGAAGG (SEQ ID NO: 125). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 143. In some embodiments, a minimal promoter is derived from lateADE. In some embodiments, the minimal promoter comprises the nucleotide sequence: AGACGCTAGCGGGGGGCTATAAAAGGGGGTGGGGGCGTTCGTCCTCACTCT (SEQ ID NO: 126). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 144. In some embodiments, a minimal promoter is derived from minCMV. In some embodiments, a minimal promoter comprises the nucleotide sequence: TAGGCGTGTACGGTGGGAGGCCTATATAAGCAGAGCTCGTTTAGTGAACCGTCAGA TCGCCTGGA (SEQ ID NO: 127). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 145.

In some embodiments, an enhancer region comprises the nucleotide sequence: GGTTACACAACCAGGCGGGGAGGGGCCCCGGGGCGGGGAGGGGGCCGGCCCGCGG GCCGCGCAGCCGGAAGCCGGGACCGCCACCGGCCCCCGGCGAGGGGAGCCCGGCTC CAGGCCCCGCCCCCTGGCGGGCTGGCCCCGCCCCCGCGCCGCGCCGCGCGATCGGC CCGCGCCCATTGGCTCTCCGGCCCGCCGCTCACCGCCCCTCCTCCGCACCGCCCCTA CCCGCAGGCCGCGGCGGGCTGTCGGCGCGGGGCACCCTGGGACTTGTAGTCCAAGC CGCTTGCCACCTGCCGGCTGCAAACGGCGGAGGGACTACGAAGCCCAGAGGTCCCT GCGGCCCTGCCCGCCCACCCGGACACCCCACCCCTTCCCCCTCCTTTCCGAAGCCCC CCTCCCTGTTTTTTCAGCCCCCTC (SEQ ID NO: 2). In some embodiments, a minimal promoter is derived from minTK. In some embodiments, a minimal promoter comprises the nucleotide sequence: TTCGCATATTAAGGTGACGCGTGTGGCCTCGAACACCGAGCGACCCTGCAGCGACC CGCTTAA (SEQ ID NO: 123). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 146. In some embodiments, a minimal promoter is derived from minIL2.2. In some embodiments, a minimal promoter comprises the nucleotide sequence: CAGAATTAACAGTATAAATTGCATCTCTTGTTCAAGAGTTCCCTATCACTCT (SEQ ID NO: 124). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the sequence of SEQ ID NO: 147. In some embodiments, a minimal promoter is derived from lateADE. In some embodiments, a minimal promoter comprises the nucleotide sequence: AGACGCTAGCGGGGGGCTATAAAAGGGGGTGGGGGCGTTCGTCCTCACTCT (SEQ ID NO: 126). In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to the sequence of SEQ ID NO: 148.

The present disclosure also provides for engineered photoreceptor-specific regulatory elements comprising an enhancer region, wherein the enhancer region comprises an ablation of at least one nucleotide motif within a wild-type enhancer region, and wherein the engineered photoreceptor-specific regulatory element has greater activity than the same regulatory element without the ablation in photoreceptor cells as compared to non-photoreceptor cells.

In some embodiments, an engineered photoreceptor-specific regulatory element further comprises a minimal promoter.

In some embodiments, an engineered photoreceptor-specific regulatory element further comprises a spacer, wherein spacer is operably linked to an enhancer region and a minimal promoter. In some embodiments, an engineered photoreceptor-specific regulatory element further comprises a spacer, wherein the spacer is located between an enhancer region and a minimal promoter.

In some embodiments, a wild-type enhancer region comprises the nucleotide sequence:

(SEQ ID NO: 1)
TGTTGAGAGCTCAAGCTCTTTTTAACGCTTTGCCTTGCTGTCTCCAAAG
TATTGCCTTCATCCTCATAGTTCAAAGTGTCCACCATCACATTCACGTT
TCAGCCAATAGGAAGAAGGAAAGAGAAAAACAGGAAAAGAGTTTACATG
CCACTCCTGCTATTGGTCAGAATGTGTCACGTGACCATACTCAGCTGCA
AGGGTTGGTGGGAAATGTAGTCTTTTTTTCTGGGAGGCCATGTGTTCGG
CTTAAAATTGTCTTTTTTTCTTTTTCTTTATTTCTTTTTTCTTTTCTTT
CTTTTTTTTAGACGG.

In some embodiments, an ablation comprises a substitution or deletion of one or more nucleotides of the at least one nucleotide motif.

In some embodiments, an at least one nucleotide motif comprises a motif having a sequence within the nucleotide sequence of SEQ ID NO: 1.

In some embodiments, an ablation comprises a nucleotide substitution of the motif comprising the nucleotide sequence: TGTTGAGAGCTCAAGCTCTTTTTAACGCTT (SEQ ID NO: 5).

In some embodiments, an ablation comprises a nucleotide substitution of the motif comprising the nucleotide sequence: TGCCTTGCTGTCTCCAAAGTATTGCCTTCA (SEQ ID NO: 7).

In some embodiments, an ablation comprises a nucleotide substitution of the motif comprising the nucleotide sequence: TCCTCATAGTTCAAAGTGTCCACCATCACA (SEQ ID NO: 9).

In some embodiments, an ablation comprises a nucleotide substitution of the motif comprising the nucleotide sequence: TTCACGTTTCAGCCAATAGGAAGAAGGAAA (SEQ ID NO: 11).

In some embodiments, an ablation comprises a nucleotide substitution of the motif comprising the nucleotide sequence: GAGAAAAACAGGAAAAGAGTTTACATGCCA (SEQ ID NO: 13).

In some embodiments, an ablation comprises a nucleotide substitution of the motif comprising the nucleotide sequence: CTTTATTTCTTTTTTCTTTTCTTTCTTTTT (SEQ ID NO: 15).

In some embodiments, an ablation comprises a nucleotide substitution of the motif comprising the nucleotide sequence: TTTAGACGG (SEQ ID NO: 17).

In some embodiments, a nucleotide substitution comprises an inert sequence.

In some embodiments, a wild-type enhancer region comprises the nucleotide sequence:

(SEQ ID NO: 2)
GGTTACACAACCAGGCGGGGAGGGGCCCCGGGGGGGGGAGGGGGCCGGC
CCGCGGGCCGCGCAGCCGGAAGCCGGGACCGCCACCGGCCCCCGGCGAG
GGGAGCCCGGCTCCAGGCCCCGCCCCCTGGCGGGCTGGCCCCGCCCCCG
CGCCGCGCCGCGCGATCGGCCCGCGCCCATTGGCTCTCCGGCCCGCCGC
TCACCGCCCCTCCTCCGCACCGCCCCTACCCGCAGGCCGCGGCGGGCTG
TCGGCGCGGGGCACCCTGGGACTTGTAGTCCAAGCCGCTTGCCACCTGC
CGGCTGCAAACGGCGGAGGGACTACGAAGCCCAGAGGTCCCTGCGGCCC
TGCCCGCCCACCCGGACACCCCACCCCTTCCCCCTCCTTTCCGAAGCCC
CCCTCCCTGTTTTTTCAGCCCCCTC.

In some embodiments, an ablation comprises a substitution or deletion of one or more nucleotides of the at least one nucleotide motif.

In some embodiments, an at least one nucleotide motif comprises a motif having a sequence within the nucleotide sequence of SEQ ID NO: 2.

In some embodiments, an ablation comprises a nucleotide substitution of the motif comprising the nucleotide sequence: CCCCCTGGCGGGCTGGCCCCGCCCCCGCGC (SEQ ID NO: 20).

In some embodiments, an ablation comprises a nucleotide substitution of the motif comprising the nucleotide sequence: CCGGCGAGGGGAGCCCGGCTCCAGGCCCCG (SEQ ID NO: 19).

In some embodiments, a wild-type enhancer region comprises the nucleotide sequence:

(SEQ ID NO: 3)
CAGGCGGGGAGGGGCCCCGGGGCGGGGAGGGGGCCGGCCCGCGGGCCGC
GCAGCCGGAAGCCGGGACCGCCACCGGCCCCCGGCGAGGGGAGCCCGGC
TCCAGGCCCCGCCCCCTGGCGGGCTGGCCCCGCCCCCGCGCCGCGCCGC
GCGATCGGCCCGCGCCCATTGGCTCTCCGGCCCGCCGCTCACCGCCCCT
CCTCCGCACCGCCCCTACCCGCAGGCCGCGGCGGGCTGTCGGCGCGGGG
CACCCTGGGACTTGTAGTCCAAGCCGCTTGCCACCTGCCGGCTGCAAAC
GGCGGAGGGACTACGAAGCCCAGAGGTCCCTGCGGCCCTGCCCGCCCAC
CCGGACACCCCACCCCTTCCCCCTCCTTTCCGAAGCCCCCCTCCCTGTT
TTTTCAGCCCCCTCCCCCCCATCCCCCATGGAGC.

In some embodiments, an ablation comprises a substitution or deletion of one or more nucleotides of the at least one nucleotide motif.

In some embodiments, an at least one nucleotide motif comprises a motif having a sequence within the nucleotide sequence of SEQ ID NO: 3.

In some embodiments, an ablation comprises a nucleotide substitution of the motif comprising the nucleotide sequence: AGCCCGGCTCCAGGCCCCGCCCCCTGGCGG (SEQ ID NO: 22).

In some embodiments, an ablation comprises a nucleotide substitution of the motif comprising the nucleotide sequence: GCTGGCCCCGCCCCCGCGCCGCGCCGCGCG (SEQ ID NO: 23).

In some embodiments, an ablation comprises a nucleotide substitution of the motif comprising the nucleotide sequence: CTACCCGCAGGCCGCGGCGGGCTGTCGGCG (SEQ ID NO: 24).

In some embodiments, an ablation comprises a nucleotide substitution of the motif comprising the nucleotide sequence: GCTTGCCACCTGCCGGCTGCAAACGGCGGA (SEQ ID NO: 26).

In some embodiments, an ablation comprises a nucleotide substitution of the motif comprising the nucleotide sequence: CCTGCCCGCCCACCCGGACACCCCACCCCT (SEQ ID NO: 27).

In some embodiments, an ablation comprises a nucleotide substitution of the motif comprising the nucleotide sequence: TCCCCCTCCTTTCCGAAGCCCCCCTCCCTG (SEQ ID NO: 29).

In some embodiments, an enhancer region comprises: (i) at least one transcription factor binding site, (ii) at least one nucleotide motif, (iii) at least one ablation patch, (iv) at least one transcription factor binding site array, or (v) any combination thereof.

In some embodiments, an engineered photoreceptor-specific regulatory element comprises higher activity in photoreceptor cells of a mammalian retina as compared to non-photoreceptor cells of the same mammalian retina.

In some embodiments, an engineered photoreceptor-specific regulatory element as described herein further comprises a linker sequence, wherein the linker sequence is operably linked to the enhancer region and the minimal promoter. In some embodiments, a linker sequence is selected from the group consisting of: SEQ ID Nos: 107-110.

In some embodiments, an enhancer region comprises at least 75%, at least 80%, at least 85%, at least 90%, at least 95%, at least 96%, at least 97%, at least 98%, at least 99%, or 100% sequence identity to a wild-type sequence motif, ablation sequence motif, or regulatory element component nucleotide sequence selected from Table 2 or Table 4.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 164.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 165.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 166.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 167.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 168.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 169.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 170.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 171.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 172.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 173.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 174.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 175.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 176.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 177.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 178.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 179.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 180.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 181.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 182.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 183.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 184.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 185.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 186.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 187.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 188.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 189.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 190.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 191.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 192.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 193.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 194.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 195.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 196.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 197.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 198.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 199.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 200.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 201.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 202.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 203.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 204.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 205.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 206.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 207.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 208.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 209.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 210.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 211.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 212.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 213.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 214.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 215.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 216.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 217.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 218.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 219.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 220.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 221.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 222.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 223.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 224.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 225.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 226.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 227.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 228.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 229.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 230.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 231.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 232.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 233.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 234.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 235.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 236.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 237.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 238.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 239.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 240.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 241.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 242.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 243.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 244.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 245.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 246.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 247.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 276.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 277.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 278.

In some embodiments, higher activity is characterized by at least 100-fold, at least 200-fold, at least 300-fold, at least 400-fold, at least 500-fold, at least 600-fold, at least 700-fold, at least 800-fold, at least 900-fold, at least 1000-fold, at least 1100-fold, at least 1200-fold, or at least 1300-fold greater expression of one or more genes operably linked to the engineered photoreceptor-specific regulatory element.

In some embodiments, non-photoreceptor cells comprise muller cells and retinal pigment epithelial cells.

In some embodiments, an engineered photoreceptor-specific regulatory element comprises the polynucleotide sequence of SEQ ID NO: 138.

In some embodiments, an engineered photoreceptor-specific regulatory element comprises the polynucleotide sequence of SEQ ID NO: 139.

In some embodiments, an engineered photoreceptor-specific regulatory element comprises the polynucleotide sequence of SEQ ID NO: 140.

In some embodiments, an engineered photoreceptor-specific regulatory element comprises the polynucleotide sequence of SEQ ID NO: 141.

In some embodiments, an engineered photoreceptor-specific regulatory element comprises the polynucleotide sequence of SEQ ID NO: 142.

In some embodiments, an engineered photoreceptor-specific regulatory element comprises the polynucleotide sequence of SEQ ID NO: 143.

In some embodiments, an engineered photoreceptor-specific regulatory element comprises the polynucleotide sequence of SEQ ID NO: 144.

In some embodiments, an engineered photoreceptor-specific regulatory element comprises the polynucleotide sequence of SEQ ID NO: 145.

In some embodiments, an engineered photoreceptor-specific regulatory element comprises the polynucleotide sequence of SEQ ID NO: 146.

In some embodiments, an engineered photoreceptor-specific regulatory element comprises the polynucleotide sequence of SEQ ID NO: 147.

In some embodiments, an engineered photoreceptor-specific regulatory element comprises the polynucleotide sequence of SEQ ID NO: 148.

In some embodiments, an engineered photoreceptor-specific regulatory element comprises the polynucleotide sequence of SEQ ID NO: 149.

In some embodiments, an engineered photoreceptor-specific regulatory element comprises the polynucleotide sequence of SEQ ID NO: 150.

In some embodiments, an engineered photoreceptor-specific regulatory element comprises the polynucleotide sequence of SEQ ID NO: 151.

In some embodiments, an engineered photoreceptor-specific regulatory element comprises the polynucleotide sequence of SEQ ID NO: 152.

In some embodiments, an engineered photoreceptor-specific regulatory element comprises the polynucleotide sequence of SEQ ID NO: 153.

In some embodiments, an engineered photoreceptor-specific regulatory element comprises the polynucleotide sequence of SEQ ID NO: 154.

In some embodiments, an engineered photoreceptor-specific regulatory element comprises the polynucleotide sequence of SEQ ID NO: 155.

In some embodiments, an engineered photoreceptor-specific regulatory element comprises the polynucleotide sequence of SEQ ID NO: 156.

In some embodiments, an engineered photoreceptor-specific regulatory element comprises the polynucleotide sequence of SEQ ID NO: 157.

In some embodiments, an engineered photoreceptor-specific regulatory element comprises the polynucleotide sequence of SEQ ID NO: 158.

In some embodiments, an engineered photoreceptor-specific regulatory element comprises the polynucleotide sequence of SEQ ID NO: 159.

In some embodiments, an engineered photoreceptor-specific regulatory element comprises the polynucleotide sequence of SEQ ID NO: 160.

In some embodiments, an engineered photoreceptor-specific regulatory element comprises the polynucleotide sequence of SEQ ID NO: 161.

In some embodiments, an engineered photoreceptor-specific regulatory element comprises the polynucleotide sequence of SEQ ID NO: 162.

In some embodiments, an engineered photoreceptor-specific regulatory element comprises the polynucleotide sequence of SEQ ID NO: 163.

In some embodiments, an engineered photoreceptor-specific regulatory element comprises the polynucleotide sequence of SEQ ID NO: 164.

In some embodiments, an engineered photoreceptor-specific regulatory element comprises the polynucleotide sequence of SEQ ID NO: 165.

In some embodiments, an engineered photoreceptor-specific regulatory element comprises the polynucleotide sequence of SEQ ID NO: 166.

In some embodiments, an engineered photoreceptor-specific regulatory element comprises the polynucleotide sequence of SEQ ID NO: 167.

In some embodiments, an engineered photoreceptor-specific regulatory element comprises the polynucleotide sequence of SEQ ID NO: 168.

In some embodiments, an engineered photoreceptor-specific regulatory element comprises the polynucleotide sequence of SEQ ID NO: 169.

In some embodiments, an engineered photoreceptor-specific regulatory element comprises the polynucleotide sequence of SEQ ID NO: 170.

In some embodiments, an engineered photoreceptor-specific regulatory element comprises the polynucleotide sequence of SEQ ID NO: 171.

In some embodiments, an engineered photoreceptor-specific regulatory element comprises the polynucleotide sequence of SEQ ID NO: 172.

In some embodiments, an engineered photoreceptor-specific regulatory element comprises the polynucleotide sequence of SEQ ID NO: 173.

In some embodiments, an engineered photoreceptor-specific regulatory element comprises the polynucleotide sequence of SEQ ID NO: 174.

In some embodiments, an engineered photoreceptor-specific regulatory element comprises the polynucleotide sequence of SEQ ID NO: 175.

In some embodiments, an engineered photoreceptor-specific regulatory element comprises the polynucleotide sequence of SEQ ID NO: 176.

In some embodiments, an engineered photoreceptor-specific regulatory element comprises the polynucleotide sequence of SEQ ID NO: 177.

In some embodiments, an engineered photoreceptor-specific regulatory element comprises the polynucleotide sequence of SEQ ID NO: 178.

In some embodiments, an engineered photoreceptor-specific regulatory element comprises the polynucleotide sequence of SEQ ID NO: 179.

In some embodiments, an engineered photoreceptor-specific regulatory element comprises the polynucleotide sequence of SEQ ID NO: 180.

In some embodiments, an engineered photoreceptor-specific regulatory element comprises the polynucleotide sequence of SEQ ID NO: 181.

In some embodiments, an engineered photoreceptor-specific regulatory element comprises the polynucleotide sequence of SEQ ID NO: 182.

In some embodiments, an engineered photoreceptor-specific regulatory element comprises the polynucleotide sequence of SEQ ID NO: 183.

In some embodiments, an engineered photoreceptor-specific regulatory element comprises the polynucleotide sequence of SEQ ID NO: 184.

In some embodiments, an engineered photoreceptor-specific regulatory element comprises the polynucleotide sequence of SEQ ID NO: 185.

In some embodiments, an engineered photoreceptor-specific regulatory element comprises the polynucleotide sequence of SEQ ID NO: 186.

In some embodiments, an engineered photoreceptor-specific regulatory element comprises the polynucleotide sequence of SEQ ID NO: 187.

In some embodiments, an engineered photoreceptor-specific regulatory element comprises the polynucleotide sequence of SEQ ID NO: 188.

In some embodiments, an engineered photoreceptor-specific regulatory element comprises the polynucleotide sequence of SEQ ID NO: 189.

In some embodiments, an engineered photoreceptor-specific regulatory element comprises the polynucleotide sequence of SEQ ID NO: 190.

In some embodiments, an engineered photoreceptor-specific regulatory element comprises the polynucleotide sequence of SEQ ID NO: 191.

In some embodiments, an engineered photoreceptor-specific regulatory element comprises the polynucleotide sequence of SEQ ID NO: 192.

In some embodiments, an engineered photoreceptor-specific regulatory element comprises the polynucleotide sequence of SEQ ID NO: 193.

In some embodiments, an engineered photoreceptor-specific regulatory element comprises the polynucleotide sequence of SEQ ID NO: 194.

In some embodiments, an engineered photoreceptor-specific regulatory element comprises the polynucleotide sequence of SEQ ID NO: 195.

In some embodiments, an engineered photoreceptor-specific regulatory element comprises the polynucleotide sequence of SEQ ID NO: 196.

In some embodiments, an engineered photoreceptor-specific regulatory element comprises the polynucleotide sequence of SEQ ID NO: 197.

In some embodiments, an engineered photoreceptor-specific regulatory element comprises the polynucleotide sequence of SEQ ID NO: 198.

In some embodiments, an engineered photoreceptor-specific regulatory element comprises the polynucleotide sequence of SEQ ID NO: 199.

In some embodiments, an engineered photoreceptor-specific regulatory element comprises the polynucleotide sequence of SEQ ID NO: 200.

In some embodiments, an engineered photoreceptor-specific regulatory element comprises the polynucleotide sequence of SEQ ID NO: 201.

In some embodiments, an engineered photoreceptor-specific regulatory element comprises the polynucleotide sequence of SEQ ID NO: 202.

In some embodiments, an engineered photoreceptor-specific regulatory element comprises the polynucleotide sequence of SEQ ID NO: 203.

In some embodiments, an engineered photoreceptor-specific regulatory element comprises the polynucleotide sequence of SEQ ID NO: 204.

In some embodiments, an engineered photoreceptor-specific regulatory element comprises the polynucleotide sequence of SEQ ID NO: 205.

In some embodiments, an engineered photoreceptor-specific regulatory element comprises the polynucleotide sequence of SEQ ID NO: 206.

In some embodiments, an engineered photoreceptor-specific regulatory element comprises the polynucleotide sequence of SEQ ID NO: 207.

In some embodiments, an engineered photoreceptor-specific regulatory element comprises the polynucleotide sequence of SEQ ID NO: 208.

In some embodiments, an engineered photoreceptor-specific regulatory element comprises the polynucleotide sequence of SEQ ID NO: 209.

In some embodiments, an engineered photoreceptor-specific regulatory element comprises the polynucleotide sequence of SEQ ID NO: 210.

In some embodiments, an engineered photoreceptor-specific regulatory element comprises the polynucleotide sequence of SEQ ID NO: 211.

In some embodiments, an engineered photoreceptor-specific regulatory element comprises the polynucleotide sequence of SEQ ID NO: 212.

In some embodiments, an engineered photoreceptor-specific regulatory element comprises the polynucleotide sequence of SEQ ID NO: 213.

In some embodiments, an engineered photoreceptor-specific regulatory element comprises the polynucleotide sequence of SEQ ID NO: 214.

In some embodiments, an engineered photoreceptor-specific regulatory element comprises the polynucleotide sequence of SEQ ID NO: 215.

In some embodiments, an engineered photoreceptor-specific regulatory element comprises the polynucleotide sequence of SEQ ID NO: 216.

In some embodiments, an engineered photoreceptor-specific regulatory element comprises the polynucleotide sequence of SEQ ID NO: 217.

In some embodiments, an engineered photoreceptor-specific regulatory element comprises the polynucleotide sequence of SEQ ID NO: 218.

In some embodiments, an engineered photoreceptor-specific regulatory element comprises the polynucleotide sequence of SEQ ID NO: 219.

In some embodiments, an engineered photoreceptor-specific regulatory element comprises the polynucleotide sequence of SEQ ID NO: 220.

In some embodiments, an engineered photoreceptor-specific regulatory element comprises the polynucleotide sequence of SEQ ID NO: 221.

In some embodiments, an engineered photoreceptor-specific regulatory element comprises the polynucleotide sequence of SEQ ID NO: 222.

In some embodiments, an engineered photoreceptor-specific regulatory element comprises the polynucleotide sequence of SEQ ID NO: 223.

In some embodiments, an engineered photoreceptor-specific regulatory element comprises the polynucleotide sequence of SEQ ID NO: 224.

In some embodiments, an engineered photoreceptor-specific regulatory element comprises the polynucleotide sequence of SEQ ID NO: 225.

In some embodiments, an engineered photoreceptor-specific regulatory element comprises the polynucleotide sequence of SEQ ID NO: 226.

In some embodiments, an engineered photoreceptor-specific regulatory element comprises the polynucleotide sequence of SEQ ID NO: 227.

In some embodiments, an engineered photoreceptor-specific regulatory element comprises the polynucleotide sequence of SEQ ID NO: 228.

In some embodiments, an engineered photoreceptor-specific regulatory element comprises the polynucleotide sequence of SEQ ID NO: 229.

In some embodiments, an engineered photoreceptor-specific regulatory element comprises the polynucleotide sequence of SEQ ID NO: 230.

In some embodiments, an engineered photoreceptor-specific regulatory element comprises the polynucleotide sequence of SEQ ID NO: 231.

In some embodiments, an engineered photoreceptor-specific regulatory element comprises the polynucleotide sequence of SEQ ID NO: 232.

In some embodiments, an engineered photoreceptor-specific regulatory element comprises the polynucleotide sequence of SEQ ID NO: 233.

In some embodiments, an engineered photoreceptor-specific regulatory element comprises the polynucleotide sequence of SEQ ID NO: 234.

In some embodiments, an engineered photoreceptor-specific regulatory element comprises the polynucleotide sequence of SEQ ID NO: 235.

In some embodiments, an engineered photoreceptor-specific regulatory element comprises the polynucleotide sequence of SEQ ID NO: 236.

In some embodiments, an engineered photoreceptor-specific regulatory element comprises the polynucleotide sequence of SEQ ID NO: 237.

In some embodiments, an engineered photoreceptor-specific regulatory element comprises the polynucleotide sequence of SEQ ID NO: 238.

In some embodiments, an engineered photoreceptor-specific regulatory element comprises the polynucleotide sequence of SEQ ID NO: 239.

In some embodiments, an engineered photoreceptor-specific regulatory element comprises the polynucleotide sequence of SEQ ID NO: 240.

In some embodiments, an engineered photoreceptor-specific regulatory element comprises the polynucleotide sequence of SEQ ID NO: 241.

In some embodiments, an engineered photoreceptor-specific regulatory element comprises the polynucleotide sequence of SEQ ID NO: 242.

In some embodiments, an engineered photoreceptor-specific regulatory element comprises the polynucleotide sequence of SEQ ID NO: 243.

In some embodiments, an engineered photoreceptor-specific regulatory element comprises the polynucleotide sequence of SEQ ID NO: 244.

In some embodiments, an engineered photoreceptor-specific regulatory element comprises the polynucleotide sequence of SEQ ID NO: 245.

In some embodiments, an engineered photoreceptor-specific regulatory element comprises the polynucleotide sequence of SEQ ID NO: 246.

In some embodiments, an engineered photoreceptor-specific regulatory element comprises the polynucleotide sequence of SEQ ID NO: 247.

In some embodiments, an engineered photoreceptor-specific regulatory element comprises the polynucleotide sequence of SEQ ID NO: 276.

In some embodiments, an engineered photoreceptor-specific regulatory element comprises the polynucleotide sequence of SEQ ID NO: 277.

In some embodiments, an engineered photoreceptor-specific regulatory element comprises the polynucleotide sequence of SEQ ID NO: 278.

The present disclosure further provides for heterologous constructs comprising an engineered photoreceptor-specific regulatory element, as described herein, operably linked to a polynucleotide, wherein the polynucleotide comprises a polynucleotide sequence encoding a polypeptide.

In some embodiments, a polypeptide comprises at least one effector molecule. In some embodiments, a polypeptide comprises a first effector molecule and a second effector molecule. In some embodiments, a polynucleotide comprises a polynucleotide sequence encoding the first effector molecule, a linker polynucleotide sequence, and a polynucleotide sequence encoding the second effector. In some embodiments, a linker polynucleotide sequence encodes one or more 2A ribosome skipping elements. In some embodiments, one or more 2A ribosome skipping elements comprise elements that are each selected from the group consisting of: P2A, T2A, E2A, and F2A.

In some embodiments, at least one effector molecule belongs to a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme. In some embodiments, a first effector molecule and the second effector molecule is from a separate therapeutic class.

In some embodiments, at least one effector molecule is a human-derived effector molecule.

The present disclosure provides vectors comprising a heterologous construct, as described herein.

The present disclosure further provides dual expression vectors comprising a heterologous construct as described herein and a second construct comprising a polynucleotide sequence encoding a second effector protein.

The present disclosure also provides for photoreceptor cell comprising a heterologous construct as described herein, a vector as described herein, or a dual expression vector as described herein.

In some embodiments, a photoreceptor cell is a rod cell or a cone cell. In some embodiments, a photoreceptor cell is a cone cell. In some embodiments, a photoreceptor cell is a rod cell.

In some embodiments, a photoreceptor cell expresses at least one effector molecule.

The present disclosure provides for pharmaceutical compositions comprising an engineered photoreceptor-specific regulatory element as described herein, a heterologous construct as described herein, a vector as described herein, a dual expression vector as described herein, or the photoreceptor cell as described herein, and a pharmaceutically acceptable carrier, pharmaceutically acceptable excipient, or a combination thereof.

The present disclosure provides for methods of increasing expression of a target gene, the method comprising use of an engineered photoreceptor-specific regulatory element as described herein, a heterologous construct as described herein, a vector as described herein, a dual expression vector as described herein, or a photoreceptor cell as described herein, to increase expression of the target gene.

The present disclosure provides for methods of treating a subject in need thereof, the method comprising administering an engineered photoreceptor-specific regulatory element as described herein, a heterologous construct as described herein, a vector as described herein, or a pharmaceutical composition as described herein.

The present disclosure provides for methods of treating a subject having an ocular disease or disorder, the method comprising administering an engineered photoreceptor-specific regulatory element as described herein, a heterologous construct as described herein, a vector as described herein, or a pharmaceutical composition as described herein.

The present disclosure provides for kits for treating and/or preventing a tumor, comprising a pharmaceutical composition as described herein. In some embodiments, a kit further comprises written instructions for using the pharmaceutical composition for treating and/or preventing a tumor in a subject.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 248. In some embodiments, an enhancer region further comprises (b) a second enhancer segment.

The present disclosure also provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 259. In some embodiments, an enhancer region further comprises (b) a second enhancer segment.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 249; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 250.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 251; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 252.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 253; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 254.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 255; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 256.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 257; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 258.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 260; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 261.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 262; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 263.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 264; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 265.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 266; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 267.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 268; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 269.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 270; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 271.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 272; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 273.

The present disclosure provides for an engineered photoreceptor-specific regulatory element comprising: (a) an enhancer region, wherein the enhancer region comprises: (i) a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 274; (ii) a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 275.

In some embodiments, an engineered photoreceptor-specific regulatory element exhibits greater activity in photoreceptor cells of a mammalian retina as compared to non-photoreceptor cells of the same mammalian retina.

In some embodiments, a first enhancer segment and a second enhancer segment are contiguous.

In some embodiments, a first enhancer segment and a second enhancer segment are non-contiguous.

In some embodiments, an enhancer region further comprises an ablation motif.

In some embodiments, an ablation motif is selected from any ablation motif shown in Table 2.

In some embodiments, a nucleotide sequence of about 1, about 5, about 10, about 15, about 20, about 30, about 40, about 50, about 60, about 70, about 80, about 90, about 100, about 120, about 140, about 160, about 180, or about 200 nucleotides is between the first enhancer segment and the second enhancer segment.

These and other aspects and features of the invention are described in the following detailed description and claims.

### BRIEF DESCRIPTION OF THE DRAWING

FIG. 1 shows a bar graph depicting promoter strength of various exemplary engineered expression elements including SB05201, SB05201 variants (1.A, 1.B, 1.C, and 1.D), SB07143, SB07140, SB07141, SB07149, SB07144, SB07142, and SB07150. Promoter strength is measured as % CAG promoter.

FIG. 2 shows a bar graph depicting promoter strength of various exemplary engineered expression elements including Exemplary Promoter 27 and Exemplary Promoter 27 variants (27.A, 27.B, 27.C, 27.D, 27.E, 27.F, 27.G, 27.1, 27.J, 27.K, 27.L, 27.M, and 27.N). Promoter strength is measured as % CAG promoter.

FIG. 3 shows a bar graph depicting promoter strength of various exemplary engineered expression elements including Exemplary Promoter 28 and Exemplary Promoter 28 variants (28.A, 28.B, 28.C, 28.D, 28.E, 28.F, 28.G, 28.1, 28.J, 28.K, 28.L, 28.M, and 28.N). Promoter strength is measured as % CAG promoter.

FIG. 4 shows a bar graph depicting promoter strength of various exemplary engineered expression elements including Exemplary Promoter 29 and Exemplary Promoter 29 variants (29.A, 29.B, 29.C, 29.D, 29.E, 29.F, 29.G, 29.1, 29.J, 29.K, 29.L, 29.M, and 29.N). Promoter strength is measured as % CAG promoter.

FIG. 5 shows a bar graph depicting promoter strength of various exemplary engineered expression elements including SB05241, SB05241 variants (41.A, 41.B, 41.C, 41.D, 41.E, 41.F, 41.G, 41.1, 41.J, 41.K, 41.L, and 41.M), SB07207, and SB07208. Promoter strength is measured as % CAG promoter.

FIG. 6 shows a graph depicting promoter strength of exemplary engineered expression elements including SB05245, SB05245 variants (45.A, 45.B, 45.C, 45.D, 45.E, 45.F, 45.G, 45.1, and 45.J), SB07227, SB07230, SB07221, SB07225, SB07229, and SB07222. Promoter strength is measured as % CAG promoter.

FIG. 7 shows a graph depicting promoter strength of exemplary engineered expression elements including Exemplary promoter 51 and Exemplary promoter 51 variants (51.A, 51.B, 51.C, 51.D, 51.E, 51.F, 51.G, 51.1, 51.J, 51.K, 51.L, 51.M, 51.N, 51.0, and 51.P). Promoter strength is measured as % CAG promoter.

FIG. 8 shows a graph depicting promoter strength of exemplary engineered expression elements including Exemplary promoter 76 and Exemplary promoter 76 variants (76.A, 76.B, 76.C, 76.D, 76.E, 76.F, 76.G, 76.1, 76.J, 76.K, 76.L, 76.M, 76.N, 76.0, 76.P, and 76.Q). Promoter strength is measured as % CAG promoter.

### DETAILED DESCRIPTION

### I. Definitions

Unless defined otherwise, all technical and scientific terms used herein have the same meaning as is commonly understood by one of skill in the art to which the disclosed subject-matter belongs. Generally, nomenclatures utilized in connection with, and techniques of, immunology, oncology, cell and tissue culture, molecular biology, and protein and oligo- or polynucleotide chemistry and hybridization described herein are those well-known and commonly used in the art. It is to be understood that the foregoing general description and the following detailed description are exemplary and explanatory only and are not restrictive of any subject-matter claimed or otherwise provided herein. The section headings used herein are for organizational purposes only and are not to be construed as limiting the subject-matter described.

As used herein, singular forms “a,” “an,” and “the” include plural referents unless the context clearly indicates otherwise.

As used herein, all numerical values or numerical ranges include whole integers within or encompassing such ranges and fractions of the values or the integers within or encompassing ranges unless the context clearly indicates otherwise. Thus, for example, reference to a range of 90% to 100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

Ablation: As used herein, an “ablation” refers to a deletion of a nucleotide sequence segment (e.g., a nucleotide sequence motif), using any means of nucleotide deletion known in the art (e.g., molecular cloning, CRISPR, etc.). Ablation may further comprise replacement of a deleted nucleotide sequence segment with a transcriptionally inert nucleotide sequence, or segment, of the same length. Ablation may further comprise replacement of a deleted nucleotide sequence segment of a certain length with a transcriptionally inert nucleotide sequence, or segment, of a different length (e.g., longer, or shorter). In some embodiments, ablation of a nucleotide motif within a nucleotide sequence (e.g., an enhancer region) increases activity (e.g., transcriptional levels downstream of the regulatory element following stimulation) and/or selectivity (e.g., transcriptional activity in one type of cell as compared to a different type of cell) of a regulatory element, wherein the increased activity and/or selectivity is relative to a nucleotide sequence lacking such ablation. Methods of quantifying transcriptional levels are known in the art and include, for example and without limitation, mRNA analysis by reverse-transcriptase quantitative polymerase chain reaction (RT-qPCR), fluorescent reporters, colorimetric reporters, etc. In some embodiments, an ablation increases inducibility, or transcriptional activity, of a coding sequence (e.g., a gene), by at least 0.5-fold, at least 1-fold, at least 2-fold, at least 3-fold, at least 4-fold, at least 5-fold, at least 6-fold, at least 7-fold, or at least 8-fold as compared to a comparable control (e.g., a regulatory element, e.g., an enhancer, without an ablation, or a commonly used regulatory element for comparison, e.g., a CAG promoter). In some embodiments, induction of transcription, or transcriptional activity, of an engineered regulatory element (e.g., an ablated enhancer regulatory element) is measured as a percentage of induction of transcription or transcriptional activity by a known promoter, e.g., a CAG promoter. In some embodiments, an ablation increases inducibility, or transcriptional activity, of a coding sequence (e.g., a gene), by a certain percentage as compared to a comparable control. In some embodiments, an ablation increases selectivity for transcriptional activity in a particular cell as compared to a different cell by at least 10 fold, at least 20 fold, at least 30 fold, at least 40 fold, at least 50 fold, at least 60 fold, at least 70 fold, at least 80 fold, at least 100 fold, at least 200 fold, at least 300 fold, at least 400 fold, at least 500 fold, or at least 600 fold. It is contemplated herein that changes in inducibility or selectivity of an engineered nucleic acid as described herein may depend on a test system, e.g., cell type comprising said engineered nucleic acid. In some embodiments, an ablation comprises a substitution of at least one, at least two, at least three, at least four, at least five, at least six, at least seven, at least eight, at least nine, at least ten, or more nucleotide motifs within a nucleotide sequence. In some embodiments, introduction of at least one nucleotide motif in an ablation site does not introduce new regulatory sites in an engineered nucleic acid (e.g., an engineered nucleic acid as provided herein). Such engineered nucleic acids comprising an ablation comprising a deletion or a substitution of at least one nucleotide motif may be referred to herein as “ablation variants.” Enhancer regions comprising an ablation may be referred to as “ablated enhancer regions,” “engineered enhancer regions,” or “modified enhancer regions.”

Agent: The term “agent” as used herein may refer to a compound, molecule, or entity of any chemical class including, for example, polypeptides, nucleic acids (e.g., engineered nucleic acids as described herein), saccharides, lipids, small molecules, metals, or combinations thereof. In some embodiments, an agent is or comprises a natural product in that it is found in and/or is obtained from nature. In some embodiments, an agent is or comprises one or more entities that is man-made in that it is designed, engineered, modified, and/or produced through action of the hand of man and/or is not found in nature. In many aspects, the present disclosure provides for engineered nucleic acids comprising particular transcriptional regulatory elements as described herein. In some embodiments, an agent may be utilized in isolated or pure form (e.g., an isolated polynucleotide); in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, nucleic acids (e.g., siRNAs, shRNAs, DNA/RNA hybrids, antisense oligonucleotides, ribozymes), peptides, peptide mimetics. In some embodiments, an agent as described herein is encoded on a coding nucleotide sequence that is operably linked to an engineered regulatory element as provided herein (e.g., an engineered photoreceptor-specific regulatory element). In some embodiments, an agent is an effector molecule as described herein.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to an agent or substance that has activity in a biological system (e.g., an isolated cell, a cell in a particular tissue, a cell in culture, or a cell in an organism, etc.). For instance, an agent or substance that, when administered to an organism, has a biological effect on that organism, is considered to be biologically active, or have biological activity (e.g., a biologically active agent, bioactive agent, bioactive molecule, etc.). It will be appreciated by those skilled in the art that often only a portion or fragment of a biologically active substance is required (e.g., is necessary and sufficient) for an activity to be present; in such circumstances, that portion or fragment is considered to be a “biologically active” portion or fragment.

Expression cassette: As used herein, an “expression cassette” is a polynucleotide construct, generated recombinantly or synthetically, comprising regulatory sequences operably linked to a selected polynucleotide (e.g., a coding sequence, such as a gene) to facilitate expression of said selected polynucleotide in a host cell, or in a cell-free environment. Heterologous constructs as described herein may include one or more expression cassettes.

Heterologous: The term “heterologous” as used herein with respect to a nucleotide sequence, amino acid sequence, or polypeptide, refers to a compound or agent which is either foreign (e.g., exogenous, such as it is not found in nature) to a given host cell, or which is naturally found in a given host cell (e.g., is endogenous), however, said compound or agent is in the context of a heterologous construct, e.g., employing heterologous nucleic acid, as described herein. A heterologous nucleotide sequence as found endogenously may also be produced in an unnatural, e.g., greater than expected or greater than naturally found, amount in a cell. A heterologous nucleotide sequence, or a nucleic acid comprising a heterologous nucleotide sequence, possibly differs in sequence from an endogenous nucleotide sequence but encodes the same protein as found endogenously. Specifically, heterologous nucleotide sequences are those not found in the same relationship to a host cell in nature. Any recombinant or artificial nucleotide sequence is understood to be heterologous. A non-limiting example of a heterologous polynucleotide, e.g., an engineered regulatory element as described herein, is a nucleotide sequence such as an enhancer, or enhancer region, operably linked to a promoter with which it is not natively associated. Heterologous polynucleotides comprising such engineered regulatory elements may further be used to control expression of coding sequences (e.g., genes) in place of the native, or wild-type, promoter of said coding sequence.

Identity: As used herein, the term “identity” refers to the overall relatedness between polymeric molecules, e.g., between nucleic acid molecules (e.g., DNA molecules and/or RNA molecules) and/or between polypeptide molecules. Accordingly, reference to “identity” or “homology” in the context of a particular sequence (e.g., nucleotide sequence, amino acid sequence, etc.) have the same meaning unless the context indicates otherwise. Calculation of the percent identity of two nucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 75%, at least 80%, at least 85%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0) using a PAM120 weight residue table, a length penalty of 12, and a gap penalty of 4. The percent identity between two nucleotide sequences can, alternatively, be determined using the GAP program in the GCG software package using an NWSgapdna.CMP matrix. Optimal alignment of sequences for comparison can also be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al.). Another example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

Inert Sequence: As used herein, “inert sequence,” refers to a polynucleotide sequence that is devoid of transcription factor binding sites within said polynucleotide sequence. In addition, an inert sequence may be further modified such that 5′ and 3′ ends of the inert sequence also do not contain transcription factor binding sites when joined to an enhancer sequence of the of the present disclosure (e.g., via an ablation). The presence of an inert sequence in a regulatory element (e.g., an enhancer region) does not render the entire regulatory element inert.

Isolated: As used herein, the term “isolated” refers to a substance and/or entity that has been: (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

Minimal promoter: As used herein, a “minimal promoter” refers to a minimal sequence of a native, or wild-type, promoter that maintains the ability to initiate, or induce, transcription of a target coding sequence, e.g., a gene. Accordingly, a minimal promoter can be a heterologous, engineered promoter.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising individual nucleic acid residues. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA.” “RNA.” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present disclosure. The terms “nucleotide sequence encoding an amino acid sequence” or “coding sequence” includes all nucleotide sequences that are degenerate versions of each other and/or encode the same amino acid sequence. Nucleotide sequences that encode proteins and/or RNA may include introns. A coding sequence may also refer to a nucleotide sequence that produces a bioactive nucleic acids (e.g., a mRNA, miRNA, siRNA, shRNA, etc.). Nucleic acids can be isolated or purified from natural sources, produced using recombinant expression systems and optionally isolated or purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. The term “nucleic acid segment” is used herein to refer to a nucleic acid sequence that is a portion of a longer nucleic acid sequence. In many embodiments, a nucleic acid segment comprises at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, or more residues. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present disclosure is directed to “unmodified nucleic acids,” meaning nucleic acids (e.g., polynucleotides and residues, including nucleotides and/or nucleosides) that have not been chemically modified in order to facilitate or achieve delivery.

Operably linked: As used herein the term “operably linked” refers to polynucleotide sequences or amino acid sequences placed into a functional relationship with one another. For instance, a regulatory sequence or regulatory element (e.g., a promoter and/or an enhancer region) is operably linked to a coding sequence if it regulates, or contributes to modulation of, the transcription of the coding sequence. Operably linked DNA sequences encoding regulatory sequences are typically contiguous to a coding sequence. However, enhancers (e.g., enhancer regions) can function when separated from a promoter by up to several kilobases or more. Additionally, multi-cistronic constructs can include multiple coding sequences which use only one regulatory element by including a 2A self-cleaving peptide, an IRES element, etc. as described herein. Accordingly, some polynucleotide elements (e.g., engineered regulatory elements provided herein) may be operably linked to one or more coding sequences, but not contiguous with said one or more coding sequences.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a biologically active portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids.

Specific: The term “specific”, when used herein with reference to an agent or entity having an activity, or inducing an activity (e.g., an engineered nucleic acid as provided herein), is understood by those skilled in the art to mean that the agent or entity discriminates between potential targets or states. In many aspects, provided agents or entities display specific activity in a particular cell type (e.g., a photoreceptor cell) as compared to a different or alternative cell type (e.g., a non-photoreceptor cell). In many aspects of the present disclosure, an agent or entity is an engineered nucleic acid (e.g., an engineered photoreceptor-specific regulatory element) that specifically induces transcription of one or more coding sequences in a particular cell type as compared to a different cell type; that is to say that induced transcription of the one or more coding sequence is higher in a particular cell type as compared to a different cell type. In some embodiments, an agent is said to bind “specifically” to its target if it binds preferentially with that target in the presence of competing alternative targets. In some embodiments, the agent or entity does not detectably bind to the competing alternative target under conditions of binding to its target. In some embodiments, the agent or entity binds with higher on-rate, lower off-rate, increased affinity, decreased dissociation, and/or increased stability to its target as compared with the competing alternative target(s).

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Subject: As used herein, the term “subject” means a mammal (e.g., a human, in some embodiments including prenatal human forms, a rodent, a mouse, a rat, a rabbit, a monkey, a dog, a cat, a sheep, cattle, a primate, and/or a pig). In some embodiments, a subject is suffering from a relevant disease, disorder or condition (e.g., an eye disease or disorder). In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. A subject can be a patient, which refers to a human presenting to a medical provider for diagnosis or treatment of a disease. In some embodiments, a subject is an individual to whom therapy is administered, e.g., a recipient. In many aspects of the present disclosure, a subject has, or is susceptible to, an eye disease or disorder. In some embodiments, an eye disease or disorder is cancer. In some embodiments, an eye disease or disorder is macular degeneration, inherited macular dystrophy, cone-rod dystrophy (CRD), rod-cone dystrophy (also referred to as retinitis pigmentosa, or RP), Usher syndrome, Stargardt disease, achromatopsia (ACHM), Leber congenital amaurosis (LCA), choroideremia (CHM), Bardet-Biedl syndrome (BBS), or retinoblastoma.

Therapeutic agent: As used herein, the phrase “therapeutic agent” in general refers to any agent that elicits a desired pharmacological effect when administered to an organism (e.g., a subject). In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” refers to an amount of a therapeutic protein (e.g., an effector molecule) which confers a therapeutic effect on the treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic protein or composition effective to treat, ameliorate, or prevent a desired disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

Treatment: As used herein, the term “treatment” (also “treat” or “treating”) refers to any administration of a substance that partially or completely alleviates, ameliorates, relives, inhibits, delays onset of, reduces severity of, and/or reduces incidence of one or more symptoms, features, and/or causes of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

Vector: As used herein, “vector” refers to a nucleic acid molecule capable of transporting another nucleic acid to which it is associated. The terms “vector” and “plasmid” may be used interchangeably. In some embodiment, vectors are capable of extra-chromosomal replication and/or expression of nucleic acids to which they are linked in a host cell such as a eukaryotic and/or prokaryotic cell. Vectors capable of directing the expression of operatively linked coding sequences (e.g., genes) are referred to herein as “expression vectors.” An expression vector typically comprises an expression cassette. Vectors and plasmids include, but are not limited to, replication vectors, probe generation vectors, sequencing vectors, integrating vectors, phagemids, prokaryotic plasmids, eukaryotic plasmids, plant synthetic chromosomes, episomes, viral vectors (e.g., animal virus vectors), cosmids, and artificial chromosomes.

### II. Engineered Photoreceptor-Specific Regulatory Elements

Among other things, the present disclosure provides methods and compositions comprising engineered nucleic acids that comprise engineered regulatory elements which allow for selective and efficient induction of transcription of an operably linked polynucleotide (e.g., a gene) in photoreceptor cells as compared to non-photoreceptor cells. In many embodiments, an engineered nucleic acid provided by the present disclosure is or comprises an engineered photoreceptor-specific regulatory element, as described herein. In some embodiments, an engineered photoreceptor-specific regulatory element is provided in an expression cassette, a heterologous construct, a vector, or other polynucleotide sequence.

In some embodiments, an engineered photoreceptor-specific regulatory element comprises a native genomic enhancer region, that when operably linked to a heterologous promoter (e.g., a minimal promoter), exhibits remarkable selectivity for inducing transcriptional activity in photoreceptor cells as compared to non-photoreceptor cells.

In some embodiments, an engineered photoreceptor-specific regulatory element comprises an engineered enhancer region. In some embodiments, an engineered enhancer region is engineered in that it includes a portion where at least one nucleotide motif therein is modified, e.g., ablated. In some embodiments, an engineered enhancer region allows for high selectivity for inducing transcriptional activity of a particular coding sequence in photoreceptor cells over non-photoreceptor cells. In some embodiments, an engineered photoreceptor-specific regulatory element comprises an engineered enhancer region that is operably linked to a promoter (e.g., a minimal promoter).

In some embodiments, an enhancer region is or comprises a wild-type enhancer or engineered enhancer (e.g., an ablated enhancer, or synthetic enhancer comprising one or more engineered regulatory components). In some embodiments, an enhancer region is or comprises one or more enhancer segments. In some embodiments, one or more enhancer segments of an enhancer region are contiguous. In some embodiments, one or more enhancer segments of an enhancer region are non-contiguous. In some embodiments, on or more enhancer segments of an enhancer region are contiguous, and one or more enhancer regions of the same enhancer region are non-contiguous. In some embodiments, a nucleotide sequence of about 1, about 5, about 10, about 15, about 20, about 30, about 40, about 50, about 60, about 70, about 80, about 90, about 100, about 120, about 140, about 160, about 180, or about 200 nucleotides is located between the first enhancer segment and the second enhancer segment. In some embodiments, a nucleotide sequence of about 1 to about 200, about 1 to about 100, about 1 to about 50, about 1 to about 25, about 10 to about 200, about 10 to about 100, about 10 to about 50, about 10 to about 25, about 25 to about 200, about 25 to about 100, about 25 to about 50, about 50 to about 200, about 50 to about 150, about 50 to about 100, about 50 to about 75, about 100 to about 200, about 100 to about 150, about 25 to about 100, about 30 to about 100, about 40 to about 100, about 25 to about 75, or about 25 to about 50 nucleotides is located between the first enhancer segment and the second enhancer segment.

In some embodiments, an engineered photoreceptor-specific regulatory element induces expression of an operably linked coding sequence (e.g., a gene) in a photoreceptor cell at a level of specificity of at least about 5, at least about 10, at least 15, at least about 20, at least about 25 at least about 30, at least about 35, at least about 40, at least about 45, at least about 50, at least about 60, at least about 70, at least about 80, at least about 90, at least about 100, at least about 150, at least about 200, at least about 350, at least about 400, at least about 450, at least about 500, at least about 600, at least about 700, at least about 800, at least about 900, at least about 1000, at least about 1250, at least about 1500, at least about 2000, at least about 2500, at least about 3000, at least about 4000, at least about 5000, at least about 6000, at least about 7000, at least about 8000, at least about 9000, at least about 10,000, at least about 15,000, at least about 20,000, at least about 25,000, at least about 30,000 times, or more than induced expression observed in a non-photoreceptor cell.

In some embodiments, an engineered photoreceptor-specific regulatory element induces expression of an operably linked coding sequence (e.g., a gene) at a comparable level, or strength, to an alternative regulatory element, e.g., as measured by percentage of transcriptional activity as compared to said alternative regulatory element (e.g., a promoter, such as a CAG promoter). In some embodiments, an engineered photoreceptor-specific regulatory element induces expression of an operably linked coding sequence at a strength of 1%, 2%, 3%, 4%, 5%, 10%, 15%, 20%, 30%, 40%, 50%, 60%, 70%, 80%, 90%, 100%, or more as compared to an alternative regulatory element (e.g., a CAG promoter).

In some embodiments, an engineered photoreceptor-specific regulatory element comprises an enhancer region, such as a native enhancer region or an engineered enhancer region (e.g., an ablated enhancer region) that is about 50 base pairs, about 60 base pairs, about 70 base pairs, about 80 base pairs, about 90 base pairs, about 100 base pairs, about 110 base pairs, about 120 base pairs, about 130 base pairs, about 140 base pairs, about 150 base pairs, about 160 base pairs, about 170 base pairs, about 180 base pairs, about 190 base pairs, about 200 base pairs, about 220 base pairs, about 240 base pairs, about 260 base pairs, about 280 base pairs, about 300 base pairs, about 320 base pairs, about 340 base pairs, about 360 base pairs, about 380 base pairs, about 400 base pairs, about 420 base pairs, about 440 base pairs, about 460 base pairs, about 480 base pairs, about 500 base pairs, about 520 base pairs, about 540 base pairs, about 560 base pairs, about 580 base pairs, or about 600 base pairs in length.

In some embodiments, an engineered photoreceptor-specific regulatory element comprises an enhancer region, such as a native enhancer region or an engineered enhancer region (e.g., an ablated enhancer region) that is at least about 50 base pairs, at least about 60 base pairs, at least about 70 base pairs, at least about 80 base pairs, at least about 90 base pairs, at least about 100 base pairs, at least about 110 base pairs, at least about 120 base pairs, at least about 130 base pairs, at least about 140 base pairs, at least about 150 base pairs, at least about 160 base pairs, at least about 170 base pairs, at least about 180 base pairs, at least about 190 base pairs, at least about 200 base pairs, at least about 220 base pairs, at least about 240 base pairs, at least about 260 base pairs, at least about 280 base pairs, at least about 300 base pairs, at least about 320 base pairs, at least about 340 base pairs, at least about 360 base pairs, at least about 380 base pairs, at least about 400 base pairs, at least about 420 base pairs, at least about 440 base pairs, at least about 460 base pairs, at least about 480 base pairs, at least about 500 base pairs, at least about 520 base pairs, at least about 540 base pairs, at least about 560 base pairs, at least about 580 base pairs, or at least about 600 base pairs in length.

In some embodiments, an engineered photoreceptor-specific regulatory element comprises an enhancer region, such as a native enhancer region or an engineered enhancer region (e.g., an ablated enhancer region) that is up to about 50 base pairs, up to about 60 base pairs, up to about 70 base pairs, up to about 80 base pairs, up to about 90 base pairs, up to about 100 base pairs, up to about 110 base pairs, up to about 120 base pairs, up to about 130 base pairs, up to about 140 base pairs, up to about 150 base pairs, up to about 160 base pairs, up to about 170 base pairs, up to about 180 base pairs, up to about 190 base pairs, up to about 200 base pairs, up to about 220 base pairs, up to about 240 base pairs, up to about 260 base pairs, up to about 280 base pairs, up to about 300 base pairs, up to about 320 base pairs, up to about 340 base pairs, up to about 360 base pairs, up to about 380 base pairs, up to about 400 base pairs, up to about 420 base pairs, up to about 440 base pairs, up to about 460 base pairs, up to about 480 base pairs, up to about 500 base pairs, up to about 520 base pairs, up to about 540 base pairs, up to about 560 base pairs, up to about 580 base pairs, or up to about 600 base pairs in length.

In some embodiments, an engineered photoreceptor-specific regulatory element comprises an enhancer region, such as a native enhancer region or an engineered enhancer region (e.g., an ablated enhancer region) that is about 50 to about 600 base pairs in length, about 100 to about 600 base pairs in length, about 200 to about 600 base pairs in length, about 300 to about 600 base pairs in length, about 400 to about 600 base pairs in length, about 500 to about 600 base pairs in length, about 50 to about 500 base pairs in length, about 50 to about 400 base pairs in length, about 50 to about 300 base pairs in length, about 50 to about 200 base pairs in length, about 50 to about 175 base pairs in length, about 50 to about 150 base pairs in length, about 50 to about 125 base pairs in length, or about 50 to about 100 base pairs in length.

In some embodiments, an engineered photoreceptor-specific regulatory element comprises a promoter (e.g., a minimal promoter) selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, lateADE, minIL2.2, SMP, inducer molecule responsive promoters, CMV, EFS, SFFV, SV40, MND, PGK, UbC, hEFlaV1, hCAGG, hEFlaV2, hACTb, heIF4Al, hGAPDH, hGRP78, hGRP94, hHSP70, hKINb, hUBIb, and tandem repeats thereof.

In some embodiments, an engineered photoreceptor-specific regulatory element as described herein comprises at least one spacer sequence. In some embodiments, an engineered photoreceptor-specific regulatory element comprises at least one spacer located between an enhancer region (e.g., a native enhancer region, or engineered enhancer region) and a promoter (e.g., a minimal promoter). In some embodiments, a spacer is selected from the group consisting of SEQ ID Nos 107-110. In some embodiments, a spacer comprises the nucleotide sequence CCTGCAGG (SEQ ID NO: 107). In some embodiments, a spacer comprises the nucleotide sequence TAGTAAGGTA (SEQ ID NO: 108). In some embodiments, a spacer comprises the nucleotide sequence TTTGCGCGTA (SEQ ID NO: 109). In some embodiments, a spacer comprises the nucleotide sequence AGTCCGGGTA (SEQ ID NO: 110).

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

In some embodiments, engineered nucleic acids (e.g., heterologous constructs, as described herein) are configured to produce multiple agents (e.g., one or more effector molecules) that can be encoded in one or more coding sequences that are operably linked to an engineered photoreceptor-specific regulatory element, as provided herein. For example, engineered nucleic acids may be configured to produce 2-20 different agents. In some embodiments, engineered nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19,7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 agents. In some embodiments, nucleic acids are configured to produce 1, 2, 3, 4, 5, 6, 7, 8, 9, 10, 11, 12, 13, 14, 15, 16, 17, 18, 19, or 20 agents. Provided agents are, in many cases, therapeutic agents, such as those described herein.

In some embodiments, engineered nucleic acids (e.g., heterologous constructs, as described herein) may comprise multicistronic regions, i.e., more than one separate polypeptide (e.g., therapeutic agents, effector molecules, and the like) can be produced from a single mRNA transcript transcribed from said multicistronic region. Multicistronic regions may be created through the use of various linkers, e.g., a first coding sequence can be linked to a second coding sequence with a linker, for example creating a construct comprising, from 5′ to 3′, a first coding sequence, a linker, and a second coding sequence. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage. In some embodiments, a multicistronic region comprises up to two, about to three, up to four, up to five, up to six, up to seven, up to eight, up to nine, up to ten, up to fifteen, up to twenty, or more coding sequences each linked by a linker (e.g., a first linker, a second linker, a third linker, a fourth linker, and so on).

### A. Native Enhancer & Heterologous Promoter Regulatory Elements

The present disclosure provides for, among other things, engineered photoreceptor-specific regulatory elements that comprise a genomic (i.e., native) enhancer region that is operably linked to a heterologous promoter (e.g., a minimal promoter). In many embodiments, provided engineered photoreceptor-specific regulatory elements comprising a native enhancer region operably linked to a heterologous promoter exhibit remarkable selectivity for activity in photoreceptor cells as compared to non-photoreceptor cells. In some embodiments, a native enhancer region comprises a nucleotide sequence that is found in a native genomic sequence of a genome (e.g., a mammalian genome).

In some embodiments, an engineered photoreceptor-specific regulatory element is selected from Table 1. In some embodiments, an engineered photoreceptor-specific regulatory element is selected from any one of SEQ ID NOs: 138-148.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 1. In some embodiments, a native enhancer region comprising a nucleotide sequence as set forth in SEQ ID NO: 1. In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 2. In some embodiments, a native enhancer region comprises a nucleotide sequence as set forth in SEQ ID NO: 2. In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99%, sequence identity to SEQ ID NO: 3. In some embodiments, a native enhancer region comprises a nucleotide sequence as set forth in SEQ ID NO: 3. In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 4. In some embodiments, a native enhancer region comprises a nucleotide sequence as set forth in SEQ ID NO: 4.

In some embodiments, an engineered photoreceptor-specific regulatory element comprises a promoter. In some embodiments, a promoter is a minimal promoter. In some embodiments a minimal promoter is selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, lateADE, minIL2.2, SMP, inducer molecule responsive promoters, CMV, EFS, SFFV, SV40, MND, PGK, UbC, hEFlaV1, hCAGG, hEFlaV2, hACTb, heIF4Al, hGAPDH, hGRP78, hGRP94, hHSP70, hKINb, hUBIb, and tandem repeats thereof. In some embodiments, a minimal promoter is derived from a YB-TATA promoter. In some embodiments, a YB-TATA promoter comprises a sequence as set forth in SEQ ID NO: 122. In some embodiments, a minimal promoter is derived from a minTK promoter. In some embodiments, a minTK promoter comprises a sequence as set forth in SEQ ID NO: 123. In some embodiments, a minimal promoter is derived from a minIL2.2 promoter. In some embodiments, a minIL2.2 promoter comprises a sequence as set forth in SEQ ID NO: 124. In some embodiments, a minimal promoter is derived from a SMP promoter. In some embodiments, a SMP promoter comprises a sequence as set forth in SEQ ID NO: 125. In some embodiments, a minimal promoter is derived from a lateADE promoter. In some embodiments, a lateADE promoter comprises a sequence as set forth in SEQ ID NO: 126. In some embodiments, a minimal promoter is derived from a minCMV promoter. In some embodiments, a minCMV promoter comprises a sequence as set forth in SEQ ID NO: 127.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 1, operably linked to a heterologous promoter (e.g., a minimal promoter) derived from a YB-TATA promoter. In some embodiments, a native enhancer region comprises a nucleotide sequence as set forth in SEQ ID NO: 1, operably linked to a heterologous promoter derived from a YB-TATA promoter. In some embodiments, a native enhancer region comprises a nucleotide sequence as set forth in SEQ ID NO: 1, operably linked to a YB-TATA promoter comprising a sequence as set forth in SEQ ID NO: 122.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 138.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 2, operably linked to a heterologous promoter (e.g., a minimal promoter) derived from a YB-TATA promoter. In some embodiments, a native enhancer region comprises a nucleotide sequence as set forth in SEQ ID NO: 2, operably linked to a heterologous promoter derived from a YB-TATA promoter. In some embodiments, a native enhancer region comprises a nucleotide sequence as set forth in SEQ ID NO: 2, operably linked to a YB-TATA promoter comprising a sequence as set forth in SEQ ID NO: 122.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 139.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 3, operably linked to a heterologous promoter (e.g., a minimal promoter) derived from a YB-TATA promoter. In some embodiments, a native enhancer region comprises a nucleotide sequence as set forth in SEQ ID NO: 3, operably linked to a heterologous promoter derived from a YB-TATA promoter. In some embodiments, a native enhancer region comprises a nucleotide sequence as set forth in SEQ ID NO: 3, operably linked to a YB-TATA promoter comprising a sequence as set forth in SEQ ID NO: 122.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 140.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 1, operably linked to a heterologous promoter (e.g., a minimal promoter) derived from a minTK promoter. In some embodiments, a native enhancer region comprises a nucleotide sequence as set forth in SEQ ID NO: 1, operably linked to a heterologous promoter derived from a minTK promoter. In some embodiments, a native enhancer region comprises a nucleotide sequence as set forth in SEQ ID NO: 1, operably linked to a minTK promoter comprising a sequence as set forth in SEQ ID NO: 123.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 141.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 1, operably linked to a heterologous promoter (e.g., a minimal promoter) derived from a lateADE promoter. In some embodiments, a native enhancer region comprises a nucleotide sequence as set forth in SEQ ID NO: 1, operably linked to a heterologous promoter derived from a lateADE promoter. In some embodiments, a native enhancer region comprises a nucleotide sequence as set forth in SEQ ID NO: 1, operably linked to a lateADE promoter comprising a sequence as set forth in SEQ ID NO: 126.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 142.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 4, operably linked to a heterologous promoter (e.g., a minimal promoter) derived from a SMP promoter. In some embodiments, a native enhancer region comprises a nucleotide sequence as set forth in SEQ ID NO: 4, operably linked to a heterologous promoter derived from a SMP promoter. In some embodiments, a native enhancer region comprises a nucleotide sequence as set forth in SEQ ID NO: 4, operably linked to a SMP promoter comprising a sequence as set forth in SEQ ID NO: 125.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 143.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 4, operably linked to a heterologous promoter (e.g., a minimal promoter) derived from a lateADE promoter. In some embodiments, a native enhancer region comprises a nucleotide sequence as set forth in SEQ ID NO: 4, operably linked to a heterologous promoter derived from a lateADE promoter. In some embodiments, a native enhancer region comprises a nucleotide sequence as set forth in SEQ ID NO: 4, operably linked to a lateADE promoter comprising a sequence as set forth in SEQ ID NO: 126.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 144.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 4, operably linked to a heterologous promoter (e.g., a minimal promoter) derived from a minCMV promoter. In some embodiments, a native enhancer region comprises a nucleotide sequence as set forth in SEQ ID NO: 4, operably linked to a heterologous promoter derived from a minCMV promoter. In some embodiments, a native enhancer region comprises a nucleotide sequence as set forth in SEQ ID NO: 4, operably linked to a minCMV promoter comprising a sequence as set forth in SEQ ID NO: 127.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 145.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 2, operably linked to a heterologous promoter (e.g., a minimal promoter) derived from a minTK promoter. In some embodiments, a native enhancer region comprises a nucleotide sequence as set forth in SEQ ID NO: 2, operably linked to a heterologous promoter derived from a minTK promoter. In some embodiments, a native enhancer region comprises a nucleotide sequence as set forth in SEQ ID NO: 2, operably linked to a minTK promoter comprising a sequence as set forth in SEQ ID NO: 123.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 146.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 2, operably linked to a heterologous promoter (e.g., a minimal promoter) derived from a minIL2.2 promoter. In some embodiments, a native enhancer region comprises a nucleotide sequence as set forth in SEQ ID NO: 2, operably linked to a heterologous promoter derived from a minIL2.2 promoter. In some embodiments, a native enhancer region comprises a nucleotide sequence as set forth in SEQ ID NO: 2, operably linked to a minIL2.2 promoter comprising a sequence as set forth in SEQ ID NO: 124.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 147.

In some embodiments, an engineered photoreceptor-specific regulatory element comprises a native enhancer region comprising a nucleotide sequence having at least 75%, at least 80%, at least 85%, at least 90%, at least 95%, at least 96%, at least 97%, at least 98%, or at least 99% sequence identity to SEQ ID NO: 2, operably linked to a heterologous promoter (e.g., a minimal promoter) derived from a lateADE promoter. In some embodiments, a native enhancer region comprises a nucleotide sequence as set forth in SEQ ID NO: 2, operably linked to a heterologous promoter derived from a lateADE promoter. In some embodiments, a native enhancer region comprises a nucleotide sequence as set forth in SEQ ID NO: 2, operably linked to a lateADE promoter comprising a sequence as set forth in SEQ ID NO: 126.

In some embodiments, an engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 148.

### B. Engineered Enhancer Regulatory Elements

In some embodiments, an engineered photoreceptor-specific regulatory element comprises an engineered enhancer region. In some embodiments, an engineered enhancer region is engineered in that it includes a portion where at least one nucleotide motif therein is modified, e.g., ablated. In some embodiments, an engineered enhance region is engineered in that it in includes one or more components from different regulatory elements. In some embodiments, an engineered enhancer region allows for high selectivity for inducing transcriptional activity of a particular coding sequence in photoreceptor cells over non-photoreceptor cells. In some embodiments, an engineered photoreceptor-specific regulatory element comprises an engineered enhancer region that is operably linked to a promoter (e.g., a minimal promoter).

In some embodiments, an engineered photoreceptor-specific regulatory element of the present disclosure comprises an engineered enhancer region comprising an ablation of at least one, at least two, at least three, at least four, at least five, at least six, at least seven, at least eight, at least nine, at least ten, or more nucleotide motifs relative to a nucleotide sequence as set forth in SEQ ID NO: 1.

In some embodiments, an engineered photoreceptor-specific regulatory element of the present disclosure comprises an engineered enhancer region comprising an ablation of at least one, at least two, at least three, at least four, at least five, at least six, at least seven, at least eight, at least nine, at least ten, or more nucleotide motifs relative to a nucleotide sequence as set forth in SEQ ID NO: 2.

In some embodiments, an engineered photoreceptor-specific regulatory element of the present disclosure comprises an engineered enhancer region comprising an ablation of at least one, at least two, at least three, at least four, at least five, at least six, at least seven, at least eight, at least nine, at least ten, or more nucleotide motifs relative to a nucleotide sequence as set forth in SEQ ID NO: 3.

In some embodiments, an engineered photoreceptor-specific regulatory element of the present disclosure comprises an engineered enhancer region comprising an ablation of at least one, at least two, at least three, at least four, at least five, at least six, at least seven, at least eight, at least nine, at least ten, or more nucleotide motifs relative to a nucleotide sequence as set forth in SEQ ID NO: 4.

In some embodiments, a nucleotide motif is selected from Table 2. In some embodiments, an engineered enhancer comprises an ablation and substitution of a nucleotide motif as presented in Table 2. In some embodiments, an engineered enhancer comprises an enhancer sequence as shown in SEQ ID NOs: 149-163, but linked (e.g., contiguously, or non-contiguously) to a different linker and/or promoter. In some embodiments, an engineered photoreceptor-specific regulatory element comprises a sequence as presented in Table 2 and/or Table 3. In many embodiments, an engineered photoreceptor-specific regulatory element comprises at least one, at least two, at least three, at least four, at least five, at least six, at least seven, at least eight, at least nine, at least ten, at least eleven, at least twelve, at least thirteen, at least fourteen, at least fifteen, at least sixteen, at least seventeen, at least eighteen, at least nineteen, at least twenty, or more regulatory element component sequences as shown in Table 4.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 149.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 150.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 151.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 152.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 153.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 154.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 155.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 156.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 157.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 158.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 159.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 160.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 161.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 162.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 163.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 164.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 165.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 166.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 167.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 168.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 169.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 170.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 171.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 172.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 173.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 174.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 175.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 176.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 177.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 178.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 179.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 180.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 181.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 182.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 183.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 184.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 185.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 186.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 187.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 188.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 189.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 190.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 191.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 192.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 193.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 194.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 195.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 196.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 197.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 198.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 199.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 200.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 201.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 202.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 203.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 204.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 205.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 206.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 207.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 208.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 209.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 210.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 211.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 212.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 213.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 214.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 215.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 216.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 217.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 218.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 219.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 220.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 221.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 222.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 223.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 224.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 225.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 226.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 227.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 228.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 229.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 230.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 231.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 232.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 233.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 234.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 235.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 236.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 237.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 238.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 239.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 240.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 241.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 242.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 243.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 244.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 245.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 246.

In some embodiments, an engineered photoreceptor-specific regulatory element comprises a nucleotide sequence having at least 75%, at least 80%, at least 85%, at least 90%, at least 95%, at least 96%, at least 97%, at least 98%, at least 99%, or 100% sequence identity to SEQ ID NO: 247.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 276.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 277.

In some embodiments, an engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 278.

### III. Heterologous Constructs

Certain aspects of the present disclosure relate to polynucleotides (e.g., isolated polynucleotides) encoding one or more engineered photoreceptor-specific regulatory elements as described herein to produce a heterologous construct. In some embodiments, a provided heterologous construct is or comprises one or more expression cassettes.

In some embodiments, a heterologous construct comprises an engineered photoreceptor-specific regulatory element that is operably linked to a coding polynucleotide sequence (e.g., a gene, or coding sequence for a bioactive molecule). In some embodiments, a heterologous construct comprises an engineered photoreceptor specific regulatory element that is operably linked to a polynucleotide sequence encoding at least one effector molecule (e.g., a first effector molecule, a second effector molecule, a third effector molecule, and so forth). In some embodiments, an effector molecule comprises a bioactive molecule. In some embodiments, an effector molecule comprises a polypeptide. In some embodiments, an effector molecule comprises a polynucleotide (e.g., a mRNA, miRNA, siRNA, shRNA, etc.). In some embodiments, an effector molecule is a human-derived effector molecule.

In some embodiments, a heterologous construct comprises a nucleotide sequence encoding two or more effector molecules under the transcriptional control of an engineered photoreceptor-specific regulatory element of the present disclosure. In some embodiments, a heterologous construct comprises a nucleotide sequence encoding two or more effector molecules each under the transcriptional control of separate engineered photoreceptor-specific regulatory elements.

In some embodiments, a heterologous construct comprises a nucleotide sequence encoding two or more effector molecules that are in the same reading frame and are expressed as a single polypeptide chain. In some embodiments, two or more effector molecules that are expressed as a single polypeptide chain may comprise one or more peptide cleavage sites (e.g., auto-cleavage sites, or cleavage sites for an intracellular protease) which when cleaved separate the two or more effector molecules. Suitable peptide cleavage sites may include, without limitation, a T2A peptide cleavage site, a P2A peptide cleavage site, an E2A peptide cleavage sire, and an F2A peptide cleavage site.

In some embodiments, two or more effector molecules that are expressed as a single polypeptide chain comprise a T2A peptide cleavage site. In some embodiments, two or more effector molecules that are expressed as a single polypeptide chain comprise an E2A peptide cleavage site. In some embodiments, two or more effector molecules that are expressed as a single polypeptide chain comprise a T2A and an E2A peptide cleavage site.

In some embodiments, a polynucleotide sequence encoding a first effector molecule is linked to a polynucleotide sequence encoding a second effector molecule by a linker polynucleotide sequence. In some embodiments, a linker polynucleotide sequence comprises a polynucleotide sequence encoding at least one 2A ribosome skipping element. In some embodiments, a 2A ribosome skipping element is a T2A, a P2A, a E2A, or a F2A element.

Any suitable effector molecule known in the art can be encoded in or expressed by a polynucleotide within a provided heterologous construct. In some embodiments, an effector molecule (e.g., a first effector molecule, a second effector molecule, a third effector molecule, and so forth) is a therapeutic molecule. Suitable effector molecules can be grouped into therapeutic classes based on structure similarity, sequence similarity, or function. Effector molecule therapeutic classes include, but are not limited to, cytokines, chemokines, homing molecules, growth factors, receptors, ligands, antibodies, polynucleotides, peptides, shRNAs, miRNAs, and enzymes. Accordingly, in some embodiments, an effector molecule (e.g., a first effector molecule, a second effector molecule, a third effector molecule, and so forth) belongs to a therapeutic class selected from the group consisting of: a cytokine, a chemokine, a homing molecule, a growth factor, a receptor, a ligand, an antibody, a peptide, an RNA molecule (e.g., mRNA, miRNA, siRNA, shRNA, etc.), and an enzyme.

In some embodiments, an effector molecule is a chemokine. Chemokines are small cytokines or signaling proteins secreted by cells that can induce directed chemotaxis in cells.

Chemokines can be classified into four main subfamilies: CXC, CC, CX3C and XC, all of which exert biological effects by binding selectively to chemokine receptors located on the surface of target cells. Non-limiting examples of chemokines that may be encoded by polynucleotides in a heterologous construct of the present disclosure include: CCL1, CCL2, CCL3, CCL4, CCL5, CCL6, CCL7, CCL8, CCL9/CCL10, CCL11, CCL12, CCL13, CCL14, CCL15, CCL16, CCL17, CCL18, CCL19, CCL20, CCL21, CCL22, CCL23, CCL24, CCl25, CCL26, CCL27, CCL28, CXCL1, CXCL2, CXCL3, CXCL4, CXCL5 CXCL6, CXCL7, CXCL8, CXCL9, CXCL10, CXCL11, CXCL12, CXCL13, CXCL14, CXCL15, CXCL16, CXCL17, XCL1, XCL2, CX3CL1, or combinations thereof.

In some embodiments, an effector molecule is a cytokine. Non-limiting examples of cytokines that may be encoded by polynucleotides in a heterologous construct of the present disclosure include: IL-1-alpha, IL-1-beta, IL-2, IL-3, IL-4, IL-5, IL-6, IL-7, IL-9, IL-10, IL-11, IL-12, IL-13, IL-15, IL-16, IL-17, IL-17A, IL-17B/C/D, IL-17E (IL-25), IL-17F, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, IL-36-alpha, IL-36-beta, IL-36-gamma, IL-37, IL-38, IFN-alpha, IFN-beta, IFN-gamma, TGF-beta, GM-CSF, CSF-1/M-CSF, G-CSF, SCF, TNF-alpha, TNF-beta, growth hormone (GH), prolactin (PRL), erythropoietin (EPO), leptin, FLT3 ligand, or combinations thereof.

In some embodiments, an effector molecule is a tumor microenvironment modifier. Suitable tumor microenvironment modifiers for use as an effector molecule include, but are not limited to, adenosine deaminase, TGF-beta inhibitors, immune checkpoint inhibitors, VEGF inhibitors, and HPGE2, or any combination thereof.

In some embodiments, a heterologous construct of the present disclosure is configured to produce an effector molecule comprising at least one TGF-beta inhibitor. Suitable TGF-beta inhibitors for use as an effector molecule include, but are not limited to, an anti-TGF-beta peptide, an anti-TGF-beta antibody, a TGF-beta-TRAP, or combinations thereof.

In some embodiments, a heterologous construct of the present disclosure is configured to produce an effector molecule comprising at least one immune checkpoint inhibitor. Suitable immune checkpoint inhibitors for use as an effector molecule include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNF-alpha antibodies, anti-TREM1 antibodies, and anti-TREM2 antibodies, or combinations and/or functional fragments thereof.

Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NCI), avelumab (anti-PD-L1; Bavencio®—Pfizer), durvalumab (anti-PD-L1; MEDI4736/Imfinzi®-Medimmune/AstraZeneca), atezolizumab (anti-PD-L1; Tecentriq®—Roche/Genentech), BMS-936559 (anti-PD-L1—BMS), tremelimumab (anti-CTLA-4; Medimmune/AstraZeneca), ipilimumab (anti-CTLA-4; Yervoy @—BMS), lirilumab (anti-KIR; BMS), monalizumab (anti-NKG2A; Innate Pharma/AstraZeneca).

In some embodiments, a heterologous construct of the present disclosure is configured to produce an effector molecule comprising at least one VEGF inhibitor. Suitable VEGF inhibitors for use as an effector molecule include, but are not limited to, anti-VEGF antibodies, anti-VEGF peptides, or combinations thereof. In some embodiments, the VEGF inhibitors comprise anti-VEGF antibodies, anti-VEGF peptides, or combinations thereof.

In some embodiments, a heterologous construct of the present disclosure is configured to produce an effector molecule comprising at least one therapy that treats an eye disease or disorder. In some embodiments, an eye disease or disorder is macular degeneration, inherited macular dystrophy, cone-rod dystrophy (CRD), rod-cone dystrophy (also referred to as retinitis pigmentosa, or RP), Usher syndrome, Stargardt disease, achromatopsia (ACHM), Leber congenital amaurosis (LCA), choroideremia (CHM), Bardet-Biedl syndrome (BBS), or retinoblastoma.

In some embodiments, a heterologous construct of the present disclosure is configured to produce an effector molecule comprising at least one gene replacement therapy. A gene therapy is a therapy that replaces a non-functional, partially functional, or missing disease-related gene with an engineered version of the same disease-related gene, thereby providing a therapeutically relevant amount of the disease-related gene product. In some embodiments, a gene replacement therapy is a therapy that treats an eye disease or disorder. In some embodiments, an eye disease or disorder treated by a gene replacement therapy includes macular degeneration, inherited macular dystrophy, cone-rod dystrophy (CRD), rod-cone dystrophy (also referred to as retinitis pigmentosa, or RP), Usher syndrome, Stargardt disease, achromatopsia (ACHM), Leber congenital amaurosis (LCA), choroideremia (CHM), Bardet-Biedl syndrome (BBS), or retinoblastoma.

In some embodiments, an effector molecule as described may comprise a secretion signal peptide (also referred to as a signal peptide or signal sequence) at the effector molecule's N-terminus. Without wishing to be bound by theory, a secretion signal peptide or signal sequence is understood to direct newly synthesized proteins destined for secretion or membrane insertion to the proper protein processing pathways. In embodiments with two or more effector molecules, each effector molecule can comprise a secretion signal.

In some embodiments, a secretion signal peptide operably associated with a effector molecule can be a native secretion signal peptide (e.g., a secretion signal peptide that is natively associated with the given effector molecule). In some embodiments, a secretion signal peptide operably associated with an effector molecule can be a non-native secretion signal peptide native secretion signal peptide. Non-native secretion signal peptides can promote improved expression and function, such as maintained secretion, in particular environments of interest, e.g., those related to ocular diseases or disorders.

### IV. Vectors/Plasmids

Another aspect of the disclosure relates to a vector comprising a nucleotide sequence encoding an engineered photoreceptor specific regulatory element (e.g., a heterologous construct), as described herein. In some embodiments, a vector is an expression vector. Such an expression vector comprises a nucleotide sequence encoding any engineered photoreceptor-specific regulatory element disclosed herein operably linked a coding nucleotide sequence (e.g., for an effector molecule, e.g., as described herein) to allow for expression of said coding nucleotide sequence in a cell or cell-free extract. A wide variety of expression vectors can be employed for expressing a nucleic acid molecule encoding engineered photoreceptor-specific regulatory elements of the present disclosure including, without limitation, viral expression vectors, prokaryotic expression vectors, eukaryotic expression vectors (e.g., yeast expression vectors, insect expression vectors, mammalian expression vectors, etc.), and cell-free extract expression vectors.

It is further understood that expression vectors useful to practice aspects of methods described herein may include additional promoters (e.g., inducible, constitutive, cell-specific), enhancer elements, or both. Expression vectors may include polynucleotides encoding protein tags, or epitope tags, to aid in isolation, purification or selection (e.g., poly-His tags, FLAG-tags, hemagglutinin tags, fluorescent protein tags, bioluminescent tags, and nuclear localization tags).

As described herein, coding sequences for such protein tags, or epitope tags, can be fused to a coding sequence or can be included in a separate expression cassette. Non-limiting examples of expression vectors, along with well-established reagents and conditions for making and using an expression construct from such expression vectors are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; EMD Biosciences-Novagen, Madison, Wis.; QIAGEN, Inc., Valencia, Calif.; and Stratagene, La Jolla, Calif. The selection, making and use of an appropriate expression vector are routine procedures well within the scope of one skilled in the art and from the teachings herein.

In some embodiments, a vector comprises a transposon/transposase system to incorporate a nucleotides of the present disclosure into a host cell genome. In some embodiments, a transposon system used in accordance with the present disclosure is a Sleeping Beauty transposon/transposase or the piggyBac transposon/transposase.

In some embodiments, an expression vector of the present disclosure may be provided to a cell in the form of a viral vector. Suitable viral vector systems are well known in the art. For example, viral vectors may be derived from retroviruses, adenoviruses, adeno-associated viruses, herpes viruses, and lentiviruses. In some embodiments, a vector of the present disclosure is a lentiviral vector. Lentiviral vectors are suitable for long-term gene transfer as such vectors allow long-term, stable integration of a transgene and its propagation in daughter cells. Lentiviral vectors are also advantageous over vectors derived from onco-retroviruses (e.g., murine leukemia viruses) in that lentiviral vectors can transduce non-proliferating cells. In some embodiments, a vector of the present disclosure is an adenoviral vector (A5/35).

In some embodiments, a vector of the present disclosure contains an origin of replication functional in at least one organism, a promoter sequence, convenient restriction endonuclease sites, and one or more selectable markers (e.g., WO 01/96584; WO 01/29058; and U.S. Pat. No. 6,326,193). A number of viral based systems have been developed for gene transfer into mammalian cells. A selected gene (e.g., alone, or within an expression cassette, or heterologous construct) can be inserted into a vector and packaged in retroviral particles using techniques known in the art. A recombinant virus can then be isolated and delivered to mammalian cells either in vivo or ex vivo. A number of retroviral systems are known in the art.

In some embodiments, vectors of the present disclosure comprise regulatory elements such as enhancers that regulate the frequency of transcriptional initiation. Enhancers are typically located in a region that is 30 bp to 110 bp upstream of a transcription start site, although a number of promoters have been shown to contain functional elements downstream of the start site as well. In general, the spacing between regulatory elements may be flexible, so that transcriptional function is preserved when regulatory elements are inverted or moved relative to one another. For example, in the thymidine kinase (tk) promoter the spacing between promoter elements can be increased to 50 bp apart before activity begins to decline. Depending on the promoter, individual elements may function either cooperatively or independently to activate transcription. Exemplary promoters may include, without limitation, a SFFV gene promoter, an EFS gene promoter, a CMV IE gene promoter, an EF1a promoter, a ubiquitin C promoter, a phosphoglycerokinase (PGK) promoter, or functional fragments or combinations thereof.

In some embodiments, a vector of the present disclosure may further comprise a signal sequence to facilitate secretion, a polyadenylation signal and transcription terminator, an element allowing episomal replication, and/or elements allowing for selection.

In some embodiments, a vector of the present disclosure can further comprise a selectable marker gene and/or a reporter gene to facilitate identification and selection of certain cells (e.g., those comprising an engineered photoreceptor-specific regulatory element as described herein) from a population of cells that have been transduced with said vector. In some embodiments, a selectable marker may be encoded by a polynucleotide that is separate from a vector and used in a co-transfection procedure. A selectable marker or a reporter gene may be flanked with appropriate regulatory sequences to allow expression in a host cell. In some embodiments, a selectable marker comprises an antibiotic resistance gene. Examples of antibiotic resistance genes include, without limitation, kanamycin, spectinomycin, streptomycin, ampicillin, carbenicillin, bleomycin, erythromycin, polymyxin B, tetracycline, chloramphenicaol, neomycin, and combinations thereof.

In some embodiments, a reporter gene may be used for identifying transduced cells and for evaluating the functionality of regulatory sequences. As disclosed herein, a reporter gene is a gene that is not present in or expressed by the recipient organism or tissue and that encodes a polypeptide that has an easily detectable property, such as enzymatic activity or fluorescence. Expression of a reporter gene can be assayed at a suitable time after a polynucleotide comprising said reporter gene has been introduced into a recipient or host cell that is capable of expressing said reporter gene. Examples of reporter genes include, without limitation, genes encoding for luciferase (or variants or fragments of luciferase, e.g., nanoluciferase), genes encoding for beta-galactosidase, genes encoding for chloramphenicol acetyl transferase, genes encoding for secreted alkaline phosphatase, and genes encoding for green fluorescent protein (GFP) (or common variants or fragments of GFP, e.g., RFP, YFP, etc.). Suitable expression systems are well known in the art and may be prepared using known techniques or obtained commercially.

### V. Engineering Photoreceptor Cells

The present disclosure further provides for methods and compositions for preparing and using engineered photoreceptor cells comprising a heterologous construct comprising an engineered regulatory element as provided herein.

In some embodiments, engineered photoreceptor cells comprise an engineered regulatory element.

Also provided herein are compositions and methods for engineering photoreceptor cells that are capable of producing one or more effectors molecules (e.g., a first effector molecule, a second effector molecule, a third effector molecule, and so forth). Effector molecules of the present disclosure may be encoded in one or more heterologous constructs or expression cassettes as described herein or otherwise known in the art.

In some embodiments, photoreceptor cells of the present disclosure are engineered to produce effector molecules through introduction (i.e., delivery) of one or more polynucleotides that encode for one or more effector molecules (e.g., those provided herein). For example, polynucleotide heterologous constructs or expression cassettes encoding one or more effector molecules can be any of the engineered nucleic acids described herein. Delivery methods include, but are not limited to, viral-mediated delivery, lipid-mediated transfection, nanoparticle delivery, electroporation, sonication, and cell membrane deformation by physical means (e.g., including all methods described herein). One skilled in the art will appreciate the choice of delivery method can depend on the specific cell type to be engineered.

### A. Viral-Mediated Delivery

Viral vector-based delivery platforms can be engineered to deliver certain nucleic acids of interest into a host cell in order to create an engineered cell as described herein. In general, a viral vector-based delivery platform can be used to create an engineered cell by introducing (i.e., delivering, or transducing) a heterologous nucleic acid, e.g., a transgene, expression cassette, or a heterologous construct as described herein, into a particular host cell. In many embodiments of the present disclosure, a viral vector-based delivery platform delivers a nucleic acid comprising an engineered photoreceptor-specific regulatory element as described herein, or a heterologous construct as described herein. In some embodiments, a transduced nucleic acid is integrated into a host cell genome. Viruses created for use in viral vector-based delivery platforms can be referred to as recombinant viruses, or engineered viruses. It is understood that a recombinant virus may encode one or more viral genes needed for viral infectivity and/or viral production (e.g., capsid proteins, envelope proteins, viral polymerases, viral transcriptases, etc.), in some cases referred to as cis-acting elements or cis-acting genes, in addition to nucleic acids to be delivered to a host cell.

In some embodiments, a recombinant viruses may be used to deliver nucleic acids encoding one or more genes (e.g., transgenes), expression cassettes, or heterologous constructs. In many embodiments, delivered nucleic acids comprise one or more nucleotide sequences comprising engineered photoreceptor-specific element as described herein. In some embodiments, delivered nucleic acids (e.g., heterologous constructs) are configured to express one or more effector molecules.

A viral vector-based delivery platform can comprise more than one viral vector, such as separate viral vectors encoding genes (e.g., transgenes), expression cassettes, or heterologous constructs described herein, and referred to as trans-acting elements or trans-acting genes. For example, a helper-dependent viral vector-based delivery platform can provide additional genes needed for viral infectivity and/or viral production on one or more additional separate vectors in addition to a vector encoding a transgene expression cassette, or heterologous construct. The number of viral vectors used can depend on the packaging capacity of the above-mentioned viral vector-based platforms, and one skilled in the art can select the appropriate number of viral vectors.

In general, any of viral vector-based system can be used for the in vitro expression of engineered nucleic acids (e.g., heterologous constructs), e.g., for the production of molecules, such as effector molecules, or used in vivo and ex vivo gene therapy procedures, e.g., for in vivo delivery of the engineered nucleic acids comprising nucleotide sequences encoding one or more effector molecules under transcriptional control of an engineered photoreceptor-specific regulatory element. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can utilize RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

Provided engineered nucleic acid sequences may be preceded with one or more nucleic acid sequences that by themselves, or via their encoded polypeptide sequence, target a subcellular compartment. Upon introduction (i.e. delivery) of engineered nucleic acids into a host cell, infected cells (i.e., engineered cells) can express polypeptides encoded by said engineered nucleic acids (e.g., one or more effector molecules), and in some case secrete, said polypeptides. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms described herein can utilize a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more engineered nucleic acids (e.g., genes, expression cassettes, heterologous constructs, etc.).

In some embodiments, a recombinant virus is created from a virus selected from: a lentivirus, a retrovirus, an oncolytic virus, an adenovirus, an adeno-associated virus (AAV), and a virus-like particle (VLP).

A viral vector-based delivery platform as used in accordance with the present disclosure can be retrovirus-based. In general, retroviral vectors are comprised of cis-acting long terminal repeats with packaging capacity for up to 6-10 kb of foreign sequence. The minimum cis-acting LTRs are sufficient for replication and packaging of said vectors, which are then used to integrate the one or more engineered nucleic acids (e.g., genes, expression cassettes, heterologous constructs, etc.) into a target cell to provide permanent integration and/or expression of said engineered nucleic acids. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et al, J. Virol. 66:1635-1640 (1992); Sommnerfelt et al., Virol. 176:58-59 (1990); Wilson et al, J. Virol. 63:2374-2378 (1989); Miller et al, J, Virol. 65:2220-2224 (1991); PCT/US94/05700). Other retroviral systems include the Phoenix retrovirus system.

A viral vector-based delivery platform as used in accordance with the present disclosure can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

A viral vector-based delivery platform as used in accordance with the present disclosure can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO 96/13597, each herein incorporated by reference for all purposes.

A viral vector-based delivery platform as used in accordance with the present disclosure can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of an engineered nucleic acids, e.g., those encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003/0138772, US 2007/0036760, and US 2009/0197338; Gao, et al., J. Virol, 78(12):6381-6388 (June 2004); Gao, et al., Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each of which are herein incorporated by reference for all purposes). Exemplary methods for constructing recombinant AAV vectors are described in more detail in U.S. Pat. No. 5,173,414; Tratschin et al., Mol. Cell. Biol. 5:3251-3260 (1985); Tratschin et al., Mol. Cell, Biol. 4:2072-2081 (1984); Muzyczka, PNAS 81:64666470 (1984); and Samuiski et al., J. Virol. 63:03822-3828 (1989), each of which are herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

A viral vector-based delivery platform as used in accordance with the present disclosure can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

A viral vector-based delivery platform as used in accordance with the present disclosure can be engineered to target (i.e., infect or transduce) a range of cells, target a narrow subset of cells, or target a specific cell. In general, an envelope protein chosen for a viral vector-based delivery platform will determine viral tropism. A virus used in a viral vector-based delivery platform can be pseudotyped to target a specific cell of interest. A viral vector-based delivery platform can be pantropic and infect a range of cells. For example, pantropic viral vector-based delivery platforms can include the VSV-G envelope. A viral vector-based delivery platform can be amphotropic and infect mammalian cells. Accordingly, one skilled in the art can select the appropriate tropism, pseudotype, and/or envelope protein for targeting a desired cell type.

### B. Lipid Structure Delivery Systems

Engineered nucleic acids of the present disclosure (e.g., any of engineered nucleic acid as described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any engineered nucleic acid as described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each of which are herein incorporated by reference for all purposes

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, a engineered nucleic acid, such as any engineered nucleic acid as described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with a lipid bilayer.

A liposome used in accordance with the present disclosure can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each of which are hereby incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO 03/015757A1, WO 04029213A2, and WO 02/100435A1, each of which are herein incorporated by reference in their entirety

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each of which are herein incorporated by reference for all purposes.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any engineered nucleic acid as described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any engineered nucleic acid as described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any engineered nucleic acid as described herein), a sugar (e.g., a simple sugar, polysaccharide, or glycan) or other molecules. A nanovesicle, once it is derived from a producer cell according to said manipulation, may be isolated from the producer cell based on its size, density, biochemical parameters, or a combination thereof.

Lipid nanoparticles (LNPs), in general, are engineered lipid structures that rely on the amphiphilic nature of lipids to form membranes and vesicle like structures (Riley 2017). In general, these vesicles deliver cargo/payloads, such as any engineered nucleic acid or viral system described herein, by absorbing into a membrane of a target cell and releasing the cargo into the cytosol. Lipids used in LNP formation can be cationic, anionic, or neutral. The lipids can be engineered or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity, which will be appreciated by those skilled in the art.

Micelles, in general, are spherical engineered lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of a nucleic acid by serum nucleases or off-target stimulation of an immune system by free nucleic acids. Similarly, viral delivery systems exposed directly to serum can trigger an undesired immune response and/or neutralization of a viral delivery system. Therefore, encapsulation of a engineered nucleic acid and/or viral delivery system can be used to avoid degradation, while also avoiding potential off-target affects. In certain examples, an engineered nucleic acid and/or viral delivery system is fully encapsulated within a delivery vehicle, such as within an aqueous interior of an LNP, or other vesicle or lipid system as described herein. Encapsulation of an engineered nucleic acid and/or viral delivery system within an LNP or other lipid system can be carried out by techniques well-known to those skilled in the art, such as microfluidic mixing and droplet generation carried out on a microfluidic droplet generating device. Such devices include, but are not limited to, standard T-junction devices or flow-focusing devices. In an example, a desired lipid formulation, such as MC3 or MC3-like containing compositions, is provided to a droplet generating device in parallel with an engineered nucleic acid or viral delivery system and any other desired agents, such that a delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, delivery vehicles encapsulating a cargo/payload (e.g., an engineered nucleic acid and/or viral delivery system) can be further treated or engineered to prepare them for administration.

### C. Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to a delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), each of which are herein incorporated by reference for all purposes.

### D. Genomic Editing Systems

A genomic editing system can be used to engineer a host genome to encode a engineered nucleic acid, such as any engineered nucleic acid described herein. In general, a “genomic editing system” refers to any system for integrating an exogenous gene into a host cell's genome. Genomic editing systems include, but are not limited to, a transposon system, a nuclease genomic editing system, and a viral vector-based delivery platform (e.g., those described herein).

A transposon system can be used to integrate an engineered nucleic acid, e.g., an engineered nucleic acid as described herein, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes

A nuclease genomic editing system can be used to engineer a host genome to encode a engineered nucleic acid, such as an engineered nucleic acid of the present disclosure. Without wishing to be bound by theory, in general, nuclease-mediated gene editing systems used to introduce an exogenous gene, or nucleic acid, take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into a cell to be used as a homologous recombination template (HRT or HR template). In general, any additional exogenous sequence not originally found in the chromosome with a lesion that is included between the 5′ and 3′ complimentary ends within a HRT (e.g., a gene or a portion of a gene, or engineered nucleic acid as described herein) can be incorporated (i.e., “integrated”) into a given genomic locus during templated HDR. Thus, a typical HR template for a given genomic locus has a nucleotide sequence identical to a first region of an endogenous genomic target locus, a nucleotide sequence identical to a second region of an endogenous genomic target locus, and a nucleotide sequence encoding a cargo/payload nucleic acid (e.g., any engineered nucleic acid as described herein, e.g., those encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template

The identical, or substantially identical, sequences found at the 5′ and 3′ ends of a HR template, with respect to an exogenous sequence to be introduced, are generally referred to as arms (HR arms). HR arms can be identical to regions of an endogenous genomic target locus (i.e., 100% identical). HR arms in some examples can be substantially identical to regions of an endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid as described herein, e.g., those encoding one or more effector molecules described herein. CRISPR systems are described in more detail in M. Adli (“The CRISPR tool kit for genome editing and beyond” Nature Communications; volume 9 (2018), Article number: 1911), herein incorporated by reference for all purposes. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

Engineered cells described herein can be engineered using non-viral methods, e.g., nuclease and/or CRISPR mediated gene editing systems described herein can be delivered to a cell using non-viral methods. The engineered cells described herein can be engineered using viral methods, e.g., nuclease and/or CRISPR mediated gene editing systems described herein can be delivered to a cell using viral methods such as adenoviral, retroviral, lentiviral, or any of other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is a engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### E. Other Engineering Delivery Systems

Various additional means to introduce engineered nucleic acids (e.g., any engineered nucleic acid as described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of a target cell or entity. In general, the method involves placing cells or target entities between two electrodes in a solution containing a cargo of interest (e.g., any of engineered nucleic acid as described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, type of cell or other recipient entity, cargo to be delivered, efficiency of internalization desired, and viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of engineered nucleic acid as described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each of which are herein incorporated by reference for all purposes.

### VI. Methods of Use

Methods and compositions for treating a subject with a disease or disorder are encompassed by the present disclosure. Provided methods include administering to a subject with a disease or disorder a therapeutically effective amount of any engineered nucleic acid (e.g., a heterologous construct) or engineered cell (e.g., an isolated engineered cell) described herein.

In some embodiments, methods and compositions provided herein may be used to treat an eye disease or disorder. In some embodiments, an eye disease or disorder is cancer. In some embodiments, an eye disease or disorder is macular degeneration, inherited macular dystrophy, cone-rod dystrophy (CRD), rod-cone dystrophy (also referred to as retinitis pigmentosa, or RP), Usher syndrome, Stargardt disease, achromatopsia (ACHM), Leber congenital amaurosis (LCA), choroideremia (CHM), Bardet-Biedl syndrome (BBS), or retinoblastoma.

### VII. Pharmaceutical Compositions

Provided engineered nucleic acids or engineered cells can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or engineered oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Composition provided herein can be administered alone or in combination with other treatments, either simultaneously or sequentially dependent upon the condition to be treated.

### VIII. Kits

Certain aspects of the present disclosure relate to kits for the treatment and/or prevention of disease or disorder. In certain embodiments, a disease or disorder is a cancer (e.g., solid tumors). In certain embodiments, a kit includes a therapeutic or prophylactic composition comprising an effective amount of one or more engineered nucleic acids (e.g., isolate engineered nucleic acids) of the present disclosure, vectors of the present disclosure, and/or engineered cells of the present disclosure. In some embodiments, a kit comprises a sterile container. In some embodiments, such containers can be boxes, ampules, bottles, vials, tubes, bags, pouches, blister-packs, or other suitable container forms known in the art. A container may be made of plastic, glass, laminated paper, metal foil, or other materials suitable for holding medicaments

In some embodiments, a therapeutic or prophylactic composition is provided together with instructions for administering said therapeutic or prophylactic composition to a subject having or at risk of developing a particular disease or disorder (e.g., an eye disease or disorder). In some embodiments, the instructions may include information about use of a composition for the treatment and/or prevention of a disease or disorder. In some embodiments, the instructions include, without limitation, a description of a therapeutic or prophylactic composition, a dosage schedule, an administration schedule for treatment or prevention of a disease or disorder or a symptom thereof, precautions, warnings, indications, counter-indications, over-dosage information, adverse reactions, animal pharmacology, clinical studies, and/or references. In some embodiments, the instructions can be printed directly on a container (when present), or as a label applied to a container, or as a separate sheet, pamphlet, card, or folder supplied in or with a container.

Throughout the description, where agents, compounds, entities, and so forth (e.g., provided engineered nucleic acids) are described as having, including, or comprising specific components, or where processes and methods are described as having, including, or comprising specific steps, it is contemplated that, additionally, there are agents, compounds, entities, and so forth of the present invention that consist essentially of, or consist of, the recited components, and that there are processes and methods according to the present invention that consist essentially of, or consist of, the recited processing steps.

The use of the alternative (e.g., “or”) should be understood to mean either one, both, or any combination thereof of the alternatives.

Practice of the invention will be more fully understood from the foregoing examples, which are presented herein for illustrative purposes only, and should not be construed as limiting the invention in any way.

### Enumerated Embodiments

Embodiment 1: An engineered photoreceptor-specific regulatory element comprising an enhancer region operably linked to a minimal promoter, wherein the enhancer region is heterologous to the minimal promoter, and wherein the engineered photoreceptor-specific regulatory element exhibits greater activity in photoreceptor cells of a mammalian retina as compared to non-photoreceptor cells of the same mammalian retina.

Embodiment 2: The engineered photoreceptor-specific regulatory element of embodiment 1, wherein the enhancer region is about 50 to about 600 base pairs in length, about 100 to about 600 base pairs in length, about 200 to about 600 base pairs in length, about 300 to about 600 base pairs in length, about 400 to about 600 base pairs in length, about 500 to about 600 base pairs in length, about 50 to about 500 base pairs in length, about 50 to about 400 base pairs in length, about 50 to about 300 base pairs in length, about 50 to about 200 base pairs in length, about 50 to about 175 base pairs in length, about 50 to about 150 base pairs in length, about 50 to about 125 base pairs in length, or about 50 to about 100 base pairs in length.

Embodiment 3: The engineered photoreceptor-specific regulatory element of embodiment 1 or embodiment 2, wherein the minimal promoter is selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, lateADE, minIL2.2, SMP, inducer molecule responsive promoters, CMV, EFS, SFFV, SV40, MND, PGK, UbC, hEFlaV1, hCAGG, hEFlaV2, hACTb, heIF4Al, hGAPDH, hGRP78, hGRP94, hHSP70, hKINb, hUBIb, and tandem repeats thereof.

Embodiment 4: The engineered photoreceptor-specific regulatory element of any one of embodiments 1-3, further comprising a spacer sequence, wherein the spacer is operably linked to the enhancer region and the minimal promoter.

Embodiment 5: The engineered photoreceptor-specific regulatory element of embodiment 4, wherein the spacer is selected from the group consisting of SEQ ID Nos 107-110.

Embodiment 6: The engineered photoreceptor-specific regulatory element of embodiment 4, wherein the spacer comprises the nucleotide sequence: CCTGCAGG (SEQ ID NO: 107).

Embodiment 7: The engineered photoreceptor-specific regulatory element of any one of embodiments 1-6, wherein the enhancer region comprises the nucleotide sequence:

(SEQ ID NO: 1)
TGTTGAGAGCTCAAGCTCTTTTTAACGCTTTGCCTTGCTGTCTCCAAAG
TATTGCCTTCATCCTCATAGTTCAAAGTGTCCACCATCACATTCACGTT
TCAGCCAATAGGAAGAAGGAAAGAGAAAAACAGGAAAAGAGTTTACATG
CCACTCCTGCTATTGGTCAGAATGTGTCACGTGACCATACTCAGCTGCA
AGGGTTGGTGGGAAATGTAGTCTTTTTTTCTGGGAGGCCATGTGTTCGG
CTTAAAATTGTCTTTTTTTCTTTTTCTTTATTTCTTTTTTCTTTTCTTT
CTTTTTTTTAGACGG.

Embodiment 8: The engineered photoreceptor-specific regulatory element of embodiment 7, wherein the minimal promoter is derived from YB TATA.

Embodiment 9: The engineered photoreceptor-specific regulatory element of embodiment 8, wherein the minimal promoter comprises the nucleotide sequence:

(SEQ ID NO: 122)
TCTAGAGGGTATATAATGGGGGCCA.

Embodiment 10: The engineered photoreceptor-specific regulatory element of any one of embodiments 7-9, wherein the engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 138.

Embodiment 11: The engineered photoreceptor-specific regulatory element of any one of embodiments 1-6, wherein the enhancer region comprises the nucleotide sequence:

(SEQ ID NO: 2)
GGTTACACAACCAGGCGGGGAGGGGCCCCGGGGGGGGGAGGGGGCCGGC
CCGCGGGCCGCGCAGCCGGAAGCCGGGACCGCCACCGGCCCCCGGCGAG
GGGAGCCCGGCTCCAGGCCCCGCCCCCTGGCGGGCTGGCCCCGCCCCCG
CGCCGCGCCGCGCGATCGGCCCGCGCCCATTGGCTCTCCGGCCCGCCGC
TCACCGCCCCTCCTCCGCACCGCCCCTACCCGCAGGCCGCGGCGGGCTG
TCGGCGCGGGGCACCCTGGGACTTGTAGTCCAAGCCGCTTGCCACCTGC
CGGCTGCAAACGGCGGAGGGACTACGAAGCCCAGAGGTCCCTGCGGCCC
TGCCCGCCCACCCGGACACCCCACCCCTTCCCCCTCCTTTCCGAAGCCC
CCCTCCCTGTTTTTTCAGCCCCCTC.

Embodiment 12: The engineered photoreceptor-specific regulatory element of embodiment 11, wherein the minimal promoter is derived from YB TATA.

Embodiment 13: The engineered photoreceptor-specific regulatory element of embodiment 12, wherein the minimal promoter comprises the nucleotide sequence:

(SEQ ID NO: 122)
TCTAGAGGGTATATAATGGGGGCCA.

Embodiment 14: The engineered photoreceptor-specific regulatory element of any one of embodiments 11-13, wherein the engineered photoreceptor-specific promoter comprises at least 75%, at least 80%, at least 85%, at least 90%, at least 95%, at least 96%, at least 97%, at least 98%, at least 99%, or 100% sequence identity to the nucleotide sequence of SEQ ID NO: 139.

Embodiment 15: The engineered photoreceptor-specific regulatory element of any one of embodiments 1-6, wherein the enhancer region comprises the nucleotide sequence:

(SEQ ID NO: 3)
CAGGCGGGGAGGGGCCCCGGGGCGGGGAGGGGGCCGGCCCGCGGGCCGC
GCAGCCGGAAGCCGGGACCGCCACCGGCCCCCGGCGAGGGGAGCCCGGC
TCCAGGCCCCGCCCCCTGGCGGGCTGGCCCCGCCCCCGCGCCGCGCCGC
GCGATCGGCCCGCGCCCATTGGCTCTCCGGCCCGCCGCTCACCGCCCCT
CCTCCGCACCGCCCCTACCCGCAGGCCGCGGCGGGCTGTCGGCGCGGGG
CACCCTGGGACTTGTAGTCCAAGCCGCTTGCCACCTGCCGGCTGCAAAC
GGCGGAGGGACTACGAAGCCCAGAGGTCCCTGCGGCCCTGCCCGCCCAC
CCGGACACCCCACCCCTTCCCCCTCCTTTCCGAAGCCCCCCTCCCTGTT
TTTTCAGCCCCCTCCCCCCCATCCCCCATGGAGC.

Embodiment 16: The engineered photoreceptor-specific regulatory element of embodiment 15, wherein the minimal promoter is derived from YB TATA.

Embodiment 17: The engineered photoreceptor-specific regulatory element of embodiment 16, wherein the minimal promoter comprises the nucleotide sequence:

(SEQ ID NO: 122)
TCTAGAGGGTATATAATGGGGGCCA.

Embodiment 18: The engineered photoreceptor-specific regulatory element of any one of embodiments 15-17, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 140.

Embodiment 19: The engineered photoreceptor-specific regulatory element of any one of embodiments 1-6, wherein the enhancer region comprises the nucleotide sequence:

(SEQ ID NO: 1)
TGTTGAGAGCTCAAGCTCTTTTTAACGCTTTGCCTTGCTGTCTCCAAAG
TATTGCCTTCATCCTCATAGTTCAAAGTGTCCACCATCACATTCACGTT
TCAGCCAATAGGAAGAAGGAAAGAGAAAAACAGGAAAAGAGTTTACATG
CCACTCCTGCTATTGGTCAGAATGTGTCACGTGACCATACTCAGCTGCA
AGGGTTGGTGGGAAATGTAGTCTTTTTTTCTGGGAGGCCATGTGTTCGG
CTTAAAATTGTCTTTTTTTCTTTTTCTTTATTTCTTTTTTCTTTTCTTT
CTTTTTTTTAGACGG

Embodiment 20: The engineered photoreceptor-specific regulatory element of embodiment 19, wherein the minimal promoter is derived from minTK.

Embodiment 21: The engineered photoreceptor-specific regulatory element of embodiment 20, wherein the minimal promoter comprises the nucleotide sequence:

(SEQ ID NO: 123)
TTCGCATATTAAGGTGACGCGTGTGGCCTCGAACACCGAGCGACCCTGC
AGCGACCCGCTTAA.

Embodiment 22: The engineered photoreceptor-specific regulatory element of any one of embodiments 19-21, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 141.

Embodiment 23: The engineered photoreceptor-specific regulatory element of embodiment 19, wherein the minimal promoter is derived from lateADE.

Embodiment 24: The engineered photoreceptor-specific regulatory element of embodiment 23, wherein the minimal promoter comprises the nucleotide sequence:

(SEQ ID NO: 126)
AGACGCTAGCGGGGGGCTATAAAAGGGGGTGGGGGCGTTCGTCCTCACT
CT.

Embodiment 25: The engineered photoreceptor-specific regulatory element of any one of embodiments 19, 23, or 24, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 142.

Embodiment 26: The engineered photoreceptor-specific regulatory element of any one of embodiments 1-6, wherein the enhancer region comprises the nucleotide sequence:

(SEQ ID NO: 4)
AGCACGCGCGAGCTGTCACCAGGGCGCGAGACAACCGAACACGGCCCCG
CCCCCTGCCCGTCTATTGGCCCGCCCGCTGGAGCCCCGCCCCTCAGGCC
CTGCATTGCGCCAACGGCGCAGCGCTGGGCCGCGACCCCGGCACCGGCA
CCCGTTCCGAGGGTTCGCGCCGCAGGCGCAAAGTTTAGGGTCAGCCACA
AACGCGCGGCCGCTTTGAGCCTGGCGTAAGGGGCGGCGGCGTAGGGGGA
CGTTGTGAATAGCTGGTCGAAGTTTGGTTGAAGCACAGGAGGGTAAATA
AGAAGGTTTCCAGACAAAGAGACTCTTAAAGGTACAGACTCTTATGTCT
GTCCCTCCTCCTTAAAGGGCCAGAGACGCTCCCGAGCCCATCTC.

Embodiment 27: The engineered photoreceptor-specific regulatory element of embodiment 26, wherein the minimal promoter is derived from SMP.

Embodiment 28: The engineered photoreceptor-specific regulatory element of embodiment 27, wherein the minimal promoter comprises the nucleotide sequence:

(SEQ ID NO: 125)
AAAATGTGCGCATGTGCAGCCATTGCCTGGGACGCATGCGTAGGGAGCC
GCGCGACAAACTGAGCCATTGCGGCAAGACTAGCGCAGAGAGGAGAGGG
AGCCGGAGATGCCAGACGCTTGGTTCTGAGGAGTGATTTGCAACGCAAT
GGAGCGAGGAAGG.

Embodiment 29: The engineered photoreceptor-specific regulatory element of any one of embodiments 26-28, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 143.

Embodiment 30: The engineered photoreceptor-specific regulatory element of embodiment 26, wherein the minimal promoter is derived from lateADE.

Embodiment 31: The engineered photoreceptor-specific regulatory element of embodiment 30, wherein the minimal promoter comprises the nucleotide sequence:

(SEQ ID NO: 126)
AGACGCTAGCGGGGGGCTATAAAAGGGGGTGGGGGCGTTCGTCCTCACT
CT.

Embodiment 32: The engineered photoreceptor-specific regulatory element of embodiment 26, 30 or 31, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 144.

Embodiment 33: The engineered photoreceptor-specific regulatory element of embodiment 26, wherein the minimal promoter is derived from minCMV.

Embodiment 34: The engineered photoreceptor-specific regulatory element of embodiment 33, wherein the minimal promoter comprises the nucleotide sequence:

(SEQ ID NO: 127)
TAGGCGTGTACGGTGGGAGGCCTATATAAGCAGAGCTCGTTTAGTGAAC
CGTCAGATCGCCTGGA.

Embodiment 35: The engineered photoreceptor-specific regulatory element of any one of embodiments 26, 33, or 34, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 145.

Embodiment 36: The engineered photoreceptor-specific regulatory element of any one of embodiments 1-6, wherein the enhancer region comprises the nucleotide sequence:

(SEQ ID NO: 2)
GGTTACACAACCAGGCGGGGAGGGGCCCCGGGGGGGGAGGGGGCCGGCC
CGCGGGCCGCGCAGCCGGAAGCCGGGACCGCCACCGGCCCCCGGCGAGG
GGAGCCCGGCTCCAGGCCCCGCCCCCTGGCGGGCTGGCCCCGCCCCCGC
GCCGCGCCGCGCGATCGGCCCGCGCCCATTGGCTCTCCGGCCCGCCGCT
CACCGCCCCTCCTCCGCACCGCCCCTACCCGCAGGCCGCGGCGGGCTGT
CGGCGCGGGGCACCCTGGGACTTGTAGTCCAAGCCGCTTGCCACCTGCC
GGCTGCAAACGGCGGAGGGACTACGAAGCCCAGAGGTCCCTGCGGCCCT
GCCCGCCCACCCGGACACCCCACCCCTTCCCCCTCCTTTCCGAAGCCCC
CCTCCCTGTTTTTTCAGCCCCCTC.

Embodiment 37: The engineered photoreceptor-specific regulatory element of embodiment 36, wherein the minimal promoter is derived from minTK.

Embodiment 38: The engineered photoreceptor-specific regulatory element of embodiment 37, wherein the minimal promoter comprises the nucleotide sequence:

(SEQ ID NO: 123)
TTCGCATATTAAGGTGACGCGTGTGGCCTCGAACACCGAGCGACCCTGC
AGCGACCCGCTTAA.

Embodiment 39: The engineered photoreceptor-specific regulatory element of any one of embodiments 36-38, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the nucleotide sequence of SEQ ID NO: 146.

The engineered photoreceptor-specific regulatory element of embodiment 36, wherein the minimal promoter is derived from minIL2.2.

Embodiment 40: The engineered photoreceptor-specific regulatory element of embodiment 40, wherein the minimal promoter comprises the nucleotide sequence:

(SEQ ID NO: 124)
CAGAATTAACAGTATAAATTGCATCTCTTGTTCAAGAGTTCCCTATCAC
TCT.

Embodiment 41: The engineered photoreceptor-specific regulatory element of any one of embodiments 36, 40, or 41, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the sequence of SEQ ID NO: 147.

Embodiment 42: The engineered photoreceptor-specific regulatory element of embodiment 36, wherein the minimal promoter is derived from lateADE.

Embodiment 43: The engineered photoreceptor-specific promoter of embodiment 43, wherein the minimal promoter comprises the nucleotide sequence:

(SEQ ID NO: 126)
AGACGCTAGCGGGGGGCTATAAAAGGGGGTGGGGGCGTTCGTCCTCACT
CT.

Embodiment 44: The engineered photoreceptor-specific regulatory element of any one of embodiments 36, 43, or 44, wherein the engineered photoreceptor-specific regulatory element comprises at least 75%, at least 80%, at least 85%, at least 90%, at least 95%, at least 96%, at least 97%, at least 98%, at least 99%, or 100% sequence identity to the sequence of SEQ ID NO: 148.

Embodiment 45: An engineered photoreceptor-specific regulatory element comprising an enhancer region, wherein the enhancer region comprises an ablation of at least one nucleotide motif within a wild-type enhancer region, and wherein the engineered photoreceptor-specific regulatory element has greater activity than the same regulatory element without the ablation in photoreceptor cells as compared to non-photoreceptor cells.

Embodiment 46: The engineered photoreceptor-specific regulatory element of embodiment 46, wherein the engineered photoreceptor-specific regulatory element further comprises a minimal promoter.

Embodiment 47: The engineered photoreceptor-specific regulatory element of embodiment 47, wherein the engineered photoreceptor-specific regulatory element further comprises a spacer, wherein spacer is operably linked to the enhancer region and the minimal promoter.

Embodiment 48: The engineered photoreceptor-specific regulatory element of any one of embodiments 46-48, wherein the wild-type enhancer region comprises the nucleotide sequence:

(SEQ ID NO: 1)
tgttgagagctcaagctctttttaacgctttgccttgctgtctccaaag
tattgccttcatcctcatagttcaaagtgtccaccatcacattcacgtt
tcagccaataggaagaaggaaagagaaaaacaggaaaagagtttacatg
ccactcctgctattggtcagaatgtgtcacgtgaccatactcagctgca
agggttggtgggaaatgtagtctttttttctgggaggccatgtgttcgg
cttaaaattgtctttttttctttttctttatttcttttttcttttcttt
cttttttttagacgg.

Embodiment 49: The engineered photoreceptor-specific regulatory element of any one of embodiments 46-49, wherein the ablation comprises a substitution or deletion of one or more nucleotides of the at least one nucleotide motif.

Embodiment 50: The engineered photoreceptor-specific regulatory elements of any one of embodiments 46-50, wherein the at least one nucleotide motif comprises a motif having a sequence within the nucleotide sequence of SEQ ID NO: 1.

Embodiment 51: The engineered photoreceptor-specific regulatory element of embodiment 51, wherein the ablation comprises a nucleotide substitution of the motif comprising the nucleotide sequence: tgttgagagctcaagctctttttaacgctt (SEQ ID NO: 5).

Embodiment 52: The engineered photoreceptor-specific regulatory element of embodiment 51, wherein the ablation comprises a nucleotide substitution of the motif comprising the nucleotide sequence: TGCCTTGCTGTCTCCAAAGTATTGCCTTCA (SEQ ID NO: 7).

Embodiment 53: The engineered photoreceptor-specific regulatory element of embodiment 51, wherein the ablation comprises a nucleotide substitution of the motif comprising the nucleotide sequence: TCCTCATAGTTCAAAGTGTCCACCATCACA (SEQ ID NO: 9).

Embodiment 54: The engineered photoreceptor-specific regulatory element of embodiment 51, wherein the ablation comprises a nucleotide substitution of the motif comprising the nucleotide sequence: TTCACGTTTCAGCCAATAGGAAGAAGGAAA (SEQ ID NO: 11).

Embodiment 55: The engineered photoreceptor-specific regulatory element of embodiment 51, wherein the ablation comprises a nucleotide substitution of the motif comprising the nucleotide sequence: GAGAAAAACAGGAAAAGAGTTTACATGCCA (SEQ ID NO: 13).

Embodiment 56: The engineered photoreceptor-specific regulatory element of embodiment 51, wherein the ablation comprises a nucleotide substitution of the motif comprising the nucleotide sequence: CTTTATTTCTTTTTTCTTTTCTTTCTTTTT (SEQ ID NO: 15).

Embodiment 57: 58. The engineered photoreceptor-specific regulatory element of embodiment 51, wherein the ablation comprises a nucleotide substitution of the motif comprising the nucleotide sequence: TTTAGACGG (SEQ ID NO: 17).

Embodiment 58: The engineered photoreceptor-specific regulatory element of any one of embodiments 50-58, wherein the nucleotide substitution comprises an inert sequence.

Embodiment 59: The engineered photoreceptor-specific regulatory element of any one of embodiments 46-48, wherein the wild-type enhancer region comprises the nucleotide sequence:

(SEQ ID NO: 2)
GGTTACACAACCAGGCGGGGAGGGGCCCCGGGGGGGGGAGGGGGCCGGC
CCGCGGGCCGCGCAGCCGGAAGCCGGGACCGCCACCGGCCCCCGGCGAG
GGGAGCCCGGCTCCAGGCCCCGCCCCCTGGCGGGCTGGCCCCGCCCCCG
CGCCGCGCCGCGCGATCGGCCCGCGCCCATTGGCTCTCCGGCCCGCCGC
TCACCGCCCCTCCTCCGCACCGCCCCTACCCGCAGGCCGCGGCGGGCTG
TCGGCGCGGGGCACCCTGGGACTTGTAGTCCAAGCCGCTTGCCACCTGC
CGGCTGCAAACGGCGGAGGGACTACGAAGCCCAGAGGTCCCTGCGGCCC
TGCCCGCCCACCCGGACACCCCACCCCTTCCCCCTCCTTTCCGAAGCCC
CCCTCCCTGTTTTTTCAGCCCCCTC.

Embodiment 60: The engineered photoreceptor-specific regulatory element of any one of embodiments 46-48, or 60, wherein the ablation comprises a substitution or deletion of one or more nucleotides of the at least one nucleotide motif.

Embodiment 61: The engineered photoreceptor-specific regulatory element of any one of embodiments 46-48, 60, or 61, wherein the at least one nucleotide motif comprises a motif having a sequence within the nucleotide sequence of SEQ ID NO: 2.

Embodiment 62: The engineered photoreceptor-specific regulatory element of embodiment 62, wherein the ablation comprises a nucleotide substitution of the motif comprising the nucleotide sequence: CCCCCTGGCGGGCTGGCCCCGCCCCCGCGC (SEQ ID NO: 20).

Embodiment 63: The engineered photoreceptor-specific regulatory element of embodiment 62, wherein the ablation comprises a nucleotide substitution of the motif comprising the nucleotide sequence: CCGGCGAGGGGAGCCCGGCTCCAGGCCCCG (SEQ ID NO: 19).

Embodiment 64: The engineered photoreceptor-specific regulatory element of any one of embodiments 46-48, wherein the wild-type enhancer region comprises the nucleotide sequence:

(SEQ ID NO: 3)
CAGGCGGGGAGGGGCCCCGGGGGGGGGAGGGGGCCGGCCCGCGGGCCGC
GCAGCCGGAAGCCGGGACCGCCACCGGCCCCCGGCGAGGGGAGCCCGGC
TCCAGGCCCCGCCCCCTGGCGGGCTGGCCCCGCCCCCGCGCCGCGCCGC
GCGATCGGCCCGCGCCCATTGGCTCTCCGGCCCGCCGCTCACCGCCCCT
CCTCCGCACCGCCCCTACCCGCAGGCCGCGGCGGGCTGTCGGCGCGGGG
CACCCTGGGACTTGTAGTCCAAGCCGCTTGCCACCTGCCGGCTGCAAAC
GGCGGAGGGACTACGAAGCCCAGAGGTCCCTGCGGCCCTGCCCGCCCAC
CCGGACACCCCACCCCTTCCCCCTCCTTTCCGAAGCCCCCCTCCCTGTT
TTTTCAGCCCCCTCCCCCCCATCCCCCATGGAGC.

Embodiment 65: The engineered photoreceptor-specific regulatory element of any one of embodiments 46-48, or 65, wherein the ablation comprises a substitution or deletion of one or more nucleotides of the at least one nucleotide motif.

Embodiment 66: The engineered photoreceptor-specific regulatory element of any one of embodiments 46-48, 65, or 66, wherein the at least one nucleotide motif comprises a motif having a sequence within the nucleotide sequence of SEQ ID NO: 3.

Embodiment 67: The engineered photoreceptor-specific regulatory element of embodiment 67, wherein the ablation comprises a nucleotide substitution of the motif comprising the nucleotide sequence: AGCCCGGCTCCAGGCCCCGCCCCCTGGCGG (SEQ ID NO: 22).

Embodiment 68: The engineered photoreceptor-specific regulatory element of embodiment 67, wherein the ablation comprises a nucleotide substitution of the motif comprising the nucleotide sequence: GCTGGCCCCGCCCCCGCGCCGCGCCGCGCG (SEQ ID NO: 23).

Embodiment 69: The engineered photoreceptor-specific regulatory element of embodiment 67, wherein the ablation comprises a nucleotide substitution of the motif comprising the nucleotide sequence: CTACCCGCAGGCCGCGGCGGGCTGTCGGCG (SEQ ID NO: 24).

Embodiment 70: The engineered photoreceptor-specific regulatory element of embodiment 67, wherein the ablation comprises a nucleotide substitution of the motif comprising the nucleotide sequence: GCTTGCCACCTGCCGGCTGCAAACGGCGGA (SEQ ID NO: 26).

Embodiment 71: The engineered photoreceptor-specific regulatory element of embodiment 67, wherein the ablation comprises a nucleotide substitution of the motif comprising the nucleotide sequence: CCTGCCCGCCCACCCGGACACCCCACCCCT (SEQ ID NO: 27).

Embodiment 72: The engineered photoreceptor-specific regulatory element of embodiment 67, wherein the ablation comprises a nucleotide substitution of the motif comprising the nucleotide sequence: TCCCCCTCCTTTCCGAAGCCCCCCTCCCTG (SEQ ID NO: 29).

Embodiment 73: The engineered photoreceptor-specific regulatory element of embodiment 1, wherein the enhancer region comprises:
(i) at least one transcription factor binding site,
(ii) at least one nucleotide motif,
(iii) at least one ablation patch,
(iv) at least one transcription factor binding site array, or
(v) any combination thereof.

Embodiment 74: The engineered photoreceptor-specific regulatory element of embodiment 74, wherein the engineered photoreceptor-specific regulatory element comprises higher activity in photoreceptor cells of a mammalian retina as compared to non-photoreceptor cells of the same mammalian retina.

Embodiment 75: The engineered photoreceptor-specific regulatory element of embodiment 74 or embodiment 75, further comprising a linker sequence, wherein the linker sequence is operably linked to the enhancer region and the minimal promoter.

Embodiment 76: The engineered photoreceptor-specific regulatory element of any one of embodiments 74-76, wherein the linker sequence is selected from the group consisting of: SEQ ID NOs: 107-110.

Embodiment 77: The engineered photoreceptor-specific regulatory element of any one of embodiments 74-77, wherein the enhancer region comprises at least 75%, at least 80%, at least 85%, at least 90%, at least 95%, at least 96%, at least 97%, at least 98%, at least 99%, or 100% sequence identity to a wild-type sequence motif, ablation sequence motif, or regulatory element component nucleotide sequence selected from Table 2 or Table 4

Embodiment 78: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 164

Embodiment 79: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 165.

Embodiment 80: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 166.

Embodiment 81: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 167.

Embodiment 82: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 168.

Embodiment 83: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 169.

Embodiment 84: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 170.

Embodiment 85: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 171.

Embodiment 86: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 172.

Embodiment 87: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 173.

Embodiment 88: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 174.

Embodiment 89: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 175.

Embodiment 90: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 176.

Embodiment 91: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 177.

Embodiment 92: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 178.

Embodiment 93: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 179.

Embodiment 94: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 180.

Embodiment 95: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 181.

Embodiment 96: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 182.

Embodiment 97: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 183.

Embodiment 98: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 184.

Embodiment 99: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 185.

Embodiment 100: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 186.

Embodiment 101: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 187.

Embodiment 102: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 188.

Embodiment 103: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 189.

Embodiment 104: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 190.

Embodiment 105: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 191.

Embodiment 106: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 192.

Embodiment 107: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 193.

Embodiment 108: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 194.

Embodiment 109: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 195.

Embodiment 110: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 196.

Embodiment 111: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 197.

Embodiment 112: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 198.

Embodiment 113: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 199.

Embodiment 114: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 200.

Embodiment 115: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 201.

Embodiment 116: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 202.

Embodiment 117: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 203.

Embodiment 118: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 204.

Embodiment 119: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 205.

Embodiment 120: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 206.

Embodiment 121: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 207.

Embodiment 122: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 208.

Embodiment 123: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 209.

Embodiment 124: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 210.

Embodiment 125: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 211.

Embodiment 126: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 212.

Embodiment 127: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 213.

Embodiment 128: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 214.

Embodiment 129: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 215.

Embodiment 130: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 216.

Embodiment 131: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 217.

Embodiment 132: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 218.

Embodiment 133: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 219.

Embodiment 134: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 220.

Embodiment 135: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 221.

Embodiment 136: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 222.

Embodiment 137: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 223.

Embodiment 138: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 224.

Embodiment 139: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 225.

Embodiment 140: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 226.

Embodiment 141: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 227.

Embodiment 142: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 228.

Embodiment 143: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 229.

Embodiment 144: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 230.

Embodiment 145: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 231.

Embodiment 146: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 232.

Embodiment 147: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 233.

Embodiment 148: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 234.

Embodiment 149: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 235.

Embodiment 150: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 236.

Embodiment 151: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 237.

Embodiment 152: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 238.

Embodiment 153: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 239.

Embodiment 154: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 240.

Embodiment 155: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 241.

Embodiment 156: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 242.

Embodiment 157: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 243.

Embodiment 158: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 244.

Embodiment 159: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 245.

Embodiment 160: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 246.

Embodiment 161: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 247.

Embodiment 162: The engineered photoreceptor-specific regulatory element of any one of embodiments 75-162, wherein higher activity is characterized by at least 100-fold, at least 200-fold, at least 300-fold, at least 400-fold, at least 500-fold, at least 600-fold, at least 700-fold, at least 800-fold, at least 900-fold, at least 1000-fold, at least 1100-fold, at least 1200-fold, or at least 1300-fold greater expression of one or more genes operably linked to the engineered photoreceptor-specific regulatory element.

Embodiment 163: The engineered photoreceptor-specific regulatory element of any one of embodiments 74-163, wherein the non-photoreceptor cells comprise muller cells and retinal pigment epithelial cells.

Embodiment 164: An engineered photoreceptor-specific regulatory element comprising the polynucleotide sequence of SEQ ID NO: 138.

Embodiment 165: An engineered photoreceptor-specific regulatory element comprising the polynucleotide sequence of SEQ ID NO: 139.

Embodiment 166: An engineered photoreceptor-specific regulatory element comprising the polynucleotide sequence of SEQ ID NO: 140.

Embodiment 167: An engineered photoreceptor-specific regulatory element comprising the polynucleotide sequence of SEQ ID NO: 141.

Embodiment 168: An engineered photoreceptor-specific regulatory element comprising the polynucleotide sequence of SEQ ID NO: 142.

Embodiment 169: An engineered photoreceptor-specific regulatory element comprising the polynucleotide sequence of SEQ ID NO: 143.

Embodiment 170: An engineered photoreceptor-specific regulatory element comprising the polynucleotide sequence of SEQ ID NO: 144.

Embodiment 171: An engineered photoreceptor-specific regulatory element comprising the polynucleotide sequence of SEQ ID NO: 145.

Embodiment 172: An engineered photoreceptor-specific regulatory element comprising the polynucleotide sequence of SEQ ID NO: 146.

Embodiment 173: An engineered photoreceptor-specific regulatory element comprising the polynucleotide sequence of SEQ ID NO: 147.

Embodiment 174: An engineered photoreceptor-specific regulatory element comprising the polynucleotide sequence of SEQ ID NO: 148.

Embodiment 175: 176. An engineered photoreceptor-specific regulatory element comprising the polynucleotide sequence of SEQ ID NO: 149.

Embodiment 176: An engineered photoreceptor-specific regulatory element comprising the polynucleotide sequence of SEQ ID NO: 150.

Embodiment 177: An engineered photoreceptor-specific regulatory element comprising the polynucleotide sequence of SEQ ID NO: 151.

Embodiment 178: An engineered photoreceptor-specific regulatory element comprising the polynucleotide sequence of SEQ ID NO: 152.

Embodiment 179: An engineered photoreceptor-specific regulatory element comprising the polynucleotide sequence of SEQ ID NO: 153.

Embodiment 180: An engineered photoreceptor-specific regulatory element comprising the polynucleotide sequence of SEQ ID NO: 154.

Embodiment 181: An engineered photoreceptor-specific regulatory element comprising the polynucleotide sequence of SEQ ID NO: 155.

Embodiment 182: An engineered photoreceptor-specific regulatory element comprising the polynucleotide sequence of SEQ ID NO: 156.

Embodiment 183: An engineered photoreceptor-specific regulatory element comprising the polynucleotide sequence of SEQ ID NO: 157.

Embodiment 184: An engineered photoreceptor-specific regulatory element comprising the polynucleotide sequence of SEQ ID NO: 158.

Embodiment 185: An engineered photoreceptor-specific regulatory element comprising the polynucleotide sequence of SEQ ID NO: 159.

Embodiment 186: An engineered photoreceptor-specific regulatory element comprising the polynucleotide sequence of SEQ ID NO: 160.

Embodiment 187: An engineered photoreceptor-specific regulatory element comprising the polynucleotide sequence of SEQ ID NO: 161.

Embodiment 188: An engineered photoreceptor-specific regulatory element comprising the polynucleotide sequence of SEQ ID NO: 162.

Embodiment 189: An engineered photoreceptor-specific regulatory element comprising the polynucleotide sequence of SEQ ID NO: 163.

Embodiment 190: An engineered photoreceptor-specific regulatory element comprising the polynucleotide sequence of SEQ ID NO: 164.

Embodiment 191: An engineered photoreceptor-specific regulatory element comprising the polynucleotide sequence of SEQ ID NO: 165.

Embodiment 192: An engineered photoreceptor-specific regulatory element comprising the polynucleotide sequence of SEQ ID NO: 166.

Embodiment 193: An engineered photoreceptor-specific regulatory element comprising the polynucleotide sequence of SEQ ID NO: 167.

Embodiment 194: An engineered photoreceptor-specific regulatory element comprising the polynucleotide sequence of SEQ ID NO: 168.

Embodiment 195: An engineered photoreceptor-specific regulatory element comprising the polynucleotide sequence of SEQ ID NO: 169.

Embodiment 196: An engineered photoreceptor-specific regulatory element comprising the polynucleotide sequence of SEQ ID NO: 170.

Embodiment 197: An engineered photoreceptor-specific regulatory element comprising the polynucleotide sequence of SEQ ID NO: 171.

Embodiment 198: An engineered photoreceptor-specific regulatory element comprising the polynucleotide sequence of SEQ ID NO: 172.

Embodiment 199: An engineered photoreceptor-specific regulatory element comprising the polynucleotide sequence of SEQ ID NO: 173.

Embodiment 200: An engineered photoreceptor-specific regulatory element comprising the polynucleotide sequence of SEQ ID NO: 174.

Embodiment 201: An engineered photoreceptor-specific regulatory element comprising the polynucleotide sequence of SEQ ID NO: 175.

Embodiment 202: An engineered photoreceptor-specific regulatory element comprising the polynucleotide sequence of SEQ ID NO: 176.

Embodiment 203: An engineered photoreceptor-specific regulatory element comprising the polynucleotide sequence of SEQ ID NO: 177.

Embodiment 204: An engineered photoreceptor-specific regulatory element comprising the polynucleotide sequence of SEQ ID NO: 178.

Embodiment 205: An engineered photoreceptor-specific regulatory element comprising the polynucleotide sequence of SEQ ID NO: 179.

Embodiment 206: An engineered photoreceptor-specific regulatory element comprising the polynucleotide sequence of SEQ ID NO: 180.

Embodiment 207: An engineered photoreceptor-specific regulatory element comprising the polynucleotide sequence of SEQ ID NO: 181.

Embodiment 208: An engineered photoreceptor-specific regulatory element comprising the polynucleotide sequence of SEQ ID NO: 182.

Embodiment 209: An engineered photoreceptor-specific regulatory element comprising the polynucleotide sequence of SEQ ID NO: 183.

Embodiment 210: An engineered photoreceptor-specific regulatory element comprising the polynucleotide sequence of SEQ ID NO: 184.

Embodiment 211: 212. An engineered photoreceptor-specific regulatory element comprising the polynucleotide sequence of SEQ ID NO: 185.

Embodiment 212: An engineered photoreceptor-specific regulatory element comprising the polynucleotide sequence of SEQ ID NO: 186.

Embodiment 213: An engineered photoreceptor-specific regulatory element comprising the polynucleotide sequence of SEQ ID NO: 187.

Embodiment 214: An engineered photoreceptor-specific regulatory element comprising the polynucleotide sequence of SEQ ID NO: 188.

Embodiment 215: An engineered photoreceptor-specific regulatory element comprising the polynucleotide sequence of SEQ ID NO: 189.

Embodiment 216: An engineered photoreceptor-specific regulatory element comprising the polynucleotide sequence of SEQ ID NO: 190.

Embodiment 217: An engineered photoreceptor-specific regulatory element comprising the polynucleotide sequence of SEQ ID NO: 191.

Embodiment 218: An engineered photoreceptor-specific regulatory element comprising the polynucleotide sequence of SEQ ID NO: 192.

Embodiment 219: An engineered photoreceptor-specific regulatory element comprising the polynucleotide sequence of SEQ ID NO: 193.

Embodiment 220: An engineered photoreceptor-specific regulatory element comprising the polynucleotide sequence of SEQ ID NO: 194.

Embodiment 221: An engineered photoreceptor-specific regulatory element comprising the polynucleotide sequence of SEQ ID NO: 195.

Embodiment 222: An engineered photoreceptor-specific regulatory element comprising the polynucleotide sequence of SEQ ID NO: 196.

Embodiment 223: An engineered photoreceptor-specific regulatory element comprising the polynucleotide sequence of SEQ ID NO: 197.

Embodiment 224: An engineered photoreceptor-specific regulatory element comprising the polynucleotide sequence of SEQ ID NO: 198.

Embodiment 225: An engineered photoreceptor-specific regulatory element comprising the polynucleotide sequence of SEQ ID NO: 199.

Embodiment 226: An engineered photoreceptor-specific regulatory element comprising the polynucleotide sequence of SEQ ID NO: 200.

Embodiment 227: An engineered photoreceptor-specific regulatory element comprising the polynucleotide sequence of SEQ ID NO: 201.

Embodiment 228: An engineered photoreceptor-specific regulatory element comprising the polynucleotide sequence of SEQ ID NO: 202.

Embodiment 229: An engineered photoreceptor-specific regulatory element comprising the polynucleotide sequence of SEQ ID NO: 203.

Embodiment 230: An engineered photoreceptor-specific regulatory element comprising the polynucleotide sequence of SEQ ID NO: 204.

Embodiment 231: An engineered photoreceptor-specific regulatory element comprising the polynucleotide sequence of SEQ ID NO: 205.

Embodiment 232: An engineered photoreceptor-specific regulatory element comprising the polynucleotide sequence of SEQ ID NO: 206.

Embodiment 233: An engineered photoreceptor-specific regulatory element comprising the polynucleotide sequence of SEQ ID NO: 207.

Embodiment 234: An engineered photoreceptor-specific regulatory element comprising the polynucleotide sequence of SEQ ID NO: 208.

Embodiment 235: An engineered photoreceptor-specific regulatory element comprising the polynucleotide sequence of SEQ ID NO: 209.

Embodiment 236: An engineered photoreceptor-specific regulatory element comprising the polynucleotide sequence of SEQ ID NO: 210.

Embodiment 237: An engineered photoreceptor-specific regulatory element comprising the polynucleotide sequence of SEQ ID NO: 211.

Embodiment 238: An engineered photoreceptor-specific regulatory element comprising the polynucleotide sequence of SEQ ID NO: 212.

Embodiment 239: An engineered photoreceptor-specific regulatory element comprising the polynucleotide sequence of SEQ ID NO: 213.

Embodiment 240: An engineered photoreceptor-specific regulatory element comprising the polynucleotide sequence of SEQ ID NO: 214.

Embodiment 241: An engineered photoreceptor-specific regulatory element comprising the polynucleotide sequence of SEQ ID NO: 215.

Embodiment 242: An engineered photoreceptor-specific regulatory element comprising the polynucleotide sequence of SEQ ID NO: 216.

Embodiment 243: An engineered photoreceptor-specific regulatory element comprising the polynucleotide sequence of SEQ ID NO: 217.

Embodiment 244: An engineered photoreceptor-specific regulatory element comprising the polynucleotide sequence of SEQ ID NO: 218.

Embodiment 245: An engineered photoreceptor-specific regulatory element comprising the polynucleotide sequence of SEQ ID NO: 219.

Embodiment 246: An engineered photoreceptor-specific regulatory element comprising the polynucleotide sequence of SEQ ID NO: 220.

Embodiment 247: An engineered photoreceptor-specific regulatory element comprising the polynucleotide sequence of SEQ ID NO: 221.

Embodiment 248: An engineered photoreceptor-specific regulatory element comprising the polynucleotide sequence of SEQ ID NO: 222.

Embodiment 249: An engineered photoreceptor-specific regulatory element comprising the polynucleotide sequence of SEQ ID NO: 223.

Embodiment 250: An engineered photoreceptor-specific regulatory element comprising the polynucleotide sequence of SEQ ID NO: 224.

Embodiment 251: An engineered photoreceptor-specific regulatory element comprising the polynucleotide sequence of SEQ ID NO: 225.

Embodiment 252: An engineered photoreceptor-specific regulatory element comprising the polynucleotide sequence of SEQ ID NO: 226.

Embodiment 253: An engineered photoreceptor-specific regulatory element comprising the polynucleotide sequence of SEQ ID NO: 227.

Embodiment 254: An engineered photoreceptor-specific regulatory element comprising the polynucleotide sequence of SEQ ID NO: 228.

Embodiment 255: An engineered photoreceptor-specific regulatory element comprising the polynucleotide sequence of SEQ ID NO: 229.

Embodiment 256: An engineered photoreceptor-specific regulatory element comprising the polynucleotide sequence of SEQ ID NO: 230.

Embodiment 257: An engineered photoreceptor-specific regulatory element comprising the polynucleotide sequence of SEQ ID NO: 231.

Embodiment 258: An engineered photoreceptor-specific regulatory element comprising the polynucleotide sequence of SEQ ID NO: 232.

Embodiment 259: An engineered photoreceptor-specific regulatory element comprising the polynucleotide sequence of SEQ ID NO: 233.

Embodiment 260: An engineered photoreceptor-specific regulatory element comprising the polynucleotide sequence of SEQ ID NO: 234.

Embodiment 261: An engineered photoreceptor-specific regulatory element comprising the polynucleotide sequence of SEQ ID NO: 235.

Embodiment 262: An engineered photoreceptor-specific regulatory element comprising the polynucleotide sequence of SEQ ID NO: 236.

Embodiment 263: An engineered photoreceptor-specific regulatory element comprising the polynucleotide sequence of SEQ ID NO: 237.

Embodiment 264: An engineered photoreceptor-specific regulatory element comprising the polynucleotide sequence of SEQ ID NO: 238.

Embodiment 265: An engineered photoreceptor-specific regulatory element comprising the polynucleotide sequence of SEQ ID NO: 239.

Embodiment 266: An engineered photoreceptor-specific regulatory element comprising the polynucleotide sequence of SEQ ID NO: 240.

Embodiment 267: An engineered photoreceptor-specific regulatory element comprising the polynucleotide sequence of SEQ ID NO: 241.

Embodiment 268: An engineered photoreceptor-specific regulatory element comprising the polynucleotide sequence of SEQ ID NO: 242.

Embodiment 269: An engineered photoreceptor-specific regulatory element comprising the polynucleotide sequence of SEQ ID NO: 243.

Embodiment 270: An engineered photoreceptor-specific regulatory element comprising the polynucleotide sequence of SEQ ID NO: 244.

Embodiment 271: An engineered photoreceptor-specific regulatory element comprising the polynucleotide sequence of SEQ ID NO: 245.

Embodiment 272: An engineered photoreceptor-specific regulatory element comprising the polynucleotide sequence of SEQ ID NO: 246.

Embodiment 273: An engineered photoreceptor-specific regulatory element comprising the polynucleotide sequence of SEQ ID NO: 247.

Embodiment 274: A heterologous construct comprising the engineered photoreceptor-specific regulatory element according to any one of embodiments 1-274, or 319-324 operably linked to a polynucleotide, wherein the polynucleotide comprises a polynucleotide sequence encoding a polypeptide.

Embodiment 275: The heterologous construct of embodiment 275, wherein the polypeptide comprises at least one effector molecule.

Embodiment 276: The heterologous construct of embodiment 275 or embodiment 276, wherein the polypeptide comprises a first effector molecule and a second effector molecule.

Embodiment 277: The heterologous construct of any one of embodiments 275-276, wherein the polynucleotide comprises a polynucleotide sequence encoding the first effector molecule, a linker polynucleotide sequence, and a polynucleotide sequence encoding the second effector.

Embodiment 278: The heterologous construct of embodiment 278, wherein the linker polynucleotide sequence encodes one or more 2A ribosome skipping elements.

Embodiment 279: The heterologous construct of embodiment 279, wherein the one or more 2A ribosome skipping elements comprise elements that are each selected from the group consisting of: P2A, T2A, E2A, and F2A.

Embodiment 280: The heterologous construct of any one of embodiments 275-280, wherein the at least one effector molecule belongs to a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme.

Embodiment 281: The heterologous construct of embodiment 281, wherein each of the first effector molecule and the second effector molecule is from a separate therapeutic class.

Embodiment 282: The heterologous construct of any one of embodiments 275-282, wherein each of the at least one effector molecule is a human-derived effector molecule.

Embodiment 283: A vector comprising the heterologous construct according to any one of embodiments 275-283.

Embodiment 284: A dual expression vector comprising the heterologous construct according to any one of embodiments 275-283 and a second construct comprising a polynucleotide sequence encoding a second effector protein.

Embodiment 285: An photoreceptor cell comprising the heterologous construct according to any one of embodiments 275-283, the vector according to embodiment 284, or the dual expression vector according to embodiment 285.

Embodiment 286: The photoreceptor cell of embodiment 286, wherein the photoreceptor cell is a rod cell or a cone cell.

Embodiment 287: The photoreceptor cell of embodiment 287, wherein the photoreceptor cells is a cone cell.

Embodiment 288: The photoreceptor cell of embodiment 287, wherein the photoreceptor cell is a rod cell.

Embodiment 289: The photoreceptor cell of any one of embodiments 286-289, wherein the photoreceptor cell expresses at least one effector molecule.

Embodiment 290: A pharmaceutical composition comprising the engineered photoreceptor-specific regulatory element according to any one of embodiments 1-274 or 319-324, the heterologous construct according to any one of embodiments 275-283, the vector of embodiment 284, the dual expression vector according to embodiment 281, or the photoreceptor cell according to any one of embodiments 286-290, and a pharmaceutically acceptable carrier, pharmaceutically acceptable excipient, or a combination thereof.

Embodiment 291: A method of increasing expression of a target gene, the method comprising use of the engineered photoreceptor-specific regulatory element of any one of embodiments 1-274 or 319-324, the heterologous construct according to any one of embodiments 275-283, the vector of embodiment 284, the dual expression vector according to embodiment 285, or the photoreceptor cell according to any one of embodiments 286-290, to increase expression of the target gene.

Embodiment 292: A method of treating a subject in need thereof, the method comprising administering the engineered photoreceptor-specific regulatory element according to any one of embodiments 1-274 or 319-324, the heterologous construct according to any one of embodiments 275-283, the vector of embodiment 284, or the pharmaceutical composition according to embodiment 291.

Embodiment 293: A method of treating a subject having an ocular disease or disorder, the method comprising administering the engineered photoreceptor-specific regulatory element according to any one of embodiments 1-274 or 319-324, the heterologous construct according to any one of embodiments 275-283, the vector of embodiment 284, or the pharmaceutical composition according to embodiment 291 to the subject in need thereof.

Embodiment 294: A kit for treating and/or preventing a tumor, comprising the pharmaceutical composition according to embodiment 291.

Embodiment 295: The kit according to embodiment 295, wherein the kit further comprises written instructions for using the pharmaceutical composition for treating and/or preventing a tumor in a subject.

Embodiment 296: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 248.

Embodiment 297: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 259.

Embodiment 298: The engineered photoreceptor-specific regulatory element of embodiment 297 or embodiment 298, wherein the enhancer region further comprises (b) a second enhancer segment.

Embodiment 299: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 249;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 250.

Embodiment 300: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 251;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 252.

Embodiment 301: An engineered photoreceptor-specific regulatory element comprising:
an enhancer region, wherein the enhancer region comprises: a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 253;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 254.

Embodiment 302: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 255;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 256.

Embodiment 303: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 257;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 258.

Embodiment 304: An engineered photoreceptor-specific regulatory element comprising an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 260;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 261.

Embodiment 305: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 262;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 263.

Embodiment 306: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 264;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 265.

Embodiment 307: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 266;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 267.

Embodiment 308: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 268;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 269.

Embodiment 309: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 270;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 271.

Embodiment 310: An engineered photoreceptor-specific regulatory element comprising: an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 272;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 273.

Embodiment 311: 312. An engineered photoreceptor-specific regulatory element comprising:
(a) an enhancer region, wherein the enhancer region comprises:
a first enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 274;
a second enhancer segment comprising a nucleotide sequence with at least 75%, at least 80%, at least 85%, at least 90%, at least 95%, at least 96%, at least 97%, at least 98%, at least 99%, or 100% sequence identity to SEQ ID NO: 275.

Embodiment 312: The engineered photoreceptor-specific regulatory element of any one of embodiments 297-312, wherein the engineered photoreceptor-specific regulatory element exhibits greater activity in photoreceptor cells of a mammalian retina as compared to non-photoreceptor cells of the same mammalian retina.

Embodiment 313: The engineered photoreceptor-specific regulatory element of any one of embodiments 297-313, wherein the first enhancer segment and the second enhancer segment are contiguous.

Embodiment 314: The engineered photoreceptor-specific regulatory element of any one of embodiments 297-313, wherein the first enhancer segment and the second enhancer segment are non-contiguous.

Embodiment 315: The engineered photoreceptor-specific regulatory element of any one of embodiments 297-315, wherein the enhancer region further comprising an ablation motif.

Embodiment 316: The engineered photoreceptor-specific regulatory element of embodiment 316, wherein the ablation motif is selected from any ablation motif shown in Table 2.

Embodiment 317: The engineered photoreceptor-specific regulatory element of any one of embodiments 297-315, wherein a nucleotide sequence of about 1, about 5, about 10, about 15, about 20, about 30, about 40, about 50, about 60, about 70, about 80, about 90, about 100, about 120, about 140, about 160, about 180, or about 200 nucleotides is between the first enhancer segment and the second enhancer segment.

Embodiment 318: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 276.

Embodiment 319: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 277.

Embodiment 320: The engineered photoreceptor-specific regulatory element of any one of embodiments 74-78, wherein engineered photoreceptor-specific regulatory element comprises at least 75%, at least 80%, at least 85%, at least 90%, at least 95%, at least 96%, at least 97%, at least 98%, at least 99%, or 100% sequence identity to SEQ ID NO: 278.

Embodiment 321: An engineered photoreceptor-specific regulatory element comprising the polynucleotide sequence of SEQ ID NO: 276.

Embodiment 322: An engineered photoreceptor-specific regulatory element comprising the polynucleotide sequence of SEQ ID NO: 277.

Embodiment 323: An engineered photoreceptor-specific regulatory element comprising the polynucleotide sequence of SEQ ID NO: 278.

SEQUENCE LISTING

| SEQ | | |
|---|---|---|
| ID NO | Name | Sequence |
| 1 | Native Enhancer | TGTTGAGAGCTCAAGCTCTTTTTAACGCTTTGCCTTGCTGTCTCCAA |
| | Sequence 1 | AGTATTGCCTTCATCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGG |
| 2 | Native Enhancer | GGTTACACAACCAGGCGGGGAGGGGCCCCGGGGCGGGGAGGGGGC |
| | Sequence 2 | CGGCCCGCGGGCCGCGCAGCCGGAAGCCGGGACCGCCACCGGCCC |
| | | CCGGCGAGGGGAGCCCGGCTCCAGGCCCCGCCCCCTGGCGGGCTGG |
| | | CCCCGCCCCCGCGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TC |
| 3 | Native Enhancer | CAGGCGGGGAGGGGCCCCGGGGGGGGGAGGGGGCCGGCCCGCGGG |
| | Sequence 3 | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCCCTGCCCGCCCACCCGGACACCCCACCCCTTCCCCC |
| | | TCCTTTCCGAAGCCCCCCTCCCTGTTTTTTCAGCCCCCTCCCCCCCAT |
| | | CCCCCATGGAGC |
| 4 | Native Enhancer | AGCACGCGCGAGCTGTCACCAGGGCGCGAGACAACCGAACACGGC |
| | Sequence 4 | CCCGCCCCCTGCCCGTCTATTGGCCCGCCCGCTGGAGCCCCGCCCCT |
| | | CAGGCCCTGCATTGCGCCAACGGCGCAGCGCTGGGCCGCGACCCCG |
| | | GCACCGGCACCCGTTCCGAGGGTTCGCGCCGCAGGCGCAAAGTTTA |
| | | GGGTCAGCCACAAACGCGCGGCCGCTTTGAGCCTGGCGTAAGGGGC |
| | | GGCGGCGTAGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTC |
| 5 | WT Sequence Motif | TGTTGAGAGCTCAAGCTCTTTTTAACGCTT |
| 6 | Ablation Sequence | TTACCTACTAGGTTAATTCGTCCGATAGAT |
| | Motif | |
| 7 | WT Sequence Motif | TGCCTTGCTGTCTCCAAAGTATTGCCTTCA |
| 8 | Ablation Sequence | ACTACGAATTGCGAGCTTCTAAGTCCAATT |
| | Motif | |
| 9 | WT Sequence Motif | TCCTCATAGTTCAAAGTGTCCACCATCACA |
| 10 | Ablation Sequence | TTCGGTATTCGAGTCGTCAGACTCAATTAT |
| | Motif | |
| 11 | WT Sequence Motif | TTCACGTTTCAGCCAATAGGAAGAAGGAAA |
| 12 | Ablation Sequence | TACACGGTTAAGACTCAATCCGGTAAGTAA |
| | Motif | |
| 13 | WT Sequence Motif | GAGAAAAACAGGAAAAGAGTTTACATGCCA |
| 14 | Ablation Sequence | TACTGTGATCAGGTGTCTACAAGACTAGTA |
| | Motif | |
| 15 | WT Sequence Motif | CTTTATTTCTTTTTTCTTTTCTTTCTTTTT |
| 16 | Ablation Sequence | ACTATCACGTATATACCAGCGAGTTCGATA |
| | Motif | |
| 17 | WT Sequence Motif | TTTAGACGG |
| 18 | Ablation Sequence | ATACACTCT |
| | Motif | |
| 19 | WT Sequence Motif | CCGGCGAGGGGAGCCCGGCTCCAGGCCCCG |
| 20 | WT Sequence Motif | CCCCCTGGCGGGCTGGCCCCGCCCCCGCGC |
| 21 | Ablation Sequence | TGCCTAAAACGAGATTCTGTACGATAAAGC |
| | Motif | |
| 22 | WT Sequence Motif | AGCCCGGCTCCAGGCCCCGCCCCCTGGCGG |
| 23 | WT Sequence Motif | GCTGGCCCCGCCCCCGCGCCGCGCCGCGCG |
| 24 | WT Sequence Motif | CTACCCGCAGGCCGCGGCGGGCTGTCGGCG |
| 25 | Ablation Sequence | TACGGTCTCGCTAATAGGAGTAAGATACAT |
| | Motif | |
| 26 | WT Sequence Motif | GCTTGCCACCTGCCGGCTGCAAACGGCGGA |
| 27 | WT Sequence Motif | CCTGCCCGCCCACCCGGACACCCCACCCCT |
| 28 | Ablation Sequence | TGTTAAGCATACTAAACTGTAAAGAAGCGA |
| | Motif | |
| 29 | WT Sequence Motif | TCCCCCTCCTTTCCGAAGCCCCCCTCCCTG |
| 30 | Ablation Sequence | GTTTCGAGCGACGCTTAATATTGCTAGATA |
| | Motif | |
| 31 | T-34 | AGCGCTGACAGATGGTTCACGG |
| 32 | T-3 | GGGCCAGACAGATGGTTCCCAATAC |
| 33 | SB05201 | TGTTGAGAGCTCAAGCTCTTTTTAACGCTT |
| 34 | SB05201 | CTTTATTTCTTTTTTCTTTTCTTTCTTTTT |
| 35 | Ablation patch | ACTATCACGTATATACCAGCGAGTTCGATA |
| 36 | SB05201 | TTTAGACGG |
| 37 | Ablation patch | ATACACTCT |
| 38 | Ablation patch | TTACCTACTAGGTTAATTCGTCCGATAGAT |
| 39 | SB05201 | TGCCTTGCTGTCTCCAAAGTATTGCCTTCA |
| 40 | Ablation patch | ACTACGAATTGCGAGCTTCTAAGTCCAATT |
| 41 | SB05201 | TCCTCATAGTTCAAAGTGTCCACCATCACA |
| 42 | Ablation patch | TTCGGTATTCGAGTCGTCAGACTCAATTAT |
| 43 | SB05201 | TTCACGTTTCAGCCAATAGGAAGAAGGAAA |
| 44 | Ablation patch | TACACGGTTAAGACTCAATCCGGTAAGTAA |
| 45 | SB05201 | GAGAAAAACAGGAAAAGAGTTTACATGCCA |
| 46 | Ablation patch | TACTGTGATCAGGTGTCTACAAGACTAGTA |
| 47 | SB05201 | CTCCTGCTATTGGTCAGAATGTGTCACGTG |
| 48 | SB05201 | ACCATACTCAGCTGCAAGGGTTGGTGGGAA |
| 49 | SB05201 | ATGTAGTCTTTTTTTCTGGGAGGCCATGTG |
| 50 | SB05201 | TTCGGCTTAAAATTGTCTTTTTTTCTTTTT |
| 51 | SB05227 | AGTAGCATGGTTGGAGCTTAACCCCAAGTC |
| 52 | SB05227 | TAATTTTATTCTGCCTCTTTATTTAAAAAG |
| 53 | SB05227 | GCAAGGAAGATTATTTTGAGTCACTGCACC |
| 54 | SB05227 | CTTTCTGAACAATAGCTGTTACCAAGGTCT |
| 55 | SB05227 | CAGGAGCATTAGGCGCTCAATTCTATTTTA |
| 56 | SB05228 | ACGCTATCACAGAGAGCCTCTTAGAAGCAC |
| 57 | SB05228 | GCTCGCATCCCCAGCACGTTAAATTACCCA |
| 58 | SB05228 | AGAAATGTCTCTGGCCAAACCTTTCAAGTA |
| 59 | SB05228 | ACGTGCAGACAAGAGAAAATCAGAG |
| 60 | SB05228 | GAAACAAAAATTTAAAAATAGCTCTACAAG |
| 61 | SB05228 | GGTAACCCGATTAGTCAGGCACCTCTTTCT |
| 62 | SB05228 | CCTCAAATGACCTTGAGGTTGTAAATACGA |
| 63 | SB05228 | TCTGTGGATGCCCCCTGGTGGCCCTAAATA |
| 64 | SB05228 | GAATGGCTTCAATGGATGGAAATTTAACGT |
| 65 | SB05229 | GGTTGAAGCACAGGAGGGTAAATAAGAAGG |
| 66 | SB05229 | TTTCCAGACAAAGAGACTCTTAAAGGTACA |
| 67 | SB05229 | GACTCTTATGTCTGTCCCTCCTCCTTAAAG |
| 68 | SB05229 | GGCCAGAGACGCTCCCGAGCCCATCTC |
| 69 | SB05229 | CTCAGGCCCTGCATTGCGCCAACGGCGCAG |
| 70 | SB05229 | CGCTGGGCCGCGACCCCGGCACCGGCACCC |
| 71 | SB05229 | GTTCCGAGGGTTCGCGCCGCAGGCGCAAAG |
| 72 | Ablation patch | CCACATTGCTATAGTGCTGTATAGCGATTA |
| 73 | SB05229 | TTGAGCCTGGCGTAAGGGGCGGCGGCGTAG |
| 74 | SB05229 | GGGGACGTTGTGAATAGCTGGTCGAAGTTT |
| 75 | SB05241 | GGTTACACAACCAGGCGGGGAGGGGCCCCG |
| 76 | SB05241 | TAGTCCAAGCCGCTTGCCACCTGCCGGCTG |
| 77 | SB05241 | CAAACGGCGGAGGGACTACGAAGCCCAGAG |
| 78 | SB05241 | GTCCCTGCGGCCCTGCCCGCCCACCCGGAC |
| 79 | SB05241 | ACCCCACCCCTTCCCCCTCCTTTCCGAAGC |
| 80 | SB05241 | CCCCCTCCCTGTTTTTTCAGCCCCCTC |
| 81 | SB05241 | GGGCGGGGAGGGGGCCGGCCCGCGGGCCGC |
| 82 | SB05241 | GCAGCCGGAAGCCGGGACCGCCACCGGCCC |
| 83 | SB05241 | CCGGCGAGGGGAGCCCGGCTCCAGGCCCCG |
| 84 | Ablation patch | TGCCTAAAACGAGATTCTGTACGATAAAGC |
| 85 | SB05241 | CGCGCCGCGCGATCGGCCCGCGCCCATTGG |
| 86 | SB05241 | CTCTCCGGCCCGCCGCTCACCGCCCCTCCT |
| 87 | SB05241 | CCGCACCGCCCCTACCCGCAGGCCGCGGCG |
| 88 | SB05241 | GGCTGTCGGCGCGGGGCACCCTGGGACTTG |
| 89 | SB05245 | CAGGCGGGGAGGGGCCCCGGGGCGGGGAGG |
| 90 | SB05245 | GCTTGCCACCTGCCGGCTGCAAACGGCGGA |
| 91 | SB05245 | GGGACTACGAAGCCCAGAGGTCCCTGCGGC |
| 92 | SB05245 | TCCCCCTCCTTTCCGAAGCCCCCCTCCCTG |
| 93 | SB05245 | TTTTTTCAGCCCCCTCCCCCCCATCCCCCA |
| 94 | SB05245 | TGGAGC |
| 95 | SB05245 | GGGCCGGCCCGCGGGCCGCGCAGCCGGAAG |
| 96 | SB05245 | CCGGGACCGCCACCGGCCCCCGGCGAGGGG |
| 97 | SB05245 | AGCCCGGCTCCAGGCCCCGCCCCCTGGCGG |
| 98 | SB05245 | ATCGGCCCGCGCCCATTGGCTCTCCGGCCC |
| 99 | SB05245 | GCCGCTCACCGCCCCTCCTCCGCACCGCCC |
| 100 | SB05245 | CGGGGCACCCTGGGACTTGTAGTCCAAGCC |
| 101 | SB05251 | TGGGGTGAGTCCTGCTCCTTTGTTCTTCCC |
| 102 | SB05251 | GGTCCCTCCCTACCTCTGCCCCGCGCTCTG |
| 103 | SB05251 | CCTTTGATCCTCTGCTCGGCTCTGAGCCAT |
| 104 | SB05276 | TCGAGGCGCCGGCGGGGCGGCCCGGCGGCC |
| 105 | SB05276 | GGAAGTGCACCCTGCTCGGGCTTTGCGCCC |
| 106 | SB05276 | CCGGCAGCTTCCTCCGCCCGCGAGG |
| 107 | Spacer 1 | CCTGCAGG |
| 108 | Spacer 2 | TAGTAAGGTA |
| 109 | Spacer 3 | TTTGCGCGTA |
| 110 | Spacer 4 | AGTCCGGGTA |
| 111 | T-34 | ATTCGTCTAGTACTAATCCTCTCGGCAGCCGATCTAATCCTCAGTCT |
| | | AGCGCTGACAGATGGTTCACGGGTGGA |
| 112 | T-13 | ACCATCTGTCAATCGGCTTAACCATCTGTCAAGTATCTATACCATCT |
| | | GTC |
| 113 | T-13 | ACCATCTGTCAAGTATCTATACCATCTGTC |
| 114 | T-13 | ACCATCTGTC |
| 115 | T-3 | TACGGGCCAGACAGATGGTTCCCAATACGACTAATCCTCATAGAAG |
| | | ACGCACTAATCCTC |
| 116 | T-34 | TAGTACTAATCCTCTCGGCAGCCGATCTAATCCTCAGTCTAGCGCTG |
| | | ACAGATGGTTCAC |
| 117 | T-34 | CTAATCCTCTCGGCCCGATCTAATCCTCAGTCTCGCTGACAGATGGT |
| 118 | T-3 | GACAGATGGTTCCCAACGACTAATCCTCATAGAACGCACTAATCCT |
| | | C |
| 119 | T-34 | CTAAGCCACATTCGTCTAGTACTAATCCTCTCGGCAGCCGATCTAAT |
| | | CCTCAGTCTAGCGCTGACAGATGGTTCACGGGTGGATTAATTGGTG |
| | | TTCCGAACA |
| 120 | T-3 | GTGAGTGACAGGCGACCAGTACTATAAATAGATACGGGCCAGACA |
| | | GATGGTTCCCAATACGACTAATCCTCATAGAAGACGCACTAATCCT |
| | | C |
| 121 | T-34 | CTAATCCTCTCGGCCGATCTAATCCTCAGTCTGCTGACAGATGGT |
| 122 | YB-TATA | TCTAGAGGGTATATAATGGGGGCCA |
| 123 | minTK | TTCGCATATTAAGGTGACGCGTGTGGCCTCGAACACCGAGCGACCC |
| | | TGCAGCGACCCGCTTAA |
| 124 | minIL2.2 | CAGAATTAACAGTATAAATTGCATCTCTTGTTCAAGAGTTCCCTATC |
| | | ACTCT |
| 125 | SMP | AAAATGTGCGCATGTGCAGCCATTGCCTGGGACGCATGCGTAGGGA |
| | | GCCGCGCGACAAACTGAGCCATTGCGGCAAGACTAGCGCAGAGAG |
| | | GAGAGGGAGCCGGAGATGCCAGACGCTTGGTTCTGAGGAGTGATTT |
| | | GCAACGCAATGGAGCGAGGAAGG |
| 126 | lateADE | AGACGCTAGCGGGGGGCTATAAAAGGGGGTGGGGGCGTTCGTCCTC |
| | | ACTCT |
| 127 | minCMV | TAGGCGTGTACGGTGGGAGGCCTATATAAGCAGAGCTCGTTTAGTG |
| | | AACCGTCAGATCGCCTGGA |
| 128 | MPRA screen | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| 129 | MPRA screen | AAGGTCATGACCTTAAAACCATGCTGACTCAGCATGCACGCATTGT |
| | | GGCTTAGAATTGCGCCTATCTAATCCTCTATCAGACAGAACCATCTG |
| | | TC |
| 130 | MPRA screen | GACAGATGGTTTGTCGAGTCACTAATCCTCATCCTTAGAGAAGAGG |
| | | ATTAGTTGAACAGTATGCTGACTCAGCAAGTGTCGCAACCATCTGT |
| | | C |
| 131 | MPRA screen | GTGAGTGACAGGCGAACCAGTACTATAAATAGATACGGGCCAGAC |
| | | AGATGGTTCCCAATACGACTAATCCTCATAGAAGACGCACTAATCC |
| | | TC |
| 132 | MPRA screen | GACAGATGGTTTTGACGCCTTGTGGCTTAGAGGCACATCATGCTGA |
| | | GTCAGCAACCGACGGCTGAGGATTAGTTTGCGAGTGTACCATCTGT |
| | | C |
| 133 | MPRA screen | CTAAGCCACATTCGTCTAGTACTAATCCTCTCGGCAGCCGATCTAAT |
| | | CCTCAGTCTAGCGCTGACAGATGGTTCACGGGTGGATTAATTGG |
| 134 | MPRA screen | TGCTGACTCAGCAACGACTATAAGTGGCTTAGAACATAACGTCTCT |
| | | AATCCTCAGACTGCGCGTGACAGATGGTTTCGTCCCGTACCATCTGT |
| | | C |
| 135 | MPRA screen | TGCTGAGTCAGCATAGGAAGAGTCTAAGCCACAGCACGCGTAGTCT |
| | | AATCCTCAGCCTACTTAACTATAAATAGATCGAGCAATACCATCTGT |
| | | C |
| 136 | MPRA screen | ACTATAAATAGAAACTACAACTGAGGATTAGAGTACACACTTGCTG |
| | | AGTCAGCAAATCTTTAGTCTAAGCCACTTAGATCGCCAACCATCTGT |
| | | C |
| 137 | MPRA screen | CTAAGCCACAGTAACATGCGACTAATCCTCTCTGTCTACAGAGTGG |
| | | CTTAGTCTATCGTGTTGCTGACTCAGCATTAGATAGTGACAGATGGT |
| 279 | MPRA screen | GTTAATCCCCTAGCTTAGATTCTATTTATAGTTATTACTATGC |
| | | TGACTCAGCAATAGACGGTAGAGGATTAGAGCACTCGTTTGA |
| | | CAGATGGT |
| 280 | MPRA screen | GTGGCTTAGAAAGGTCCGATGCTGAGTCAGCAAGCAAGTACT |
| | | CTAATCCTCTATAGGACAAACTATAAATAGAATACGAGGAGT |
| | | TAATCCCCT |
| 138 | Native Enhancer & | TGTTGAGAGCTCAAGCTCTTTTTAACGCTTTGCCTTGCTGTCTCCAA |
| | Promoter Regulatory | AGTATTGCCTTCATCCTCATAGTTCAAAGTGTCCACCATCACATTCA |
| | Element-SB05201 | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTCTAGAGGGTATA |
| | | TAATGGGGGCCA |
| 139 | Native Enhancer & | GGTTACACAACCAGGCGGGGAGGGGCCCCGGGGCGGGGAGGGGGC |
| | Promoter Regulatory | CGGCCCGCGGGCCGCGCAGCCGGAAGCCGGGACCGCCACCGGCCC |
| | Element-SB05241 | CCGGCGAGGGGAGCCCGGCTCCAGGCCCCGCCCCCTGGCGGGCTGG |
| | | CCCCGCCCCCGCGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGTCTAGAGGGTATATAATGGGGGCCA |
| 140 | Native Enhancer & | CAGGCGGGGAGGGGCCCCGGGGGGGGGAGGGGGCCGGCCCGCGGG |
| | Promoter Regulatory | CCGCGCAGCCGGAAGCCGGGACCGCCACCGGCCCCCGGCGAGGGG |
| | Element-SB05245 | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCCCTGCCCGCCCACCCGGACACCCCACCCCTTCCCCC |
| | | TCCTTTCCGAAGCCCCCCTCCCTGTTTTTTCAGCCCCCTCCCCCCCAT |
| | | CCCCCATGGAGCCCTGCAGGTCTAGAGGGTATATAATGGGGGCCA |
| 141 | Native Enhancer & | TGTTGAGAGCTCAAGCTCTTTTTAACGCTTTGCCTTGCTGTCTCCAA |
| | Promoter Regulatory | AGTATTGCCTTCATCCTCATAGTTCAAAGTGTCCACCATCACATTCA |
| | Element-SB07596 | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTTCGCATATTAAG |
| | | GTGACGCGTGTGGCCTCGAACACCGAGCGACCCTGCAGCGACCCGC |
| | | TTAA |
| 142 | Native Enhancer & | TGTTGAGAGCTCAAGCTCTTTTTAACGCTTTGCCTTGCTGTCTCCAA |
| | Promoter Regulatory | AGTATTGCCTTCATCCTCATAGTTCAAAGTGTCCACCATCACATTCA |
| | Element-SB07600 | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGAGACGCTAGCGG |
| | | GGGGCTATAAAAGGGGGTGGGGGCGTTCGTCCTCACTCT |
| 143 | Native Enhancer & | AGCACGCGCGAGCTGTCACCAGGGCGCGAGACAACCGAACACGGC |
| | Promoter Regulatory | CCCGCCCCCTGCCCGTCTATTGGCCCGCCCGCTGGAGCCCCGCCCCT |
| | Element-SB07623 | CAGGCCCTGCATTGCGCCAACGGCGCAGCGCTGGGCCGCGACCCCG |
| | | GCACCGGCACCCGTTCCGAGGGTTCGCGCCGCAGGCGCAAAGTTTA |
| | | GGGTCAGCCACAAACGCGCGGCCGCTTTGAGCCTGGCGTAAGGGGC |
| | | GGCGGCGTAGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTCCCTGCAGGAAAATGTGCGCATGTGCA |
| | | GCCATTGCCTGGGACGCATGCGTAGGGAGCCGCGCGACAAACTGAG |
| | | CCATTGCGGCAAGACTAGCGCAGAGAGGAGAGGGAGCCGGAGATG |
| | | CCAGACGCTTGGTTCTGAGGAGTGATTTGCAACGCAATGGAGCGAG |
| | | GAAGG |
| 144 | Native Enhancer & | AGCACGCGCGAGCTGTCACCAGGGCGCGAGACAACCGAACACGGC |
| | Promoter Regulatory | CCCGCCCCCTGCCCGTCTATTGGCCCGCCCGCTGGAGCCCCGCCCCT |
| | Element-SB07624 | CAGGCCCTGCATTGCGCCAACGGCGCAGCGCTGGGCCGCGACCCCG |
| | | GCACCGGCACCCGTTCCGAGGGTTCGCGCCGCAGGCGCAAAGTTTA |
| | | GGGTCAGCCACAAACGCGCGGCCGCTTTGAGCCTGGCGTAAGGGGC |
| | | GGCGGCGTAGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTCCCTGCAGGAGACGCTAGCGGGGGGCT |
| | | ATAAAAGGGGGTGGGGGCGTTCGTCCTCACTCT |
| 145 | Native Enhancer & | AGCACGCGCGAGCTGTCACCAGGGCGCGAGACAACCGAACACGGC |
| | Promoter Regulatory | CCCGCCCCCTGCCCGTCTATTGGCCCGCCCGCTGGAGCCCCGCCCCT |
| | Element-SB07625 | CAGGCCCTGCATTGCGCCAACGGCGCAGCGCTGGGCCGCGACCCCG |
| | | GCACCGGCACCCGTTCCGAGGGTTCGCGCCGCAGGCGCAAAGTTTA |
| | | GGGTCAGCCACAAACGCGCGGCCGCTTTGAGCCTGGCGTAAGGGGC |
| | | GGCGGCGTAGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTCCCTGCAGGTAGGCGTGTACGGTGGGA |
| | | GGCCTATATAAGCAGAGCTCGTTTAGTGAACCGTCAGATCGCCTGG |
| | | A |
| 146 | Native Enhancer & | GGTTACACAACCAGGCGGGGAGGGGCCCCGGGGGGGGGAGGGGGC |
| | Promoter Regulatory | CGGCCCGCGGGCCGCGCAGCCGGAAGCCGGGACCGCCACCGGCCC |
| | Element-SB07632 | CCGGCGAGGGGAGCCCGGCTCCAGGCCCCGCCCCCTGGCGGGCTGG |
| | | CCCCGCCCCCGCGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGTTCGCATATTAAGGTGACGCGTGTGGCCTCGAACAC |
| | | CGAGCGACCCTGCAGCGACCCGCTTAA |
| 147 | Native Enhancer & | GGTTACACAACCAGGCGGGGAGGGGCCCCGGGGCGGGGAGGGGGC |
| | Promoter Regulatory | CGGCCCGCGGGCCGCGCAGCCGGAAGCCGGGACCGCCACCGGCCC |
| | Element-SB07634 | CCGGCGAGGGGAGCCCGGCTCCAGGCCCCGCCCCCTGGCGGGCTGG |
| | | CCCCGCCCCCGCGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGCAGAATTAACAGTATAAATTGCATCTCTTGTTCAAGA |
| | | GTTCCCTATCACTCT |
| 148 | Native Enhancer & | GGTTACACAACCAGGCGGGGAGGGGCCCCGGGGGGGGGAGGGGGC |
| | Promoter Regulatory | CGGCCCGCGGGCCGCGCAGCCGGAAGCCGGGACCGCCACCGGCCC |
| | Element-SB07636 | CCGGCGAGGGGAGCCCGGCTCCAGGCCCCGCCCCCTGGCGGGCTGG |
| | | CCCCGCCCCCGCGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGAGACGCTAGCGGGGGGCTATAAAAGGGGGTGGGGG |
| | | CGTTCGTCCTCACTCT |
| 149 | Ablation Regulatory | TTACCTACTAGGTTAATTCGTCCGATAGATTGCCTTGCTGTCTCCAA |
| | Element-SB07140 | AGTATTGCCTTCATCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTCTAGAGGGTATA |
| | | TAATGGGGGCCA |
| 150 | Ablation Regulatory | TGTTGAGAGCTCAAGCTCTTTTTAACGCTTACTACGAATTGCGAGCT |
| | Element-SB07141 | TCTAAGTCCAATTTCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTCTAGAGGGTATA |
| | | TAATGGGGGCCA |
| 151 | Ablation Regulatory | TGTTGAGAGCTCAAGCTCTTTTTAACGCTTTGCCTTGCTGTCTCCAA |
| | Element-SB07142 | AGTATTGCCTTCATTCGGTATTCGAGTCGTCAGACTCAATTATTTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTCTAGAGGGTATA |
| | | TAATGGGGGCCA |
| 152 | Ablation Regulatory | TGTTGAGAGCTCAAGCTCTTTTTAACGCTTTGCCTTGCTGTCTCCAA |
| | Element-SB07143 | AGTATTGCCTTCATCCTCATAGTTCAAAGTGTCCACCATCACATACA |
| | | CGGTTAAGACTCAATCCGGTAAGTAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTCTAGAGGGTATA |
| | | TAATGGGGGCCA |
| 153 | Ablation Regulatory | TGTTGAGAGCTCAAGCTCTTTTTAACGCTTTGCCTTGCTGTCTCCAA |
| | Element-SB07144 | AGTATTGCCTTCATCCTCATAGTTCAAAGTGTCCACCATCACATTCA |
| | | CGTTTCAGCCAATAGGAAGAAGGAAATACTGTGATCAGGTGTCTAC |
| | | AAGACTAGTACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTTTTAGACGGCCTGCAGGTCTAGAGGGTATA |
| | | TAATGGGGGCCA |
| 154 | Ablation Regulatory | TGTTGAGAGCTCAAGCTCTTTTTAACGCTTTGCCTTGCTGTCTCCAA |
| | Element-SB07149 | AGTATTGCCTTCATCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTACTATCACGTA |
| | | TATACCAGCGAGTTCGATATTTAGACGGCCTGCAGGTCTAGAGGGT |
| | | ATATAATGGGGGCCA |
| 155 | Ablation Regulatory | TGTTGAGAGCTCAAGCTCTTTTTAACGCTTTGCCTTGCTGTCTCCAA |
| | Element-SB07150 | AGTATTGCCTTCATCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTTATACACTCTCCTGCAGGTCTAGAGGGTATA |
| | | TAATGGGGGCCA |
| 156 | Ablation Regulatory | GGTTACACAACCAGGCGGGGAGGGGCCCCGGGGGGGGGAGGGGGC |
| | Element-SB07207 | CGGCCCGCGGGCCGCGCAGCCGGAAGCCGGGACCGCCACCGGCCC |
| | | TACACGGTTAAGACTCAATCCGGTAAGTAACCCCCTGGCGGGCTGG |
| | | CCCCGCCCCCGCGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGTCTAGAGGGTATATAATGGGGGCCA |
| 157 | Ablation Regulatory | GGTTACACAACCAGGCGGGGAGGGGCCCCGGGGGGGGGAGGGGGC |
| | Element-SB07208 | CGGCCCGCGGGCCGCGCAGCCGGAAGCCGGGACCGCCACCGGCCC |
| | | CCGGCGAGGGGAGCCCGGCTCCAGGCCCCGTGCCTAAAACGAGATT |
| | | CTGTACGATAAAGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGTCTAGAGGGTATATAATGGGGGCCA |
| 158 | Ablation Regulatory | CAGGCGGGGAGGGGCCCCGGGGGGGGGAGGGGGCCGGCCCGCGGG |
| | Element-SB07221 | CCGCGCAGCCGGAAGCCGGGACCGCCACCGGCCCCCGGCGAGGGG |
| | | TACACGGTTAAGACTCAATCCGGTAAGTAAGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCCCTGCCCGCCCACCCGGACACCCCACCCCTTCCCCC |
| | | TCCTTTCCGAAGCCCCCCTCCCTGTTTTTTCAGCCCCCTCCCCCCCAT |
| | | CCCCCATGGAGCCCTGCAGGTCTAGAGGGTATATAATGGGGGCCA |
| 159 | Ablation Regulatory | CAGGCGGGGAGGGGCCCCGGGGGGGGGAGGGGGCCGGCCCGCGGG |
| | Element-SB07222 | CCGCGCAGCCGGAAGCCGGGACCGCCACCGGCCCCCGGCGAGGGG |
| | | AGCCCGGCTCCAGGCCCCGCCCCCTGGCGGTGCCTAAAACGAGATT |
| | | CTGTACGATAAAGCATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCCCTGCCCGCCCACCCGGACACCCCACCCCTTCCCCC |
| | | TCCTTTCCGAAGCCCCCCTCCCTGTTTTTTCAGCCCCCTCCCCCCCAT |
| | | CCCCCATGGAGCCCTGCAGGTCTAGAGGGTATATAATGGGGGCCA |
| 160 | Ablation Regulatory | CAGGCGGGGAGGGGCCCCGGGGGGGGGAGGGGGCCGGCCCGCGGG |
| | Element-SB07225 | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCTACGGTCTCGCTAATAGGA |
| | | GTAAGATACATCGGGGCACCCTGGGACTTGTAGTCCAAGCCGCTTG |
| | | CCACCTGCCGGCTGCAAACGGCGGAGGGACTACGAAGCCCAGAGG |
| | | TCCCTGCGGCCCTGCCCGCCCACCCGGACACCCCACCCCTTCCCCCT |
| | | CCTTTCCGAAGCCCCCCTCCCTGTTTTTTCAGCCCCCTCCCCCCCATC |
| | | CCCCATGGAGCCCTGCAGGTCTAGAGGGTATATAATGGGGGCCA |
| 161 | Ablation Regulatory | CAGGCGGGGAGGGGCCCCGGGGGGGGGAGGGGGCCGGCCCGCGGG |
| | Element-SB07227 | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCACTA |
| | | TCACGTATATACCAGCGAGTTCGATAGGGACTACGAAGCCCAGAGG |
| | | TCCCTGCGGCCCTGCCCGCCCACCCGGACACCCCACCCCTTCCCCCT |
| | | CCTTTCCGAAGCCCCCCTCCCTGTTTTTTCAGCCCCCTCCCCCCCATC |
| | | CCCCATGGAGCCCTGCAGGTCTAGAGGGTATATAATGGGGGCCA |
| 162 | Ablation Regulatory | CAGGCGGGGAGGGGCCCCGGGGGGGGGAGGGGGCCGGCCCGCGGG |
| | Element-SB07229 | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCTGTTAAGCATACTAAACTGTAAAGAAGCGATCCCC |
| | | CTCCTTTCCGAAGCCCCCCTCCCTGTTTTTTCAGCCCCCTCCCCCCCA |
| | | TCCCCCATGGAGCCCTGCAGGTCTAGAGGGTATATAATGGGGGCCA |
| 163 | Ablation Regulatory | CAGGCGGGGAGGGGCCCCGGGGCGGGGAGGGGGCCGGCCCGCGGG |
| | Element-SB07230 | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCCCTGCCCGCCCACCCGGACACCCCACCCCTGTTTCG |
| | | AGCGACGCTTAATATTGCTAGATATTTTTTCAGCCCCCTCCCCCCCA |
| | | TCCCCCATGGAGCCCTGCAGGTCTAGAGGGTATATAATGGGGGCCA |
| 164 | SB08160 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CCTGCAGGTCTAGAGGGTATATAATGGGGGCCA |
| 165 | SB08163 | TGCTGAGTCAGCATAGGAAGAGTCTAAGCCACAGCACGCGTAGTCT |
| | | AATCCTCAGCCTACTTAACTATAAATAGATCGAGCAATACCATCTGT |
| | | CCCTGCAGGTCTAGAGGGTATATAATGGGGGCCA |
| 166 | SB08165 | GACAGATGGTTTTGACGCCTTGTGGCTTAGAGGCACATCATGCTGA |
| | | GTCAGCAACCGACGGCTGAGGATTAGTTTGCGAGTGTACCATCTGT |
| | | CCCTGCAGGTCTAGAGGGTATATAATGGGGGCCA |
| 167 | SB08166 | TGCTGACTCAGCAACGACTATAAGTGGCTTAGAACATAACGTCTCT |
| | | AATCCTCAGACTGCGCGTGACAGATGGTTTCGTCCCGTACCATCTGT |
| | | CCCTGCAGGTCTAGAGGGTATATAATGGGGGCCA |
| 168 | SB08179 | ACTATAAATAGAAACTACAACTGAGGATTAGAGTACACACTTGCTG |
| | | AGTCAGCAAATCTTTAGTCTAAGCCACTTAGATCGCCAACCATCTGT |
| | | CCCTGCAGGTCTAGAGGGTATATAATGGGGGCCA |
| 169 | SB08182 | CTAAGCCACATTCGTCTAGTACTAATCCTCTCGGCAGCCGATCTAAT |
| | | CCTCAGTCTAGCGCTGACAGATGGTTCACGGGTGGATTAATTGGCC |
| | | TGCAGGTCTAGAGGGTATATAATGGGGGCCA |
| 170 | SB08183 | GACAGATGGTTTGTCGAGTCACTAATCCTCATCCTTAGAGAAGAGG |
| | | ATTAGTTGAACAGTATGCTGACTCAGCAAGTGTCGCAACCATCTGT |
| | | CCCTGCAGGTCTAGAGGGTATATAATGGGGGCCA |
| 171 | SB08184 | AAGGTCATGACCTTAAAACCATGCTGACTCAGCATGCACGCATTGT |
| | | GGCTTAGAATTGCGCCTATCTAATCCTCTATCAGACAGAACCATCTG |
| | | TCCCTGCAGGTCTAGAGGGTATATAATGGGGGCCA |
| 172 | SB08209 | GGTTACACAACCAGGCGGGGAGGGGCCCCGGGGCGGGGAGGGGGC |
| | | CGGCCCGCGGGCCGCGGGACTACGAAGCCCAGAGGTCCCTGCGGCT |
| | | GGGGTGAGTCCTGCTCCTTTGTTCTTCCCATCGGCCCGCGCCCATTG |
| | | GCTCTCCGGCCCGCCGCTCACCGCCCCTCCTCCGCACCGCCCGGGAC |
| | | TACGAAGCCCAGAGGTCCCTGCGGCCGGGGCACCCTGGGACTTGTA |
| | | GTCCAAGCCTGGGGTGAGTCCTGCTCCTTTGTTCTTCCCGGGACTAC |
| | | GAAGCCCAGAGGTCCCTGCGGCTGGGGTGAGTCCTGCTCCTTTGTTC |
| | | TTCCCGGGACTACGAAGCCCAGAGGTCCCTGCGGCTTTTTTCAGCCC |
| | | CCTCCCCCCCATCCCCCATGGAGCCCTGCAGGTCTAGAGGGTATAT |
| | | AATGGGGGCCA |
| 173 | SB08211 | CAGGCGGGGAGGGGCCCCGGGGGGGGGAGGGGGCCGGCCCGCGGG |
| | | CCGCGCAGCCGGAAGCCGGGACCGCCACCGGCCCCCGGCGAGGGG |
| | | GGTCCCTCCCTACCTCTGCCCCGCGCTCTGCCTTTGATCCTCTGCTCG |
| | | GCTCTGAGCCATATCGGCCCGCGCCCATTGGCTCTCCGGCCCGCCGC |
| | | TCACCGCCCCTCCTCCGCACCGCCCTGGGGTGAGTCCTGCTCCTTTG |
| | | TTCTTCCCCGGGGCACCCTGGGACTTGTAGTCCAAGCCGCTTGCCAC |
| | | CTGCCGGCTGCAAACGGCGGAGGGACTACGAAGCCCAGAGGTCCCT |
| | | GCGGCCTCCTGCTATTGGTCAGAATGTGTCACGTGACCATACTCAGC |
| | | TGCAAGGGTTGGTGGGAATTTTTTCAGCCCCCTCCCCCCCATCCCCC |
| | | ATGGAGCCCTGCAGGTCTAGAGGGTATATAATGGGGGCCA |
| 174 | SB08212 | CAGGCGGGGAGGGGCCCCGGGGCGGGGAGGGGGCCGGCCCGCGGG |
| | | CCGCGCAGCCGGAAGCCGGGACCGCCACCGGCCCCCGGCGAGGGG |
| | | CTCCTGCTATTGGTCAGAATGTGTCACGTGACCATACTCAGCTGCAA |
| | | GGGTTGGTGGGAAATCGGCCCGCGCCCATTGGCTCTCCGGCCCGCC |
| | | GCTCACCGCCCCTCCTCCGCACCGCCCTGGGGTGAGTCCTGCTCCTT |
| | | TGTTCTTCCCCGGGGCACCCTGGGACTTGTAGTCCAAGCCTGGGGTG |
| | | AGTCCTGCTCCTTTGTTCTTCCCGGGACTACGAAGCCCAGAGGTCCC |
| | | TGCGGCCTCCTGCTATTGGTCAGAATGTGTCACGTGACCATACTCAG |
| | | CTGCAAGGGTTGGTGGGAAATGTAGTCTTTTTTTCTGGGAGGCCATG |
| | | TGTTCGGCTTAAAATTGTCTTTTTTTCTTTTTCCTGCAGGTCTAGAGG |
| | | GTATATAATGGGGGCCA |
| 175 | SB08213 | CAGGCGGGGAGGGGCCCCGGGGGGGGGAGGGGGCCGGCCCGCGGG |
| | | CCGCGCAGCCGGAAGCCGGGACCGCCACCGGCCCCCGGCGAGGGG |
| | | CGGGGCACCCTGGGACTTGTAGTCCAAGCCGGGACTACGAAGCCCA |
| | | GAGGTCCCTGCGGCATCGGCCCGCGCCCATTGGCTCTCCGGCCCGC |
| | | CGCTCACCGCCCCTCCTCCGCACCGCCCGGGACTACGAAGCCCAGA |
| | | GGTCCCTGCGGCCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCATCGGCCCGCGCCCATTGGCTCTCCGGCCCGCCGCT |
| | | CACCGCCCCTCCTCCGCACCGCCCTTTTTTCAGCCCCCTCCCCCCCA |
| | | TCCCCCATGGAGCCCTGCAGGTCTAGAGGGTATATAATGGGGGCCA |
| 176 | SB08214 | CGGGGCACCCTGGGACTTGTAGTCCAAGCCGGGACTACGAAGCCCA |
| | | GAGGTCCCTGCGGCATCGGCCCGCGCCCATTGGCTCTCCGGCCCGC |
| | | CGCTCACCGCCCCTCCTCCGCACCGCCCCGGGGCACCCTGGGACTT |
| | | GTAGTCCAAGCCGGGACTACGAAGCCCAGAGGTCCCTGCGGCATCG |
| | | GCCCGCGCCCATTGGCTCTCCGGCCCGCCGCTCACCGCCCCTCCTCC |
| | | GCACCGCCCCGGGGCACCCTGGGACTTGTAGTCCAAGCCGGGACTA |
| | | CGAAGCCCAGAGGTCCCTGCGGCATCGGCCCGCGCCCATTGGCTCT |
| | | CCGGCCCGCCGCTCACCGCCCCTCCTCCGCACCGCCCCGGGGCACC |
| | | CTGGGACTTGTAGTCCAAGCCGGGACTACGAAGCCCAGAGGTCCCT |
| | | GCGGCTTTTTTCAGCCCCCTCCCCCCCATCCCCCATGGAGCCCTGCA |
| | | GGTCTAGAGGGTATATAATGGGGGCCA |
| 177 | SB08215 | GGTTACACAACCAGGCGGGGAGGGGCCCCGGGGCGGGGAGGGGGC |
| | | CGGCCCGCGGGCCGCATCGGCCCGCGCCCATTGGCTCTCCGGCCCG |
| | | CCGCTCACCGCCCCTCCTCCGCACCGCCCGGGACTACGAAGCCCAG |
| | | AGGTCCCTGCGGCGGTCCCTCCCTACCTCTGCCCCGCGCTCTGTGGG |
| | | GTGAGTCCTGCTCCTTTGTTCTTCCCGGTTACACAACCAGGCGGGGA |
| | | GGGGCCCCGGGGGGGGAGGGGGCCGGCCCGCGGGCCGCATCGGC |
| | | CCGCGCCCATTGGCTCTCCGGCCCGCCGCTCACCGCCCCTCCTCCGC |
| | | ACCGCCCGGGACTACGAAGCCCAGAGGTCCCTGCGGCGGTCCCTCC |
| | | CTACCTCTGCCCCGCGCTCTGTGGGGTGAGTCCTGCTCCTTTGTTCTT |
| | | CCCTTTTTTCAGCCCCCTCCCCCCCATCCCCCATGGAGCCCTGCAGG |
| | | TCTAGAGGGTATATAATGGGGGCCA |
| 178 | SB08216 | CTCCTGCTATTGGTCAGAATGTGTCACGTGACCATACTCAGCTGCAA |
| | | GGGTTGGTGGGAAATGTAGTCTTTTTTTCTGGGAGGCCATGTGTTCG |
| | | GCTTAAAATTGTCTTTTTTTCTTTTTGGTCCCTCCCTACCTCTGCCCC |
| | | GCGCTCTGTGGGGTGAGTCCTGCTCCTTTGTTCTTCCCCTCCTGCTAT |
| | | TGGTCAGAATGTGTCACGTGACCATACTCAGCTGCAAGGGTTGGTG |
| | | GGAAATGTAGTCTTTTTTTCTGGGAGGCCATGTGTTCGGCTTAAAAT |
| | | TGTCTTTTTTTCTTTTTGGTCCCTCCCTACCTCTGCCCCGCGCTCTGT |
| | | GGGGTGAGTCCTGCTCCTTTGTTCTTCCCCTCCTGCTATTGGTCAGA |
| | | ATGTGTCACGTGACCATACTCAGCTGCAAGGGTTGGTGGGAAATGT |
| | | AGTCTTTTTTTCTGGGAGGCCATGTGTTCGGCTTAAAATTGTCTTTTT |
| | | TTCTTTTTCCTGCAGGTCTAGAGGGTATATAATGGGGGCCA |
| 179 | SB08217 | CGGGGCACCCTGGGACTTGTAGTCCAAGCCGGGACTACGAAGCCCA |
| | | GAGGTCCCTGCGGCATCGGCCCGCGCCCATTGGCTCTCCGGCCCGC |
| | | CGCTCACCGCCCCTCCTCCGCACCGCCCCGGGGCACCCTGGGACTT |
| | | GTAGTCCAAGCCGGGACTACGAAGCCCAGAGGTCCCTGCGGCATCG |
| | | GCCCGCGCCCATTGGCTCTCCGGCCCGCCGCTCACCGCCCCTCCTCC |
| | | GCACCGCCCCGGGGCACCCTGGGACTTGTAGTCCAAGCCGGGACTA |
| | | CGAAGCCCAGAGGTCCCTGCGGCCTCCTGCTATTGGTCAGAATGTG |
| | | TCACGTGACCATACTCAGCTGCAAGGGTTGGTGGGAAATGTAGTCT |
| | | TTTTTTCTGGGAGGCCATGTGTTCGGCTTAAAATTGTCTTTTTTTCTT |
| | | TTTGGTCCCTCCCTACCTCTGCCCCGCGCTCTGTGGGGTGAGTCCTG |
| | | CTCCTTTGTTCTTCCCCCTGCAGGTCTAGAGGGTATATAATGGGGGC |
| | | CA |
| 180 | SB08218 | TGGGGTGAGTCCTGCTCCTTTGTTCTTCCCGGGACTACGAAGCCCAG |
| | | AGGTCCCTGCGGCATCGGCCCGCGCCCATTGGCTCTCCGGCCCGCC |
| | | GCTCACCGCCCCTCCTCCGCACCGCCCTGGGGTGAGTCCTGCTCCTT |
| | | TGTTCTTCCCGGGACTACGAAGCCCAGAGGTCCCTGCGGCATCGGC |
| | | CCGCGCCCATTGGCTCTCCGGCCCGCCGCTCACCGCCCCTCCTCCGC |
| | | ACCGCCCTGGGGTGAGTCCTGCTCCTTTGTTCTTCCCGGGACTACGA |
| | | AGCCCAGAGGTCCCTGCGGCCTCCTGCTATTGGTCAGAATGTGTCA |
| | | CGTGACCATACTCAGCTGCAAGGGTTGGTGGGAAGGGACTACGAAG |
| | | CCCAGAGGTCCCTGCGGCTGGGGTGAGTCCTGCTCCTTTGTTCTTCC |
| | | CTTTTTTCAGCCCCCTCCCCCCCATCCCCCATGGAGCCCTGCAGGTC |
| | | TAGAGGGTATATAATGGGGGCCA |
| 181 | SB08322 | CAGGCGGGGAGGGGCCCCGGGGCGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGATCGGCCCGCGCCCAT |
| | | TGGCTCTCCGGCCCGCCGCTCACCGCCCCTCCTCCGCACCGCCCCGG |
| | | GGCACCCTGGGACTTGTAGTCCAAGCCGCTTGCCACCTGCCGGCTG |
| | | CAAACGGCGGAGGGACTACGAAGCCCAGAGGTCCCTGCGGCTCCCC |
| | | CTCCTTTCCGAAGCCCCCCTCCCTGTTTTTTCAGCCCCCTCCCCCCCA |
| | | TCCCCCATGGAGCGACAGATGGTTTTGACGCCTTGTGGCTTAGAGG |
| | | CACATCATGCTGAGTCAGCAACCGACGGCTGAGGATTAGTTTGCGA |
| | | GTGTACCATCTGTCCCTGCAGGTTCGCATATTAAGGTGACGCGTGTG |
| | | GCCTCGAACACCGAGCGACCCTGCAGCGACCCGCTTAA |
| 182 | SB08323 | CAGGCGGGGAGGGGCCCCGGGGCGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGATCGGCCCGCGCCCAT |
| | | TGGCTCTCCGGCCCGCCGCTCACCGCCCCTCCTCCGCACCGCCCCGG |
| | | GGCACCCTGGGACTTGTAGTCCAAGCCGCTTGCCACCTGCCGGCTG |
| | | CAAACGGCGGAGGGACTACGAAGCCCAGAGGTCCCTGCGGCTCCCC |
| | | CTCCTTTCCGAAGCCCCCCTCCCTGTTTTTTCAGCCCCCTCCCCCCCA |
| | | TCCCCCATGGAGCTGCTGAGTCAGCAAGTATACGAACTAAGCCACA |
| | | GGAGTCTTGTACCATCTGTCAATCGGCTTAACCATCTGTCAAGTATC |
| | | TATACCATCTGTCCCTGCAGGTTCGCATATTAAGGTGACGCGTGTGG |
| | | CCTCGAACACCGAGCGACCCTGCAGCGACCCGCTTAA |
| 183 | SB08324 | CAGGCGGGGAGGGGCCCCGGGGGGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGATCGGCCCGCGCCCAT |
| | | TGGCTCTCCGGCCCGCCGCTCACCGCCCCTCCTCCGCACCGCCCCGG |
| | | GGCACCCTGGGACTTGTAGTCCAAGCCGCTTGCCACCTGCCGGCTG |
| | | CAAACGGCGGAGGGACTACGAAGCCCAGAGGTCCCTGCGGCTCCCC |
| | | CTCCTTTCCGAAGCCCCCCTCCCTGTTTTTTCAGCCCCCTCCCCCCCA |
| | | TCCCCCATGGAGCCTAAGCCACAGTAACATGCGACTAATCCTCTCT |
| | | GTCTACAGAGTGGCTTAGTCTATCGTGTTGCTGACTCAGCATTAGAT |
| | | AGTGACAGATGGTCCTGCAGGTCTAGAGGGTATATAATGGGGGCCA |
| 184 | SB08326 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACACCCCA |
| | | CCCCTTCCCCCTCCTTTCCGAAGCCCCCCTCCCTGTTTTTTCAGCCCC |
| | | CTCGACAGATGGTTTTGACGCCTTGTGGCTTAGAGGCACATCATGCT |
| | | GAGTCAGCAACCGACGGCTGAGGATTAGTTTGCGAGTGTACCATCT |
| | | GTCCCTGCAGGTTCGCATATTAAGGTGACGCGTGTGGCCTCGAACA |
| | | CCGAGCGACCCTGCAGCGACCCGCTTAA |
| 185 | SB08327 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACACCCCA |
| | | CCCCTTCCCCCTCCTTTCCGAAGCCCCCCTCCCTGTTTTTTCAGCCCC |
| | | CTCTGCTGAGTCAGCAAGTATACGAACTAAGCCACAGGAGTCTTGT |
| | | ACCATCTGTCAATCGGCTTAACCATCTGTCAAGTATCTATACCATCT |
| | | GTCCCTGCAGGTTCGCATATTAAGGTGACGCGTGTGGCCTCGAACA |
| | | CCGAGCGACCCTGCAGCGACCCGCTTAA |
| 186 | SB08328 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACACCCCA |
| | | CCCCTTCCCCCTCCTTTCCGAAGCCCCCCTCCCTGTTTTTTCAGCCCC |
| | | CTCCTAAGCCACAGTAACATGCGACTAATCCTCTCTGTCTACAGAGT |
| | | GGCTTAGTCTATCGTGTTGCTGACTCAGCATTAGATAGTGACAGATG |
| | | GTCCTGCAGGTCTAGAGGGTATATAATGGGGGCCA |
| 187 | SB08329 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACACCCCA |
| | | CCCCTTCCCCCTCCTTTCCGAAGCCCCCCTCCCTGTTTTTTCAGCCCC |
| | | CTCCCTGCAGGTCTAGAGGGTATATAATGGGGGCCA |
| 188 | SB08330 | GGTTACACAACCAGGCGGGGAGGGGCCCCGGGGGGGGAGGGGGC |
| | | CGGCCCGCGGGCCGCGCAGCCGGAAGCCGGGACCGCCACCGGCCC |
| | | CCGGCGAGGGGAGCCCGGCTCCAGGCCCCGTGCCTAAAACGAGATT |
| | | CTGTACGATAAAGCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGTCTAGAGGGTATATAATGGGGGCCA |
| 189 | SB11066 | GACAGATGGTTTTGACGCCTTGTGGCTTAGAGGCACATCATGCTGA |
| | | GTCAGCAACCGACGGCTGAGGATTAGTTTGCGAGTGTACCATCTGT |
| | | CTGCTGAGTCAGCAAGTATACGAACTAAGCCACAGGAGTCTTGTAC |
| | | CATCTGTCAATCGGCTTAACCATCTGTCAAGTATCTATACCATCTGT |
| | | CCTCCTGCTATTGGTCAGAATGTGTCACGTGACCATACTCAGCTGCA |
| | | AGGGTTGGTGGGAACTAAGCCACAGTAACATGCGACTAATCCTCTC |
| | | TGTCTACAGAGTGGCTTAGTCTATCGTGTTGCTGACTCAGCATTAGA |
| | | TAGTGACAGATGGTCCTGCAGGTCTAGAGGGTATATAATGGGGGCC |
| | | A |
| 190 | SB11067 | GACAGATGGTTTTGACGCCTTGTGGCTTAGAGGCACATCATGCTGA |
| | | GTCAGCAACCGACGGCTGAGGATTAGTTTGCGAGTGTACCATCTGT |
| | | CTACACGGTTAAGACTCAATCCGGTAAGTAAGAGAAAAACAGGAA |
| | | AAGAGTTTACATGCCACTCCTGCTATTGGTCAGAATGTGTCACGTGA |
| | | CCATACTCAGCTGCAAGGGTTGGTGGGAAATGTAGTCTTTTTTTCTG |
| | | GGAGGCCATGTGTTCGGCTTAAAATTGTCTTTTTTTCTTTTTCTAAGC |
| | | CACAGTAACATGCGACTAATCCTCTCTGTCTACAGAGTGGCTTAGTC |
| | | TATCGTGTTGCTGACTCAGCATTAGATAGTGACAGATGGTCCTGCA |
| | | GGTCTAGAGGGTATATAATGGGGGCCA |
| 191 | SB11068 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TACACGGTTAAGACTCAATCCGGTAAGTAAGAGAAAAACAGGAAA |
| | | AGAGTTTACATGCCACTCCTGCTATTGGTCAGAATGTGTCACGTGAC |
| | | CATACTCAGCTGCAAGGGTTGGTGGGAAATGTAGTCTTTTTTTCTGG |
| | | GAGGCCATGTGTTCGGCTTAAAATTGTCTTTTTTTCTTTTTCTAAGCC |
| | | ACAGTAACATGCGACTAATCCTCTCTGTCTACAGAGTGGCTTAGTCT |
| | | ATCGTGTTGCTGACTCAGCATTAGATAGTGACAGATGGTCCTGCAG |
| | | GTCTAGAGGGTATATAATGGGGGCCA |
| 192 | SB08338 | CTAAGCCACAGTAACATGCGACTAATCCTCTCTGTCTACAGAGTGG |
| | | CTTAGTCTATCGTGTTGCTGACTCAGCATTAGATAGTGACAGATGGT |
| | | TTACCTACTAGGTTAATTCGTCCGATAGATACTACGAATTGCGAGCT |
| | | TCTAAGTCCAATTTTCGGTATTCGAGTCGTCAGACTCAATTATTACA |
| | | CGGTTAAGACTCAATCCGGTAAGTAATACTGTGATCAGGTGTCTAC |
| | | AAGACTAGTACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTACTATCACGTA |
| | | TATACCAGCGAGTTCGATAATACACTCTCCTGCAGGTCTAGAGGGT |
| | | ATATAATGGGGGCCA |
| 193 | SB08478 | CTCAGGCCCTGCATTGCGCCAACGGCGCAGCGCTGGGCCGCGACCC |
| | | CGGCACCGGCACCCGGGGACGTTGTGAATAGCTGGTCGAAGTTTGG |
| | | TTGAAGCACAGGAGGGTAAATAAGAAGGTTTCCAGACAAAGAGAC |
| | | TCTTAAAGGTACAGGCCAGAGACGCTCCCGAGCCCATCTCCTCCTG |
| | | CTATTGGTCAGAATGTGTCACGTGACCATACTCAGCTGCAAGGGTT |
| | | GGTGGGAAATGTAGTCTTTTTTTCTGGGAGGCCATGTGTTCGGCTTA |
| | | AAATTGTCTTTTTTTCTTTTTCTTTATTTCTTTTTTCTTTTCTTTCTTTT |
| | | TATACACTCTCCTGCAGGAGACGCTAGCGGGGGGCTATAAAAGGGG |
| | | GTGGGGGCGTTCGTCCTCACTCT |
| 194 | SB08479 | CTCAGGCCCTGCATTGCGCCAACGGCGCAGCGCTGGGCCGCGACCC |
| | | CGGCACCGGCACCCCTCCTGCTATTGGTCAGAATGTGTCACGTGAC |
| | | CATACTCAGCTGCAAGGGTTGGTGGGAAATGTAGTCTTTTTTTCTGG |
| | | GAGGCCATGTGTTCGGCTTAAAATTGTCTTTTTTTCTTTTTGGGGAC |
| | | GTTGTGAATAGCTGGTCGAAGTTTGGTTGAAGCACAGGAGGGTAAA |
| | | TAAGAAGGTTTCCAGACAAAGAGACTCTTAAAGGTACAGACTCTTA |
| | | TGTCTGTCCCTCCTCCTTAAAGGGCCAGAGACGCTCCCGAGCCCATC |
| | | TCCCTGCAGGAGACGCTAGCGGGGGGCTATAAAAGGGGGTGGGGG |
| | | CGTTCGTCCTCACTCT |
| 195 | SB08480 | CTAAGCCACAGTAACATGCGACTAATCCTCTCTGTCTACAGAGTGG |
| | | CTTAGTCTATCGTGTTGCTGACTCAGCATTAGATAGTGACAGATGGT |
| | | CTCAGGCCCTGCATTGCGCCAACGGCGCAGCGCTGGGCCGCGACCC |
| | | CGGCACCGGCACCCGTTCCGAGGGTTCGCGCCGCAGGCGCAAAGCC |
| | | ACATTGCTATAGTGCTGTATAGCGATTATTGAGCCTGGCGTAAGGG |
| | | GCGGCGGCGTAGGGGGACGTTGTGAATAGCTGGTCGAAGTTTGGTT |
| | | GAAGCACAGGAGGGTAAATAAGAAGGTTTCCAGACAAAGAGACTC |
| | | TTAAAGGTACAGACTCTTATGTCTGTCCCTCCTCCTTAAAGGGCCAG |
| | | AGACGCTCCCGAGCCCATCTCCCTGCAGGAGACGCTAGCGGGGGGC |
| | | TATAAAAGGGGGTGGGGGCGTTCGTCCTCACTCT |
| 196 | SB08482 | AGCACGCGCGAGCTGTCACCAGGGCGCGAGACTACGAATTGCGAG |
| | | CTTCTAAGTCCAATTTCTATTGGCCCGCCCGCTGGAGCCCCGCCCCT |
| | | CAGGCCCTGCATTGCGCCAACGGCGCAGCGCTGGGCCGCGACCCCG |
| | | GCACCGGCACCCGTTCCGAGGGTTCGCGCCGCAGGCGCAAAGTTTA |
| | | GGGTCAGCCACAAACGCGCGGCCGCTTTGAGCCTGGCGTAAGGGGC |
| | | GGCGGCGTAGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTCCCTGCAGGAGACGCTAGCGGGGGGCT |
| | | ATAAAAGGGGGTGGGGGCGTTCGTCCTCACTCT |
| 197 | SB08483 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACGCAAGG |
| | | AAGATTATTTTGAGTCACTGCACCCTTTCTGAACAATAGCTGTTACC |
| | | AAGGTCTCAGGAGCATTAGGCGCTCAATTCTATTTTAAGTAGCATG |
| | | GTTGGAGCTTAACCCCAAGTCTAATTTTATTCTGCCTCTTTATTTAA |
| | | AAAGGACAGATGGTTCCCAACGACTAATCCTCATAGAACGCACTAA |
| | | TCCTCCCTGCAGGTCTAGAGGGTATATAATGGGGGCCA |
| 198 | SB08484 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACGCAAGG |
| | | AAGATTATTTTGAGTCACTGCACCCTTTCTGAACAATAGCTGTTACC |
| | | AAGGTCTCAGGAGCATTAGGCGCTCAATTCTATTTTAAGTAGCATG |
| | | GTTGGAGCTTAACCCCAAGTCTAATTTTATTCTGCCTCTTTATTTAA |
| | | AAAGCTAATCCTCTCGGCCCGATCTAATCCTCAGTCTCGCTGACAGA |
| | | TGGTCCTGCAGGTCTAGAGGGTATATAATGGGGGCCA |
| 199 | SB08485 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACGACAGA |
| | | TGGTTCCCAACGACTAATCCTCATAGAACGCACTAATCCTCGCAAG |
| | | GAAGATTATTTTGAGTCACTGCACCCTTTCTGAACAATAGCTGTTAC |
| | | CAAGGTCTCAGGAGCATTAGGCGCTCAATTCTATTTTAAGTAGCAT |
| | | GGTTGGAGCTTAACCCCAAGTCTAATTTTATTCTGCCTCTTTATTTA |
| | | AAAAGCCTGCAGGTCTAGAGGGTATATAATGGGGGCCA |
| 200 | SB08486 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACCTAATC |
| | | CTCTCGGCCCGATCTAATCCTCAGTCTCGCTGACAGATGGTGCAAG |
| | | GAAGATTATTTTGAGTCACTGCACCCTTTCTGAACAATAGCTGTTAC |
| | | CAAGGTCTCAGGAGCATTAGGCGCTCAATTCTATTTTAAGTAGCAT |
| | | GGTTGGAGCTTAACCCCAAGTCTAATTTTATTCTGCCTCTTTATTTA |
| | | AAAAGCCTGCAGGTCTAGAGGGTATATAATGGGGGCCA |
| 201 | SB08487 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACAGCGCT |
| | | GACAGATGGTTCACGGGCAAGGAAGATTATTTTGAGTCACTGCACC |
| | | CTTTCTGAACAATAGCTGTTACCAAGGTCTCAGGAGCATTAGGCGC |
| | | TCAATTCTATTTTAAGTAGCATGGTTGGAGCTTAACCCCAAGTCTAA |
| | | TTTTATTCTGCCTCTTTATTTAAAAAGGGGCCAGACAGATGGTTCCC |
| | | AATACCCTGCAGGTCTAGAGGGTATATAATGGGGGCCA |
| 202 | SB08490 | GGTTACACAACCAGGCGGGGAGGGGCCCCGGGGCGGGGAGGGGGC |
| | | CGGCCCGCGGGCCGCGCAGCCGGAAGCCGGGACCGCCACCGGCCC |
| | | TACGGGCCAGACAGATGGTTCCCAATACGACTAATCCTCATAGAAG |
| | | ACGCACTAATCCTCCGCGCCGCGCGATCGGCCCGCGCCCATTGGCT |
| | | CTCCGGCCCGCCGCTCACCGCCCCTCCTCCGCACCGCCCCTACCCGC |
| | | AGGCCGCGGCGGGCTGTCGGCGCGGGGCACCCTGGGACTTGTAGTC |
| | | CAAGCCGCTTGCCACCTGCCGGCTGCAAACGGCGGAGGGACTACGA |
| | | AGCCCAGAGGTCCCTGCGGCCCTGCCCGCCCACCCGGACACCCCAC |
| | | CCCTTCCCCCTCCTTTCCGAAGCCCCCCTCCCTGTTTTTTCAGCCCCC |
| | | TCCCTGCAGGTTCGCATATTAAGGTGACGCGTGTGGCCTCGAACAC |
| | | CGAGCGACCCTGCAGCGACCCGCTTAA |
| 203 | SB08493 | GGTTACACAACCAGGCGGGGAGGGGCCCCGGGGGGGGAGGGGGC |
| | | CGGCCCGCGGGCCGCGCAGCCGGAAGCCGGGACCGCCACCGGCCC |
| | | ATTCGTCTAGTACTAATCCTCTCGGCAGCCGATCTAATCCTCAGTCT |
| | | AGCGCTGACAGATGGTTCACGGGTGGACGCGCCGCGCGATCGGCCC |
| | | GCGCCCATTGGCTCTCCGGCCCGCCGCTCACCGCCCCTCCTCCGCAC |
| | | CGCCCCTACCCGCAGGCCGCGGCGGGCTGTCGGCGCGGGGCACCCT |
| | | GGGACTTGTAGTCCAAGCCGCTTGCCACCTGCCGGCTGCAAACGGC |
| | | GGAGGGACTACGAAGCCCAGAGGTCCCTGCGGCCCTGCCCGCCCAC |
| | | CCGGACACCCCACCCCTTCCCCCTCCTTTCCGAAGCCCCCCTCCCTG |
| | | TTTTTTCAGCCCCCTCCCTGCAGGTCTAGAGGGTATATAATGGGGGC |
| | | CA |
| 204 | SB08494 | TACGGGCCAGACAGATGGTTCCCAATACGACTAATCCTCATAGAAG |
| | | ACGCACTAATCCTCGTGAGTGACAGGCGAACCAGTACTATAAATAG |
| | | ATACGGGCCAGACAGATGGTTCCCAATACGACTAATCCTCATAGAA |
| | | GACGCACTAATCCTCACTGTCGAGTTGTGAGTGACAGGCGAACCAG |
| | | TACTATAAATAGATACGGGCCAGACAGATGGTTCCCAATACGACTA |
| | | ATCCTCATAGAAGACGCACTAATCCTCACTGTCGAGTTGTGAGTGA |
| | | CAGGCGAACCAGTACTATAAATAGATACGGGCCAGACAGATGGTTC |
| | | CCAATACGACTAATCCTCATAGAAGACGCACTAATCCTCACTGTCG |
| | | AGTTGTGAGTGACAGGCGAACCAGTACTATAAATAGATACGGGCCA |
| | | GACAGATGGTTCCCAATACGACTAATCCTCATAGAAGACGCACTAA |
| | | TCCTCCCTGCAGGTCTAGAGGGTATATAATGGGGGCCA |
| 205 | SB08495 | TAGTACTAATCCTCTCGGCAGCCGATCTAATCCTCAGTCTAGCGCTG |
| | | ACAGATGGTTCACCTAAGCCACATTCGTCTAGTACTAATCCTCTCGG |
| | | CAGCCGATCTAATCCTCAGTCTAGCGCTGACAGATGGTTCACGGGT |
| | | GGATTAATTGGTGTTCCGAACACTAAGCCACATTCGTCTAGTACTAA |
| | | TCCTCTCGGCAGCCGATCTAATCCTCAGTCTAGCGCTGACAGATGGT |
| | | TCACGGGTGGATTAATTGGTGTTCCGAACACTAAGCCACATTCGTCT |
| | | AGTACTAATCCTCTCGGCAGCCGATCTAATCCTCAGTCTAGCGCTGA |
| | | CAGATGGTTCACGGGTGGATTAATTGGTGTTCCGAACACTAAGCCA |
| | | CATTCGTCTAGTACTAATCCTCTCGGCAGCCGATCTAATCCTCAGTC |
| | | TAGCGCTGACAGATGGTTCACGGGTGGATTAATTGGCCTGCAGGTC |
| | | TAGAGGGTATATAATGGGGGCCA |
| 206 | SB08496 | GTGAGTGACAGGCGACCAGTACTATAAATAGATACGGGCCAGACA |
| | | GATGGTTCCCAATACGACTAATCCTCATAGAAGACGCACTAATCCT |
| | | CCTAAGCCACATTCGTCTAGTACTAATCCTCTCGGCAGCCGATCTAA |
| | | TCCTCAGTCTAGCGCTGACAGATGGTTCACGGGTGGATTAATTGGG |
| | | TGAGTGACAGGCGAACCAGTACTATAAATAGATACGGGCCAGACA |
| | | GATGGTTCCCAATACGACTAATCCTCATAGAAGACGCACTAATCCT |
| | | CCTAAGCCACATTCGTCTAGTACTAATCCTCTCGGCAGCCGATCTAA |
| | | TCCTCAGTCTAGCGCTGACAGATGGTTCACGGGTGGATTAATTGGC |
| | | CTGCAGGTCTAGAGGGTATATAATGGGGGCCA |
| 207 | SB08497 | CTAAGCCACATTCGTCTAGTACTAATCCTCTCGGCAGCCGATCTAAT |
| | | CCTCAGTCTAGCGCTGACAGATGGTTCACGGGTGGATTAATTGGCT |
| | | AAGCCACATTCGTCTAGTACTAATCCTCTCGGCAGCCGATCTAATCC |
| | | TCAGTCTAGCGCTGACAGATGGTTCACGGGTGGATTAATTGGCTAA |
| | | GCCACATTCGTCTAGTACTAATCCTCTCGGCAGCCGATCTAATCCTC |
| | | AGTCTAGCGCTGACAGATGGTTCACGGGTGGATTAATTGGCTAAGC |
| | | CACATTCGTCTAGTACTAATCCTCTCGGCAGCCGATCTAATCCTCAG |
| | | TCTAGCGCTGACAGATGGTTCACGGGTGGATTAATTGGCCTGCAGG |
| | | TCTAGAGGGTATATAATGGGGGCCA |
| 208 | SB08570 | CTAAGCCACAGTAACATGCGACTAATCCTCTCTGTCTACAGAGTGG |
| | | CTTAGTCTATCGTGTTGCTGACTCAGCATTAGATAGTGACAGATGGT |
| | | TAGTAAGGTACTAAGCCACATTCGTCTAGTACTAATCCTCTCGGCAG |
| | | CCGATCTAATCCTCAGTCTAGCGCTGACAGATGGTTCACGGGTGGA |
| | | TTAATTGGGGGGACGTTGTGAATAGCTGGTCGAAGTTTGGTTGAAG |
| | | CACAGGAGGGTAAATAAGAAGGTTTCCAGACAAAGAGACTCTTAA |
| | | AGGTACAGACTCTTATGTCTGTCCCTCCTCCTTAAAGGGCCAGAGAC |
| | | GCTCCCGAGCCCATCTCCCTGCAGGAGACGCTAGCGGGGGGCTATA |
| | | AAAGGGGGTGGGGGCGTTCGTCCTCACTCT |
| 209 | SB08571 | GTGAGTGACAGGCGAACCAGTACTATAAATAGATACGGGCCAGAC |
| | | AGATGGTTCCCAATACGACTAATCCTCATAGAAGACGCACTAATCC |
| | | TCTAGTAAGGTACTAAGCCACATTCGTCTAGTACTAATCCTCTCGGC |
| | | AGCCGATCTAATCCTCAGTCTAGCGCTGACAGATGGTTCACGGGTG |
| | | GATTAATTGGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTCCCTGCAGGAGACGCTAGCGGGGGGCT |
| | | ATAAAAGGGGGTGGGGGCGTTCGTCCTCACTCT |
| 210 | SB08572 | CTAAGCCACATTCGTCTAGTACTAATCCTCTCGGCAGCCGATCTAAT |
| | | CCTCAGTCTAGCGCTGACAGATGGTTCACGGGTGGATTAATTGGTA |
| | | GTAAGGTACTAAGCCACATTCGTCTAGTACTAATCCTCTCGGCAGCC |
| | | GATCTAATCCTCAGTCTAGCGCTGACAGATGGTTCACGGGTGGATT |
| | | AATTGGGGGGACGTTGTGAATAGCTGGTCGAAGTTTGGTTGAAGCA |
| | | CAGGAGGGTAAATAAGAAGGTTTCCAGACAAAGAGACTCTTAAAG |
| | | GTACAGACTCTTATGTCTGTCCCTCCTCCTTAAAGGGCCAGAGACGC |
| | | TCCCGAGCCCATCTCCCTGCAGGAGACGCTAGCGGGGGGCTATAAA |
| | | AGGGGGTGGGGGCGTTCGTCCTCACTCT |
| 211 | SB08573 | CTAAGCCACATTCGTCTAGTACTAATCCTCTCGGCAGCCGATCTAAT |
| | | CCTCAGTCTAGCGCTGACAGATGGTTCACGGGTGGATTAATTGGCT |
| | | CAGGCCCTGCATTGCGCCAACGGCGCAGCGCTGGGCCGCGACCCCG |
| | | GCACCGGCACCCGTTCCGAGGGTTCGCGCCGCAGGCGCAAAGCCAC |
| | | ATTGCTATAGTGCTGTATAGCGATTATTGAGCCTGGCGTAAGGGGC |
| | | GGCGGCGTAGGGGGACGTTGTGAATAGCTGGTCGAAGTTTGGTTGA |
| | | AGCACAGGAGGGTAAATAAGAAGGTTTCCAGACAAAGAGACTCTT |
| | | AAAGGTACAGACTCTTATGTCTGTCCCTCCTCCTTAAAGGGCCAGA |
| | | GACGCTCCCGAGCCCATCTCCCTGCAGGAGACGCTAGCGGGGGGCT |
| | | ATAAAAGGGGGTGGGGGCGTTCGTCCTCACTCT |
| 212 | SB08574 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACACCCCA |
| | | CCCCTTCCCCCTCCTTTCCGAAGCCCCCCTCCCTGTTTTTTCAGCCCC |
| | | CTCGTGAGTGACAGGCGAACCAGTACTATAAATAGATACGGGCCAG |
| | | ACAGATGGTTCCCAATACGACTAATCCTCATAGAAGACGCACTAAT |
| | | CCTCCCTGCAGGTTCGCATATTAAGGTGACGCGTGTGGCCTCGAAC |
| | | ACCGAGCGACCCTGCAGCGACCCGCTTAA |
| 213 | SB08575 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACACCCCA |
| | | CCCCTTCCCCCTCCTTTCCGAAGCCCCCCTCCCTGTTTTTTCAGCCCC |
| | | CTCCTAAGCCACATTCGTCTAGTACTAATCCTCTCGGCAGCCGATCT |
| | | AATCCTCAGTCTAGCGCTGACAGATGGTTCACGGGTGGATTAATTG |
| | | GCCTGCAGGTTCGCATATTAAGGTGACGCGTGTGGCCTCGAACACC |
| | | GAGCGACCCTGCAGCGACCCGCTTAA |
| 214 | SB08576 | GGTTACACAACCAGGCGGGGAGGGGCCCCGGGGGGGGAGGGGGC |
| | | CGGCCCGCGGGCCGCGCAGCCGGAAGCCGGGACCGCCACCGGCCC |
| | | CGCGCCGCGCGATCGGCCCGCGCCCATTGGCTCTCCGGCCCGCCGC |
| | | TCACCGCCCCTCCTCCGCACCGCCCCTACCCGCAGGCCGCGGCGGG |
| | | CTGTCGGCGCGGGGCACCCTGGGACTTGTAGTCCAAGCCGCTTGCC |
| | | ACCTGCCGGCTGCAAACGGCGGAGGGACTACGAAGCCCAGAGGTC |
| | | CCTGCGGCCCTGCCCGCCCACCCGGACACCCCACCCCTTCCCCCTCC |
| | | TTTCCGAAGCCCCCCTCCCTGTTTTTTCAGCCCCCTCGTGAGTGACA |
| | | GGCGAACCAGTACTATAAATAGATACGGGCCAGACAGATGGTTCCC |
| | | AATACGACTAATCCTCATAGAAGACGCACTAATCCTCCCTGCAGGT |
| | | CTAGAGGGTATATAATGGGGGCCA |
| 215 | SB08577 | GGTTACACAACCAGGCGGGGAGGGGCCCCGGGGCGGGGAGGGGGC |
| | | CGGCCCGCGGGCCGCGCAGCCGGAAGCCGGGACCGCCACCGGCCC |
| | | CGCGCCGCGCGATCGGCCCGCGCCCATTGGCTCTCCGGCCCGCCGC |
| | | TCACCGCCCCTCCTCCGCACCGCCCCTACCCGCAGGCCGCGGCGGG |
| | | CTGTCGGCGCGGGGCACCCTGGGACTTGTAGTCCAAGCCGCTTGCC |
| | | ACCTGCCGGCTGCAAACGGCGGAGGGACTACGAAGCCCAGAGGTC |
| | | CCTGCGGCCCTGCCCGCCCACCCGGACACCCCACCCCTTCCCCCTCC |
| | | TTTCCGAAGCCCCCCTCCCTGTTTTTTCAGCCCCCTCCTAAGCCACA |
| | | TTCGTCTAGTACTAATCCTCTCGGCAGCCGATCTAATCCTCAGTCTA |
| | | GCGCTGACAGATGGTTCACGGGTGGATTAATTGGCCTGCAGGTCTA |
| | | GAGGGTATATAATGGGGGCCA |
| 216 | SB08579 | GGTTACACAACCAGGCGGGGAGGGGCCCCGGCAGCCGGAAGCCGG |
| | | GACCGCCACCGGCCCCTAAGCCACATTCGTCTAGTACTAATCCTCTC |
| | | GGCAGCCGATCTAATCCTCAGTCTAGCGCTGACAGATGGTTCACGG |
| | | GTGGATTAATTGGCGCGCCGCGCGATCGGCCCGCGCCCATTGGCTC |
| | | TCCGGCCCGCCGCTCACCGCCCCTCCTCCGCACCGCCCCTACCCGCA |
| | | GGCCGCGGCGGGCTGTCGGCGCGGGGCACCCTGGGACTTGTAGTCC |
| | | AAGCCGCTTGCCACCTGCCGGCTGCAAACGGCGGAGGGACTACGAA |
| | | GCCCAGAGGTCCCTGCGGCCCTGCCCGCCCACCCGGACACCCCACC |
| | | CCTTCCCCCTCCTTTCCGAAGCCCCCCTCCCTGTTTTTTCAGCCCCCT |
| | | CCCTGCAGGTTCGCATATTAAGGTGACGCGTGTGGCCTCGAACACC |
| | | GAGCGACCCTGCAGCGACCCGCTTAA |
| 217 | SB08580 | GGTTACACAACCAGGGGGGAGGGGCCCCGGGGGGGGAGGGGGC |
| | | CGGCCCGCGGGCCGCGCAGCCGGAAGCCGGGACCGCCACCGGCCC |
| | | GTGAGTGACAGGCGAACCAGTACTATAAATAGATACGGGCCAGAC |
| | | AGATGGTTCCCAATACGACTAATCCTCATAGAAGACGCACTAATCC |
| | | TCCGCGCCGCGCGATCGGCCCGCGCCCATTGGCTCTCCGGCCCGCC |
| | | GCTCACCGCCCCTCCTCCGCACCGCCCCTACCCGCAGGCCGCGGCG |
| | | GGCTGTCGGCGCGGGGCACCCTGGGACTTGTAGTCCAAGCCGCTTG |
| | | CCACCTGCCGGCTGCAAACGGCGGAGGGACTACGAAGCCCAGAGG |
| | | TCCCTGCGGCCCTGCCCGCCCACCCGGACACCCCACCCCTTCCCCCT |
| | | CCTTTCCGAAGCCCCCCTCCCTGTTTTTTCAGCCCCCTCCCTGCAGGT |
| | | CTAGAGGGTATATAATGGGGGCCA |
| 218 | SB08581 | GGTTACACAACCAGGCGGGGAGGGGCCCCGGGGCGGGGAGGGGGC |
| | | CGGCCCGCGGGCCGCGCAGCCGGAAGCCGGGACCGCCACCGGCCC |
| | | CTAAGCCACATTCGTCTAGTACTAATCCTCTCGGCAGCCGATCTAAT |
| | | CCTCAGTCTAGCGCTGACAGATGGTTCACGGGTGGATTAATTGGCG |
| | | CGCCGCGCGATCGGCCCGCGCCCATTGGCTCTCCGGCCCGCCGCTC |
| | | ACCGCCCCTCCTCCGCACCGCCCCTACCCGCAGGCCGCGGCGGGCT |
| | | GTCGGCGCGGGGCACCCTGGGACTTGTAGTCCAAGCCGCTTGCCAC |
| | | CTGCCGGCTGCAAACGGCGGAGGGACTACGAAGCCCAGAGGTCCCT |
| | | GCGGCCCTGCCCGCCCACCCGGACACCCCACCCCTTCCCCCTCCTTT |
| | | CCGAAGCCCCCCTCCCTGTTTTTTCAGCCCCCTCCCTGCAGGTCTAG |
| | | AGGGTATATAATGGGGGCCA |
| 219 | SB08582 | CTCCTGCTATTGGTCAGAATGTGTCACGTGACCATACTCAGCTGCAA |
| | | GGGTTGGTGGGAACTAAGCCACATTCGTCTAGTACTAATCCTCTCG |
| | | GCAGCCGATCTAATCCTCAGTCTAGCGCTGACAGATGGTTCACGGG |
| | | TGGATTAATTGGCTCCTGCTATTGGTCAGAATGTGTCACGTGACCAT |
| | | ACTCAGCTGCAAGGGTTGGTGGGAACTAAGCCACATTCGTCTAGTA |
| | | CTAATCCTCTCGGCAGCCGATCTAATCCTCAGTCTAGCGCTGACAGA |
| | | TGGTTCACGGGTGGATTAATTGGCTCCTGCTATTGGTCAGAATGTGT |
| | | CACGTGACCATACTCAGCTGCAAGGGTTGGTGGGAACTAAGCCACA |
| | | TTCGTCTAGTACTAATCCTCTCGGCAGCCGATCTAATCCTCAGTCTA |
| | | GCGCTGACAGATGGTTCACGGGTGGATTAATTGGCCTGCAGGTCTA |
| | | GAGGGTATATAATGGGGGCCA |
| 220 | SB08583 | CTAATCCTCTCGGCCGATCTAATCCTCAGTCTGCTGACAGATGGTCT |
| | | CCTGCTATTGGTCAGAATGTGTCACGTGACCATACTCAGCTGCAAG |
| | | GGTTGGTGGGAAATGTAGTCTTTTTTTCTGGGAGGCCATGTGTTCGG |
| | | CTTAAAATTGTCTTTTTTTCTTTTTCTAAGCCACATTCGTCTAGTACT |
| | | AATCCTCTCGGCAGCCGATCTAATCCTCAGTCTAGCGCTGACAGAT |
| | | GGTTCACGGGTGGATTAATTGGCTCCTGCTATTGGTCAGAATGTGTC |
| | | ACGTGACCATACTCAGCTGCAAGGGTTGGTGGGAAATGTAGTCTTT |
| | | TTTTCTGGGAGGCCATGTGTTCGGCTTAAAATTGTCTTTTTTTCTTTT |
| | | TCTAAGCCACATTCGTCTAGTACTAATCCTCTCGGCAGCCGATCTAA |
| | | TCCTCAGTCTAGCGCTGACAGATGGTTCACGGGTGGATTAATTGGC |
| | | CTGCAGGTCTAGAGGGTATATAATGGGGGCCA |
| 221 | SB08584 | CTAAGCCACAGTAACATGCGACTAATCCTCTCTGTCTACAGAGTGG |
| | | CTTAGTCTATCGTGTTGCTGACTCAGCATTAGATAGTGACAGATGGT |
| | | TACACGGTTAAGACTCAATCCGGTAAGTAAGAGAAAAACAGGAAA |
| | | AGAGTTTACATGCCACTCCTGCTATTGGTCAGAATGTGTCACGTGAC |
| | | CATACTCAGCTGCAAGGGTTGGTGGGAAATGTAGTCTTTTTTTCTGG |
| | | GAGGCCATGTGTTCGGCTTAAAATTGTCTTTTTTTCTTTTTCTAAGCC |
| | | ACATTCGTCTAGTACTAATCCTCTCGGCAGCCGATCTAATCCTCAGT |
| | | CTAGCGCTGACAGATGGTTCACGGGTGGATTAATTGGCCTGCAGGT |
| | | CTAGAGGGTATATAATGGGGGCCA |
| 222 | SB08585 | GTGAGTGACAGGCGAACCAGTACTATAAATAGATACGGGCCAGAC |
| | | AGATGGTTCCCAATACGACTAATCCTCATAGAAGACGCACTAATCC |
| | | TCTACACGGTTAAGACTCAATCCGGTAAGTAAGAGAAAAACAGGA |
| | | AAAGAGTTTACATGCCACTCCTGCTATTGGTCAGAATGTGTCACGTG |
| | | ACCATACTCAGCTGCAAGGGTTGGTGGGAAATGTAGTCTTTTTTTCT |
| | | GGGAGGCCATGTGTTCGGCTTAAAATTGTCTTTTTTTCTTTTTCTAAG |
| | | CCACATTCGTCTAGTACTAATCCTCTCGGCAGCCGATCTAATCCTCA |
| | | GTCTAGCGCTGACAGATGGTTCACGGGTGGATTAATTGGCCTGCAG |
| | | GTCTAGAGGGTATATAATGGGGGCCA |
| 223 | SB08586 | CTAAGCCACAGTAACATGCGACTAATCCTCTCTGTCTACAGAGTGG |
| | | CTTAGTCTATCGTGTTGCTGACTCAGCATTAGATAGTGACAGATGGT |
| | | TGTTGAGAGCTCAAGCTCTTTTTAACGCTTTGCCTTGCTGTCTCCAA |
| | | AGTATTGCCTTCATCCTCATAGTTCAAAGTGTCCACCATCACATACA |
| | | CGGTTAAGACTCAATCCGGTAAGTAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTAAGCCACAT |
| | | TCGTCTAGTACTAATCCTCTCGGCAGCCGATCTAATCCTCAGTCTAG |
| | | CGCTGACAGATGGTTCACGGGTGGATTAATTGGCCTGCAGGTCTAG |
| | | AGGGTATATAATGGGGGCCA |
| 224 | SB08590 | CTAAGCCACATTCGTCTAGTACTAATCCTCTCGGCAGCCGATCTAAT |
| | | CCTCAGTCTAGCGCTGACAGATGGTTCACGGGTGGATTAATTGGTT |
| | | ACCTACTAGGTTAATTCGTCCGATAGATACTACGAATTGCGAGCTTC |
| | | TAAGTCCAATTTTCGGTATTCGAGTCGTCAGACTCAATTATTACACG |
| | | GTTAAGACTCAATCCGGTAAGTAATACTGTGATCAGGTGTCTACAA |
| | | GACTAGTACTCCTGCTATTGGTCAGAATGTGTCACGTGACCATACTC |
| | | AGCTGCAAGGGTTGGTGGGAAATGTAGTCTTTTTTTCTGGGAGGCC |
| | | ATGTGTTCGGCTTAAAATTGTCTTTTTTTCTTTTTACTATCACGTATA |
| | | TACCAGCGAGTTCGATAATACACTCTCCTGCAGGTCTAGAGGGTAT |
| | | ATAATGGGGGCCA |
| 225 | SB08591 | CTAAGCCACATTCGTCTAGTACTAATCCTCTCGGCAGCCGATCTAAT |
| | | CCTCAGTCTAGCGCTGACAGATGGTTCACGGGTGGATTAATTGGTG |
| | | TTGAGAGCTCAAGCTCTTTTTAACGCTTTGCCTTGCTGTCTCCAAAG |
| | | TATTGCCTTCATCCTCATAGTTCAAAGTGTCCACCATCACATACACG |
| | | GTTAAGACTCAATCCGGTAAGTAAGAGAAAAACAGGAAAAGAGTT |
| | | TACATGCCACTCCTGCTATTGGTCAGAATGTGTCACGTGACCATACT |
| | | CAGCTGCAAGGGTTGGTGGGAAATGTAGTCTTTTTTTCTGGGAGGC |
| | | CATGTGTTCGGCTTAAAATTGTCTTTTTTTCTTTTTCTTTATTTCTTTT |
| | | TTCTTTTCTTTCTTTTTATACACTCTCCTGCAGGTCTAGAGGGTATAT |
| | | AATGGGGGCCA |
| 226 | SB08592 | CTAAGCCACATTCGTCTAGTACTAATCCTCTCGGCAGCCGATCTAAT |
| | | CCTCAGTCTAGCGCTGACAGATGGTTCACGGGTGGATTAATTGGTG |
| | | TTGAGAGCTCAAGCTCTTTTTAACGCTTTGCCTTGCTGTCTCCAAAG |
| | | TATTGCCTTCATCCTCATAGTTCAAAGTGTCCACCATCACATTCACG |
| | | TTTCAGCCAATAGGAAGAAGGAAAGAGAAAAACAGGAAAAGAGTT |
| | | TACATGCCACTCCTGCTATTGGTCAGAATGTGTCACGTGACCATACT |
| | | CAGCTGCAAGGGTTGGTGGGAAATGTAGTCTTTTTTTCTGGGAGGC |
| | | CATGTGTTCGGCTTAAAATTGTCTTTTTTTCTTTTTCTTTATTTCTTTT |
| | | TTCTTTTCTTTCTTTTTTTTAGACGGCCTGCAGGTCTAGAGGGTATAT |
| | | AATGGGGGCCA |
| 227 | SB08593 | TAGTACTAATCCTCTCGGCAGCCGATCTAATCCTCAGTCTAGCGCTG |
| | | ACAGATGGTTCACTAGTAAGGTACTAAGCCACATTCGTCTAGTACT |
| | | AATCCTCTCGGCAGCCGATCTAATCCTCAGTCTAGCGCTGACAGAT |
| | | GGTTCACGGGTGGATTAATTGGTTTGCGCGTACTAAGCCACATTCGT |
| | | CTAGTACTAATCCTCTCGGCAGCCGATCTAATCCTCAGTCTAGCGCT |
| | | GACAGATGGTTCACGGGTGGATTAATTGGAGTCCGGGTACTAAGCC |
| | | ACATTCGTCTAGTACTAATCCTCTCGGCAGCCGATCTAATCCTCAGT |
| | | CTAGCGCTGACAGATGGTTCACGGGTGGATTAATTGGCCTGCAGGT |
| | | CTAGAGGGTATATAATGGGGGCCA |
| 228 | SB08594 | CTAAGCCACATTCGTCTAGTACTAATCCTCTCGGCAGCCGATCTAAT |
| | | CCTCAGTCTAGCGCTGACAGATGGTTCACGGGTGGATTAATTGGGT |
| | | GAGTGACAGGCGACCAGTACTATAAATAGATACGGGCCAGACAGA |
| | | TGGTTCCCAATACGACTAATCCTCATAGAAGACGCACTAATCCTCCT |
| | | AAGCCACATTCGTCTAGTACTAATCCTCTCGGCAGCCGATCTAATCC |
| | | TCAGTCTAGCGCTGACAGATGGTTCACGGGTGGATTAATTGGGTGA |
| | | GTGACAGGCGAACCAGTACTATAAATAGATACGGGCCAGACAGAT |
| | | GGTTCCCAATACGACTAATCCTCATAGAAGACGCACTAATCCTCCCT |
| | | GCAGGTCTAGAGGGTATATAATGGGGGCCA |
| 229 | SB08595 | GTGAGTGACAGGCGACCAGTACTATAAATAGATACGGGCCAGACA |
| | | GATGGTTCCCAATACGACTAATCCTCATAGAAGACGCACTAATCCT |
| | | CGTGAGTGACAGGCGAACCAGTACTATAAATAGATACGGGCCAGA |
| | | CAGATGGTTCCCAATACGACTAATCCTCATAGAAGACGCACTAATC |
| | | CTCCTAAGCCACATTCGTCTAGTACTAATCCTCTCGGCAGCCGATCT |
| | | AATCCTCAGTCTAGCGCTGACAGATGGTTCACGGGTGGATTAATTG |
| | | GCTAAGCCACATTCGTCTAGTACTAATCCTCTCGGCAGCCGATCTAA |
| | | TCCTCAGTCTAGCGCTGACAGATGGTTCACGGGTGGATTAATTGGC |
| | | CTGCAGGTCTAGAGGGTATATAATGGGGGCCA |
| 230 | SB08725 | CTAAGCCACATTCGTCTAGTACTAATCCTCTCGGCAGCCGATCTAAT |
| | | CCTCAGTCTAGCGCTGACAGATGGTTCACGGGTGGATTAATTGGTG |
| | | CTGAGTCAGCATAGGAAGAGTCTAAGCCACAGCACGCGTAGTCTAA |
| | | TCCTCAGCCTACTTAACTATAAATAGATCGAGCAATACCATCTGTCT |
| | | GCTGACTCAGCAACGACTATAAGTGGCTTAGAACATAACGTCTCTA |
| | | ATCCTCAGACTGCGCGTGACAGATGGTTTCGTCCCGTACCATCTGTC |
| | | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TCTAGAGGGTATATAATGGGGGCCA |
| 231 | SB08726 | ACCATCTGTCAATCGGCTTAACCATCTGTCAAGTATCTATACCATCT |
| | | GTCAGTCCGGGTATGCTGAGTCAGCATAGGAAGAGTCTAAGCCACA |
| | | GCACGCGTAGTCTAATCCTCAGCCTACTTAACTATAAATAGATCGA |
| | | GCAATACCATCTGTCTTTGCGCGTATGCTGACTCAGCAACGACTATA |
| | | AGTGGCTTAGAACATAACGTCTCTAATCCTCAGACTGCGCGTGACA |
| | | GATGGTTTCGTCCCGTACCATCTGTCTAGTAAGGTATGCTGAGTCAG |
| | | CAAGTATACGAACTAAGCCACAGGAGTCTTGTACCATCTGTCAATC |
| | | GGCTTAACCATCTGTCAAGTATCTATACCATCTGTCCCTGCAGGTCT |
| | | AGAGGGTATATAATGGGGGCCA |
| 232 | SB08727 | ACCATCTGTCAATCGGCTTAACCATCTGTCAAGTATCTATACCATCT |
| | | GTCAGTCCGGGTATGCTGAGTCAGCAAGTATACGAACTAAGCCACA |
| | | GGAGTCTTGTACCATCTGTCAATCGGCTTAACCATCTGTCAAGTATC |
| | | TATACCATCTGTCTTTGCGCGTATGCTGAGTCAGCAAGTATACGAAC |
| | | TAAGCCACAGGAGTCTTGTACCATCTGTCAATCGGCTTAACCATCTG |
| | | TCAAGTATCTATACCATCTGTCTAGTAAGGTATGCTGAGTCAGCAA |
| | | GTATACGAACTAAGCCACAGGAGTCTTGTACCATCTGTCAATCGGC |
| | | TTAACCATCTGTCAAGTATCTATACCATCTGTCCCTGCAGGTCTAGA |
| | | GGGTATATAATGGGGGCCA |
| 233 | SB08728 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TCTAGAGGGTATATAATGGGGGCCA |
| 234 | SB08729 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CTCAGGCCCTGCATTGCGCCAACGGCGCAGCGCTGGGCCGCGACCC |
| | | CGGCACCGGCACCCGGGGACGTTGTGAATAGCTGGTCGAAGTTTGG |
| | | TTGAAGCACAGGAGGGTAAATAAGAAGGTTTCCAGACAAAGAGAC |
| | | TCTTAAAGGTACAGACTCTTATGTCTGTCCCTCCTCCTTAAAGGGCC |
| | | AGAGACGCTCCCGAGCCCATCTCCTCCTGCTATTGGTCAGAATGTGT |
| | | CACGTGACCATACTCAGCTGCAAGGGTTGGTGGGAAATGTAGTCTT |
| | | TTTTTCTGGGAGGCCATGTGTTCGGCTTAAAATTGTCTTTTTTTCTTT |
| | | TTCCTGCAGGAGACGCTAGCGGGGGGCTATAAAAGGGGGTGGGGG |
| | | CGTTCGTCCTCACTCT |
| 235 | SB08730 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACCCCCCT |
| | | CCCTGTTTTTTCAGCCCCCTCTGCTGAGTCAGCAAGTATACGAACTA |
| | | AGCCACAGGAGTCTTGTACCATCTGTCAATCGGCTTAACCATCTGTC |
| | | AAGTATCTATACCATCTGTCTCGAGGCGCCGGCGGGGCGGCCCGGC |
| | | GGCCGGAAGTGCACCCTGCTCGGGCTTTGCGCCCCCGGCAGCTTCC |
| | | TCCGCCCGCGAGGTCTAGAGGGTATATAATGGGGGCCA |
| 236 | SB08731 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACCCCCCT |
| | | CCCTGTTTTTTCAGCCCCCTCTCGAGGCGCCGGCGGGGCGGCCCGGC |
| | | GGCCGGAAGTGCACCCTGCTCGGGCTTTGCGCCCCCGGCAGCTTCC |
| | | TCCGCCCGCGAGGTCTAGAGGGTATATAATGGGGGCCA |
| 237 | SB08732 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACCCCCCT |
| | | CCCTGTTTTTTCAGCCCCCTCTCGAGGCGCCGGCGGGGCGGCCCGGC |
| | | GGCCGGAAGTGCACCCTGCTCGGGCTTTGCGCCCCCGGCAGCTTCC |
| | | TCCGCCCGCGAGGCCTGCAGGTTCGCATATTAAGGTGACGCGTGTG |
| | | GCCTCGAACACCGAGCGACCCTGCAGCGACCCGCTTAA |
| 238 | SB08733 | GGTTACACAACCAGGCGGGGAGGGGCCCCGGCAGCCGGAAGCCGG |
| | | GACCGCCACCGGCCCCGCGCCGCGCGATCGGCCCGCGCCCATTGGC |
| | | TCTCCGGCCCGCCGCTCACCGCCCCTCCTCCGCACCGCCCCTACCCG |
| | | CAGGCCGCGGCGGGCTGTCGGCGCGGGGCACCCTGGGACTTGTAGT |
| | | CCAAGCCGCTTGCCACCTGCCGGCTGCAAACGGCGGAGGGACTACG |
| | | AAGCCCAGAGGTCCCTGCGGCCCTGCCCGCCCACCCGGACCCCCCT |
| | | CCCTGTTTTTTCAGCCCCCTCTCGAGGCGCCGGCGGGGCGGCCCGGC |
| | | GGCCGGAAGTGCACCCTGCTCGGGCTTTGCGCCCCCGGCAGCTTCC |
| | | TCCGCCCGCGAGGCCTGCAGGTCTAGAGGGTATATAATGGGGGCCA |
| 239 | SB08850 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CTCCTGCTATTGGTCAGAATGTGTCACGTGACCATACTCAGCTGCAA |
| | | GGGTTGGTGGGAAATGTAGTCTTTTTTTCTGGGAGGCCATGTGTTCG |
| | | GCTTAAAATTGTCTTTTTTTCTTTTTCTAAGCCACAGTAACATGCGA |
| | | CTAATCCTCTCTGTCTACAGAGTGGCTTAGTCTATCGTGTTGCTGAC |
| | | TCAGCATTAGATAGTGACAGATGGTCCTGCAGGTCTAGAGGGTATA |
| | | TAATGGGGGCCA |
| 240 | SB08851 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | TACACGGTTAAGACTCAATCCGGTAAGTAAGAGAAAAACAGGAAA |
| | | AGAGTTTACATGCCACTCCTGCTATTGGTCAGAATGTGTCACGTGAC |
| | | CATACTCAGCTGCAAGGGTTGGTGGGAAATGTAGTCTTTTTTTCTGG |
| | | GAGGCCATGTGTTCGGCTTAAAATTGTCTTTTTTTCTTTTTCTAAGCC |
| | | ACAGTAACATGCGACTAATCCTCTCTGTCTACAGAGTGGCTTAGTCT |
| | | ATCGTGTTGCTGACTCAGCATTAGATAGTGACAGATGGTCCTGCAG |
| | | GAGACGCTAGCGGGGGGCTATAAAAGGGGGTGGGGGCGTTCGTCC |
| | | TCACTCT |
| 241 | SB08854 | ACCATCTGTCTAGTAAGGTATGCTGAGTCAGCAAGTATACGAACTA |
| | | AGCCACAGGAGTCTTGTACCATCTGTCAATCGGCTTAACCATCTGTC |
| | | AAGTATCTATACCATCTGTCCCTGCAGGTCTAGAGGGTATATAATG |
| | | GGGGCCA |
| 242 | SB08855 | ACCATCTGTCAAGTATCTATACCATCTGTCTAGTAAGGTATGCTGAG |
| | | TCAGCAAGTATACGAACTAAGCCACAGGAGTCTTGTACCATCTGTC |
| | | AATCGGCTTAACCATCTGTCAAGTATCTATACCATCTGTCCCTGCAG |
| | | GTCTAGAGGGTATATAATGGGGGCCA |
| 243 | SB08856 | ACCATCTGTCAATCGGCTTAACCATCTGTCAAGTATCTATACCATCT |
| | | GTCTAGTAAGGTATGCTGAGTCAGCAAGTATACGAACTAAGCCACA |
| | | GGAGTCTTGTACCATCTGTCAATCGGCTTAACCATCTGTCAAGTATC |
| | | TATACCATCTGTCCCTGCAGGTCTAGAGGGTATATAATGGGGGCCA |
| 244 | SB08857 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CCTGCAGGTAGGCGTGTACGGTGGGAGGCCTATATAAGCAGAGCTC |
| | | GTTTAGTGAACCGTCAGATCGCCTGGA |
| 245 | SB08858 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CCTGCAGGAGACGCTAGCGGGGGGCTATAAAAGGGGGTGGGGGCG |
| | | TTCGTCCTCACTCT |
| 246 | SB08859 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CCTGCAGGAAAATGTGCGCATGTGCAGCCATTGCCTGGGACGCATG |
| | | CGTAGGGAGCCGCGCGACAAACTGAGCCATTGCGGCAAGACTAGC |
| | | GCAGAGAGGAGAGGGAGCCGGAGATGCCAGACGCTTGGTTCTGAG |
| | | GAGTGATTTGCAACGCAATGGAGCGAGGAAGG |
| 247 | SB08860 | TGCTGAGTCAGCAAGTATACGAACTAAGCCACAGGAGTCTTGTACC |
| | | ATCTGTCAATCGGCTTAACCATCTGTCAAGTATCTATACCATCTGTC |
| | | CCTGCAGGCAGAATTAACAGTATAAATTGCATCTCTTGTTCAAGAG |
| | | TTCCCTATCACTCT |
| 276 | SB08331 | GTGGCTTAGAAAGGTCCGATGCTGAGTCAGCAAGCAAGTACTCTAA |
| | | TCCTCTATAGGACAAACTATAAATAGAATACGAGGAGTTAATCCCC |
| | | TGTTAATCCCCTAGCTTAGATTCTATTTATAGTTATTACTATGCTGAC |
| | | TCAGCAATAGACGGTAGAGGATTAGAGCACTCGTTTGACAGATGGT |
| | | CTCCTGCTATTGGTCAGAATGTGTCACGTGACCATACTCAGCTGCAA |
| | | GGGTTGGTGGGAACTAAGCCACAGTAACATGCGACTAATCCTCTCT |
| | | GTCTACAGAGTGGCTTAGTCTATCGTGTTGCTGACTCAGCATTAGAT |
| | | AGTGACAGATGGTCCTGCAGGTCTAGAGGGTATATAATGGGGGCCA |
| 277 | SB08332 | GTGGCTTAGAAAGGTCCGATGCTGAGTCAGCAAGCAAGTACTCTAA |
| | | TCCTCTATAGGACAAACTATAAATAGAATACGAGGAGTTAATCCCC |
| | | TTACACGGTTAAGACTCAATCCGGTAAGTAAGAGAAAAACAGGAA |
| | | AAGAGTTTACATGCCACTCCTGCTATTGGTCAGAATGTGTCACGTGA |
| | | CCATACTCAGCTGCAAGGGTTGGTGGGAAATGTAGTCTTTTTTTCTG |
| | | GGAGGCCATGTGTTCGGCTTAAAATTGTCTTTTTTTCTTTTTCTAAGC |
| | | CACAGTAACATGCGACTAATCCTCTCTGTCTACAGAGTGGCTTAGTC |
| | | TATCGTGTTGCTGACTCAGCATTAGATAGTGACAGATGGTCCTGCA |
| | | GGTCTAGAGGGTATATAATGGGGGCCA |
| 278 | SB08333 | GTTAATCCCCTAGCTTAGATTCTATTTATAGTTATTACTATGCTGACT |
| | | CAGCAATAGACGGTAGAGGATTAGAGCACTCGTTTGACAGATGGTT |
| | | ACACGGTTAAGACTCAATCCGGTAAGTAAGAGAAAAACAGGAAAA |
| | | GAGTTTACATGCCACTCCTGCTATTGGTCAGAATGTGTCACGTGACC |
| | | ATACTCAGCTGCAAGGGTTGGTGGGAAATGTAGTCTTTTTTTCTGGG |
| | | AGGCCATGTGTTCGGCTTAAAATTGTCTTTTTTTCTTTTTCTAAGCCA |
| | | CAGTAACATGCGACTAATCCTCTCTGTCTACAGAGTGGCTTAGTCTA |
| | | TCGTGTTGCTGACTCAGCATTAGATAGTGACAGATGGTCCTGCAGG |
| | | TCTAGAGGGTATATAATGGGGGCCA |
| 248 | Enhancer segment | TGCCTTGCTGTCTCCAAAGTATTGCCTTCATCCTCATAGTTCAAAGT |
| | | GTCCACCATCACATTCACGTTTCAGCCAATAGGAAGAAGGAAAGAG |
| | | AAAAACAGGAAAAGAGTTTACATGCCACTCCTGCTATTGGTCAGAA |
| | | TGTGTCACGTGACCATACTCAGCTGCAAGGGTTGGTGGGAAATGTA |
| | | GTCTTTTTTTCTGGGAGGCCATGTGTTCGGCTTAAAATTGTCTTTTTT |
| | | TCTTTTTCTTTATTTCTTTTTTCTTTTCTTTCTTTTTTTTAGACGG |
| 249 | Enhancer segment | TGTTGAGAGCTCAAGCTCTTTTTAACGCTT |
| 250 | Enhancer segment | TCCTCATAGTTCAAAGTGTCCACCATCACATTCACGTTTCAGCCAAT |
| | | AGGAAGAAGGAAAGAGAAAAACAGGAAAAGAGTTTACATGCCACT |
| | | CCTGCTATTGGTCAGAATGTGTCACGTGACCATACTCAGCTGCAAG |
| | | GGTTGGTGGGAAATGTAGTCTTTTTTTCTGGGAGGCCATGTGTTCGG |
| | | CTTAAAATTGTCTTTTTTTCTTTTTCTTTATTTCTTTTTTCTTTTCTTTC |
| | | TTTTTTTTAGACGG |
| 251 | Enhancer segment | TGTTGAGAGCTCAAGCTCTTTTTAACGCTTTGCCTTGCTGTCTCCAA |
| | | AGTATTGCCTTCA |
| 252 | Enhancer segment | TTCACGTTTCAGCCAATAGGAAGAAGGAAAGAGAAAAACAGGAAA |
| | | AGAGTTTACATGCCACTCCTGCTATTGGTCAGAATGTGTCACGTGAC |
| | | CATACTCAGCTGCAAGGGTTGGTGGGAAATGTAGTCTTTTTTTCTGG |
| | | GAGGCCATGTGTTCGGCTTAAAATTGTCTTTTTTTCTTTTTCTTTATT |
| | | TCTTTTTTCTTTTCTTTCTTTTTTTTAGACGG |
| 253 | Enhancer segment | TGTTGAGAGCTCAAGCTCTTTTTAACGCTTTGCCTTGCTGTCTCCAA |
| | | AGTATTGCCTTCATCCTCATAGTTCAAAGTGTCCACCATCACA |
| 254 | Enhancer segment | GAGAAAAACAGGAAAAGAGTTTACATGCCACTCCTGCTATTGGTCA |
| | | GAATGTGTCACGTGACCATACTCAGCTGCAAGGGTTGGTGGGAAAT |
| | | GTAGTCTTTTTTTCTGGGAGGCCATGTGTTCGGCTTAAAATTGTCTTT |
| | | TTTTCTTTTTCTTTATTTCTTTTTTCTTTTCTTTCTTTTTTTTAGACGG |
| 255 | Enhancer segment | TGTTGAGAGCTCAAGCTCTTTTTAACGCTTTGCCTTGCTGTCTCCAA |
| | | AGTATTGCCTTCATCCTCATAGTTCAAAGTGTCCACCATCACATTCA |
| | | CGTTTCAGCCAATAGGAAGAAGGAAA |
| 256 | Enhancer segment | CTCCTGCTATTGGTCAGAATGTGTCACGTGACCATACTCAGCTGCAA |
| | | GGGTTGGTGGGAAATGTAGTCTTTTTTTCTGGGAGGCCATGTGTTCG |
| | | GCTTAAAATTGTCTTTTTTTCTTTTTCTTTATTTCTTTTTTCTTTTCTTT |
| | | CTTTTTTTTAGACGG |
| 257 | Enhancer segment | TGTTGAGAGCTCAAGCTCTTTTTAACGCTTTGCCTTGCTGTCTCCAA |
| | | AGTATTGCCTTCATCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTT |
| 258 | Enhancer segment | TTTAGACGG |
| 259 | Enhancer segment | TGTTGAGAGCTCAAGCTCTTTTTAACGCTTTGCCTTGCTGTCTCCAA |
| | | AGTATTGCCTTCATCCTCATAGTTCAAAGTGTCCACCATCACATTCA |
| | | CGTTTCAGCCAATAGGAAGAAGGAAAGAGAAAAACAGGAAAAGAG |
| | | TTTACATGCCACTCCTGCTATTGGTCAGAATGTGTCACGTGACCATA |
| | | CTCAGCTGCAAGGGTTGGTGGGAAATGTAGTCTTTTTTTCTGGGAGG |
| | | CCATGTGTTCGGCTTAAAATTGTCTTTTTTTCTTTTTCTTTATTTCTTT |
| | | TTTCTTTTCTTTCTTTTT |
| 260 | Enhancer segment | GGTTACACAACCAGGCGGGGAGGGGCCCCGGGGGGGGGAGGGGGC |
| | | CGGCCCGCGGGCCGCGCAGCCGGAAGCCGGGACCGCCACCGGCCC |
| 261 | Enhancer segment | CCCCCTGGCGGGCTGGCCCCGCCCCCGCGCCGCGCCGCGCGATCGG |
| | | CCCGCGCCCATTGGCTCTCCGGCCCGCCGCTCACCGCCCCTCCTCCG |
| | | CACCGCCCCTACCCGCAGGCCGCGGCGGGCTGTCGGCGCGGGGCAC |
| | | CCTGGGACTTGTAGTCCAAGCCGCTTGCCACCTGCCGGCTGCAAAC |
| | | GGCGGAGGGACTACGAAGCCCAGAGGTCCCTGCGGCCCTGCCCGCC |
| | | CACCCGGACACCCCACCCCTTCCCCCTCCTTTCCGAAGCCCCCCTCC |
| | | CTGTTTTTTCAGCCCCCTC |
| 262 | Enhancer segment | GGTTACACAACCAGGCGGGGAGGGGCCCCGGGGGGGGAGGGGGC |
| | | CGGCCCGCGGGCCGCGCAGCCGGAAGCCGGGACCGCCACCGGCCC |
| | | CCGGCGAGGGGAGCCCGGCTCCAGGCCCCG |
| 263 | Enhancer segment | CGCGCCGCGCGATCGGCCCGCGCCCATTGGCTCTCCGGCCCGCCGC |
| | | TCACCGCCCCTCCTCCGCACCGCCCCTACCCGCAGGCCGCGGCGGG |
| | | CTGTCGGCGCGGGGCACCCTGGGACTTGTAGTCCAAGCCGCTTGCC |
| | | ACCTGCCGGCTGCAAACGGCGGAGGGACTACGAAGCCCAGAGGTC |
| | | CCTGCGGCCCTGCCCGCCCACCCGGACACCCCACCCCTTCCCCCTCC |
| | | TTTCCGAAGCCCCCCTCCCTGTTTTTTCAGCCCCCTC |
| 264 | Enhancer segment | CAGGCGGGGAGGGGCCCCGGGGGGGGGAGGGGGCCGGCCCGCGGG |
| | | CCGCGCAGCCGGAAGCCGGGACCGCCACCGGCCCCCGGCGAGGGG |
| 265 | Enhancer segment | GCTGGCCCCGCCCCCGCGCCGCGCCGCGCGATCGGCCCGCGCCCAT |
| | | TGGCTCTCCGGCCCGCCGCTCACCGCCCCTCCTCCGCACCGCCCCTA |
| | | CCCGCAGGCCGCGGCGGGCTGTCGGCGCGGGGCACCCTGGGACTTG |
| | | TAGTCCAAGCCGCTTGCCACCTGCCGGCTGCAAACGGCGGAGGGAC |
| | | TACGAAGCCCAGAGGTCCCTGCGGCCCTGCCCGCCCACCCGGACAC |
| | | CCCACCCCTTCCCCCTCCTTTCCGAAGCCCCCCTCCCTGTTTTTTCAG |
| | | CCCCCTCCCCCCCATCCCCCATGGAGC |
| 266 | Enhancer segment | CAGGCGGGGAGGGGCCCCGGGGGGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGG |
| 267 | Enhancer segment | ATCGGCCCGCGCCCATTGGCTCTCCGGCCCGCCGCTCACCGCCCCTC |
| | | CTCCGCACCGCCCCTACCCGCAGGCCGCGGCGGGCTGTCGGCGCGG |
| | | GGCACCCTGGGACTTGTAGTCCAAGCCGCTTGCCACCTGCCGGCTG |
| | | CAAACGGCGGAGGGACTACGAAGCCCAGAGGTCCCTGCGGCCCTG |
| | | CCCGCCCACCCGGACACCCCACCCCTTCCCCCTCCTTTCCGAAGCCC |
| | | CCCTCCCTGTTTTTTCAGCCCCCTCCCCCCCATCCCCCATGGAGC |
| 268 | Enhancer segment | CAGGCGGGGAGGGGCCCCGGGGGGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCC |
| 269 | Enhancer segment | CGGGGCACCCTGGGACTTGTAGTCCAAGCCGCTTGCCACCTGCCGG |
| | | CTGCAAACGGCGGAGGGACTACGAAGCCCAGAGGTCCCTGCGGCC |
| | | CTGCCCGCCCACCCGGACACCCCACCCCTTCCCCCTCCTTTCCGAAG |
| | | CCCCCCTCCCTGTTTTTTCAGCCCCCTCCCCCCCATCCCCCATGGAG |
| | | C |
| 270 | Enhancer segment | CAGGCGGGGAGGGGCCCCGGGGCGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCC |
| 271 | Enhancer segment | GGGACTACGAAGCCCAGAGGTCCCTGCGGCCCTGCCCGCCCACCCG |
| | | GACACCCCACCCCTTCCCCCTCCTTTCCGAAGCCCCCCTCCCTGTTTT |
| | | TTCAGCCCCCTCCCCCCCATCCCCCATGGAGC |
| 272 | Enhancer segment | CAGGCGGGGAGGGGCCCCGGGGCGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGC |
| 273 | Enhancer segment | TCCCCCTCCTTTCCGAAGCCCCCCTCCCTGTTTTTTCAGCCCCCTCCC |
| | | CCCCATCCCCCATGGAGC |
| 274 | Enhancer segment | CAGGCGGGGAGGGGCCCCGGGGCGGGGAGGGGGCCGGCCCGCGGG |
| | | CCGCGCAGCCGGAAGCCGGGACCGCCACCGGCCCCCGGCGAGGGG |
| | | AGCCCGGCTCCAGGCCCCGCCCCCTGGCGGGCTGGCCCCGCCCCCG |
| | | CGCCGCGCCGCGCGATCGGCCCGCGCCCATTGGCTCTCCGGCCCGC |
| | | CGCTCACCGCCCCTCCTCCGCACCGCCCCTACCCGCAGGCCGCGGC |
| | | GGGCTGTCGGCGCGGGGCACCCTGGGACTTGTAGTCCAAGCCGCTT |
| | | GCCACCTGCCGGCTGCAAACGGCGGAGGGACTACGAAGCCCAGAG |
| | | GTCCCTGCGGCCCTGCCCGCCCACCCGGACACCCCACCCCT |
| 275 | Enhancer segment | TTTTTTCAGCCCCCTCCCCCCCATCCCCCATGGAGC |

### EXAMPLES

The present disclosure will be further illustrated in the following Examples which are given for illustration purposes only and are not intended to limit the disclosure in any way.

### Example 1: Identification and Screening of Native Regulatory Elements

For this study, candidate regulatory elements (e.g., promoters and/or enhancers) were identified through screening: 1) libraries derived from transcription factor binding site motifs; or 2) libraries consisting of natural (i.e., native or wild-type) enhancers and promoter sequences. Libraries were functionally screened by flow cytometry by measuring expression of GFP or mCherry as a functional readout of the candidate regulatory element.

The candidate regulatory elements were bioinformatically identified from genes that are differentially active or expressed in rod and cone photoreceptor cells (ON-target cell types) as compared to non-photoreceptor cells (retinal pigment epithelium cells and Mueller glia cells, OFF-target cell types). Selected regulatory elements were cloned into DNA libraries.

Target native regulatory element libraries were screened through clonal or MPRA screening to validate the activity to the native regulatory elements. The top regulatory elements identified in the analysis are provided in Table 1 with the respective strength (as a percentage of CAG promoter) and specificity between cell lines Y79 and ARPE-19.

### I. Cell Lines

Surrogate cell lines used in the study are: Y79, ARPE-19, and MIO-M1. Y79, rod and cone photoreceptor cell line, serves as ON-target cell line, and both ARPE-19, retinal pigment epithelium cell line, and MIO-M1, Mueller cell line, serve as OFF-target cell line.

### II. Clonal Screening

### Transfection

Cell lines were co-transfected with two DNA vectors: one vector coding for mCherry driven by CAG promoter serving as an activity control, a second vector coding for GFP driven by screened enhancer and/or promoter sequences. Transfections were performed using commonly used transfection reagents, according to the manufacturer's guidelines, with specific detailed changes found to be beneficial.

### Clonal Screening Analysis

Reporter gene expression data acquired from flow cytometry was used to calculate promoter strength in ON target cell types and specificity against OFF target cell types. Briefly, data for each promoter in each cell type was collapsed to a representative expression curve using a binned-average method; the area under the curve (AUC) for each promoter expression curve was compared to that of a benchmark promoter, CAG, to calculate strength; the AUC for each promoter in the ON target cell type was compared with the counterpart AUC in each of the OFF target cell types to calculate specificity.

### III. Identification of Candidate Promoters, Enhancers, Transcription Factors (TFs)

### Natural Promoters and Enhancers

Candidate promoters were identified by analyzing publicly available scRNAseq datasets. Candidate enhancer elements were selected from publicly available ATAC-seq data and ranked based on sequence length and distance from differentially regulated genes.

### Synthetic MPRA Library Design

MPRA libraries were designed using computational tools by combining transcription factor binding motifs (TFBM) into short synthetic enhancers. Selection of TFBMs was guided by the literature as well as TFBM enrichment analysis of natural sequences displaying the desired ON/OFF target profile. Consensus TFBM sequences were derived from the literature as well as publicly available databases. Each synthetic (i.e., engineered) enhancer was also paired with random barcode sequences to be used in the downstream analysis.

### MPRA Analysis

MPRA libraries were introduced in cells by transfection. Next generation sequencing (NGS) was used to quantify the abundance of barcodes associated with each enhancer in each of the cell types.

Enhancer performance was evaluated, and relative strength and specificity was calculated for each library member in each ON/OFF target surrogate cell line pair. Top performing hits were selected for experimental validation.

TABLE 1
Selected Native Enhancer & Promoter Regulatory Elements

| | | Spec- | | | | | |
|---|---|---|---|---|---|---|---|
| | | ificity | | Enhancer | | | Minimal |
| Construct | Strength | (Y79: | | genomic | | Spacer | promoter |
| Name | (%CAG) | ARPE-19) | Enhancer-promoter sequence | location | Enhancer sequence | sequence | sequence |
| SB05201 | 5.3 | 195 | TGTTGAGAGCTCAAGCTCTTTT | CHR19:3 | TGTTGAGAGCTCAAGCTCTTT | CCTGC | TCTAGA |
| | | | TAACGCTTTGCCTTGCTGTCTC | 990222- | TTAACGCTTTGCCTTGCTGTC | AGG | GGGTAT |
| | | | CAAAGTATTGCCTTCATCCTCA | 3990531 | TCCAAAGTATTGCCTTCATCC | (SEQ ID | ATAATG |
| | | | TAGTTCAAAGTGTCCACCATCA | | TCATAGTTCAAAGTGTCCACC | NO: 107) | GGGGCC |
| | | | CATTCACGTTTCAGCCAATAGG | | ATCACATTCACGTTTCAGCCA | | A (YB- |
| | | | AAGAAGGAAAGAGAAAAACAG | | ATAGGAAGAAGGAAAGAGA | | TATA: |
| | | | GAAAAGAGTTTACATGCCACTC | | AAAACAGGAAAAGAGTTTAC | | SEQ ID |
| | | | CTGCTATTGGTCAGAATGTGTC | | ATGCCACTCCTGCTATTGGTC | | NO: 122) |
| | | | ACGTGACCATACTCAGCTGCAA | | AGAATGTGTCACGTGACCAT | | |
| | | | GGGTTGGTGGGAAATGTAGTCT | | ACTCAGCTGCAAGGGTTGGT | | |
| | | | TTTTTTCTGGGAGGCCATGTGT | | GGGAAATGTAGTCTTTTTTTC | | |
| | | | TCGGCTTAAAATTGTCTTTTTTT | | TGGGAGGCCATGTGTTCGGC | | |
| | | | CTTTTTCTTTATTTCTTTTTTCTT | | TTAAAATTGTCTTTTTTTC | | |
| | | | TTCTTTCTTTTTTTTAGACGGCC | | TTTTTCTTTATTTCTTTTTTC | | |
| | | | TGCAGGTCTAGAGGGTATATAA | | TTTTCTTTCTTTTTTTTAGA | | |
| | | | TGGGGGCCA (SEQ ID NO: 138) | | CGG(SEQ ID NO: 1) | | |
| SB05241 | 9.1 | 142.8 | GGTTACACAACCAGGCGGGGA | CHR19:4 | GGTTACACAACCAGGCGGGG | CCTGC | TCTAGA |
| | | | GGGGCCCCGGGGCGGGGAGGG | 065783- | AGGGGCCCCGGGGCGGGGAG | AGG | GGGTAT |
| | | | GGCCGGCCCGCGGGCCGCGCA | 4066200 | GGGGCCGGCCCGCGGGCCGC | (SEQ ID | ATAATG |
| | | | GCCGGAAGCCGGGACCGCCAC | | GCAGCCGGAAGCCGGGACCG | NO: 107) | GGGGCC |
| | | | CGGCCCCCGGCGAGGGGAGCC | | CCACCGGCCCCCGGCGAGGG | | A (YB- |
| | | | CGGCTCCAGGCCCCGCCCCCTG | | GAGCCCGGCTCCAGGCCCCG | | TATA; |
| | | | GCGGGCTGGCCCCGCCCCCGCG | | CCCCCTGGCGGGCTGGCCCC | | SEQ ID |
| | | | CCGCGCCGCGCGATCGGCCCGC | | GCCCCCGCGCCGCGCCGCGC | | NO: 122) |
| | | | GCCCATTGGCTCTCCGGCCCGC | | GATCGGCCCGCGCCCATTGG | | |
| | | | CGCTCACCGCCCCTCCTCCGCA | | CTCTCCGGCCCGCCGCTCACC | | |
| | | | CCGCCCCTACCCGCAGGCCGCG | | GCCCCTCCTCCGCACCGCCCC | | |
| | | | GCGGGCTGTCGGCGCGGGGCA | | TACCCGCAGGCCGCGGCGGG | | |
| | | | CCCTGGGACTTGTAGTCCAAGC | | CTGTCGGCGCGGGGCACCCT | | |
| | | | CGCTTGCCACCTGCCGGCTGCA | | GGGACTTGTAGTCCAAGCCG | | |
| | | | AACGGCGGAGGGACTACGAAG | | CTTGCCACCTGCCGGCTGCA | | |
| | | | CCCAGAGGTCCCTGCGGCCCTG | | AACGGCGGAGGGACTACGAA | | |
| | | | CCCGCCCACCCGGACACCCCAC | | GCCCAGAGGTCCCTGCGGCC | | |
| | | | CCCTTCCCCCTCCTTTCCGAAG | | CTGCCCGCCCACCCGGACAC | | |
| | | | CCCCCCTCCCTGTTTTTTCAGCC | | CCCACCCCTTCCCCCTCCTTT | | |
| | | | CCCTCCCTGCAGGTCTAGAGGG | | CCGAAGCCCCCCTCCCTGTTT | | |
| | | | TATATAATGGGGGCCA (SEQ ID | | TTTCAGCCCCCTC (SEQ ID | | |
| | | | NO: 139) | | NO: 2) | | |
| SB05245 | 7.6 | 148.9 | CAGGCGGGGAGGGGCCCCGGG | CHR19:4 | CAGGCGGGGAGGGGCCCCGG | CCTGC | TCTAGA |
| | | | GCGGGGAGGGGGCCGGCCCGC | 065794- | GGCGGGGAGGGGGCCGGCCC | AGG | GGGTAT |
| | | | GGGCCGCGCAGCCGGAAGCCG | 4066220 | GCGGGCCGCGCAGCCGGAAG | (SEQ ID | ATAATG |
| | | | GGACCGCCACCGGCCCCCGGC | | CCGGGACCGCCACCGGCCCC | NO: 107) | GGGGCC |
| | | | GAGGGGAGCCCGGCTCCAGGC | | CGGCGAGGGGAGCCCGGCTC | | A (YB- |
| | | | CCCGCCCCCTGGCGGGCTGGCC | | CAGGCCCCGCCCCCTGGCGG | | TATA; |
| | | | CCGCCCCCGCGCCGCGCCGCGC | | GCTGGCCCCGCCCCCGCGCC | | SEQ ID |
| | | | GATCGGCCCGCGCCCATTGGCT | | GCGCCGCGCGATCGGCCCGC | | NO: 122) |
| | | | CTCCGGCCCGCCGCTCACCGCC | | GCCCATTGGCTCTCCGGCCCG | | |
| | | | CCTCCTCCGCACCGCCCCTACC | | CCGCTCACCGCCCCTCCTCCG | | |
| | | | CGCAGGCCGCGGCGGGCTGTC | | CACCGCCCCTACCCGCAGGC | | |
| | | | GGCGCGGGGCACCCTGGGACT | | CGCGGCGGGCTGTCGGCGCG | | |
| | | | TGTAGTCCAAGCCGCTTGCCAC | | GGGCACCCTGGGACTTGTAG | | |
| | | | CTGCCGGCTGCAAACGGCGGA | | TCCAAGCCGCTTGCCACCTGC | | |
| | | | GGGACTACGAAGCCCAGAGGT | | CGGCTGCAAACGGCGGAGGG | | |
| | | | CCCTGCGGCCCTGCCCGCCCAC | | ACTACGAAGCCCAGAGGTCC | | |
| | | | CCGGACACCCCACCCCTTCCCC | | CTGCGGCCCTGCCCGCCCAC | | |
| | | | CTCCTTTCCGAAGCCCCCCTCC | | CCGGACACCCCACCCCTTCCC | | |
| | | | CTGTTTTTTCAGCCCCCTCCCCC | | CCTCCTTTCCGAAGCCCCCCT | | |
| | | | CCATCCCCCATGGAGCCCTGCA | | CCCTGTTTTTTCAGCCCCCTC | | |
| | | | GGTCTAGAGGGTATATAATGG | | CCCCCCATCCCCCATGGAGC | | |
| | | | GGGCCA (SEQ ID NO: 140) | | (SEQ ID NO: 3) | | |
| SB07596 | 7.3 | 81.7 | TGTTGAGAGCTCAAGCTCTTTT | CHR19:3 | TGTTGAGAGCTCAAGCTCTTT | CCTGC | TTCGCAT |
| | | | TAACGCTTTGCCTTGCTGTCTC | 990222- | TTAACGCTTTGCCTTGCTGTC | AGG | ATTAAG |
| | | | CAAAGTATTGCCTTCATCCTCA | 3990531 | TCCAAAGTATTGCCTTCATCC | (SEQ ID | GTGACG |
| | | | TAGTTCAAAGTGTCCACCATCA | | TCATAGTTCAAAGTGTCCACC | NO: 107) | CGTGTG |
| | | | CATTCACGTTTCAGCCAATAGG | | ATCACATTCACGTTTCAGCCA | | GCCTCG |
| | | | AAGAAGGAAAGAGAAAAACAG | | ATAGGAAGAAGGAAAGAGA | | AACACC |
| | | | GAAAAGAGTTTACATGCCACTC | | AAAACAGGAAAAGAGTTTAC | | GAGCGA |
| | | | CTGCTATTGGTCAGAATGTGTC | | ATGCCACTCCTGCTATTGGTC | | CCCTGCA |
| | | | ACGTGACCATACTCAGCTGCAA | | AGAATGTGTCACGTGACCAT | | GCGACC |
| | | | GGGTTGGTGGGAAATGTAGTCT | | ACTCAGCTGCAAGGGTTGGT | | CGCTTAA |
| | | | TTTTTTCTGGGAGGCCATGTGT | | GGGAAATGTAGTCTTTTTTTC | | (MINTK; |
| | | | TCGGCTTAAAATTGTCTTTTTTT | | TGGGAGGCCATGTGTTCGGC | | SEQ ID |
| | | | CTTTTTCTTTATTTCTTTTTTCTT | | TTAAAATTGTCTTTTTTTCT | | NO: 123) |
| | | | TTCTTTCTTTTTTTTAGACGGCC | | TTTTCTTTATTTCTTTTTTC | | |
| | | | TGCAGGTTCGCATATTAAGGTG | | TTTTCTTTCTTTTTTTTAG | | |
| | | | ACGCGTGTGGCCTCGAACACCG | | ACGG (SEQ ID NO: 1) | | |
| | | | AGCGACCCTGCAGCGACCCGCT | | | | |
| | | | TAA (SEQ ID NO: 141) | | | | |
| SB07600 | 6.7 | 214.1 | TGTTGAGAGCTCAAGCTCTTTT | CHR19:3 | TGTTGAGAGCTCAAGCTCTTT | CCTGC | AGACGC |
| | | | TAACGCTTTGCCTTGCTGTCTC | 990222- | TTAACGCTTTGCCTTGCTGTC | AGG | TAGCGG |
| | | | CAAAGTATTGCCTTCATCCTCA | 3990531 | TCCAAAGTATTGCCTTCATCC | (SEQ ID | GGGGCT |
| | | | TAGTTCAAAGTGTCCACCATCA | | TCATAGTTCAAAGTGTCCACC | NO: 107) | ATAAAA |
| | | | CATTCACGTTTCAGCCAATAGG | | ATCACATTCACGTTTCAGCCA | | GGGGGT |
| | | | AAGAAGGAAAGAGAAAAACAG | | ATAGGAAGAAGGAAAGAGA | | GGGGGC |
| | | | GAAAAGAGTTTACATGCCACTC | | AAAACAGGAAAAGAGTTTAC | | GTTCGTC |
| | | | CTGCTATTGGTCAGAATGTGTC | | ATGCCACTCCTGCTATTGGTC | | CTCACTC |
| | | | ACGTGACCATACTCAGCTGCAA | | AGAATGTGTCACGTGACCAT | | T |
| | | | GGGTTGGTGGGAAATGTAGTCT | | ACTCAGCTGCAAGGGTTGGT | | (LATEAD |
| | | | TTTTTTCTGGGAGGCCATGTGT | | GGGAAATGTAGTCTTTTTTTC | | E; SEQ ID |
| | | | TCGGCTTAAAATTGTCTTTTTTT | | TGGGAGGCCATGTGTTCGGC | | NO: 126) |
| | | | CTTTTTCTTTATTTCTTTTTTCTT | | TTAAAATTGTCTTTTTTTC | | |
| | | | TTCTTTCTTTTTTTTAGACGGCC | | TTTTTCTTTATTTCTTTTTTC | | |
| | | | TGCAGGAGACGCTAGCGGGGG | | TTTTCTTTCTTTTTTTTA | | |
| | | | GCTATAAAAGGGGGTGGGGGC | | GACGG (SEQ ID NO: 1) | | |
| | | | GTTCGTCCTCACTCT (SEQ ID | | | | |
| | | | NO: 142) | | | | |
| SB07623 | 5.3 | 323.6 | AGCACGCGCGAGCTGTCACCA | CHR6:41 | AGCACGCGCGAGCTGTCACC | CCTGC | AAAATG |
| | | | GGGCGCGAGACAACCGAACAC | 895333- | AGGGCGCGAGACAACCGAAC | AGG | TGCGCAT |
| | | | GGCCCCGCCCCCTGCCCGTCTA | 41895720 | ACGGCCCCGCCCCCTGCCCG | (SEQ ID | GTGCAG |
| | | | TTGGCCCGCCCGCTGGAGCCCC | | TCTATTGGCCCGCCCGCTGGA | NO: 107) | CCATTGC |
| | | | GCCCCTCAGGCCCTGCATTGCG | | GCCCCGCCCCTCAGGCCCTG | | CTGGGA |
| | | | CCAACGGCGCAGCGCTGGGCC | | CATTGCGCCAACGGCGCAGC | | CGCATG |
| | | | GCGACCCCGGCACCGGCACCC | | GCTGGGCCGCGACCCCGGCA | | CGTAGG |
| | | | GTTCCGAGGGTTCGCGCCGCAG | | CCGGCACCCGTTCCGAGGGT | | GAGCCG |
| | | | GCGCAAAGTTTAGGGTCAGCC | | TCGCGCCGCAGGCGCAAAGT | | CGCGAC |
| | | | ACAAACGCGCGGCCGCTTTGA | | TTAGGGTCAGCCACAAACGC | | AAACTG |
| | | | GCCTGGCGTAAGGGGCGGCGG | | GCGGCCGCTTTGAGCCTGGC | | AGCCATT |
| | | | CGTAGGGGGACGTTGTGAATA | | GTAAGGGGCGGCGGCGTAGG | | GCGGCA |
| | | | GCTGGTCGAAGTTTGGTTGAAG | | GGGACGTTGTGAATAGCTGG | | AGACTA |
| | | | CACAGGAGGGTAAATAAGAAG | | TCGAAGTTTGGTTGAAGCAC | | GCGCAG |
| | | | GTTTCCAGACAAAGAGACTCTT | | AGGAGGGTAAATAAGAAGGT | | AGAGGA |
| | | | AAAGGTACAGACTCTTATGTCT | | TTCCAGACAAAGAGACTCTT | | GAGGGA |
| | | | GTCCCTCCTCCTTAAAGGGCCA | | AAAGGTACAGACTCTTATGT | | GCCGGA |
| | | | GAGACGCTCCCGAGCCCATCTC | | CTGTCCCTCCTCCTTAAAGGG | | GATGCC |
| | | | CCTGCAGGAAAATGTGCGCAT | | CCAGAGACGCTCCCGAGCCC | | AGACGC |
| | | | GTGCAGCCATTGCCTGGGACGC | | ATCTC (SEQ ID NO: 4) | | TTGGTTC |
| | | | ATGCGTAGGGAGCCGCGCGAC | | | | TGAGGA |
| | | | AAACTGAGCCATTGCGGCAAG | | | | GTGATTT |
| | | | ACTAGCGCAGAGAGGAGAGGG | | | | GCAACG |
| | | | AGCCGGAGATGCCAGACGCTT | | | | CAATGG |
| | | | GGTTCTGAGGAGTGATTTGCAA | | | | AGCGAG |
| | | | CGCAATGGAGCGAGGAAGG | | | | GAAGG |
| | | | (SEQ ID NO: 143) | | | | (SMP; SEQ |
| | | | | | | | ID NO: |
| | | | | | | | 125) |
| SB07624 | 6.1 | 383 | AGCACGCGCGAGCTGTCACCA | CHR6:41 | AGCACGCGCGAGCTGTCACC | CCTGC | AGACGC |
| | | | GGGCGCGAGACAACCGAACAC | 895333- | AGGGCGCGAGACAACCGAAC | AGG | TAGCGG |
| | | | GGCCCCGCCCCCTGCCCGTCTA | 41895720 | ACGGCCCCGCCCCCTGCCCG | (SEQ ID | GGGGCT |
| | | | TTGGCCCGCCCGCTGGAGCCCC | | TCTATTGGCCCGCCCGCTGGA | NO: 107) | ATAAAA |
| | | | GCCCCTCAGGCCCTGCATTGCG | | GCCCCGCCCCTCAGGCCCTG | | GGGGGT |
| | | | CCAACGGCGCAGCGCTGGGCC | | CATTGCGCCAACGGCGCAGC | | GGGGGC |
| | | | GCGACCCCGGCACCGGCACCC | | GCTGGGCCGCGACCCCGGCA | | GTTCGTC |
| | | | GTTCCGAGGGTTCGCGCCGCAG | | CCGGCACCCGTTCCGAGGGT | | CTCACTC |
| | | | GCGCAAAGTTTAGGGTCAGCC | | TCGCGCCGCAGGCGCAAAGT | | T |
| | | | ACAAACGCGCGGCCGCTTTGA | | TTAGGGTCAGCCACAAACGC | | (LATEAD |
| | | | GCCTGGCGTAAGGGGCGGCGG | | GCGGCCGCTTTGAGCCTGGC | | E; SEQ ID |
| | | | CGTAGGGGGACGTTGTGAATA | | GTAAGGGGCGGCGGCGTAGG | | NO: 126) |
| | | | GCTGGTCGAAGTTTGGTTGAAG | | GGGACGTTGTGAATAGCTGG | | |
| | | | CACAGGAGGGTAAATAAGAAG | | TCGAAGTTTGGTTGAAGCAC | | |
| | | | GTTTCCAGACAAAGAGACTCTT | | AGGAGGGTAAATAAGAAGGT | | |
| | | | AAAGGTACAGACTCTTATGTCT | | TTCCAGACAAAGAGACTCTT | | |
| | | | GTCCCTCCTCCTTAAAGGGCCA | | AAAGGTACAGACTCTTATGT | | |
| | | | GAGACGCTCCCGAGCCCATCTC | | CTGTCCCTCCTCCTTAAAGGG | | |
| | | | CCTGCAGGAGACGCTAGCGGG | | CCAGAGACGCTCCCGAGCCC | | |
| | | | GGGCTATAAAAGGGGGTGGGG | | ATCTC (SEQ ID NO: 4) | | |
| | | | GCGTTCGTCCTCACTCT (SEQ ID | | | | |
| | | | NO: 144) | | | | |
| SB07625 | 5.9 | 186.1 | AGCACGCGCGAGCTGTCACCA | CHR6:41 | AGCACGCGCGAGCTGTCACC | CCTGC | TAGGCG |
| | | | GGGCGCGAGACAACCGAACAC | 895333- | AGGGCGCGAGACAACCGAAC | AGG | TGTACG |
| | | | GGCCCCGCCCCCTGCCCGTCTA | 41895720 | ACGGCCCCGCCCCCTGCCCG | (SEQ ID | GTGGGA |
| | | | TTGGCCCGCCCGCTGGAGCCCC | | TCTATTGGCCCGCCCGCTGGA | NO: 107) | GGCCTAT |
| | | | GCCCCTCAGGCCCTGCATTGCG | | GCCCCGCCCCTCAGGCCCTG | | ATAAGC |
| | | | CCAACGGCGCAGCGCTGGGCC | | CATTGCGCCAACGGCGCAGC | | AGAGCT |
| | | | GCGACCCCGGCACCGGCACCC | | GCTGGGCCGCGACCCCGGCA | | CGTTTAG |
| | | | GTTCCGAGGGTTCGCGCCGCAG | | CCGGCACCCGTTCCGAGGGT | | TGAACC |
| | | | GCGCAAAGTTTAGGGTCAGCC | | TCGCGCCGCAGGCGCAAAGT | | GTCAGA |
| | | | ACAAACGCGCGGCCGCTTTGA | | TTAGGGTCAGCCACAAACGC | | TCGCCTG |
| | | | GCCTGGCGTAAGGGGCGGCGG | | GCGGCCGCTTTGAGCCTGGC | | GA |
| | | | CGTAGGGGGACGTTGTGAATA | | GTAAGGGGCGGCGGCGTAGG | | (MINCMV; |
| | | | GCTGGTCGAAGTTTGGTTGAAG | | GGGACGTTGTGAATAGCTGG | | SEQ ID |
| | | | CACAGGAGGGTAAATAAGAAG | | TCGAAGTTTGGTTGAAGCAC | | NO: 127) |
| | | | GTTTCCAGACAAAGAGACTCTT | | AGGAGGGTAAATAAGAAGGT | | |
| | | | AAAGGTACAGACTCTTATGTCT | | TTCCAGACAAAGAGACTCTT | | |
| | | | GTCCCTCCTCCTTAAAGGGCCA | | AAAGGTACAGACTCTTATGT | | |
| | | | GAGACGCTCCCGAGCCCATCTC | | CTGTCCCTCCTCCTTAAAGGG | | |
| | | | CCTGCAGGTAGGCGTGTACGGT | | CCAGAGACGCTCCCGAGCCC | | |
| | | | GGGAGGCCTATATAAGCAGAG | | ATCTC (SEQ ID NO: 4) | | |
| | | | CTCGTTTAGTGAACCGTCAGAT | | | | |
| | | | CGCCTGGA (SEQ ID NO: 145) | | | | |
| SB07632 | 12.6 | 265.5 | GGTTACACAACCAGGCGGGGA | CHR 19:4 | GGTTACACAACCAGGCGGGG | CCTGC | TTCGCAT |
| | | | GGGGCCCCGGGGCGGGGAGGG | 065783- | AGGGGCCCCGGGGGGGGAG | AGG | ATTAAG |
| | | | GGCCGGCCCGCGGGCCGCGCA | 4066200 | GGGGCCGGCCCGCGGGCCGC | (SEQ ID | GTGACG |
| | | | GCCGGAAGCCGGGACCGCCAC | | GCAGCCGGAAGCCGGGACCG | NO: 107) | CGTGTG |
| | | | CGGCCCCCGGCGAGGGGAGCC | | CCACCGGCCCCCGGCGAGGG | | GCCTCG |
| | | | CGGCTCCAGGCCCCGCCCCCTG | | GAGCCCGGCTCCAGGCCCCG | | AACACC |
| | | | GCGGGCTGGCCCCGCCCCCGCG | | CCCCCTGGCGGGCTGGCCCC | | GAGCGA |
| | | | CCGCGCCGCGCGATCGGCCCGC | | GCCCCCGCGCCGCGCCGCGC | | CCCTGCA |
| | | | GCCCATTGGCTCTCCGGCCCGC | | GATCGGCCCGCGCCCATTGG | | GCGACC |
| | | | CGCTCACCGCCCCTCCTCCGCA | | CTCTCCGGCCCGCCGCTCACC | | CGCTTAA |
| | | | CCGCCCCTACCCGCAGGCCGCG | | GCCCCTCCTCCGCACCGCCCC | | (MINTK; |
| | | | GCGGGCTGTCGGCGCGGGGCA | | TACCCGCAGGCCGCGGCGGG | | SEQ ID |
| | | | CCCTGGGACTTGTAGTCCAAGC | | CTGTCGGCGCGGGGCACCCT | | NO: 123) |
| | | | CGCTTGCCACCTGCCGGCTGCA | | GGGACTTGTAGTCCAAGCCG | | |
| | | | AACGGCGGAGGGACTACGAAG | | CTTGCCACCTGCCGGCTGCA | | |
| | | | CCCAGAGGTCCCTGCGGCCCTG | | AACGGCGGAGGGACTACGAA | | |
| | | | CCCGCCCACCCGGACACCCCAC | | GCCCAGAGGTCCCTGCGGCC | | |
| | | | CCCTTCCCCCTCCTTTCCGAAG | | CTGCCCGCCCACCCGGACAC | | |
| | | | CCCCCCTCCCTGTTTTTTCAGCC | | CCCACCCCTTCCCCCTCCTTT | | |
| | | | CCCTCCCTGCAGGTTCGCATAT | | CCGAAGCCCCCCTCCCTGTTT | | |
| | | | TAAGGTGACGCGTGTGGCCTCG | | TTTCAGCCCCCTC (SEQ ID | | |
| | | | AACACCGAGCGACCCTGCAGC | | NO: 2) | | |
| | | | GACCCGCTTAA (SEQ ID NO: | | | | |
| | | | 146) | | | | |
| SB07634 | 8.7 | 67.1 | GGTTACACAACCAGGCGGGGA | CHR19:4 | GGTTACACAACCAGGCGGGG | CCTGC | CAGAAT |
| | | | GGGGCCCCGGGGCGGGGAGGG | 065783- | AGGGGCCCCGGGGGGGGGAG | AGG | TAACAG |
| | | | GGCCGGCCCGCGGGCCGCGCA | 4066200 | GGGGCCGGCCCGCGGGCCGC | (SEQ ID | TATAAAT |
| | | | GCCGGAAGCCGGGACCGCCAC | | GCAGCCGGAAGCCGGGACCG | NO: 107) | TGCATCT |
| | | | CGGCCCCCGGCGAGGGGAGCC | | CCACCGGCCCCCGGCGAGGG | | CTTGTTC |
| | | | CGGCTCCAGGCCCCGCCCCCTG | | GAGCCCGGCTCCAGGCCCCG | | AAGAGT |
| | | | GCGGGCTGGCCCCGCCCCCGCG | | CCCCCTGGCGGGCTGGCCCC | | TCCCTAT |
| | | | CCGCGCCGCGCGATCGGCCCGC | | GCCCCCGCGCCGCGCCGCGC | | CACTCT |
| | | | GCCCATTGGCTCTCCGGCCCGC | | GATCGGCCCGCGCCCATTGG | | (MINIL2.2 |
| | | | CGCTCACCGCCCCTCCTCCGCA | | CTCTCCGGCCCGCCGCTCACC | | SEQ ID |
| | | | CCGCCCCTACCCGCAGGCCGCG | | GCCCCTCCTCCGCACCGCCCC | | NO: 124) |
| | | | GCGGGCTGTCGGCGCGGGGCA | | TACCCGCAGGCCGCGGCGGG | | |
| | | | CCCTGGGACTTGTAGTCCAAGC | | CTGTCGGCGCGGGGCACCCT | | |
| | | | CGCTTGCCACCTGCCGGCTGCA | | GGGACTTGTAGTCCAAGCCG | | |
| | | | AACGGCGGAGGGACTACGAAG | | CTTGCCACCTGCCGGCTGCA | | |
| | | | CCCAGAGGTCCCTGCGGCCCTG | | AACGGCGGAGGGACTACGAA | | |
| | | | CCCGCCCACCCGGACACCCCAC | | GCCCAGAGGTCCCTGCGGCC | | |
| | | | CCCTTCCCCCTCCTTTCCGAAG | | CTGCCCGCCCACCCGGACAC | | |
| | | | CCCCCCTCCCTGTTTTTTCAGCC | | CCCACCCCTTCCCCCTCCTTT | | |
| | | | CCCTCCCTGCAGGCAGAATTAA | | CCGAAGCCCCCCTCCCTGTTT | | |
| | | | CAGTATAAATTGCATCTCTTGT | | TTTCAGCCCCCTC (SEQ ID | | |
| | | | TCAAGAGTTCCCTATCACTCT | | NO: 2) | | |
| | | | (SEQ ID NO: 147) | | | | |
| SB07636 | 9.8 | 127.5 | GGTTACACAACCAGGCGGGGA | CHR 19:4 | GGTTACACAACCAGGCGGGG | CCTGC | AGACGC |
| | | | GGGGCCCCGGGGCGGGGAGGG | 065783- | AGGGGCCCCGGGGCGGGGAG | AGG | TAGCGG |
| | | | GGCCGGCCCGCGGGCCGCGCA | 4066200 | GGGGCCGGCCCGCGGGCCGC | (SEQ ID | GGGGCT |
| | | | GCCGGAAGCCGGGACCGCCAC | | GCAGCCGGAAGCCGGGACCG | NO: 107) | ATAAAA |
| | | | CGGCCCCCGGCGAGGGGAGCC | | CCACCGGCCCCCGGCGAGGG | | GGGGGT |
| | | | CGGCTCCAGGCCCCGCCCCCTG | | GAGCCCGGCTCCAGGCCCCG | | GGGGGC |
| | | | GCGGGCTGGCCCCGCCCCCGCG | | CCCCCTGGCGGGCTGGCCCC | | GTTCGTC |
| | | | CCGCGCCGCGCGATCGGCCCGC | | GCCCCCGCGCCGCGCCGCGC | | CTCACTC |
| | | | GCCCATTGGCTCTCCGGCCCGC | | GATCGGCCCGCGCCCATTGG | | T |
| | | | CGCTCACCGCCCCTCCTCCGCA | | CTCTCCGGCCCGCCGCTCACC | | (LATEAD |
| | | | CCGCCCCTACCCGCAGGCCGCG | | GCCCCTCCTCCGCACCGCCCC | | E; SEQ ID |
| | | | GCGGGCTGTCGGCGCGGGGCA | | TACCCGCAGGCCGCGGCGGG | | NO: 126) |
| | | | CCCTGGGACTTGTAGTCCAAGC | | CTGTCGGCGCGGGGCACCCT | | |
| | | | CGCTTGCCACCTGCCGGCTGCA | | GGGACTTGTAGTCCAAGCCG | | |
| | | | AACGGCGGAGGGACTACGAAG | | CTTGCCACCTGCCGGCTGCA | | |
| | | | CCCAGAGGTCCCTGCGGCCCTG | | AACGGCGGAGGGACTACGAA | | |
| | | | CCCGCCCACCCGGACACCCCAC | | GCCCAGAGGTCCCTGCGGCC | | |
| | | | CCCTTCCCCCTCCTTTCCGAAG | | CTGCCCGCCCACCCGGACAC | | |
| | | | CCCCCCTCCCTGTTTTTTCAGCC | | CCCACCCCTTCCCCCTCCTTT | | |
| | | | CCCTCCCTGCAGGAGACGCTAG | | CCGAAGCCCCCCTCCCTGTTT | | |
| | | | CGGGGGGCTATAAAAGGGGGT | | TTTCAGCCCCCTC (SEQ ID | | |
| | | | GGGGGCGTTCGTCCTCACTCT | | NO: 2) | | |
| | | | (SEQ ID NO: 148) | | | | |

### Example 2: Ablation Analysis of Native Enhancers

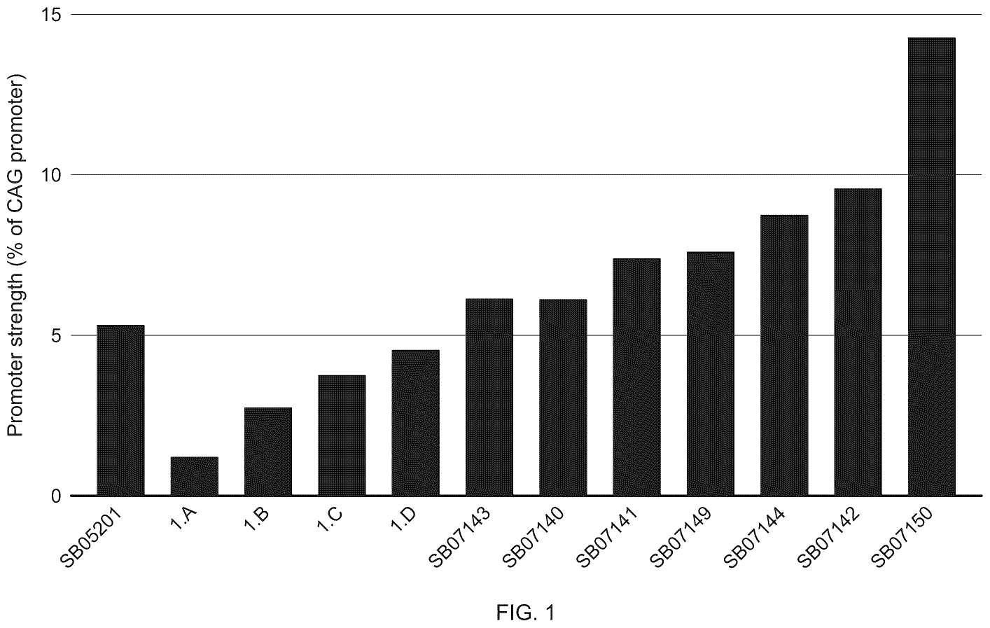

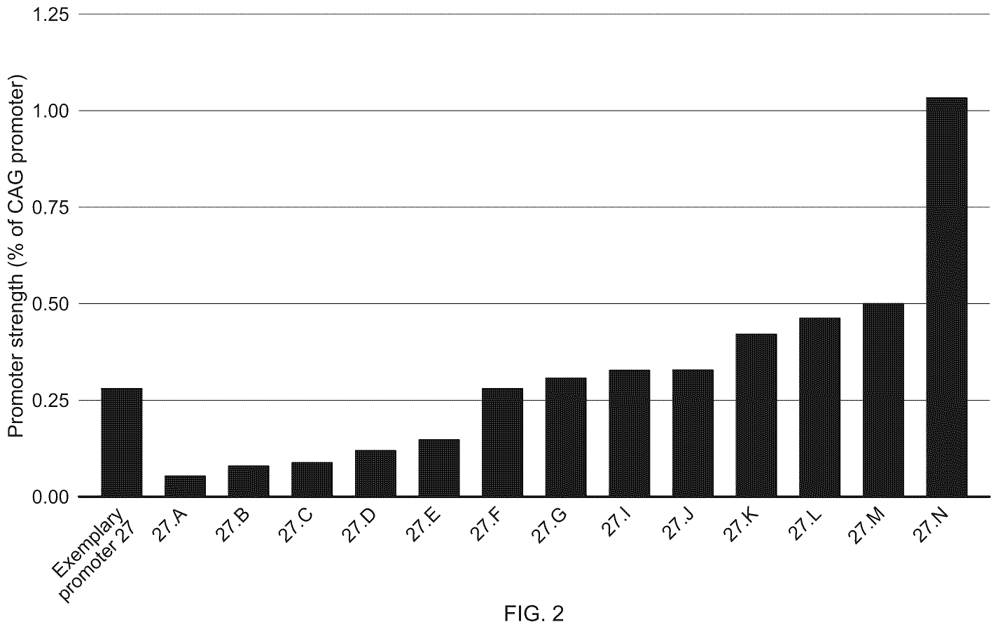

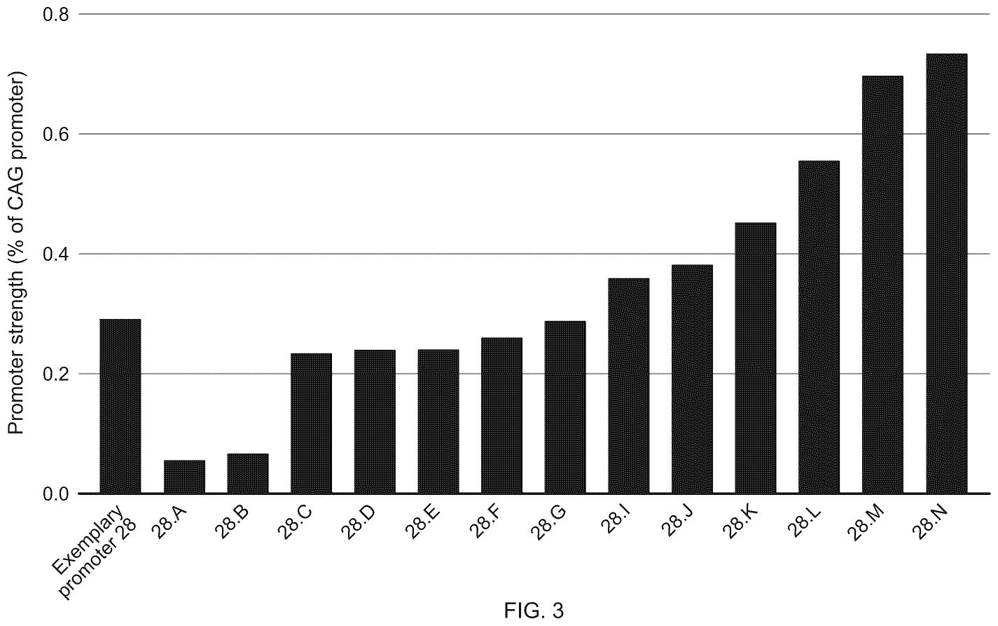

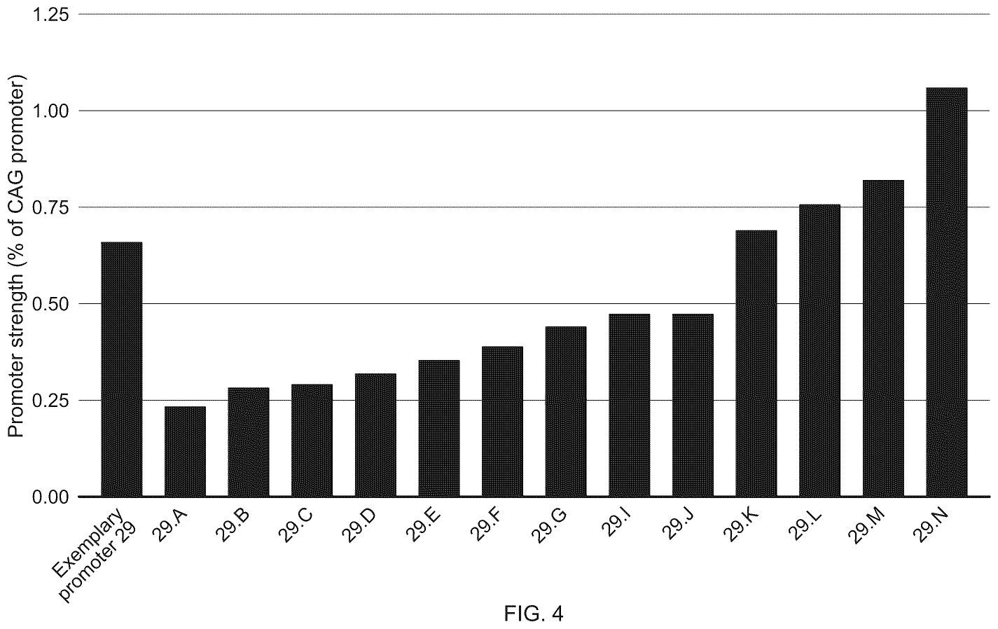

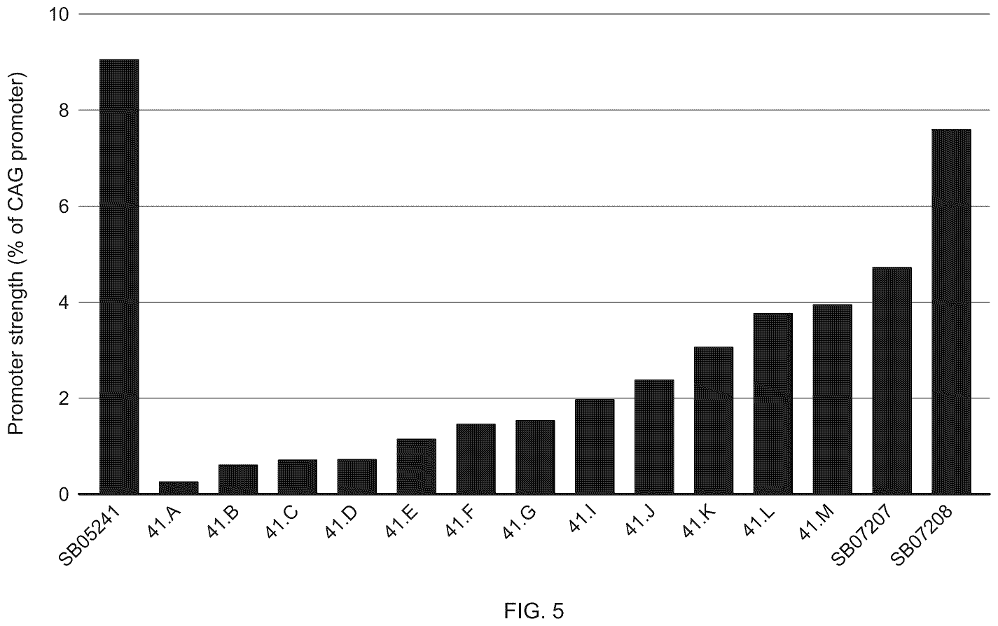

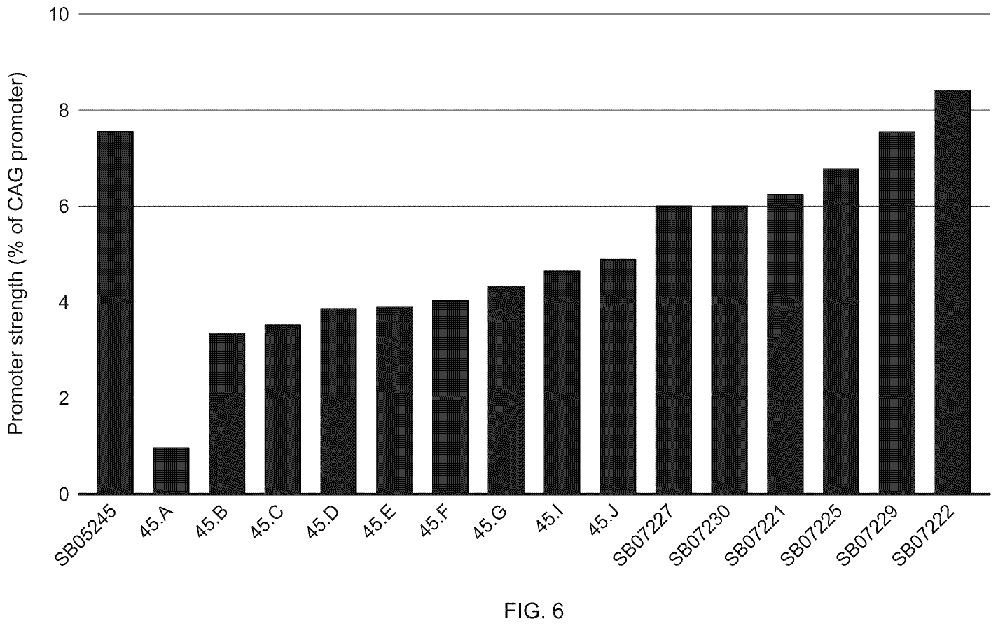

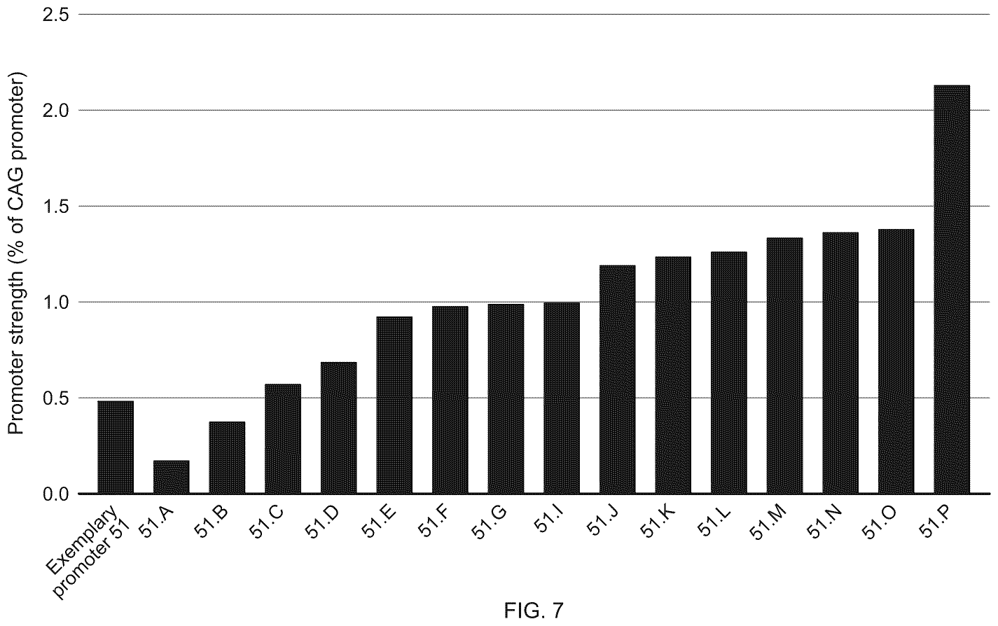

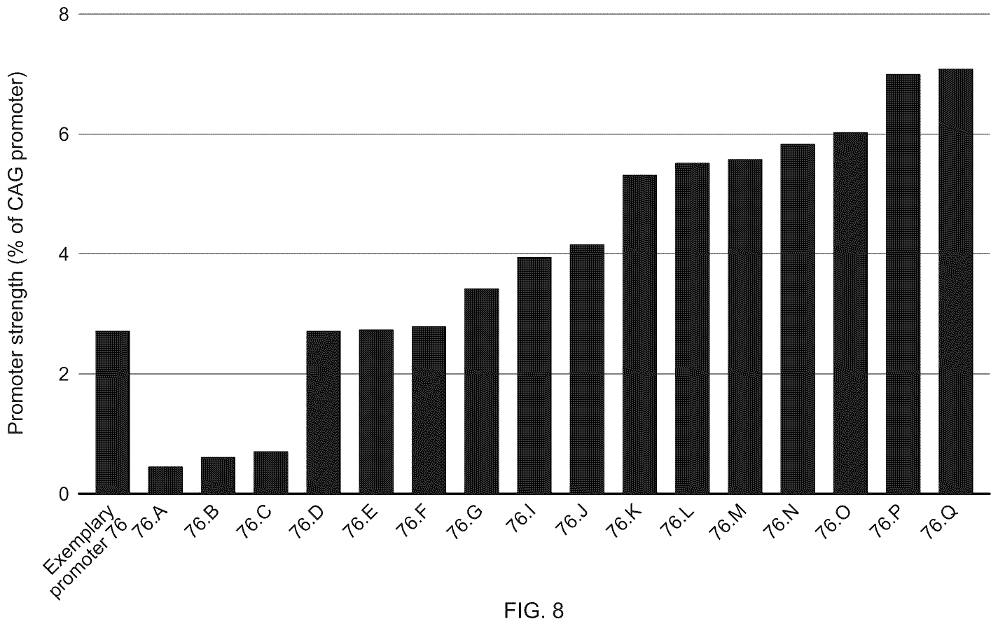

To determine whether there are critical regions present in the native enhancers that are important for strength or specificity, ablation analysis was conducted on the native regulatory elements of Example 1. The top performing enhancers, and enhancer-promoter combinations resulting from ablation experiments are provided in Table 2. Exemplary ablation motifs are also provided in Table 2. Measurement of strength and specificity of the ablation variants were carried out as described in Example 1. Summary of the ablation studies are shown in FIGS. 1-8, showing that some ablations can result in decreased promoter strength or increased promoter strength.

TABLE 2
Selected Ablation Enhancer & Promoter Regulatory Elements

| | | Spec- | | | | | |
|---|---|---|---|---|---|---|---|
| Con- | | ificity | | | Wildtype | Ablation | |
| struct | Strength | (Y79:ARP | | Ablation Regulatory | Sequence | Sequence | |
| Name | (%CAG) | E-19) | Name | Element Sequence | Motif | Motif | WT Enhancer Sequence |
| SB07140 | 6.1 | 406.6 | SB05201 | TTACCTACTAGGTTAATTC | TGTTGAG | TTACCTA | TGTTGAGAGCTCAAGCTCTT |
| | | | _+_m_00 | GTCCGATAGATTGCCTTGC | AGCTCA | CTAGGTT | TTTAACGCTTTGCCTTGCTGT |
| | | | | TGTCTCCAAAGTATTGCCTT | AGCTCTT | AATTCGT | CTCCAAAGTATTGCCTTCAT |
| | | | | CATCCTCATAGTTCAAAGT | TTTAACG | CCGATAG | CCTCATAGTTCAAAGTGTCC |
| | | | | GTCCACCATCACATTCACG | CTT (SEQ | AT (SEQ ID | ACCATCACATTCACGTTTCAG |
| | | | | TTTCAGCCAATAGGAAGAA | ID NO: 5) | NO: 6) | CCAATAGGAAGAAGGAAA |
| | | | | GGAAAGAGAAAAACAGGA | | | GAGAAAAACAGGAAAAGAGT |
| | | | | AAAGAGTTTACATGCCACT | | | TTACATGCCACTCCTGCTATT |
| | | | | CCTGCTATTGGTCAGAATG | | | GGTCAGAATGTGTCACGTGAC |
| | | | | TGTCACGTGACCATACTCA | | | CATACTCAGCTGCAAGGGTT |
| | | | | GCTGCAAGGGTTGGTGGGA | | | GGTGGGAAATGTAGTCTTTTT |
| | | | | AATGTAGTCTTTTTTTCTGG | | | TTCTGGGAGGCCATGTGTTCG |
| | | | | GAGGCCATGTGTTCGGCTT | | | GCTTAAAATTGTCTTTTTTTC |
| | | | | AAAATTGTCTTTTTTTCTTT | | | TTTTTCTTTATTTCTTTTTT |
| | | | | TTCTTTATTTCTTTTTTCTTT | | | CTTTTCTTTCTTTTTTTTAGA |
| | | | | TCTTTCTTTTTTTTAGACGG | | | CGG (SEQ ID NO: 1) |
| | | | | CCTGCAGGTCTAGAGGGTA | | | |
| | | | | TATAATGGGGGCCA (SEQ | | | |
| | | | | ID NO: 149) | | | |
| SB07141 | 7.4 | 502.9 | SB05201 | TGTTGAGAGCTCAAGCTCT | TGCCTTG | ACTACGA | TGTTGAGAGCTCAAGCTCTT |
| | | | _+_m_01 | TTTTAACGCTTACTACGAA | CTGTCTC | ATTGCGA | TTTAACGCTTTGCCTTGCTGT |
| | | | | TTGCGAGCTTCTAAGTCCA | CAAAGT | GCTTCTA | CTCCAAAGTATTGCCTTCAT |
| | | | | ATTTCCTCATAGTTCAAAG | ATTGCCT | AGTCCAA | CCTCATAGTTCAAAGTGTCC |
| | | | | TGTCCACCATCACATTCAC | TCA (SEQ | TT (SEQ ID | ACCATCACATTCACGTTTCAG |
| | | | | GTTTCAGCCAATAGGAAGA | ID NO: 7) | NO: 8) | CCAATAGGAAGAAGGAAA |
| | | | | AGGAAAGAGAAAAACAGG | | | GAGAAAAACAGGAAAAGAGT |
| | | | | AAAAGAGTTTACATGCCAC | | | TTACATGCCACTCCTGCTATT |
| | | | | TCCTGCTATTGGTCAGAAT | | | GGTCAGAATGTGTCACGTGAC |
| | | | | GTGTCACGTGACCATACTC | | | CATACTCAGCTGCAAGGGTT |
| | | | | AGCTGCAAGGGTTGGTGGG | | | GGTGGGAAATGTAGTCTTTTT |
| | | | | AAATGTAGTCTTTTTTTCTG | | | TTCTGGGAGGCCATGTGTTCG |
| | | | | GGAGGCCATGTGTTCGGCT | | | GCTTAAAATTGTCTTTTTTTC |
| | | | | TAAAATTGTCTTTTTTTCTT | | | TTTTTCTTTATTTCTTTTTT |
| | | | | TTTCTTTATTTCTTTTTTCTT | | | CTTTTCTTTCTTTTTTTTAGA |
| | | | | TTCTTTCTTTTTTTTAGACG | | | CGG (SEQ ID NO: 1) |
| | | | | GCCTGCAGGTCTAGAGGGT | | | |
| | | | | ATATAATGGGGGCCA (SEQ | | | |
| | | | | ID NO: 150) | | | |
| SB07142 | 9.6 | 401.9 | SB05201 | TGTTGAGAGCTCAAGCTCT | TCCTCAT | TTCGGTA | TGTTGAGAGCTCAAGCTCTT |
| | | | _+_m_02 | TTTTAACGCTTTGCCTTGCT | AGTTCA | TTCGAGT | TTTAACGCTTTGCCTTGCTGT |
| | | | | GTCTCCAAAGTATTGCCTT | AAGTGT | CGTCAGA | CTCCAAAGTATTGCCTTCAT |
| | | | | CATTCGGTATTCGAGTCGT | CCACCAT | CTCAATT | CCTCATAGTTCAAAGTGTCC |
| | | | | CAGACTCAATTATTTCACG | CACA | AT (SEQ ID | ACCATCACATTCACGTTTCAG |
| | | | | TTTCAGCCAATAGGAAGAA | (SEQ ID | NO: 10) | CCAATAGGAAGAAGGAAA |
| | | | | GGAAAGAGAAAAACAGGA | NO: 9) | | GAGAAAAACAGGAAAAGAGT |
| | | | | AAAGAGTTTACATGCCACT | | | TTACATGCCACTCCTGCTATT |
| | | | | CCTGCTATTGGTCAGAATG | | | GGTCAGAATGTGTCACGTGAC |
| | | | | TGTCACGTGACCATACTCA | | | CATACTCAGCTGCAAGGGTT |
| | | | | GCTGCAAGGGTTGGTGGGA | | | GGTGGGAAATGTAGTCTTTTT |
| | | | | AATGTAGTCTTTTTTTCTGG | | | TTCTGGGAGGCCATGTGTTCG |
| | | | | GAGGCCATGTGTTCGGCTT | | | GCTTAAAATTGTCTTTTTTTC |
| | | | | AAAATTGTCTTTTTTTCTTT | | | TTTTTCTTTATTTCTTTTTT |
| | | | | TTCTTTATTTCTTTTTTCTTT | | | CTTTTCTTTCTTTTTTTTAGA |
| | | | | TCTTTCTTTTTTTTAGACGG | | | CGG (SEQ ID NO: 1) |
| | | | | CCTGCAGGTCTAGAGGGTA | | | |
| | | | | TATAATGGGGGCCA (SEQ | | | |
| | | | | ID NO: 151) | | | |
| SB07143 | 6.1 | 643 | SB05201 | TGTTGAGAGCTCAAGCTCT | TTCACGT | TACACGG | TGTTGAGAGCTCAAGCTCTT |
| | | | _+_m_03 | TTTTAACGCTTTGCCTTGCT | TTCAGCC | TTAAGAC | TTTAACGCTTTGCCTTGCTGT |
| | | | | GTCTCCAAAGTATTGCCTT | AATAGG | TCAATCC | CTCCAAAGTATTGCCTTCAT |
| | | | | CATCCTCATAGTTCAAAGT | AAGAAG | GGTAAGT | CCTCATAGTTCAAAGTGTCC |
| | | | | GTCCACCATCACATACACG | GAAA | AA (SEQ | ACCATCACATTCACGTTTCAG |
| | | | | GTTAAGACTCAATCCGGTA | (SEQ ID | ID NO: 12) | CCAATAGGAAGAAGGAAA |
| | | | | AGTAAGAGAAAAACAGGA | NO: 11) | | GAGAAAAACAGGAAAAGAGT |
| | | | | AAAGAGTTTACATGCCACT | | | TTACATGCCACTCCTGCTATT |
| | | | | CCTGCTATTGGTCAGAATG | | | GGTCAGAATGTGTCACGTGAC |
| | | | | TGTCACGTGACCATACTCA | | | CATACTCAGCTGCAAGGGTT |
| | | | | GCTGCAAGGGTTGGTGGGA | | | GGTGGGAAATGTAGTCTTTTT |
| | | | | AATGTAGTCTTTTTTTCTGG | | | TTCTGGGAGGCCATGTGTTCG |
| | | | | GAGGCCATGTGTTCGGCTT | | | GCTTAAAATTGTCTTTTTTTC |
| | | | | AAAATTGTCTTTTTTTCTTT | | | TTTTTCTTTATTTCTTTTTT |
| | | | | TTCTTTATTTCTTTTTTCTTT | | | CTTTTCTTTCTTTTTTTTAGA |
| | | | | TCTTTCTTTTTTTTAGACGG | | | CGG (SEQ ID NO: 1) |
| | | | | CCTGCAGGTCTAGAGGGTA | | | |
| | | | | TATAATGGGGGCCA (SEQ | | | |
| | | | | ID NO: 152) | | | |
| SB07144 | 8.8 | 453.8 | SB05201 | TGTTGAGAGCTCAAGCTCT | GAGAAA | TACTGTG | TGTTGAGAGCTCAAGCTCTT |
| | | | _+_m_04 | TTTTAACGCTTTGCCTTGCT | AACAGG | ATCAGGT | TTTAACGCTTTGCCTTGCTGT |
| | | | | GTCTCCAAAGTATTGCCTT | AAAAGA | GTCTACA | CTCCAAAGTATTGCCTTCAT |
| | | | | CATCCTCATAGTTCAAAGT | GTTTACA | AGACTAG | CCTCATAGTTCAAAGTGTCC |
| | | | | GTCCACCATCACATTCACG | TGCCA | TA (SEQ ID | ACCATCACATTCACGTTTCAG |
| | | | | TTTCAGCCAATAGGAAGAA | (SEQ ID | NO: 14) | CCAATAGGAAGAAGGAAA |
| | | | | GGAAATACTGTGATCAGGT | NO: 13) | | GAGAAAAACAGGAAAAGAGT |
| | | | | GTCTACAAGACTAGTACTC | | | TTACATGCCACTCCTGCTATT |
| | | | | CTGCTATTGGTCAGAATGT | | | GGTCAGAATGTGTCACGTGAC |
| | | | | GTCACGTGACCATACTCAG | | | CATACTCAGCTGCAAGGGTT |
| | | | | CTGCAAGGGTTGGTGGGAA | | | GGTGGGAAATGTAGTCTTTTT |
| | | | | ATGTAGTCTTTTTTTCTGGG | | | TTCTGGGAGGCCATGTGTTCG |
| | | | | AGGCCATGTGTTCGGCTTA | | | GCTTAAAATTGTCTTTTTTTC |
| | | | | AAATTGTCTTTTTTTCTTTT | | | TTTTTCTTTATTTCTTTTTT |
| | | | | TCTTTATTTCTTTTTTCTTTT | | | CTTTTCTTTCTTTTTTTTAGA |
| | | | | CTTTCTTTTTTTTAGACGGC | | | CGG (SEQ ID NO: 1) |
| | | | | CTGCAGGTCTAGAGGGTAT | | | |
| | | | | ATAATGGGGGCCA (SEQ ID | | | |
| | | | | NO: 153) | | | |
| SB07149 | 7.6 | 194.9 | SB05201 | TGTTGAGAGCTCAAGCTCT | CTTTATT | ACTATCA | TGTTGAGAGCTCAAGCTCTT |
| | | | _+_m_09 | TTTTAACGCTTTGCCTTGCT | TCTTTTT | CGTATAT | TTTAACGCTTTGCCTTGCTGT |
| | | | | GTCTCCAAAGTATTGCCTT | TCTTTTC | ACCAGCG | CTCCAAAGTATTGCCTTCAT |
| | | | | CATCCTCATAGTTCAAAGT | TTTCTTT | AGTTCGA | CCTCATAGTTCAAAGTGTCC |
| | | | | GTCCACCATCACATTCACG | TT (SEQ | TA (SEQ ID | ACCATCACATTCACGTTTCAG |
| | | | | TTTCAGCCAATAGGAAGAA | ID NO: 15) | NO: 16) | CCAATAGGAAGAAGGAAA |
| | | | | GGAAAGAGAAAAACAGGA | | | GAGAAAAACAGGAAAAGAGT |
| | | | | AAAGAGTTTACATGCCACT | | | TTACATGCCACTCCTGCTATT |
| | | | | CCTGCTATTGGTCAGAATG | | | GGTCAGAATGTGTCACGTGAC |
| | | | | TGTCACGTGACCATACTCA | | | CATACTCAGCTGCAAGGGTT |
| | | | | GCTGCAAGGGTTGGTGGGA | | | GGTGGGAAATGTAGTCTTTTT |
| | | | | AATGTAGTCTTTTTTTCTGG | | | TTCTGGGAGGCCATGTGTTCG |
| | | | | GAGGCCATGTGTTCGGCTT | | | GCTTAAAATTGTCTTTTTTTC |
| | | | | AAAATTGTCTTTTTTTCTTT | | | TTTTTCTTTATTTCTTTTTT |
| | | | | TTACTATCACGTATATACC | | | CTTTTCTTTCTTTTTTTTAGA |
| | | | | AGCGAGTTCGATATTTAGA | | | CGG (SEQ ID NO: 1) |
| | | | | CGGCCTGCAGGTCTAGAGG | | | |
| | | | | GTATATAATGGGGGCCA | | | |
| | | | | (SEQ ID NO: 154) | | | |
| SB07150 | 14.3 | 325.4 | SB05201 | TGTTGAGAGCTCAAGCTCT | TTTAGAC | ATACACT | TGTTGAGAGCTCAAGCTCTT |
| | | | _+_m_10 | TTTTAACGCTTTGCCTTGCT | GG (SEQ | CT (SEQ ID | TTTAACGCTTTGCCTTGCTGT |
| | | | | GTCTCCAAAGTATTGCCTT | ID NO: 17) | NO: 18) | CTCCAAAGTATTGCCTTCAT |
| | | | | CATCCTCATAGTTCAAAGT | | | CCTCATAGTTCAAAGTGTCC |
| | | | | GTCCACCATCACATTCACG | | | ACCATCACATTCACGTTTCAG |
| | | | | TTTCAGCCAATAGGAAGAA | | | CCAATAGGAAGAAGGAAA |
| | | | | GGAAAGAGAAAAACAGGA | | | GAGAAAAACAGGAAAAGAGT |
| | | | | AAAGAGTTTACATGCCACT | | | TTACATGCCACTCCTGCTATT |
| | | | | CCTGCTATTGGTCAGAATG | | | GGTCAGAATGTGTCACGTGAC |
| | | | | TGTCACGTGACCATACTCA | | | CATACTCAGCTGCAAGGGTT |
| | | | | GCTGCAAGGGTTGGTGGGA | | | GGTGGGAAATGTAGTCTTTTT |
| | | | | AATGTAGTCTTTTTTTCTGG | | | TTCTGGGAGGCCATGTGTTCG |
| | | | | GAGGCCATGTGTTCGGCTT | | | GCTTAAAATTGTCTTTTTTTC |
| | | | | AAAATTGTCTTTTTTTCTTT | | | TTTTTCTTTATTTCTTTTTT |
| | | | | TTCTTTATTTCTTTTTTCTTT | | | CTTTTCTTTCTTTTTTTTAGA |
| | | | | TCTTTCTTTTTATACACTCT | | | CGG (SEQ ID NO: 1) |
| | | | | CCTGCAGGTCTAGAGGGTA | | | |
| | | | | TATAATGGGGGCCA (SEQ | | | |
| | | | | ID NO: 155) | | | |
| SB07207 | 4.7 | 290.8 | SB05241 | GGTTACACAACCAGGCGGG | CCGGCG | TACACGG | GGTTACACAACCAGGCGGGG |
| | | | _+_m_03 | GAGGGGCCCCGGGGCGGG | AGGGGA | TTAAGAC | AGGGGCCCCGGGGCGGGGA |
| | | | | GAGGGGGCCGGCCCGCGG | GCCCGG | TCAATCC | GGGGGCCGGCCCGCGGGCCG |
| | | | | GCCGCGCAGCCGGAAGCCG | CTCCAG | GGTAAGT | CGCAGCCGGAAGCCGGGACC |
| | | | | GGACCGCCACCGGCCCTAC | GCCCCG | AA (SEQ | GCCACCGGCCCCCGGCGAG |
| | | | | ACGGTTAAGACTCAATCCG | (SEQ ID | ID NO: 12) | GGGAGCCCGGCTCCAGGCCC |
| | | | | GTAAGTAACCCCCTGGCGG | NO: 19) | | CGCCCCCTGGCGGGCTGGCC |
| | | | | GCTGGCCCCGCCCCCGCGC | | | CCGCCCCCGCGCCGCGCC |
| | | | | CGCGCCGCGCGATCGGCCC | | | GCGCGATCGGCCCGCGCCC |
| | | | | GCGCCCATTGGCTCTCCGG | | | ATTGGCTCTCCGGCCCGCC |
| | | | | CCCGCCGCTCACCGCCCCT | | | GCTCACCGCCCCTCCTCCGC |
| | | | | CCTCCGCACCGCCCCTACC | | | ACCGCCCCTACCCGCAGGCC |
| | | | | CGCAGGCCGCGGCGGGCTG | | | GCGGCGGGCTGTCGGCGCG |
| | | | | TCGGCGCGGGGCACCCTGG | | | GGGCACCCTGGGACTTGTAG |
| | | | | GACTTGTAGTCCAAGCCGC | | | TCCAAGCCGCTTGCCACCT |
| | | | | TTGCCACCTGCCGGCTGCA | | | GCCGGCTGCAAACGGCGGAG |
| | | | | AACGGCGGAGGGACTACG | | | GGACTACGAAGCCCAGAGG |
| | | | | AAGCCCAGAGGTCCCTGCG | | | TCCCTGCGGCCCTGCCCGCC |
| | | | | GCCCTGCCCGCCCACCCGG | | | CACCCGGACACCCCACCCCT |
| | | | | ACACCCCACCCCTTCCCCC | | | TCCCCCTCCTTTCCGAAGCC |
| | | | | TCCTTTCCGAAGCCCCCCTC | | | CCCCTCCCTGTTTTTTCAGC |
| | | | | CCTGTTTTTTCAGCCCCCTC | | | CCCCTC (SEQ ID |
| | | | | CCTGCAGGTCTAGAGGGTA | | | NO: 2) |
| | | | | TATAATGGGGGCCA (SEQ | | | |
| | | | | ID NO: 156) | | | |
| SB07208 | 7.6 | 339.6 | SB05241 | GGTTACACAACCAGGCGGG | CCCCCTG | TGCCTAA | GGTTACACAACCAGGCGGGG |
| | | | _+_m_04 | GAGGGGCCCCGGGGCGGG | GCGGGC | AACGAGA | AGGGGCCCCGGGGCGGGGA |
| | | | | GAGGGGGCCGGCCCGCGG | TGGCCCC | TTCTGTA | GGGGGCCGGCCCGCGGGCCG |
| | | | | GCCGCGCAGCCGGAAGCCG | GCCCCC | CGATAAA | CGCAGCCGGAAGCCGGGACC |
| | | | | GGACCGCCACCGGCCCCCG | GCGC | GC (SEQ ID | GCCACCGGCCCCCGGCGAG |
| | | | | GCGAGGGGAGCCCGGCTCC | (SEQ ID | NO: 21) | GGGAGCCCGGCTCCAGGCCC |
| | | | | AGGCCCCGTGCCTAAAACG | NO: 20) | | CGCCCCCTGGCGGGCTGGCC |
| | | | | AGATTCTGTACGATAAAGC | | | CCGCCCCCGCGCCGCGCC |
| | | | | CGCGCCGCGCGATCGGCCC | | | GCGCGATCGGCCCGCGCCC |
| | | | | GCGCCCATTGGCTCTCCGG | | | ATTGGCTCTCCGGCCCGCC |
| | | | | CCCGCCGCTCACCGCCCCT | | | GCTCACCGCCCCTCCTCCGC |
| | | | | CCTCCGCACCGCCCCTACC | | | ACCGCCCCTACCCGCAGGCC |
| | | | | CGCAGGCCGCGGCGGGCTG | | | GCGGCGGGCTGTCGGCGCG |
| | | | | TCGGCGCGGGGCACCCTGG | | | GGGCACCCTGGGACTTGTAG |
| | | | | GACTTGTAGTCCAAGCCGC | | | TCCAAGCCGCTTGCCACCT |
| | | | | TTGCCACCTGCCGGCTGCA | | | GCCGGCTGCAAACGGCGGAG |
| | | | | AACGGCGGAGGGACTACG | | | GGACTACGAAGCCCAGAGG |
| | | | | AAGCCCAGAGGTCCCTGCG | | | TCCCTGCGGCCCTGCCCGCC |
| | | | | GCCCTGCCCGCCCACCCGG | | | CACCCGGACACCCCACCCCT |
| | | | | ACACCCCACCCCTTCCCCC | | | TCCCCCTCCTTTCCGAAGCC |
| | | | | TCCTTTCCGAAGCCCCCCTC | | | CCCCTCCCTGTTTTTTCAGC |
| | | | | CCTGTTTTTTCAGCCCCCTC | | | CCCCTC (SEQ ID |
| | | | | CCTGCAGGTCTAGAGGGTA | | | NO: 2) |
| | | | | TATAATGGGGGCCA (SEQ | | | |
| | | | | ID NO: 157) | | | |
| SB07221 | 6.2 | 148.9 | SB05245 | CAGGCGGGGAGGGGCCCC | AGCCCG | TACACGG | CAGGCGGGGAGGGGCCCCG |
| | | | _+_m_03 | GGGGCGGGGAGGGGGCCG | GCTCCA | TTAAGAC | GGGCGGGGAGGGGGCCGG |
| | | | | GCCCGCGGGCCGCGCAGCC | GGCCCC | TCAATCC | CCCGCGGGCCGCGCAGCCG |
| | | | | GGAAGCCGGGACCGCCACC | GCCCCCT | GGTAAGT | GAAGCCGGGACCGCCACCG |
| | | | | GGCCCCCGGCGAGGGGTAC | GGCGG | AA (SEQ | GCCCCCGGCGAGGGGAGCC |
| | | | | ACGGTTAAGACTCAATCCG | (SEQ ID | ID NO: 12) | CGGCTCCAGGCCCCGCCCCC |
| | | | | GTAAGTAAGCTGGCCCCGC | NO: 22) | | TGGCGGGCTGGCCCCGCCC |
| | | | | CCCCGCGCCGCGCCGCGCG | | | CCGCGCCGCGCCGCGCGAT |
| | | | | ATCGGCCCGCGCCCATTGG | | | CGGCCCGCGCCCATTGGC |
| | | | | CTCTCCGGCCCGCCGCTCA | | | TCTCCGGCCCGCCGCTCA |
| | | | | CCGCCCCTCCTCCGCACCG | | | CCGCCCCTCCTCCGCACCGC |
| | | | | CCCCTACCCGCAGGCCGCG | | | CCCTACCCGCAGGCCGCGG |
| | | | | GCGGGCTGTCGGCGCGGGG | | | CGGGCTGTCGGCGCGGGGC |
| | | | | CACCCTGGGACTTGTAGTC | | | ACCCTGGGACTTGTAGTCCA |
| | | | | CAAGCCGCTTGCCACCTGC | | | AGCCGCTTGCCACCTGCCG |
| | | | | CGGCTGCAAACGGCGGAG | | | GCTGCAAACGGCGGAGGGA |
| | | | | GGACTACGAAGCCCAGAG | | | CTACGAAGCCCAGAGGTC |
| | | | | GTCCCTGCGGCCCTGCCCG | | | CCTGCGGCCCTGCCCGCCC |
| | | | | CCCACCCGGACACCCCACC | | | ACCCGGACACCCCACCCCT |
| | | | | CCTTCCCCCTCCTTTCCGAA | | | TCCCCCTCCTTTCCGAAG |
| | | | | GCCCCCCTCCCTGTTTTTTC | | | CCCCCCTCCCTGTTTTTTC |
| | | | | AGCCCCCTCCCCCCCATCC | | | AGCCCCCTCCCCCCCATCC |
| | | | | CCCATGGAGCCCTGCAGGT | | | CCCATGGAGC (SEQ ID |
| | | | | CTAGAGGGTATATAATGGG | | | NO: 3) |
| | | | | GGCCA (SEQ ID NO: 158) | | | |
| SB07222 | 8.4 | 148.9 | SB05245 | CAGGCGGGGAGGGGCCCC | GCTGGC | TGCCTAA | CAGGCGGGGAGGGGCCCCG |
| | | | _+_m_04 | GGGGCGGGGAGGGGGCCG | CCCGCCC | AACGAGA | GGGCGGGGAGGGGGCCGG |
| | | | | GCCCGCGGGCCGCGCAGCC | CCGCGC | TTCTGTA | CCCGCGGGCCGCGCAGCCG |
| | | | | GGAAGCCGGGACCGCCACC | CGCGCC | CGATAAA | GAAGCCGGGACCGCCACCG |
| | | | | GGCCCCCGGCGAGGGGAG | GCGCG | GC (SEQ ID | GCCCCCGGCGAGGGGAGCC |
| | | | | CCCGGCTCCAGGCCCCGCC | (SEQ ID | NO: 21) | CGGCTCCAGGCCCCGCCCCC |
| | | | | CCCTGGCGGTGCCTAAAAC | NO: 23) | | TGGCGGGCTGGCCCCGCCC |
| | | | | GAGATTCTGTACGATAAAG | | | CCGCGCCGCGCCGCGCGAT |
| | | | | CATCGGCCCGCGCCCATTG | | | CGGCCCGCGCCCATTGGC |
| | | | | GCTCTCCGGCCCGCCGCTC | | | TCTCCGGCCCGCCGCTCA |
| | | | | ACCGCCCCTCCTCCGCACC | | | CCGCCCCTCCTCCGCACCGC |
| | | | | GCCCCTACCCGCAGGCCGC | | | CCCTACCCGCAGGCCGCGG |
| | | | | GGCGGGCTGTCGGCGCGGG | | | CGGGCTGTCGGCGCGGGGC |
| | | | | GCACCCTGGGACTTGTAGT | | | ACCCTGGGACTTGTAGTCCA |
| | | | | CCAAGCCGCTTGCCACCTG | | | AGCCGCTTGCCACCTGCCG |
| | | | | CCGGCTGCAAACGGCGGAG | | | GCTGCAAACGGCGGAGGGA |
| | | | | GGACTACGAAGCCCAGAG | | | CTACGAAGCCCAGAGGTC |
| | | | | GTCCCTGCGGCCCTGCCCG | | | CCTGCGGCCCTGCCCGCCC |
| | | | | CCCACCCGGACACCCCACC | | | ACCCGGACACCCCACCCCT |
| | | | | CCTTCCCCCTCCTTTCCGAA | | | TCCCCCTCCTTTCCGAAG |
| | | | | GCCCCCCTCCCTGTTTTTTC | | | CCCCCCTCCCTGTTTTTTC |
| | | | | AGCCCCCTCCCCCCCATCC | | | AGCCCCCTCCCCCCCATCC |
| | | | | CCCATGGAGCCCTGCAGGT | | | CCCATGGAGC (SEQ ID |
| | | | | CTAGAGGGTATATAATGGG | | | NO: 3) |
| | | | | GGCCA (SEQ ID NO: 159) | | | |
| SB07225 | 6.7 | 148.9 | SB05245 | CAGGCGGGGAGGGGCCCC | CTACCCG | TACGGTC | CAGGCGGGGAGGGGCCCCG |
| | | | _+_m_07 | GGGGCGGGGAGGGGGCCG | CAGGCC | TCGCTAA | GGGCGGGGAGGGGGCCGG |
| | | | | GCCCGCGGGCCGCGCAGCC | GCGGCG | TAGGAGT | CCCGCGGGCCGCGCAGCCG |
| | | | | GGAAGCCGGGACCGCCACC | GGCTGTC | AAGATAC | GAAGCCGGGACCGCCACCG |
| | | | | GGCCCCCGGCGAGGGGAG | GGCG | AT (SEQ ID | GCCCCCGGCGAGGGGAGCC |
| | | | | CCCGGCTCCAGGCCCCGCC | (SEQ ID | NO: 25) | CGGCTCCAGGCCCCGCCCCC |
| | | | | CCCTGGCGGGCTGGCCCCG | NO: 24) | | TGGCGGGCTGGCCCCGCCC |
| | | | | CCCCCGCGCCGCGCCGCGC | | | CCGCGCCGCGCCGCGCGAT |
| | | | | GATCGGCCCGCGCCCATTG | | | CGGCCCGCGCCCATTGGC |
| | | | | GCTCTCCGGCCCGCCGCTC | | | TCTCCGGCCCGCCGCTCA |
| | | | | ACCGCCCCTCCTCCGCACC | | | CCGCCCCTCCTCCGCACCGC |
| | | | | GCCCTACGGTCTCGCTAAT | | | CCCTACCCGCAGGCCGCGG |
| | | | | AGGAGTAAGATACATCGGG | | | CGGGCTGTCGGCGCGGGGC |
| | | | | GCACCCTGGGACTTGTAGT | | | ACCCTGGGACTTGTAGTCCA |
| | | | | CCAAGCCGCTTGCCACCTG | | | AGCCGCTTGCCACCTGCCG |
| | | | | CCGGCTGCAAACGGCGGAG | | | GCTGCAAACGGCGGAGGGA |
| | | | | GGACTACGAAGCCCAGAG | | | CTACGAAGCCCAGAGGTC |
| | | | | GTCCCTGCGGCCCTGCCCG | | | CCTGCGGCCCTGCCCGCCC |
| | | | | CCCACCCGGACACCCCACC | | | ACCCGGACACCCCACCCCT |
| | | | | CCTTCCCCCTCCTTTCCGAA | | | TCCCCCTCCTTTCCGAAG |
| | | | | GCCCCCCTCCCTGTTTTTTC | | | CCCCCCTCCCTGTTTTTTC |
| | | | | AGCCCCCTCCCCCCCATCC | | | AGCCCCCTCCCCCCCATCC |
| | | | | CCCATGGAGCCCTGCAGGT | | | CCCATGGAGC (SEQ ID |
| | | | | CTAGAGGGTATATAATGGG | | | NO: 3) |
| | | | | GGCCA (SEQ ID NO: 160) | | | |
| SB07227 | 6 | 148.9 | SB05245 | CAGGCGGGGAGGGGCCCC | GCTTGCC | ACTATCA | CAGGCGGGGAGGGGCCCCG |
| | | | _+_m_09 | GGGGCGGGGAGGGGGCCG | ACCTGCC | CGTATAT | GGGCGGGGAGGGGGCCGG |
| | | | | GCCCGCGGGCCGCGCAGCC | GGCTGC | ACCAGCG | CCCGCGGGCCGCGCAGCCG |
| | | | | GGAAGCCGGGACCGCCACC | AAACGG | AGTTCGA | GAAGCCGGGACCGCCACCG |
| | | | | GGCCCCCGGCGAGGGGAG | CGGA | TA (SEQ ID | GCCCCCGGCGAGGGGAGCC |
| | | | | CCCGGCTCCAGGCCCCGCC | (SEQ ID | NO: 16) | CGGCTCCAGGCCCCGCCCCC |
| | | | | CCCTGGCGGGCTGGCCCCG | NO: 26) | | TGGCGGGCTGGCCCCGCCC |
| | | | | CCCCCGCGCCGCGCCGCGC | | | CCGCGCCGCGCCGCGCGAT |
| | | | | GATCGGCCCGCGCCCATTG | | | CGGCCCGCGCCCATTGGC |
| | | | | GCTCTCCGGCCCGCCGCTC | | | TCTCCGGCCCGCCGCTCA |
| | | | | ACCGCCCCTCCTCCGCACC | | | CCGCCCCTCCTCCGCACCGC |
| | | | | GCCCCTACCCGCAGGCCGC | | | CCCTACCCGCAGGCCGCGG |
| | | | | GGCGGGCTGTCGGCGCGGG | | | CGGGCTGTCGGCGCGGGGC |
| | | | | GCACCCTGGGACTTGTAGT | | | ACCCTGGGACTTGTAGTCCA |
| | | | | CCAAGCCACTATCACGTAT | | | AGCCGCTTGCCACCTGCCG |
| | | | | ATACCAGCGAGTTCGATAG | | | GCTGCAAACGGCGGAGGGA |
| | | | | GGACTACGAAGCCCAGAG | | | CTACGAAGCCCAGAGGTC |
| | | | | GTCCCTGCGGCCCTGCCCG | | | CCTGCGGCCCTGCCCGCCC |
| | | | | CCCACCCGGACACCCCACC | | | ACCCGGACACCCCACCCCT |
| | | | | CCTTCCCCCTCCTTTCCGAA | | | TCCCCCTCCTTTCCGAAG |
| | | | | GCCCCCCTCCCTGTTTTTTC | | | CCCCCCTCCCTGTTTTTTC |
| | | | | AGCCCCCTCCCCCCCATCC | | | AGCCCCCTCCCCCCCATCC |
| | | | | CCCATGGAGCCCTGCAGGT | | | CCCATGGAGC (SEQ ID |
| | | | | CTAGAGGGTATATAATGGG | | | NO: 3) |
| | | | | GGCCA (SEQ ID NO: 161) | | | |
| SB07229 | 7.5 | 148.9 | SB05245 | CAGGCGGGGAGGGGCCCC | CCTGCCC | TGTTAAG | CAGGCGGGGAGGGGCCCCG |
| | | | _+_m_11 | GGGGCGGGGAGGGGGCCG | GCCCAC | CATACTA | GGGCGGGGAGGGGGCCGG |
| | | | | GCCCGCGGGCCGCGCAGCC | CCGGAC | AACTGTA | CCCGCGGGCCGCGCAGCCG |
| | | | | GGAAGCCGGGACCGCCACC | ACCCCA | AAGAAGC | GAAGCCGGGACCGCCACCG |
| | | | | GGCCCCCGGCGAGGGGAG | CCCCT | GA (SEQ | GCCCCCGGCGAGGGGAGCC |
| | | | | CCCGGCTCCAGGCCCCGCC | (SEQ ID | ID NO: 28) | CGGCTCCAGGCCCCGCCCCC |
| | | | | CCCTGGCGGGCTGGCCCCG | NO: 27) | | TGGCGGGCTGGCCCCGCCC |
| | | | | CCCCCGCGCCGCGCCGCGC | | | CCGCGCCGCGCCGCGCGAT |
| | | | | GATCGGCCCGCGCCCATTG | | | CGGCCCGCGCCCATTGGC |
| | | | | GCTCTCCGGCCCGCCGCTC | | | TCTCCGGCCCGCCGCTCA |
| | | | | ACCGCCCCTCCTCCGCACC | | | CCGCCCCTCCTCCGCACCGC |
| | | | | GCCCCTACCCGCAGGCCGC | | | CCCTACCCGCAGGCCGCGG |
| | | | | GGCGGGCTGTCGGCGCGGG | | | CGGGCTGTCGGCGCGGGGC |
| | | | | GCACCCTGGGACTTGTAGT | | | ACCCTGGGACTTGTAGTCCA |
| | | | | CCAAGCCGCTTGCCACCTG | | | AGCCGCTTGCCACCTGCCG |
| | | | | CCGGCTGCAAACGGCGGAG | | | GCTGCAAACGGCGGAGGGA |
| | | | | GGACTACGAAGCCCAGAG | | | CTACGAAGCCCAGAGGTC |
| | | | | GTCCCTGCGGCTGTTAAGC | | | CCTGCGGCCCTGCCCGCCC |
| | | | | ATACTAAACTGTAAAGAAG | | | ACCCGGACACCCCACCCCT |
| | | | | CGATCCCCCTCCTTTCCGA | | | TCCCCCTCCTTTCCGAAG |
| | | | | AGCCCCCCTCCCTGTTTTTT | | | CCCCCCTCCCTGTTTTTTC |
| | | | | CAGCCCCCTCCCCCCCATC | | | AGCCCCCTCCCCCCCATCC |
| | | | | CCCCATGGAGCCCTGCAGG | | | CCCATGGAGC (SEQ ID |
| | | | | TCTAGAGGGTATATAATGG | | | NO: 3) |
| | | | | GGGCCA (SEQ ID NO: 162) | | | |
| SB07230 | 6 | 148.9 | SB05245 | CAGGCGGGGAGGGGCCCC | TCCCCCT | GTTTCGA | CAGGCGGGGAGGGGCCCCG |
| | | | _+_m_12 | GGGGCGGGGAGGGGGCCG | CCTTTCC | GCGACGC | GGGCGGGGAGGGGGCCGG |
| | | | | GCCCGCGGGCCGCGCAGCC | GAAGCC | TTAATAT | CCCGCGGGCCGCGCAGCCG |
| | | | | GGAAGCCGGGACCGCCACC | CCCCTCC | TGCTAGA | GAAGCCGGGACCGCCACCG |
| | | | | GGCCCCCGGCGAGGGGAG | CTG (SEQ | TA (SEQ ID | GCCCCCGGCGAGGGGAGCC |
| | | | | CCCGGCTCCAGGCCCCGCC | ID NO: 29) | NO: 30) | CGGCTCCAGGCCCCGCCCCC |
| | | | | CCCTGGCGGGCTGGCCCCG | | | TGGCGGGCTGGCCCCGCCC |
| | | | | CCCCCGCGCCGCGCCGCGC | | | CCGCGCCGCGCCGCGCGAT |
| | | | | GATCGGCCCGCGCCCATTG | | | CGGCCCGCGCCCATTGGC |
| | | | | GCTCTCCGGCCCGCCGCTC | | | TCTCCGGCCCGCCGCTCA |
| | | | | ACCGCCCCTCCTCCGCACC | | | CCGCCCCTCCTCCGCACCGC |
| | | | | GCCCCTACCCGCAGGCCGC | | | CCCTACCCGCAGGCCGCGG |
| | | | | GGCGGGCTGTCGGCGCGGG | | | CGGGCTGTCGGCGCGGGGC |
| | | | | GCACCCTGGGACTTGTAGT | | | ACCCTGGGACTTGTAGTCCA |
| | | | | CCAAGCCGCTTGCCACCTG | | | AGCCGCTTGCCACCTGCCG |
| | | | | CCGGCTGCAAACGGCGGAG | | | GCTGCAAACGGCGGAGGGA |
| | | | | GGACTACGAAGCCCAGAG | | | CTACGAAGCCCAGAGGTC |
| | | | | GTCCCTGCGGCCCTGCCCG | | | CCTGCGGCCCTGCCCGCCC |
| | | | | CCCACCCGGACACCCCACC | | | ACCCGGACACCCCACCCCT |
| | | | | CCTGTTTCGAGCGACGCTT | | | TCCCCCTCCTTTCCGAAG |
| | | | | AATATTGCTAGATATTTTTT | | | CCCCCCTCCCTGTTTTTTC |
| | | | | CAGCCCCCTCCCCCCATC | | | AGCCCCCTCCCCCCCATCC |
| | | | | CCCCATGGAGCCCTGCAGG | | | CCCATGGAGC (SEQ ID |
| | | | | TCTAGAGGGTATATAATGG | | | NO: 3) |
| | | | | GGGCCA | | | |
| | | | | (SEQ ID NO: 163) | | | |

### I. Summary of Results

Candidate sequences were chosen via bioinformatics analyses based on differentially active/expressed genomic sequences and cloned into DNA libraries. There were 3 rounds of the library design and functional screenings performed: 1) target libraries consisting of native genomic sequences tested via MPRA, clonal screening, 2) based on the data from the first round, the sequences with the highest activity were chosen, and ablation studies were performed, 3) the most significant ablation DNA fragments were grouped and matched in various combinations, to build synthetic (i.e., engineered) regulatory elements. After the last round of functional analyses lead regulatory elements with high and specific activity in the target cell line were chosen.

### Example 3: Analysis of Synthetic Promoters

To determine whether stronger or more specific promoters can be developed, additional engineered photoreceptor-specific regulatory elements were generated using the results from Example 1 and Example 2. In addition, results from the MPRA library screen was combined with the results of Example 1 and Example 2 to generate engineered photoreceptor-specific regulatory elements with improved promoter strength and improved specificity for rod and cone photoreceptor cells. Table 3 shows the various exemplary constructs consisting of the engineered regulatory elements as previously described and their promoter strength (as a percentage of CAG promoter activity) and cell-type specificity. Table 4 shows the sequences of exemplary regulatory element components used to construct the exemplary enhancers of Example 3.

TABLE 3
Selected Engineered Enhancer & Promoter Regulatory Elements

| | | Specificity | | |
|---|---|---|---|---|
| | Strength | (Y79: | | SEQ ID |
| Name | (%CAG) | ARPE-19) | Regulatory element sequence | NO |
| SB08160 | 18.0 | 2722.8 | TGCTGAGTCAGCAAGTATACGAACTAAGCCACAGGAGTCT | 164 |
| | | | TGTACCATCTGTCAATCGGCTTAACCATCTGTCAAGTATC | |
| | | | TATACCATCTGTCCCTGCAGGTCTAGAGGGTATATAATGG | |
| | | | GGGCCA | |
| SB08163 | 9.1 | 913.2 | TGCTGAGTCAGCATAGGAAGAGTCTAAGCCACAGCACGCG | 165 |
| | | | TAGTCTAATCCTCAGCCTACTTAACTATAAATAGATCGAG | |
| | | | CAATACCATCTGTCCCTGCAGGTCTAGAGGGTATATAATG | |
| | | | GGGGCCA | |
| SB08165 | 5.3 | 586.5 | GACAGATGGTTTTGACGCCTTGTGGCTTAGAGGCACATCA | 166 |
| | | | TGCTGAGTCAGCAACCGACGGCTGAGGATTAGTTTGCGAG | |
| | | | TGTACCATCTGTCCCTGCAGGTCTAGAGGGTATATAATGG | |
| | | | GGGCCA | |
| SB08166 | 8.0 | 1064.0 | TGCTGACTCAGCAACGACTATAAGTGGCTTAGAACATAAC | 167 |
| | | | GTCTCTAATCCTCAGACTGCGCGTGACAGATGGTTTCGTC | |
| | | | CCGTACCATCTGTCCCTGCAGGTCTAGAGGGTATATAATG | |
| | | | GGGGCCA | |
| SB08179 | 6.0 | 480.2 | ACTATAAATAGAAACTACAACTGAGGATTAGAGTACACAC | 168 |
| | | | TTGCTGAGTCAGCAAATCTTTAGTCTAAGCCACTTAGATC | |
| | | | GCCAACCATCTGTCCCTGCAGGTCTAGAGGGTATATAATG | |
| | | | GGGGCCA | |
| SB08182 | 8.8 | 753.7 | CTAAGCCACATTCGTCTAGTACTAATCCTCTCGGCAGCCG | 169 |
| | | | ATCTAATCCTCAGTCTAGCGCTGACAGATGGTTCACGGGT | |
| | | | GGATTAATTGGCCTGCAGGTCTAGAGGGTATATAATGGGG | |
| | | | GCCA | |
| SB08183 | 7.7 | 305.5 | GACAGATGGTTTGTCGAGTCACTAATCCTCATCCTTAGAG | 170 |
| | | | AAGAGGATTAGTTGAACAGTATGCTGACTCAGCAAGTGTC | |
| | | | GCAACCATCTGTCCCTGCAGGTCTAGAGGGTATATAATGG | |
| | | | GGGCCA | |
| SB08184 | 10.4 | 598.1 | AAGGTCATGACCTTAAAACCATGCTGACTCAGCATGCACG | 171 |
| | | | CATTGTGGCTTAGAATTGCGCCTATCTAATCCTCTATCAG | |
| | | | ACAGAACCATCTGTCCCTGCAGGTCTAGAGGGTATATAAT | |
| | | | GGGGGCCA | |
| SB08209 | 21.0 | 153.3 | GGTTACACAACCAGGCGGGGAGGGGCCCCGGGGGGGGGAG | 172 |
| | | | GGGGCCGGCCCGCGGGCCGCGGGACTACGAAGCCCAGAGG | |
| | | | TCCCTGCGGCTGGGGTGAGTCCTGCTCCTTTGTTCTTCCC | |
| | | | ATCGGCCCGCGCCCATTGGCTCTCCGGCCCGCCGCTCACC | |
| | | | GCCCCTCCTCCGCACCGCCCGGGACTACGAAGCCCAGAGG | |
| | | | TCCCTGCGGCCGGGGCACCCTGGGACTTGTAGTCCAAGCC | |
| | | | TGGGGTGAGTCCTGCTCCTTTGTTCTTCCCGGGACTACGA | |
| | | | AGCCCAGAGGTCCCTGCGGCTGGGGTGAGTCCTGCTCCTT | |
| | | | TGTTCTTCCCGGGACTACGAAGCCCAGAGGTCCCTGCGGC | |
| | | | TTTTTTCAGCCCCCTCCCCCCCATCCCCCATGGAGCCCTG | |
| | | | CAGGTCTAGAGGGTATATAATGGGGGCCA | |
| SB08211 | 20.6 | 193.1 | CAGGCGGGGAGGGGCCCCGGGGCGGGGAGGGGGCCGGCCC | 173 |
| | | | GCGGGCCGCGCAGCCGGAAGCCGGGACCGCCACCGGCCCC | |
| | | | CGGCGAGGGGGGTCCCTCCCTACCTCTGCCCCGCGCTCTG | |
| | | | CCTTTGATCCTCTGCTCGGCTCTGAGCCATATCGGCCCGC | |
| | | | GCCCATTGGCTCTCCGGCCCGCCGCTCACCGCCCCTCCTC | |
| | | | CGCACCGCCCTGGGGTGAGTCCTGCTCCTTTGTTCTTCCC | |
| | | | CGGGGCACCCTGGGACTTGTAGTCCAAGCCGCTTGCCACC | |
| | | | TGCCGGCTGCAAACGGCGGAGGGACTACGAAGCCCAGAGG | |
| | | | TCCCTGCGGCCTCCTGCTATTGGTCAGAATGTGTCACGTG | |
| | | | ACCATACTCAGCTGCAAGGGTTGGTGGGAATTTTTTCAGC | |
| | | | CCCCTCCCCCCCATCCCCCATGGAGCCCTGCAGGTCTAGA | |
| | | | GGGTATATAATGGGGGCCA | |
| SB08212 | 7.9 | 407.1 | CAGGCGGGGAGGGGCCCCGGGGGGGGGAGGGGGCCGGCCC | 174 |
| | | | GCGGGCCGCGCAGCCGGAAGCCGGGACCGCCACCGGCCCC | |
| | | | CGGCGAGGGGCTCCTGCTATTGGTCAGAATGTGTCACGTG | |
| | | | ACCATACTCAGCTGCAAGGGTTGGTGGGAAATCGGCCCGC | |
| | | | GCCCATTGGCTCTCCGGCCCGCCGCTCACCGCCCCTCCTC | |
| | | | CGCACCGCCCTGGGGTGAGTCCTGCTCCTTTGTTCTTCCC | |
| | | | CGGGGCACCCTGGGACTTGTAGTCCAAGCCTGGGGTGAGT | |
| | | | CCTGCTCCTTTGTTCTTCCCGGGACTACGAAGCCCAGAGG | |
| | | | TCCCTGCGGCCTCCTGCTATTGGTCAGAATGTGTCACGTG | |
| | | | ACCATACTCAGCTGCAAGGGTTGGTGGGAAATGTAGTCTT | |
| | | | TTTTTCTGGGAGGCCATGTGTTCGGCTTAAAATTGTCTTT | |
| | | | TTTTCTTTTTCCTGCAGGTCTAGAGGGTATATAATGGGGG | |
| | | | CCA | |
| SB08213 | 15.4 | 298.7 | CAGGCGGGGAGGGGCCCCGGGGGGGGGAGGGGGCCGGCCC | 175 |
| | | | GCGGGCCGCGCAGCCGGAAGCCGGGACCGCCACCGGCCCC | |
| | | | CGGCGAGGGGCGGGGCACCCTGGGACTTGTAGTCCAAGCC | |
| | | | GGGACTACGAAGCCCAGAGGTCCCTGCGGCATCGGCCCGC | |
| | | | GCCCATTGGCTCTCCGGCCCGCCGCTCACCGCCCCTCCTC | |
| | | | CGCACCGCCCGGGACTACGAAGCCCAGAGGTCCCTGCGGC | |
| | | | CGGGGCACCCTGGGACTTGTAGTCCAAGCCGCTTGCCACC | |
| | | | TGCCGGCTGCAAACGGCGGAGGGACTACGAAGCCCAGAGG | |
| | | | TCCCTGCGGCATCGGCCCGCGCCCATTGGCTCTCCGGCCC | |
| | | | GCCGCTCACCGCCCCTCCTCCGCACCGCCCTTTTTTCAGC | |
| | | | CCCCTCCCCCCCATCCCCCATGGAGCCCTGCAGGTCTAGA | |
| | | | GGGTATATAATGGGGGCCA | |
| SB08214 | 5.9 | 127.0 | CGGGGCACCCTGGGACTTGTAGTCCAAGCCGGGACTACGA | 176 |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | CGGGGCACCCTGGGACTTGTAGTCCAAGCCGGGACTACGA | |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | CGGGGCACCCTGGGACTTGTAGTCCAAGCCGGGACTACGA | |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | CGGGGCACCCTGGGACTTGTAGTCCAAGCCGGGACTACGA | |
| | | | AGCCCAGAGGTCCCTGCGGCTTTTTTCAGCCCCCTCCCCC | |
| | | | CCATCCCCCATGGAGCCCTGCAGGTCTAGAGGGTATATAA | |
| | | | TGGGGGCCA | |
| SB08215 | 20.8 | 462.4 | GGTTACACAACCAGGCGGGGAGGGGCCCCGGGGGGGGGAG | 177 |
| | | | GGGGCCGGCCCGCGGGCCGCATCGGCCCGCGCCCATTGGC | |
| | | | TCTCCGGCCCGCCGCTCACCGCCCCTCCTCCGCACCGCCC | |
| | | | GGGACTACGAAGCCCAGAGGTCCCTGCGGCGGTCCCTCCC | |
| | | | TACCTCTGCCCCGCGCTCTGTGGGGTGAGTCCTGCTCCTT | |
| | | | TGTTCTTCCCGGTTACACAACCAGGCGGGGAGGGGCCCCG | |
| | | | GGGCGGGGAGGGGGCCGGCCCGCGGGCCGCATCGGCCCGC | |
| | | | GCCCATTGGCTCTCCGGCCCGCCGCTCACCGCCCCTCCTC | |
| | | | CGCACCGCCCGGGACTACGAAGCCCAGAGGTCCCTGCGGC | |
| | | | GGTCCCTCCCTACCTCTGCCCCGCGCTCTGTGGGGTGAGT | |
| | | | CCTGCTCCTTTGTTCTTCCCTTTTTTCAGCCCCCTCCCCC | |
| | | | CCATCCCCCATGGAGCCCTGCAGGTCTAGAGGGTATATAA | |
| | | | TGGGGGCCA | |
| SB08216 | 8.3 | 66.7 | CTCCTGCTATTGGTCAGAATGTGTCACGTGACCATACTCA | 178 |
| | | | GCTGCAAGGGTTGGTGGGAAATGTAGTCTTTTTTTCTGGG | |
| | | | AGGCCATGTGTTCGGCTTAAAATTGTCTTTTTTTCTTTTT | |
| | | | GGTCCCTCCCTACCTCTGCCCCGCGCTCTGTGGGGTGAGT | |
| | | | CCTGCTCCTTTGTTCTTCCCCTCCTGCTATTGGTCAGAAT | |
| | | | GTGTCACGTGACCATACTCAGCTGCAAGGGTTGGTGGGAA | |
| | | | ATGTAGTCTTTTTTTCTGGGAGGCCATGTGTTCGGCTTAA | |
| | | | AATTGTCTTTTTTTCTTTTTGGTCCCTCCCTACCTCTGCC | |
| | | | CCGCGCTCTGTGGGGTGAGTCCTGCTCCTTTGTTCTTCCC | |
| | | | CTCCTGCTATTGGTCAGAATGTGTCACGTGACCATACTCA | |
| | | | GCTGCAAGGGTTGGTGGGAAATGTAGTCTTTTTTTCTGGG | |
| | | | AGGCCATGTGTTCGGCTTAAAATTGTCTTTTTTTCTTTTT | |
| | | | CCTGCAGGTCTAGAGGGTATATAATGGGGGCCA | |
| SB08217 | 7.5 | 159.1 | CGGGGCACCCTGGGACTTGTAGTCCAAGCCGGGACTACGA | 179 |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | CGGGGCACCCTGGGACTTGTAGTCCAAGCCGGGACTACGA | |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | CGGGGCACCCTGGGACTTGTAGTCCAAGCCGGGACTACGA | |
| | | | AGCCCAGAGGTCCCTGCGGCCTCCTGCTATTGGTCAGAAT | |
| | | | GTGTCACGTGACCATACTCAGCTGCAAGGGTTGGTGGGAA | |
| | | | ATGTAGTCTTTTTTTCTGGGAGGCCATGTGTTCGGCTTAA | |
| | | | AATTGTCTTTTTTTCTTTTTGGTCCCTCCCTACCTCTGCC | |
| | | | CCGCGCTCTGTGGGGTGAGTCCTGCTCCTTTGTTCTTCCC | |
| | | | CCTGCAGGTCTAGAGGGTATATAATGGGGGCCA | |
| SB08218 | 36.8 | 104.7 | TGGGGTGAGTCCTGCTCCTTTGTTCTTCCCGGGACTACGA | 180 |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | TGGGGTGAGTCCTGCTCCTTTGTTCTTCCCGGGACTACGA | |
| | | | AGCCCAGAGGTCCCTGCGGCATCGGCCCGCGCCCATTGGC | |
| | | | TCTCCGGCCCGCCGCTCACCGCCCCTCCTCCGCACCGCCC | |
| | | | TGGGGTGAGTCCTGCTCCTTTGTTCTTCCCGGGACTACGA | |
| | | | AGCCCAGAGGTCCCTGCGGCCTCCTGCTATTGGTCAGAAT | |
| | | | GTGTCACGTGACCATACTCAGCTGCAAGGGTTGGTGGGAA | |
| | | | GGGACTACGAAGCCCAGAGGTCCCTGCGGCTGGGGTGAGT | |
| | | | CCTGCTCCTTTGTTCTTCCCTTTTTTCAGCCCCCTCCCCC | |
| | | | CCATCCCCCATGGAGCCCTGCAGGTCTAGAGGGTATATAA | |
| | | | TGGGGGCCA | |
| SB08322 | 6.1 | 673.5 | CAGGCGGGGAGGGGCCCCGGGGGGGGGAGGGGGCCGGCCC | 181 |
| | | | GCGGGCCGCGCAGCCGGAAGCCGGGACCGCCACCGGCCCC | |
| | | | CGGCGAGGGGAGCCCGGCTCCAGGCCCCGCCCCCTGGCGG | |
| | | | ATCGGCCCGCGCCCATTGGCTCTCCGGCCCGCCGCTCACC | |
| | | | GCCCCTCCTCCGCACCGCCCCGGGGCACCCTGGGACTTGT | |
| | | | AGTCCAAGCCGCTTGCCACCTGCCGGCTGCAAACGGCGGA | |
| | | | GGGACTACGAAGCCCAGAGGTCCCTGCGGCTCCCCCTCCT | |
| | | | TTCCGAAGCCCCCCTCCCTGTTTTTTCAGCCCCCTCCCCC | |
| | | | CCATCCCCCATGGAGCGACAGATGGTTTTGACGCCTTGTG | |
| | | | GCTTAGAGGCACATCATGCTGAGTCAGCAACCGACGGCTG | |
| | | | AGGATTAGTTTGCGAGTGTACCATCTGTCCCTGCAGGTTC | |
| | | | GCATATTAAGGTGACGCGTGTGGCCTCGAACACCGAGCGA | |
| | | | CCCTGCAGCGACCCGCTTAA | |
| SB08323 | 6.5 | 1312.3 | CAGGCGGGGAGGGGCCCCGGGGGGGGAGGGGGCCGGCCCG | 182 |
| | | | CGGGCCGCGCAGCCGGAAGCCGGGACCGCCACCGGCCCCC | |
| | | | GGCGAGGGGAGCCCGGCTCCAGGCCCCGCCCCCTGGCGGA | |
| | | | TCGGCCCGCGCCCATTGGCTCTCCGGCCCGCCGCTCACCG | |
| | | | CCCCTCCTCCGCACCGCCCCGGGGCACCCTGGGACTTGTA | |
| | | | GTCCAAGCCGCTTGCCACCTGCCGGCTGCAAACGGCGGAG | |
| | | | GGACTACGAAGCCCAGAGGTCCCTGCGGCTCCCCCTCCTT | |
| | | | TCCGAAGCCCCCCTCCCTGTTTTTTCAGCCCCCTCCCCCC | |
| | | | CATCCCCCATGGAGCTGCTGAGTCAGCAAGTATACGAACT | |
| | | | AAGCCACAGGAGTCTTGTACCATCTGTCAATCGGCTTAAC | |
| | | | CATCTGTCAAGTATCTATACCATCTGTCCCTGCAGGTTCG | |
| | | | CATATTAAGGTGACGCGTGTGGCCTCGAACACCGAGCGAC | |
| | | | CCTGCAGCGACCCGCTTAA | |
| SB08324 | 16.8 | 1982.9 | CAGGCGGGGAGGGGCCCCGGGGGGGGGAGGGGGCCGGCCC | 183 |
| | | | GCGGGCCGCGCAGCCGGAAGCCGGGACCGCCACCGGCCCC | |
| | | | CGGCGAGGGGAGCCCGGCTCCAGGCCCCGCCCCCTGGCGG | |
| | | | ATCGGCCCGCGCCCATTGGCTCTCCGGCCCGCCGCTCACC | |
| | | | GCCCCTCCTCCGCACCGCCCCGGGGCACCCTGGGACTTGT | |
| | | | AGTCCAAGCCGCTTGCCACCTGCCGGCTGCAAACGGCGGA | |
| | | | GGGACTACGAAGCCCAGAGGTCCCTGCGGCTCCCCCTCCT | |
| | | | TTCCGAAGCCCCCCTCCCTGTTTTTTCAGCCCCCTCCCCC | |
| | | | CCATCCCCCATGGAGCCTAAGCCACAGTAACATGCGACTA | |
| | | | ATCCTCTCTGTCTACAGAGTGGCTTAGTCTATCGTGTTGC | |
| | | | TGACTCAGCATTAGATAGTGACAGATGGTCCTGCAGGTCT | |
| | | | AGAGGGTATATAATGGGGGCCA | |
| SB08326 | 15.2 | 554.4 | GGTTACACAACCAGGCGGGGAGGGGCCCCGGCAGCCGGAA | 184 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACACCCCACCCC | |
| | | | TTCCCCCTCCTTTCCGAAGCCCCCCTCCCTGTTTTTTCAG | |
| | | | CCCCCTCGACAGATGGTTTTGACGCCTTGTGGCTTAGAGG | |
| | | | CACATCATGCTGAGTCAGCAACCGACGGCTGAGGATTAGT | |
| | | | TTGCGAGTGTACCATCTGTCCCTGCAGGTTCGCATATTAA | |
| | | | GGTGACGCGTGTGGCCTCGAACACCGAGCGACCCTGCAGC | |
| | | | GACCCGCTTAA | |
| SB08327 | 7.5 | 542.7 | GGTTACACAACCAGGCGGGGAGGGGCCCCGGCAGCCGGAA | 185 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACACCCCACCCC | |
| | | | TTCCCCCTCCTTTCCGAAGCCCCCCTCCCTGTTTTTTCAG | |
| | | | CCCCCTCTGCTGAGTCAGCAAGTATACGAACTAAGCCACA | |
| | | | GGAGTCTTGTACCATCTGTCAATCGGCTTAACCATCTGTC | |
| | | | AAGTATCTATACCATCTGTCCCTGCAGGTTCGCATATTAA | |
| | | | GGTGACGCGTGTGGCCTCGAACACCGAGCGACCCTGCAGC | |
| | | | GACCCGCTTAA | |
| SB08328 | 18.1 | 1340.4 | GGTTACACAACCAGGCGGGGAGGGGCCCCGGCAGCCGGAA | 186 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACACCCCACCCC | |
| | | | TTCCCCCTCCTTTCCGAAGCCCCCCTCCCTGTTTTTTCAG | |
| | | | CCCCCTCCTAAGCCACAGTAACATGCGACTAATCCTCTCT | |
| | | | GTCTACAGAGTGGCTTAGTCTATCGTGTTGCTGACTCAGC | |
| | | | ATTAGATAGTGACAGATGGTCCTGCAGGTCTAGAGGGTAT | |
| | | | ATAATGGGGGCCA | |
| SB08329 | 7.0 | 118.7 | GGTTACACAACCAGGCGGGGAGGGGCCCCGGCAGCCGGAA | 187 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACACCCCACCCC | |
| | | | TTCCCCCTCCTTTCCGAAGCCCCCCTCCCTGTTTTTTCAG | |
| | | | CCCCCTCCCTGCAGGTCTAGAGGGTATATAATGGGGGCCA | |
| SB08330 | 6.1 | 154.0 | GGTTACACAACCAGGCGGGGAGGGGCCCCGGGGGGGGGAG | 188 |
| | | | GGGGCCGGCCCGCGGGCCGCGCAGCCGGAAGCCGGGACCG | |
| | | | CCACCGGCCCCCGGCGAGGGGAGCCCGGCTCCAGGCCCCG | |
| | | | TGCCTAAAACGAGATTCTGTACGATAAAGCCGCGCCGCGC | |
| | | | GATCGGCCCGCGCCCATTGGCTCTCCGGCCCGCCGCTCAC | |
| | | | CGCCCCTCCTCCGCACCGCCCCTACCCGCAGGCCGCGGCG | |
| | | | GGCTGTCGGCGCGGGGCACCCTGGGACTTGTAGTCCAAGC | |
| | | | CGCTTGCCACCTGCCGGCTGCAAACGGCGGAGGGACTACG | |
| | | | AAGCCCAGAGGTCCCTGCGGCCCTGCCCGCCCACCCGGAC | |
| | | | ACCCCACCCCTTCCCCCTCCTTTCCGAAGCCCCCCTCCCT | |
| | | | GTTTTTTCAGCCCCCTCCCTGCAGGTCTAGAGGGTATATA | |
| | | | ATGGGGGCCA | |
| SB11066 | 34.5 | 207.5 | GACAGATGGTTTTGACGCCTTGTGGCTTAGAGGCACATCA | 189 |
| | | | TGCTGAGTCAGCAACCGACGGCTGAGGATTAGTTTGCGAG | |
| | | | TGTACCATCTGTCTGCTGAGTCAGCAAGTATACGAACTAA | |
| | | | GCCACAGGAGTCTTGTACCATCTGTCAATCGGCTTAACCA | |
| | | | TCTGTCAAGTATCTATACCATCTGTCCTCCTGCTATTGGT | |
| | | | CAGAATGTGTCACGTGACCATACTCAGCTGCAAGGGTTGG | |
| | | | TGGGAACTAAGCCACAGTAACATGCGACTAATCCTCTCTG | |
| | | | TCTACAGAGTGGCTTAGTCTATCGTGTTGCTGACTCAGCA | |
| | | | TTAGATAGTGACAGATGGTCCTGCAGGTCTAGAGGGTATA | |
| | | | TAATGGGGGCCA | |
| SB11067 | 26.8 | 535.8 | GACAGATGGTTTTGACGCCTTGTGGCTTAGAGGCACATCA | 190 |
| | | | TGCTGAGTCAGCAACCGACGGCTGAGGATTAGTTTGCGAG | |
| | | | TGTACCATCTGTCTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTCTTTTTCTAAGCC | |
| | | | ACAGTAACATGCGACTAATCCTCTCTGTCTACAGAGTGGC | |
| | | | TTAGTCTATCGTGTTGCTGACTCAGCATTAGATAGTGACA | |
| | | | GATGGTCCTGCAGGTCTAGAGGGTATATAATGGGGGCCA | |
| SB11068 | 37.9 | 1131.3 | TGCTGAGTCAGCAAGTATACGAACTAAGCCACAGGAGTCT | 191 |
| | | | TGTACCATCTGTCAATCGGCTTAACCATCTGTCAAGTATC | |
| | | | TATACCATCTGTCTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTCTTTTTCTAAGCC | |
| | | | ACAGTAACATGCGACTAATCCTCTCTGTCTACAGAGTGGC | |
| | | | TTAGTCTATCGTGTTGCTGACTCAGCATTAGATAGTGACA | |
| | | | GATGGTCCTGCAGGTCTAGAGGGTATATAATGGGGGCCA | |
| SB08338 | 6.0 | 145.8 | CTAAGCCACAGTAACATGCGACTAATCCTCTCTGTCTACA | 192 |
| | | | GAGTGGCTTAGTCTATCGTGTTGCTGACTCAGCATTAGAT | |
| | | | AGTGACAGATGGTTTACCTACTAGGTTAATTCGTCCGATA | |
| | | | GATACTACGAATTGCGAGCTTCTAAGTCCAATTTTCGGTA | |
| | | | TTCGAGTCGTCAGACTCAATTATTACACGGTTAAGACTCA | |
| | | | ATCCGGTAAGTAATACTGTGATCAGGTGTCTACAAGACTA | |
| | | | GTACTCCTGCTATTGGTCAGAATGTGTCACGTGACCATAC | |
| | | | TCAGCTGCAAGGGTTGGTGGGAAATGTAGTCTTTTTTTCT | |
| | | | GGGAGGCCATGTGTTCGGCTTAAAATTGTCTTTTTTTCTT | |
| | | | TTTACTATCACGTATATACCAGCGAGTTCGATAATACACT | |
| | | | CTCCTGCAGGTCTAGAGGGTATATAATGGGGGCCA | |
| SB08478 | 5.6 | 235.4 | CTCAGGCCCTGCATTGCGCCAACGGCGCAGCGCTGGGCCG | 193 |
| | | | CGACCCCGGCACCGGCACCCGGGGACGTTGTGAATAGCTG | |
| | | | GTCGAAGTTTGGTTGAAGCACAGGAGGGTAAATAAGAAGG | |
| | | | TTTCCAGACAAAGAGACTCTTAAAGGTACAGGCCAGAGAC | |
| | | | GCTCCCGAGCCCATCTCCTCCTGCTATTGGTCAGAATGTG | |
| | | | TCACGTGACCATACTCAGCTGCAAGGGTTGGTGGGAAATG | |
| | | | TAGTCTTTTTTTCTGGGAGGCCATGTGTTCGGCTTAAAAT | |
| | | | TGTCTTTTTTTCTTTTTCTTTATTTCTTTTTTCTTTTCTT | |
| | | | TCTTTTTATACACTCTCCTGCAGGAGACGCTAGCGGGGGG | |
| | | | CTATAAAAGGGGGTGGGGGCGTTCGTCCTCACTCT | |
| SB08479 | 19.3 | 380.6 | CTCAGGCCCTGCATTGCGCCAACGGCGCAGCGCTGGGCCG | 194 |
| | | | CGACCCCGGCACCGGCACCCCTCCTGCTATTGGTCAGAAT | |
| | | | GTGTCACGTGACCATACTCAGCTGCAAGGGTTGGTGGGAA | |
| | | | ATGTAGTCTTTTTTTCTGGGAGGCCATGTGTTCGGCTTAA | |
| | | | AATTGTCTTTTTTTCTTTTTGGGGACGTTGTGAATAGCTG | |
| | | | GTCGAAGTTTGGTTGAAGCACAGGAGGGTAAATAAGAAGG | |
| | | | TTTCCAGACAAAGAGACTCTTAAAGGTACAGACTCTTATG | |
| | | | TCTGTCCCTCCTCCTTAAAGGGCCAGAGACGCTCCCGAGC | |
| | | | CCATCTCCCTGCAGGAGACGCTAGCGGGGGGCTATAAAAG | |
| | | | GGGGTGGGGGCGTTCGTCCTCACTCT | |
| SB08480 | 20.3 | 1240.4 | CTAAGCCACAGTAACATGCGACTAATCCTCTCTGTCTACA | 195 |
| | | | GAGTGGCTTAGTCTATCGTGTTGCTGACTCAGCATTAGAT | |
| | | | AGTGACAGATGGTCTCAGGCCCTGCATTGCGCCAACGGCG | |
| | | | CAGCGCTGGGCCGCGACCCCGGCACCGGCACCCGTTCCGA | |
| | | | GGGTTCGCGCCGCAGGCGCAAAGCCACATTGCTATAGTGC | |
| | | | TGTATAGCGATTATTGAGCCTGGCGTAAGGGGCGGCGGCG | |
| | | | TAGGGGGACGTTGTGAATAGCTGGTCGAAGTTTGGTTGAA | |
| | | | GCACAGGAGGGTAAATAAGAAGGTTTCCAGACAAAGAGAC | |
| | | | TCTTAAAGGTACAGACTCTTATGTCTGTCCCTCCTCCTTA | |
| | | | AAGGGCCAGAGACGCTCCCGAGCCCATCTCCCTGCAGGAG | |
| | | | ACGCTAGCGGGGGGCTATAAAAGGGGGTGGGGGCGTTCGT | |
| | | | CCTCACTCT | |
| SB08482 | 5.1 | 285.2 | AGCACGCGCGAGCTGTCACCAGGGCGCGAGACTACGAATT | 196 |
| | | | GCGAGCTTCTAAGTCCAATTTCTATTGGCCCGCCCGCTGG | |
| | | | AGCCCCGCCCCTCAGGCCCTGCATTGCGCCAACGGCGCAG | |
| | | | CGCTGGGCCGCGACCCCGGCACCGGCACCCGTTCCGAGGG | |
| | | | TTCGCGCCGCAGGCGCAAAGTTTAGGGTCAGCCACAAACG | |
| | | | CGCGGCCGCTTTGAGCCTGGCGTAAGGGGCGGCGGCGTAG | |
| | | | GGGGACGTTGTGAATAGCTGGTCGAAGTTTGGTTGAAGCA | |
| | | | CAGGAGGGTAAATAAGAAGGTTTCCAGACAAAGAGACTCT | |
| | | | TAAAGGTACAGACTCTTATGTCTGTCCCTCCTCCTTAAAG | |
| | | | GGCCAGAGACGCTCCCGAGCCCATCTCCCTGCAGGAGACG | |
| | | | CTAGCGGGGGGCTATAAAAGGGGGTGGGGGCGTTCGTCCT | |
| | | | CACTCT | |
| SB08483 | 15.6 | 1816.4 | GGTTACACAACCAGGGGGGAGGGGCCCCGGCAGCCGGAAG | 197 |
| | | | CCGGGACCGCCACCGGCCCCGCGCCGCGCGATCGGCCCGC | |
| | | | GCCCATTGGCTCTCCGGCCCGCCGCTCACCGCCCCTCCTC | |
| | | | CGCACCGCCCCTACCCGCAGGCCGCGGCGGGCTGTCGGCG | |
| | | | CGGGGCACCCTGGGACTTGTAGTCCAAGCCGCTTGCCACC | |
| | | | TGCCGGCTGCAAACGGCGGAGGGACTACGAAGCCCAGAGG | |
| | | | TCCCTGCGGCCCTGCCCGCCCACCCGGACGCAAGGAAGAT | |
| | | | TATTTTGAGTCACTGCACCCTTTCTGAACAATAGCTGTTA | |
| | | | CCAAGGTCTCAGGAGCATTAGGCGCTCAATTCTATTTTAA | |
| | | | GTAGCATGGTTGGAGCTTAACCCCAAGTCTAATTTTATTC | |
| | | | TGCCTCTTTATTTAAAAAGGACAGATGGTTCCCAACGACT | |
| | | | AATCCTCATAGAACGCACTAATCCTCCCTGCAGGTCTAGA | |
| | | | GGGTATATAATGGGGGCCA | |
| SB08484 | 16.7 | 2041.6 | GGTTACACAACCAGGCGGGGAGGGGCCCCGGCAGCCGGAA | 198 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACGCAAGGAAGA | |
| | | | TTATTTTGAGTCACTGCACCCTTTCTGAACAATAGCTGTT | |
| | | | ACCAAGGTCTCAGGAGCATTAGGCGCTCAATTCTATTTTA | |
| | | | AGTAGCATGGTTGGAGCTTAACCCCAAGTCTAATTTTATT | |
| | | | CTGCCTCTTTATTTAAAAAGCTAATCCTCTCGGCCCGATC | |
| | | | TAATCCTCAGTCTCGCTGACAGATGGTCCTGCAGGTCTAG | |
| | | | AGGGTATATAATGGGGGCCA | |
| SB08485 | 15.4 | 2591.1 | GGTTACACAACCAGGCGGGGAGGGGCCCCGGCAGCCGGAA | 199 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACGACAGATGGT | |
| | | | TCCCAACGACTAATCCTCATAGAACGCACTAATCCTCGCA | |
| | | | AGGAAGATTATTTTGAGTCACTGCACCCTTTCTGAACAAT | |
| | | | AGCTGTTACCAAGGTCTCAGGAGCATTAGGCGCTCAATTC | |
| | | | TATTTTAAGTAGCATGGTTGGAGCTTAACCCCAAGTCTAA | |
| | | | TTTTATTCTGCCTCTTTATTTAAAAAGCCTGCAGGTCTAG | |
| | | | AGGGTATATAATGGGGGCCA | |
| SB08486 | 17.9 | 3650.0 | GGTTACACAACCAGGCGGGGAGGGGCCCCGGCAGCCGGAA | 200 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACCTAATCCTCT | |
| | | | CGGCCCGATCTAATCCTCAGTCTCGCTGACAGATGGTGCA | |
| | | | AGGAAGATTATTTTGAGTCACTGCACCCTTTCTGAACAAT | |
| | | | AGCTGTTACCAAGGTCTCAGGAGCATTAGGCGCTCAATTC | |
| | | | TATTTTAAGTAGCATGGTTGGAGCTTAACCCCAAGTCTAA | |
| | | | TTTTATTCTGCCTCTTTATTTAAAAAGCCTGCAGGTCTAG | |
| | | | AGGGTATATAATGGGGGCCA | |
| SB08487 | 7.2 | 1257.9 | GGTTACACAACCAGGCGGGGAGGGGCCCCGGCAGCCGGAA | 201 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACAGCGCTGACA | |
| | | | GATGGTTCACGGGCAAGGAAGATTATTTTGAGTCACTGCA | |
| | | | CCCTTTCTGAACAATAGCTGTTACCAAGGTCTCAGGAGCA | |
| | | | TTAGGCGCTCAATTCTATTTTAAGTAGCATGGTTGGAGCT | |
| | | | TAACCCCAAGTCTAATTTTATTCTGCCTCTTTATTTAAAA | |
| | | | AGGGGCCAGACAGATGGTTCCCAATACCCTGCAGGTCTAG | |
| | | | AGGGTATATAATGGGGGCCA | |
| SB08490 | 21.6 | 315.0 | GGTTACACAACCAGGCGGGGAGGGGCCCCGGGGGGGGGAG | 202 |
| | | | GGGGCCGGCCCGCGGGCCGCGCAGCCGGAAGCCGGGACCG | |
| | | | CCACCGGCCCTACGGGCCAGACAGATGGTTCCCAATACGA | |
| | | | CTAATCCTCATAGAAGACGCACTAATCCTCCGCGCCGCGC | |
| | | | GATCGGCCCGCGCCCATTGGCTCTCCGGCCCGCCGCTCAC | |
| | | | CGCCCCTCCTCCGCACCGCCCCTACCCGCAGGCCGCGGCG | |
| | | | GGCTGTCGGCGCGGGGCACCCTGGGACTTGTAGTCCAAGC | |
| | | | CGCTTGCCACCTGCCGGCTGCAAACGGCGGAGGGACTACG | |
| | | | AAGCCCAGAGGTCCCTGCGGCCCTGCCCGCCCACCCGGAC | |
| | | | ACCCCACCCCTTCCCCCTCCTTTCCGAAGCCCCCCTCCCT | |
| | | | GTTTTTTCAGCCCCCTCCCTGCAGGTTCGCATATTAAGGT | |
| | | | GACGCGTGTGGCCTCGAACACCGAGCGACCCTGCAGCGAC | |
| | | | CCGCTTAA | |
| SB08493 | 24.5 | 732.6 | GGTTACACAACCAGGCGGGGAGGGGCCCCGGGGGGGGGAG | 203 |
| | | | GGGGCCGGCCCGCGGGCCGCGCAGCCGGAAGCCGGGACCG | |
| | | | CCACCGGCCCATTCGTCTAGTACTAATCCTCTCGGCAGCC | |
| | | | GATCTAATCCTCAGTCTAGCGCTGACAGATGGTTCACGGG | |
| | | | TGGACGCGCCGCGCGATCGGCCCGCGCCCATTGGCTCTCC | |
| | | | GGCCCGCCGCTCACCGCCCCTCCTCCGCACCGCCCCTACC | |
| | | | CGCAGGCCGCGGCGGGCTGTCGGCGCGGGGCACCCTGGGA | |
| | | | CTTGTAGTCCAAGCCGCTTGCCACCTGCCGGCTGCAAACG | |
| | | | GCGGAGGGACTACGAAGCCCAGAGGTCCCTGCGGCCCTGC | |
| | | | CCGCCCACCCGGACACCCCACCCCTTCCCCCTCCTTTCCG | |
| | | | AAGCCCCCCTCCCTGTTTTTTCAGCCCCCTCCCTGCAGGT | |
| | | | CTAGAGGGTATATAATGGGGGCCA | |
| SB08494 | 91.0 | 17553.0 | TACGGGCCAGACAGATGGTTCCCAATACGACTAATCCTCA | 204 |
| | | | TAGAAGACGCACTAATCCTCGTGAGTGACAGGCGAACCAG | |
| | | | TACTATAAATAGATACGGGCCAGACAGATGGTTCCCAATA | |
| | | | CGACTAATCCTCATAGAAGACGCACTAATCCTCACTGTCG | |
| | | | AGTTGTGAGTGACAGGCGAACCAGTACTATAAATAGATAC | |
| | | | GGGCCAGACAGATGGTTCCCAATACGACTAATCCTCATAG | |
| | | | AAGACGCACTAATCCTCACTGTCGAGTTGTGAGTGACAGG | |
| | | | CGAACCAGTACTATAAATAGATACGGGCCAGACAGATGGT | |
| | | | TCCCAATACGACTAATCCTCATAGAAGACGCACTAATCCT | |
| | | | CACTGTCGAGTTGTGAGTGACAGGCGAACCAGTACTATAA | |
| | | | ATAGATACGGGCCAGACAGATGGTTCCCAATACGACTAAT | |
| | | | CCTCATAGAAGACGCACTAATCCTCCCTGCAGGTCTAGAG | |
| | | | GGTATATAATGGGGGCCA | |
| SB08495 | 138.2 | 1796.8 | TAGTACTAATCCTCTCGGCAGCCGATCTAATCCTCAGTCT | 205 |
| | | | AGCGCTGACAGATGGTTCACCTAAGCCACATTCGTCTAGT | |
| | | | ACTAATCCTCTCGGCAGCCGATCTAATCCTCAGTCTAGCG | |
| | | | CTGACAGATGGTTCACGGGTGGATTAATTGGTGTTCCGAA | |
| | | | CACTAAGCCACATTCGTCTAGTACTAATCCTCTCGGCAGC | |
| | | | CGATCTAATCCTCAGTCTAGCGCTGACAGATGGTTCACGG | |
| | | | GTGGATTAATTGGTGTTCCGAACACTAAGCCACATTCGTC | |
| | | | TAGTACTAATCCTCTCGGCAGCCGATCTAATCCTCAGTCT | |
| | | | AGCGCTGACAGATGGTTCACGGGTGGATTAATTGGTGTTC | |
| | | | CGAACACTAAGCCACATTCGTCTAGTACTAATCCTCTCGG | |
| | | | CAGCCGATCTAATCCTCAGTCTAGCGCTGACAGATGGTTC | |
| | | | ACGGGTGGATTAATTGGCCTGCAGGTCTAGAGGGTATATA | |
| | | | ATGGGGGCCA | |
| SB08496 | 149.2 | 243.1 | GTGAGTGACAGGCGACCAGTACTATAAATAGATACGGGCC | 206 |
| | | | AGACAGATGGTTCCCAATACGACTAATCCTCATAGAAGAC | |
| | | | GCACTAATCCTCCTAAGCCACATTCGTCTAGTACTAATCC | |
| | | | TCTCGGCAGCCGATCTAATCCTCAGTCTAGCGCTGACAGA | |
| | | | TGGTTCACGGGTGGATTAATTGGGTGAGTGACAGGCGAAC | |
| | | | CAGTACTATAAATAGATACGGGCCAGACAGATGGTTCCCA | |
| | | | ATACGACTAATCCTCATAGAAGACGCACTAATCCTCCTAA | |
| | | | GCCACATTCGTCTAGTACTAATCCTCTCGGCAGCCGATCT | |
| | | | AATCCTCAGTCTAGCGCTGACAGATGGTTCACGGGTGGAT | |
| | | | TAATTGGCCTGCAGGTCTAGAGGGTATATAATGGGGGCCA | |
| SB08497 | 110.8 | 857.1 | CTAAGCCACATTCGTCTAGTACTAATCCTCTCGGCAGCCG | 207 |
| | | | ATCTAATCCTCAGTCTAGCGCTGACAGATGGTTCACGGGT | |
| | | | GGATTAATTGGCTAAGCCACATTCGTCTAGTACTAATCCT | |
| | | | CTCGGCAGCCGATCTAATCCTCAGTCTAGCGCTGACAGAT | |
| | | | GGTTCACGGGTGGATTAATTGGCTAAGCCACATTCGTCTA | |
| | | | GTACTAATCCTCTCGGCAGCCGATCTAATCCTCAGTCTAG | |
| | | | CGCTGACAGATGGTTCACGGGTGGATTAATTGGCTAAGCC | |
| | | | ACATTCGTCTAGTACTAATCCTCTCGGCAGCCGATCTAAT | |
| | | | CCTCAGTCTAGCGCTGACAGATGGTTCACGGGTGGATTAA | |
| | | | TTGGCCTGCAGGTCTAGAGGGTATATAATGGGGGCCA | |
| SB08570 | 60.8 | 3492.0 | CTAAGCCACAGTAACATGCGACTAATCCTCTCTGTCTACA | 208 |
| | | | GAGTGGCTTAGTCTATCGTGTTGCTGACTCAGCATTAGAT | |
| | | | AGTGACAGATGGTTAGTAAGGTACTAAGCCACATTCGTCT | |
| | | | AGTACTAATCCTCTCGGCAGCCGATCTAATCCTCAGTCTA | |
| | | | GCGCTGACAGATGGTTCACGGGTGGATTAATTGGGGGGAC | |
| | | | GTTGTGAATAGCTGGTCGAAGTTTGGTTGAAGCACAGGAG | |
| | | | GGTAAATAAGAAGGTTTCCAGACAAAGAGACTCTTAAAGG | |
| | | | TACAGACTCTTATGTCTGTCCCTCCTCCTTAAAGGGCCAG | |
| | | | AGACGCTCCCGAGCCCATCTCCCTGCAGGAGACGCTAGCG | |
| | | | GGGGGCTATAAAAGGGGGTGGGGGCGTTCGTCCTCACTCT | |
| SB08571 | 75.9 | 2061.6 | GTGAGTGACAGGCGAACCAGTACTATAAATAGATACGGGC | 209 |
| | | | CAGACAGATGGTTCCCAATACGACTAATCCTCATAGAAGA | |
| | | | CGCACTAATCCTCTAGTAAGGTACTAAGCCACATTCGTCT | |
| | | | AGTACTAATCCTCTCGGCAGCCGATCTAATCCTCAGTCTA | |
| | | | GCGCTGACAGATGGTTCACGGGTGGATTAATTGGGGGGAC | |
| | | | GTTGTGAATAGCTGGTCGAAGTTTGGTTGAAGCACAGGAG | |
| | | | GGTAAATAAGAAGGTTTCCAGACAAAGAGACTCTTAAAGG | |
| | | | TACAGACTCTTATGTCTGTCCCTCCTCCTTAAAGGGCCAG | |
| | | | AGACGCTCCCGAGCCCATCTCCCTGCAGGAGACGCTAGCG | |
| | | | GGGGGCTATAAAAGGGGGTGGGGGCGTTCGTCCTCACTCT | |
| SB08572 | 60.1 | 2800.2 | CTAAGCCACATTCGTCTAGTACTAATCCTCTCGGCAGCCG | 210 |
| | | | ATCTAATCCTCAGTCTAGCGCTGACAGATGGTTCACGGGT | |
| | | | GGATTAATTGGTAGTAAGGTACTAAGCCACATTCGTCTAG | |
| | | | TACTAATCCTCTCGGCAGCCGATCTAATCCTCAGTCTAGC | |
| | | | GCTGACAGATGGTTCACGGGTGGATTAATTGGGGGGACGT | |
| | | | TGTGAATAGCTGGTCGAAGTTTGGTTGAAGCACAGGAGGG | |
| | | | TAAATAAGAAGGTTTCCAGACAAAGAGACTCTTAAAGGTA | |
| | | | CAGACTCTTATGTCTGTCCCTCCTCCTTAAAGGGCCAGAG | |
| | | | ACGCTCCCGAGCCCATCTCCCTGCAGGAGACGCTAGCGGG | |
| | | | GGGCTATAAAAGGGGGTGGGGGCGTTCGTCCTCACTCT | |
| SB08573 | 22.9 | 883.9 | CTAAGCCACATTCGTCTAGTACTAATCCTCTCGGCAGCCG | 211 |
| | | | ATCTAATCCTCAGTCTAGCGCTGACAGATGGTTCACGGGT | |
| | | | GGATTAATTGGCTCAGGCCCTGCATTGCGCCAACGGCGCA | |
| | | | GCGCTGGGCCGCGACCCCGGCACCGGCACCCGTTCCGAGG | |
| | | | GTTCGCGCCGCAGGCGCAAAGCCACATTGCTATAGTGCTG | |
| | | | TATAGCGATTATTGAGCCTGGCGTAAGGGGCGGCGGCGTA | |
| | | | GGGGGACGTTGTGAATAGCTGGTCGAAGTTTGGTTGAAGC | |
| | | | ACAGGAGGGTAAATAAGAAGGTTTCCAGACAAAGAGACTC | |
| | | | TTAAAGGTACAGACTCTTATGTCTGTCCCTCCTCCTTAAA | |
| | | | GGGCCAGAGACGCTCCCGAGCCCATCTCCCTGCAGGAGAC | |
| | | | GCTAGCGGGGGGCTATAAAAGGGGGTGGGGGCGTTCGTCC | |
| | | | TCACTCT | |
| S08574 | 24.4 | 154.9 | GGTTACACAACCAGGCGGGGAGGGGCCCCGGCAGCCGGAA | 212 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACACCCCACCCC | |
| | | | TTCCCCCTCCTTTCCGAAGCCCCCCTCCCTGTTTTTTCAG | |
| | | | CCCCCTCGTGAGTGACAGGCGAACCAGTACTATAAATAGA | |
| | | | TACGGGCCAGACAGATGGTTCCCAATACGACTAATCCTCA | |
| | | | TAGAAGACGCACTAATCCTCCCTGCAGGTTCGCATATTAA | |
| | | | GGTGACGCGTGTGGCCTCGAACACCGAGCGACCCTGCAGC | |
| | | | GACCCGCTTAA | |
| SB08575 | 30.3 | 235.3 | GGTTACACAACCAGGCGGGGAGGGGCCCCGGCAGCCGGAA | 213 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACACCCCACCCC | |
| | | | TTCCCCCTCCTTTCCGAAGCCCCCCTCCCTGTTTTTTCAG | |
| | | | CCCCCTCCTAAGCCACATTCGTCTAGTACTAATCCTCTCG | |
| | | | GCAGCCGATCTAATCCTCAGTCTAGCGCTGACAGATGGTT | |
| | | | CACGGGTGGATTAATTGGCCTGCAGGTTCGCATATTAAGG | |
| | | | TGACGCGTGTGGCCTCGAACACCGAGCGACCCTGCAGCGA | |
| | | | CCCGCTTAA | |
| SB08576 | 28.3 | 2123.0 | GGTTACACAACCAGGCGGGGAGGGGCCCCGGGGGGGGAGG | 214 |
| | | | GGGCCGGCCCGCGGGCCGCGCAGCCGGAAGCCGGGACCGC | |
| | | | CACCGGCCCCGCGCCGCGCGATCGGCCCGCGCCCATTGGC | |
| | | | TCTCCGGCCCGCCGCTCACCGCCCCTCCTCCGCACCGCCC | |
| | | | CTACCCGCAGGCCGCGGCGGGCTGTCGGCGCGGGGCACCC | |
| | | | TGGGACTTGTAGTCCAAGCCGCTTGCCACCTGCCGGCTGC | |
| | | | AAACGGCGGAGGGACTACGAAGCCCAGAGGTCCCTGCGGC | |
| | | | CCTGCCCGCCCACCCGGACACCCCACCCCTTCCCCCTCCT | |
| | | | TTCCGAAGCCCCCCTCCCTGTTTTTTCAGCCCCCTCGTGA | |
| | | | GTGACAGGCGAACCAGTACTATAAATAGATACGGGCCAGA | |
| | | | CAGATGGTTCCCAATACGACTAATCCTCATAGAAGACGCA | |
| | | | CTAATCCTCCCTGCAGGTCTAGAGGGTATATAATGGGGGC | |
| | | | CA | |
| SB08577 | 42.0 | 802.0 | GGTTACACAACCAGGCGGGGAGGGGCCCCGGGGGGGGGAG | 215 |
| | | | GGGGCCGGCCCGCGGGCCGCGCAGCCGGAAGCCGGGACCG | |
| | | | CCACCGGCCCCGCGCCGCGCGATCGGCCCGCGCCCATTGG | |
| | | | CTCTCCGGCCCGCCGCTCACCGCCCCTCCTCCGCACCGCC | |
| | | | CCTACCCGCAGGCCGCGGCGGGCTGTCGGCGCGGGGCACC | |
| | | | CTGGGACTTGTAGTCCAAGCCGCTTGCCACCTGCCGGCTG | |
| | | | CAAACGGCGGAGGGACTACGAAGCCCAGAGGTCCCTGCGG | |
| | | | CCCTGCCCGCCCACCCGGACACCCCACCCCTTCCCCCTCC | |
| | | | TTTCCGAAGCCCCCCTCCCTGTTTTTTCAGCCCCCTCCTA | |
| | | | AGCCACATTCGTCTAGTACTAATCCTCTCGGCAGCCGATC | |
| | | | TAATCCTCAGTCTAGCGCTGACAGATGGTTCACGGGTGGA | |
| | | | TTAATTGGCCTGCAGGTCTAGAGGGTATATAATGGGGGCC | |
| | | | A | |
| SB08579 | 41.4 | 690.5 | GGTTACACAACCAGGCGGGGAGGGGCCCCGGCAGCCGGAA | 216 |
| | | | GCCGGGACCGCCACCGGCCCCTAAGCCACATTCGTCTAGT | |
| | | | ACTAATCCTCTCGGCAGCCGATCTAATCCTCAGTCTAGCG | |
| | | | CTGACAGATGGTTCACGGGTGGATTAATTGGCGCGCCGCG | |
| | | | CGATCGGCCCGCGCCCATTGGCTCTCCGGCCCGCCGCTCA | |
| | | | CCGCCCCTCCTCCGCACCGCCCCTACCCGCAGGCCGCGGC | |
| | | | GGGCTGTCGGCGCGGGGCACCCTGGGACTTGTAGTCCAAG | |
| | | | CCGCTTGCCACCTGCCGGCTGCAAACGGCGGAGGGACTAC | |
| | | | GAAGCCCAGAGGTCCCTGCGGCCCTGCCCGCCCACCCGGA | |
| | | | CACCCCACCCCTTCCCCCTCCTTTCCGAAGCCCCCCTCCC | |
| | | | TGTTTTTTCAGCCCCCTCCCTGCAGGTTCGCATATTAAGG | |
| | | | TGACGCGTGTGGCCTCGAACACCGAGCGACCCTGCAGCGA | |
| | | | CCCGCTTAA | |
| SB08580 | 28.5 | 530.9 | GGTTACACAACCAGGCGGGGAGGGGCCCCGGGGGGGGGAG | 217 |
| | | | GGGGCCGGCCCGCGGGCCGCGCAGCCGGAAGCCGGGACCG | |
| | | | CCACCGGCCCGTGAGTGACAGGCGAACCAGTACTATAAAT | |
| | | | AGATACGGGCCAGACAGATGGTTCCCAATACGACTAATCC | |
| | | | TCATAGAAGACGCACTAATCCTCCGCGCCGCGCGATCGGC | |
| | | | CCGCGCCCATTGGCTCTCCGGCCCGCCGCTCACCGCCCCT | |
| | | | CCTCCGCACCGCCCCTACCCGCAGGCCGCGGCGGGCTGTC | |
| | | | GGCGCGGGGCACCCTGGGACTTGTAGTCCAAGCCGCTTGC | |
| | | | CACCTGCCGGCTGCAAACGGCGGAGGGACTACGAAGCCCA | |
| | | | GAGGTCCCTGCGGCCCTGCCCGCCCACCCGGACACCCCAC | |
| | | | CCCTTCCCCCTCCTTTCCGAAGCCCCCCTCCCTGTTTTTT | |
| | | | CAGCCCCCTCCCTGCAGGTCTAGAGGGTATATAATGGGGG | |
| | | | CCA | |
| SB08581 | 26.6 | 407.8 | GGTTACACAACCAGGCGGGGAGGGGCCCCGGGGGGGGGAG | 218 |
| | | | GGGGCCGGCCCGCGGGCCGCGCAGCCGGAAGCCGGGACCG | |
| | | | CCACCGGCCCCTAAGCCACATTCGTCTAGTACTAATCCTC | |
| | | | TCGGCAGCCGATCTAATCCTCAGTCTAGCGCTGACAGATG | |
| | | | GTTCACGGGTGGATTAATTGGCGCGCCGCGCGATCGGCCC | |
| | | | GCGCCCATTGGCTCTCCGGCCCGCCGCTCACCGCCCCTCC | |
| | | | TCCGCACCGCCCCTACCCGCAGGCCGCGGCGGGCTGTCGG | |
| | | | CGCGGGGCACCCTGGGACTTGTAGTCCAAGCCGCTTGCCA | |
| | | | CCTGCCGGCTGCAAACGGCGGAGGGACTACGAAGCCCAGA | |
| | | | GGTCCCTGCGGCCCTGCCCGCCCACCCGGACACCCCACCC | |
| | | | CTTCCCCCTCCTTTCCGAAGCCCCCCTCCCTGTTTTTTCA | |
| | | | GCCCCCTCCCTGCAGGTCTAGAGGGTATATAATGGGGGCC | |
| | | | A | |
| SB08582 | 56.7 | 798.6 | CTCCTGCTATTGGTCAGAATGTGTCACGTGACCATACTCA | 219 |
| | | | GCTGCAAGGGTTGGTGGGAACTAAGCCACATTCGTCTAGT | |
| | | | ACTAATCCTCTCGGCAGCCGATCTAATCCTCAGTCTAGCG | |
| | | | CTGACAGATGGTTCACGGGTGGATTAATTGGCTCCTGCTA | |
| | | | TTGGTCAGAATGTGTCACGTGACCATACTCAGCTGCAAGG | |
| | | | GTTGGTGGGAACTAAGCCACATTCGTCTAGTACTAATCCT | |
| | | | CTCGGCAGCCGATCTAATCCTCAGTCTAGCGCTGACAGAT | |
| | | | GGTTCACGGGTGGATTAATTGGCTCCTGCTATTGGTCAGA | |
| | | | ATGTGTCACGTGACCATACTCAGCTGCAAGGGTTGGTGGG | |
| | | | AACTAAGCCACATTCGTCTAGTACTAATCCTCTCGGCAGC | |
| | | | CGATCTAATCCTCAGTCTAGCGCTGACAGATGGTTCACGG | |
| | | | GTGGATTAATTGGCCTGCAGGTCTAGAGGGTATATAATGG | |
| | | | GGGCCA | |
| SB08583 | 58.6 | 1274.0 | CTAATCCTCTCGGCCGATCTAATCCTCAGTCTGCTGACAG | 220 |
| | | | ATGGTCTCCTGCTATTGGTCAGAATGTGTCACGTGACCAT | |
| | | | ACTCAGCTGCAAGGGTTGGTGGGAAATGTAGTCTTTTTTT | |
| | | | CTGGGAGGCCATGTGTTCGGCTTAAAATTGTCTTTTTTTC | |
| | | | TTTTTCTAAGCCACATTCGTCTAGTACTAATCCTCTCGGC | |
| | | | AGCCGATCTAATCCTCAGTCTAGCGCTGACAGATGGTTCA | |
| | | | CGGGTGGATTAATTGGCTCCTGCTATTGGTCAGAATGTGT | |
| | | | CACGTGACCATACTCAGCTGCAAGGGTTGGTGGGAAATGT | |
| | | | AGTCTTTTTTTCTGGGAGGCCATGTGTTCGGCTTAAAATT | |
| | | | GTCTTTTTTTCTTTTTCTAAGCCACATTCGTCTAGTACTA | |
| | | | ATCCTCTCGGCAGCCGATCTAATCCTCAGTCTAGCGCTGA | |
| | | | CAGATGGTTCACGGGTGGATTAATTGGCCTGCAGGTCTAG | |
| | | | AGGGTATATAATGGGGGCCA | |
| SB08584 | 70.2 | 2985.6 | CTAAGCCACAGTAACATGCGACTAATCCTCTCTGTCTACA | 221 |
| | | | GAGTGGCTTAGTCTATCGTGTTGCTGACTCAGCATTAGAT | |
| | | | AGTGACAGATGGTTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTCTTTTTCTAAGCC | |
| | | | ACATTCGTCTAGTACTAATCCTCTCGGCAGCCGATCTAAT | |
| | | | CCTCAGTCTAGCGCTGACAGATGGTTCACGGGTGGATTAA | |
| | | | TTGGCCTGCAGGTCTAGAGGGTATATAATGGGGGCCA | |
| SB08585 | 78.4 | 1719.8 | GTGAGTGACAGGCGAACCAGTACTATAAATAGATACGGGC | 222 |
| | | | CAGACAGATGGTTCCCAATACGACTAATCCTCATAGAAGA | |
| | | | CGCACTAATCCTCTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTCTTTTTCTAAGCC | |
| | | | ACATTCGTCTAGTACTAATCCTCTCGGCAGCCGATCTAAT | |
| | | | CCTCAGTCTAGCGCTGACAGATGGTTCACGGGTGGATTAA | |
| | | | TTGGCCTGCAGGTCTAGAGGGTATATAATGGGGGCCA | |
| SB08586 | 44.4 | 3311.2 | CTAAGCCACAGTAACATGCGACTAATCCTCTCTGTCTACA | 223 |
| | | | GAGTGGCTTAGTCTATCGTGTTGCTGACTCAGCATTAGAT | |
| | | | AGTGACAGATGGTTGTTGAGAGCTCAAGCTCTTTTTAACG | |
| | | | CTTTGCCTTGCTGTCTCCAAAGTATTGCCTTCATCCTCAT | |
| | | | AGTTCAAAGTGTCCACCATCACATACACGGTTAAGACTCA | |
| | | | ATCCGGTAAGTAAGAGAAAAACAGGAAAAGAGTTTACATG | |
| | | | CCACTCCTGCTATTGGTCAGAATGTGTCACGTGACCATAC | |
| | | | TCAGCTGCAAGGGTTGGTGGGAAATGTAGTCTTTTTTTCT | |
| | | | GGGAGGCCATGTGTTCGGCTTAAAATTGTCTTTTTTTCTT | |
| | | | TTTCTAAGCCACATTCGTCTAGTACTAATCCTCTCGGCAG | |
| | | | CCGATCTAATCCTCAGTCTAGCGCTGACAGATGGTTCACG | |
| | | | GGTGGATTAATTGGCCTGCAGGTCTAGAGGGTATATAATG | |
| | | | GGGGCCA | |
| SB08590 | 7.2 | 461.1 | CTAAGCCACATTCGTCTAGTACTAATCCTCTCGGCAGCCG | 224 |
| | | | ATCTAATCCTCAGTCTAGCGCTGACAGATGGTTCACGGGT | |
| | | | GGATTAATTGGTTACCTACTAGGTTAATTCGTCCGATAGA | |
| | | | TACTACGAATTGCGAGCTTCTAAGTCCAATTTTCGGTATT | |
| | | | CGAGTCGTCAGACTCAATTATTACACGGTTAAGACTCAAT | |
| | | | CCGGTAAGTAATACTGTGATCAGGTGTCTACAAGACTAGT | |
| | | | ACTCCTGCTATTGGTCAGAATGTGTCACGTGACCATACTC | |
| | | | AGCTGCAAGGGTTGGTGGGAAATGTAGTCTTTTTTTCTGG | |
| | | | GAGGCCATGTGTTCGGCTTAAAATTGTCTTTTTTTCTTTT | |
| | | | TACTATCACGTATATACCAGCGAGTTCGATAATACACTCT | |
| | | | CCTGCAGGTCTAGAGGGTATATAATGGGGGCCA | |
| SB08591 | 11.9 | 294.0 | CTAAGCCACATTCGTCTAGTACTAATCCTCTCGGCAGCCG | 225 |
| | | | ATCTAATCCTCAGTCTAGCGCTGACAGATGGTTCACGGGT | |
| | | | GGATTAATTGGTGTTGAGAGCTCAAGCTCTTTTTAACGCT | |
| | | | TTGCCTTGCTGTCTCCAAAGTATTGCCTTCATCCTCATAG | |
| | | | TTCAAAGTGTCCACCATCACATACACGGTTAAGACTCAAT | |
| | | | CCGGTAAGTAAGAGAAAAACAGGAAAAGAGTTTACATGCC | |
| | | | ACTCCTGCTATTGGTCAGAATGTGTCACGTGACCATACTC | |
| | | | AGCTGCAAGGGTTGGTGGGAAATGTAGTCTTTTTTTCTGG | |
| | | | GAGGCCATGTGTTCGGCTTAAAATTGTCTTTTTTTCTTTT | |
| | | | TCTTTATTTCTTTTTTCTTTTCTTTCTTTTTATACACTCT | |
| | | | CCTGCAGGTCTAGAGGGTATATAATGGGGGCCA | |
| SB08592 | 11.6 | 151.2 | CTAAGCCACATTCGTCTAGTACTAATCCTCTCGGCAGCCG | 226 |
| | | | ATCTAATCCTCAGTCTAGCGCTGACAGATGGTTCACGGGT | |
| | | | GGATTAATTGGTGTTGAGAGCTCAAGCTCTTTTTAACGCT | |
| | | | TTGCCTTGCTGTCTCCAAAGTATTGCCTTCATCCTCATAG | |
| | | | TTCAAAGTGTCCACCATCACATTCACGTTTCAGCCAATAG | |
| | | | GAAGAAGGAAAGAGAAAAACAGGAAAAGAGTTTACATGCC | |
| | | | ACTCCTGCTATTGGTCAGAATGTGTCACGTGACCATACTC | |
| | | | AGCTGCAAGGGTTGGTGGGAAATGTAGTCTTTTTTTCTGG | |
| | | | GAGGCCATGTGTTCGGCTTAAAATTGTCTTTTTTTCTTTT | |
| | | | TCTTTATTTCTTTTTTCTTTTCTTTCTTTTTTTTAGACGG | |
| | | | CCTGCAGGTCTAGAGGGTATATAATGGGGGCCA | |
| SB08593 | 135.7 | 4305.4 | TAGTACTAATCCTCTCGGCAGCCGATCTAATCCTCAGTCT | 227 |
| | | | AGCGCTGACAGATGGTTCACTAGTAAGGTACTAAGCCACA | |
| | | | TTCGTCTAGTACTAATCCTCTCGGCAGCCGATCTAATCCT | |
| | | | CAGTCTAGCGCTGACAGATGGTTCACGGGTGGATTAATTG | |
| | | | GTTTGCGCGTACTAAGCCACATTCGTCTAGTACTAATCCT | |
| | | | CTCGGCAGCCGATCTAATCCTCAGTCTAGCGCTGACAGAT | |
| | | | GGTTCACGGGTGGATTAATTGGAGTCCGGGTACTAAGCCA | |
| | | | CATTCGTCTAGTACTAATCCTCTCGGCAGCCGATCTAATC | |
| | | | CTCAGTCTAGCGCTGACAGATGGTTCACGGGTGGATTAAT | |
| | | | TGGCCTGCAGGTCTAGAGGGTATATAATGGGGGCCA | |
| SB08594 | 73.6 | 2917.4 | CTAAGCCACATTCGTCTAGTACTAATCCTCTCGGCAGCCG | 228 |
| | | | ATCTAATCCTCAGTCTAGCGCTGACAGATGGTTCACGGGT | |
| | | | GGATTAATTGGGTGAGTGACAGGCGACCAGTACTATAAAT | |
| | | | AGATACGGGCCAGACAGATGGTTCCCAATACGACTAATCC | |
| | | | TCATAGAAGACGCACTAATCCTCCTAAGCCACATTCGTCT | |
| | | | AGTACTAATCCTCTCGGCAGCCGATCTAATCCTCAGTCTA | |
| | | | GCGCTGACAGATGGTTCACGGGTGGATTAATTGGGTGAGT | |
| | | | GACAGGCGAACCAGTACTATAAATAGATACGGGCCAGACA | |
| | | | GATGGTTCCCAATACGACTAATCCTCATAGAAGACGCACT | |
| | | | AATCCTCCCTGCAGGTCTAGAGGGTATATAATGGGGGCCA | |
| SB08595 | 88.3 | 2635.0 | GTGAGTGACAGGCGACCAGTACTATAAATAGATACGGGCC | 229 |
| | | | AGACAGATGGTTCCCAATACGACTAATCCTCATAGAAGAC | |
| | | | GCACTAATCCTCGTGAGTGACAGGCGAACCAGTACTATAA | |
| | | | ATAGATACGGGCCAGACAGATGGTTCCCAATACGACTAAT | |
| | | | CCTCATAGAAGACGCACTAATCCTCCTAAGCCACATTCGT | |
| | | | CTAGTACTAATCCTCTCGGCAGCCGATCTAATCCTCAGTC | |
| | | | TAGCGCTGACAGATGGTTCACGGGTGGATTAATTGGCTAA | |
| | | | GCCACATTCGTCTAGTACTAATCCTCTCGGCAGCCGATCT | |
| | | | AATCCTCAGTCTAGCGCTGACAGATGGTTCACGGGTGGAT | |
| | | | TAATTGGCCTGCAGGTCTAGAGGGTATATAATGGGGGCCA | |
| SB08725 | 54.7 | 2267.6 | CTAAGCCACATTCGTCTAGTACTAATCCTCTCGGCAGCCG | 230 |
| | | | ATCTAATCCTCAGTCTAGCGCTGACAGATGGTTCACGGGT | |
| | | | GGATTAATTGGTGCTGAGTCAGCATAGGAAGAGTCTAAGC | |
| | | | CACAGCACGCGTAGTCTAATCCTCAGCCTACTTAACTATA | |
| | | | AATAGATCGAGCAATACCATCTGTCTGCTGACTCAGCAAC | |
| | | | GACTATAAGTGGCTTAGAACATAACGTCTCTAATCCTCAG | |
| | | | ACTGCGCGTGACAGATGGTTTCGTCCCGTACCATCTGTCT | |
| | | | GCTGAGTCAGCAAGTATACGAACTAAGCCACAGGAGTCTT | |
| | | | GTACCATCTGTCAATCGGCTTAACCATCTGTCAAGTATCT | |
| | | | ATACCATCTGTCTCTAGAGGGTATATAATGGGGGCCA | |
| SB08726 | 30.6 | 3933.2 | ACCATCTGTCAATCGGCTTAACCATCTGTCAAGTATCTAT | 231 |
| | | | ACCATCTGTCAGTCCGGGTATGCTGAGTCAGCATAGGAAG | |
| | | | AGTCTAAGCCACAGCACGCGTAGTCTAATCCTCAGCCTAC | |
| | | | TTAACTATAAATAGATCGAGCAATACCATCTGTCTTTGCG | |
| | | | CGTATGCTGACTCAGCAACGACTATAAGTGGCTTAGAACA | |
| | | | TAACGTCTCTAATCCTCAGACTGCGCGTGACAGATGGTTT | |
| | | | CGTCCCGTACCATCTGTCTAGTAAGGTATGCTGAGTCAGC | |
| | | | AAGTATACGAACTAAGCCACAGGAGTCTTGTACCATCTGT | |
| | | | CAATCGGCTTAACCATCTGTCAAGTATCTATACCATCTGT | |
| | | | CCCTGCAGGTCTAGAGGGTATATAATGGGGGCCA | |
| SB08727 | 19.0 | — | ACCATCTGTCAATCGGCTTAACCATCTGTCAAGTATCTAT | 232 |
| | | | ACCATCTGTCAGTCCGGGTATGCTGAGTCAGCAAGTATAC | |
| | | | GAACTAAGCCACAGGAGTCTTGTACCATCTGTCAATCGGC | |
| | | | TTAACCATCTGTCAAGTATCTATACCATCTGTCTTTGCGC | |
| | | | GTATGCTGAGTCAGCAAGTATACGAACTAAGCCACAGGAG | |
| | | | TCTTGTACCATCTGTCAATCGGCTTAACCATCTGTCAAGT | |
| | | | ATCTATACCATCTGTCTAGTAAGGTATGCTGAGTCAGCAA | |
| | | | GTATACGAACTAAGCCACAGGAGTCTTGTACCATCTGTCA | |
| | | | ATCGGCTTAACCATCTGTCAAGTATCTATACCATCTGTCC | |
| | | | CTGCAGGTCTAGAGGGTATATAATGGGGGCCA | |
| SB08728 | 14.8 | — | TGCTGAGTCAGCAAGTATACGAACTAAGCCACAGGAGTCT | 233 |
| | | | TGTACCATCTGTCAATCGGCTTAACCATCTGTCAAGTATC | |
| | | | TATACCATCTGTCTGCTGAGTCAGCAAGTATACGAACTAA | |
| | | | GCCACAGGAGTCTTGTACCATCTGTCAATCGGCTTAACCA | |
| | | | TCTGTCAAGTATCTATACCATCTGTCTGCTGAGTCAGCAA | |
| | | | GTATACGAACTAAGCCACAGGAGTCTTGTACCATCTGTCA | |
| | | | ATCGGCTTAACCATCTGTCAAGTATCTATACCATCTGTCT | |
| | | | GCTGAGTCAGCAAGTATACGAACTAAGCCACAGGAGTCTT | |
| | | | GTACCATCTGTCAATCGGCTTAACCATCTGTCAAGTATCT | |
| | | | ATACCATCTGTCTCTAGAGGGTATATAATGGGGGCCA | |
| SB08729 | 40.8 | — | TGCTGAGTCAGCAAGTATACGAACTAAGCCACAGGAGTCT | 234 |
| | | | TGTACCATCTGTCAATCGGCTTAACCATCTGTCAAGTATC | |
| | | | TATACCATCTGTCCTCAGGCCCTGCATTGCGCCAACGGCG | |
| | | | CAGCGCTGGGCCGCGACCCCGGCACCGGCACCCGGGGACG | |
| | | | TTGTGAATAGCTGGTCGAAGTTTGGTTGAAGCACAGGAGG | |
| | | | GTAAATAAGAAGGTTTCCAGACAAAGAGACTCTTAAAGGT | |
| | | | ACAGACTCTTATGTCTGTCCCTCCTCCTTAAAGGGCCAGA | |
| | | | GACGCTCCCGAGCCCATCTCCTCCTGCTATTGGTCAGAAT | |
| | | | GTGTCACGTGACCATACTCAGCTGCAAGGGTTGGTGGGAA | |
| | | | ATGTAGTCTTTTTTTCTGGGAGGCCATGTGTTCGGCTTAA | |
| | | | AATTGTCTTTTTTTCTTTTTCCTGCAGGAGACGCTAGCGG | |
| | | | GGGGCTATAAAAGGGGGTGGGGGCGTTCGTCCTCACTCT | |
| SB08730 | 53.6 | — | GGTTACACAACCAGGCGGGGAGGGGCCCCGGCAGCCGGAA | 235 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACCCCCCTCCCT | |
| | | | GTTTTTTCAGCCCCCTCTGCTGAGTCAGCAAGTATACGAA | |
| | | | CTAAGCCACAGGAGTCTTGTACCATCTGTCAATCGGCTTA | |
| | | | ACCATCTGTCAAGTATCTATACCATCTGTCTCGAGGCGCC | |
| | | | GGCGGGGCGGCCCGGCGGCCGGAAGTGCACCCTGCTCGGG | |
| | | | CTTTGCGCCCCCGGCAGCTTCCTCCGCCCGCGAGGTCTAG | |
| | | | AGGGTATATAATGGGGGCCA | |
| SB08731 | 29.7 | 1350.1 | TGCTGAGTCAGCAAGTATACGAACTAAGCCACAGGAGTCT | 236 |
| | | | TGTACCATCTGTCAATCGGCTTAACCATCTGTCAAGTATC | |
| | | | TATACCATCTGTCGGTTACACAACCAGGCGGGGAGGGGCC | |
| | | | CCGGCAGCCGGAAGCCGGGACCGCCACCGGCCCCGCGCCG | |
| | | | CGCGATCGGCCCGCGCCCATTGGCTCTCCGGCCCGCCGCT | |
| | | | CACCGCCCCTCCTCCGCACCGCCCCTACCCGCAGGCCGCG | |
| | | | GCGGGCTGTCGGCGCGGGGCACCCTGGGACTTGTAGTCCA | |
| | | | AGCCGCTTGCCACCTGCCGGCTGCAAACGGCGGAGGGACT | |
| | | | ACGAAGCCCAGAGGTCCCTGCGGCCCTGCCCGCCCACCCG | |
| | | | GACCCCCCTCCCTGTTTTTTCAGCCCCCTCTCGAGGCGCC | |
| | | | GGCGGGGCGGCCCGGCGGCCGGAAGTGCACCCTGCTCGGG | |
| | | | CTTTGCGCCCCCGGCAGCTTCCTCCGCCCGCGAGGTCTAG | |
| | | | AGGGTATATAATGGGGGCCA | |
| SB08732 | 38.2 | 75.4 | GGTTACACAACCAGGCGGGGAGGGGCCCCGGCAGCCGGAA | 237 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACCCCCCTCCCT | |
| | | | GTTTTTTCAGCCCCCTCTCGAGGCGCCGGCGGGGCGGCCC | |
| | | | GGCGGCCGGAAGTGCACCCTGCTCGGGCTTTGCGCCCCCG | |
| | | | GCAGCTTCCTCCGCCCGCGAGGCCTGCAGGTTCGCATATT | |
| | | | AAGGTGACGCGTGTGGCCTCGAACACCGAGCGACCCTGCA | |
| | | | GCGACCCGCTTAA | |
| SB08733 | 8.1 | 95.3 | GGTTACACAACCAGGCGGGGAGGGGCCCCGGCAGCCGGAA | 238 |
| | | | GCCGGGACCGCCACCGGCCCCGCGCCGCGCGATCGGCCCG | |
| | | | CGCCCATTGGCTCTCCGGCCCGCCGCTCACCGCCCCTCCT | |
| | | | CCGCACCGCCCCTACCCGCAGGCCGCGGCGGGCTGTCGGC | |
| | | | GCGGGGCACCCTGGGACTTGTAGTCCAAGCCGCTTGCCAC | |
| | | | CTGCCGGCTGCAAACGGCGGAGGGACTACGAAGCCCAGAG | |
| | | | GTCCCTGCGGCCCTGCCCGCCCACCCGGACCCCCCTCCCT | |
| | | | GTTTTTTCAGCCCCCTCTCGAGGCGCCGGCGGGGCGGCCC | |
| | | | GGCGGCCGGAAGTGCACCCTGCTCGGGCTTTGCGCCCCCG | |
| | | | GCAGCTTCCTCCGCCCGCGAGGCCTGCAGGTCTAGAGGGT | |
| | | | ATATAATGGGGGCCA | |
| SB08850 | 11.7 | 451.5 | TGCTGAGTCAGCAAGTATACGAACTAAGCCACAGGAGTCT | 239 |
| | | | TGTACCATCTGTCAATCGGCTTAACCATCTGTCAAGTATC | |
| | | | TATACCATCTGTCCTCCTGCTATTGGTCAGAATGTGTCAC | |
| | | | GTGACCATACTCAGCTGCAAGGGTTGGTGGGAAATGTAGT | |
| | | | CTTTTTTTCTGGGAGGCCATGTGTTCGGCTTAAAATTGTC | |
| | | | TTTTTTTCTTTTTCTAAGCCACAGTAACATGCGACTAATC | |
| | | | CTCTCTGTCTACAGAGTGGCTTAGTCTATCGTGTTGCTGA | |
| | | | CTCAGCATTAGATAGTGACAGATGGTCCTGCAGGTCTAGA | |
| | | | GGGTATATAATGGGGGCCA | |
| SB08851 | 54.3 | 2941.8 | TGCTGAGTCAGCAAGTATACGAACTAAGCCACAGGAGTCT | 240 |
| | | | TGTACCATCTGTCAATCGGCTTAACCATCTGTCAAGTATC | |
| | | | TATACCATCTGTCTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTCTTTTTCTAAGCC | |
| | | | ACAGTAACATGCGACTAATCCTCTCTGTCTACAGAGTGGC | |
| | | | TTAGTCTATCGTGTTGCTGACTCAGCATTAGATAGTGACA | |
| | | | GATGGTCCTGCAGGAGACGCTAGCGGGGGGCTATAAAAGG | |
| | | | GGGTGGGGGCGTTCGTCCTCACTCT | |
| SB08854 | 6.9 | 2070.9 | ACCATCTGTCTAGTAAGGTATGCTGAGTCAGCAAGTATAC | 241 |
| | | | GAACTAAGCCACAGGAGTCTTGTACCATCTGTCAATCGGC | |
| | | | TTAACCATCTGTCAAGTATCTATACCATCTGTCCCTGCAG | |
| | | | GTCTAGAGGGTATATAATGGGGGCCA | |
| SB08855 | 6.3 | 1336.0 | ACCATCTGTCAAGTATCTATACCATCTGTCTAGTAAGGTA | 242 |
| | | | TGCTGAGTCAGCAAGTATACGAACTAAGCCACAGGAGTCT | |
| | | | TGTACCATCTGTCAATCGGCTTAACCATCTGTCAAGTATC | |
| | | | TATACCATCTGTCCCTGCAGGTCTAGAGGGTATATAATGG | |
| | | | GGGCCA | |
| SB08856 | 20.5 | 4554.1 | ACCATCTGTCAATCGGCTTAACCATCTGTCAAGTATCTAT | 243 |
| | | | ACCATCTGTCTAGTAAGGTATGCTGAGTCAGCAAGTATAC | |
| | | | GAACTAAGCCACAGGAGTCTTGTACCATCTGTCAATCGGC | |
| | | | TTAACCATCTGTCAAGTATCTATACCATCTGTCCCTGCAG | |
| | | | GTCTAGAGGGTATATAATGGGGGCCA | |
| SB08857 | 20.7 | 879.4 | TGCTGAGTCAGCAAGTATACGAACTAAGCCACAGGAGTCT | 244 |
| | | | TGTACCATCTGTCAATCGGCTTAACCATCTGTCAAGTATC | |
| | | | TATACCATCTGTCCCTGCAGGTAGGCGTGTACGGTGGGAG | |
| | | | GCCTATATAAGCAGAGCTCGTTTAGTGAACCGTCAGATCG | |
| | | | CCTGGA | |
| SB08858 | 21.9 | 2516.3 | TGCTGAGTCAGCAAGTATACGAACTAAGCCACAGGAGTCT | 245 |
| | | | TGTACCATCTGTCAATCGGCTTAACCATCTGTCAAGTATC | |
| | | | TATACCATCTGTCCCTGCAGGAGACGCTAGCGGGGGGCTA | |
| | | | TAAAAGGGGGTGGGGGCGTTCGTCCTCACTCT | |
| SB08859 | 15.7 | 2242.4 | TGCTGAGTCAGCAAGTATACGAACTAAGCCACAGGAGTCT | 246 |
| | | | TGTACCATCTGTCAATCGGCTTAACCATCTGTCAAGTATC | |
| | | | TATACCATCTGTCCCTGCAGGAAAATGTGCGCATGTGCAG | |
| | | | CCATTGCCTGGGACGCATGCGTAGGGAGCCGCGCGACAAA | |
| | | | CTGAGCCATTGCGGCAAGACTAGCGCAGAGAGGAGAGGGA | |
| | | | GCCGGAGATGCCAGACGCTTGGTTCTGAGGAGTGATTTGC | |
| | | | AACGCAATGGAGCGAGGAAGG | |
| SB08860 | 15.0 | 2006.3 | TGCTGAGTCAGCAAGTATACGAACTAAGCCACAGGAGTCT | 247 |
| | | | TGTACCATCTGTCAATCG | |
| | | | GCTTAACCATCTGTCAAGTATCTATACCATCTGTCCCTGC | |
| | | | AGGCAGAATTAACAGTATAAATTGCATCTCTTGTTCAAGA | |
| | | | GTTCCCTATCACTCT | |
| SB08331 | 34.5 | 207.5 | GTGGCTTAGAAAGGTCCGATGCTGAGTCAGCAAGCAAGTA | 276 |
| | | | CTCTAATCCTCTATAGGACAAACTATAAATAGAATACGAG | |
| | | | GAGTTAATCCCCTGTTAATCCCCTAGCTTAGATTCTATTT | |
| | | | ATAGTTATTACTATGCTGACTCAGCAATAGACGGTAGAGG | |
| | | | ATTAGAGCACTCGTTTGACAGATGGTCTCCTGCTATTGGT | |
| | | | CAGAATGTGTCACGTGACCATACTCAGCTGCAAGGGTTGG | |
| | | | TGGGAACTAAGCCACAGTAACATGCGACTAATCCTCTCTG | |
| | | | TCTACAGAGTGGCTTAGTCTATCGTGTTGCTGACTCAGCA | |
| | | | TTAGATAGTGACAGATGGTCCTGCAGGTCTAGAGGGTATA | |
| | | | TAATGGGGGCCA | |
| SB08332 | 26.8 | 535.8 | GTGGCTTAGAAAGGTCCGATGCTGAGTCAGCAAGCAAGTA | 277 |
| | | | CTCTAATCCTCTATAGGACAAACTATAAATAGAATACGAG | |
| | | | GAGTTAATCCCCTTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTCTTTTTCTAAGCC | |
| | | | ACAGTAACATGCGACTAATCCTCTCTGTCTACAGAGTGGC | |
| | | | TTAGTCTATCGTGTTGCTGACTCAGCATTAGATAGTGACA | |
| | | | GATGGTCCTGCAGGTCTAGAGGGTATATAATGGGGGCCA | |
| SB08333 | 37.9 | 1131.3 | GTTAATCCCCTAGCTTAGATTCTATTTATAGTTATTACTA | 278 |
| | | | TGCTGACTCAGCAATAGACGGTAGAGGATTAGAGCACTCG | |
| | | | TTTGACAGATGGTTACACGGTTAAGACTCAATCCGGTAAG | |
| | | | TAAGAGAAAAACAGGAAAAGAGTTTACATGCCACTCCTGC | |
| | | | TATTGGTCAGAATGTGTCACGTGACCATACTCAGCTGCAA | |
| | | | GGGTTGGTGGGAAATGTAGTCTTTTTTTCTGGGAGGCCAT | |
| | | | GTGTTCGGCTTAAAATTGTCTTTTTTTTTTTTCTAAGCCA | |
| | | | CAGTAACATGCGACTAATCCTCTCTGTCTACAGAGTGGCT | |
| | | | TAGTCTATCGTGTTGCTGACTCAGCATTAGATAGTGACAG | |
| | | | ATGGTCCTGCAGGTCTAGAGGGTATATAATGGGGGCCA | |

“—” indicates to be tested

TABLE 4
Engineered Regulatory Element Components

| SEQ ID | |
|---|---|
| NO: | Component sequence |
| 31 | AGCGCTGACAGATGGTTCACGG |
| 32 | GGGCCAGACAGATGGTTCCCAATAC |
| 33 | TGTTGAGAGCTCAAGCTCTTTTTAACGCTT |
| 34 | CTTTATTTCTTTTTTCTTTTCTTTCTTTTT |
| 35 | ACTATCACGTATATACCAGCGAGTTCGATA |
| 36 | TTTAGACGG |
| 37 | ATACACTCT |
| 38 | TTACCTACTAGGTTAATTCGTCCGATAGAT |
| 39 | TGCCTTGCTGTCTCCAAAGTATTGCCTTCA |
| 40 | ACTACGAATTGCGAGCTTCTAAGTCCAATT |
| 41 | TCCTCATAGTTCAAAGTGTCCACCATCACA |
| 42 | TTCGGTATTCGAGTCGTCAGACTCAATTAT |
| 43 | TTCACGTTTCAGCCAATAGGAAGAAGGAAA |
| 44 | TACACGGTTAAGACTCAATCCGGTAAGTAA |
| 45 | GAGAAAAACAGGAAAAGAGTTTACATGCCA |
| 46 | TACTGTGATCAGGTGTCTACAAGACTAGTA |
| 47 | CTCCTGCTATTGGTCAGAATGTGTCACGTG |
| 48 | ACCATACTCAGCTGCAAGGGTTGGTGGGAA |
| 49 | ATGTAGTCTTTTTTTCTGGGAGGCCATGTG |
| 50 | TTCGGCTTAAAATTGTCTTTTTTTCTTTTT |
| 51 | AGTAGCATGGTTGGAGCTTAACCCCAAGTC |
| 52 | TAATTTTATTCTGCCTCTTTATTTAAAAAG |
| 53 | GCAAGGAAGATTATTTTGAGTCACTGCACC |
| 54 | CTTTCTGAACAATAGCTGTTACCAAGGTCT |
| 55 | CAGGAGCATTAGGCGCTCAATTCTATTTTA |
| 56 | ACGCTATCACAGAGAGCCTCTTAGAAGCAC |
| 57 | GCTCGCATCCCCAGCACGTTAAATTACCCA |
| 58 | AGAAATGTCTCTGGCCAAACCTTTCAAGTA |
| 59 | ACGTGCAGACAAGAGAAAATCAGAG |
| 60 | GAAACAAAAATTTAAAAATAGCTCTACAAG |
| 61 | GGTAACCCGATTAGTCAGGCACCTCTTTCT |
| 62 | CCTCAAATGACCTTGAGGTTGTAAATACGA |
| 63 | TCTGTGGATGCCCCCTGGTGGCCCTAAATA |
| 64 | GAATGGCTTCAATGGATGGAAATTTAACGT |
| 65 | GGTTGAAGCACAGGAGGGTAAATAAGAAGG |
| 66 | TTTCCAGACAAAGAGACTCTTAAAGGTACA |
| 67 | GACTCTTATGTCTGTCCCTCCTCCTTAAAG |
| 68 | GGCCAGAGACGCTCCCGAGCCCATCTC |
| 69 | CTCAGGCCCTGCATTGCGCCAACGGCGCAG |
| 70 | CGCTGGGCCGCGACCCCGGCACCGGCACCC |
| 71 | GTTCCGAGGGTTCGCGCCGCAGGCGCAAAG |
| 72 | CCACATTGCTATAGTGCTGTATAGCGATTA |
| 73 | TTGAGCCTGGCGTAAGGGGCGGCGGCGTAG |
| 74 | GGGGACGTTGTGAATAGCTGGTCGAAGTTT |
| 75 | GGTTACACAACCAGGCGGGGAGGGGCCCCG |
| 76 | TAGTCCAAGCCGCTTGCCACCTGCCGGCTG |
| 77 | CAAACGGCGGAGGGACTACGAAGCCCAGAG |
| 78 | GTCCCTGCGGCCCTGCCCGCCCACCCGGAC |
| 79 | ACCCCACCCCTTCCCCCTCCTTTCCGAAGC |
| 80 | CCCCCTCCCTGTTTTTTCAGCCCCCTC |
| 81 | GGGCGGGGAGGGGGCCGGCCCGCGGGCCGC |
| 82 | GCAGCCGGAAGCCGGGACCGCCACCGGCCC |
| 83 | CCGGCGAGGGGAGCCCGGCTCCAGGCCCCG |
| 84 | TGCCTAAAACGAGATTCTGTACGATAAAGC |
| 85 | CGCGCCGCGCGATCGGCCCGCGCCCATTGG |
| 86 | CTCTCCGGCCCGCCGCTCACCGCCCCTCCT |
| 87 | CCGCACCGCCCCTACCCGCAGGCCGCGGCG |
| 88 | GGCTGTCGGCGCGGGGCACCCTGGGACTTG |
| 89 | CAGGCGGGGAGGGGCCCCGGGGCGGGGAGG |
| 90 | GCTTGCCACCTGCCGGCTGCAAACGGCGGA |
| 91 | GGGACTACGAAGCCCAGAGGTCCCTGCGGC |
| 92 | TCCCCCTCCTTTCCGAAGCCCCCCTCCCTG |
| 93 | TTTTTTCAGCCCCCTCCCCCCCATCCCCCA |
| 94 | TGGAGC |
| 95 | GGGCCGGCCCGCGGGCCGCGCAGCCGGAAG |
| 96 | CCGGGACCGCCACCGGCCCCCGGCGAGGGG |
| 97 | AGCCCGGCTCCAGGCCCCGCCCCCTGGCGG |
| 98 | ATCGGCCCGCGCCCATTGGCTCTCCGGCCC |
| 99 | GCCGCTCACCGCCCCTCCTCCGCACCGCCC |
| 100 | CGGGGCACCCTGGGACTTGTAGTCCAAGCC |
| 101 | TGGGGTGAGTCCTGCTCCTTTGTTCTTCCC |
| 102 | GGTCCCTCCCTACCTCTGCCCCGCGCTCTG |
| 103 | CCTTTGATCCTCTGCTCGGCTCTGAGCCAT |
| 104 | TCGAGGCGCCGGCGGGGCGGCCCGGCGGCC |
| 105 | GGAAGTGCACCCTGCTCGGGCTTTGCGCCC |
| 106 | CCGGCAGCTTCCTCCGCCCGCGAGG |
| 107 | CCTGCAGG |
| 108 | TAGTAAGGTA |
| 109 | TTTGCGCGTA |
| 110 | AGTCCGGGTA |
| 111 | ATTCGTCTAGTACTAATCCTCTCGGCAGCCG |
| | ATCTAATCCTCAGTCTAGCGCTGACAGATGG |
| | TTCACGGGTGGA |
| 112 | ACCATCTGTCAATCGGCTTAACCATCTGTCA |
| | AGTATCTATACCATCTGTC |
| 113 | ACCATCTGTCAAGTATCTATACCATCTGTC |
| 114 | ACCATCTGTC |
| 115 | TACGGGCCAGACAGATGGTTCCCAATACGAC |
| | TAATCCTCATAGAAGACGCACTAATCCTC |
| 116 | TAGTACTAATCCTCTCGGCAGCCGATCTAAT |
| | CCTCAGTCTAGCGCTGACAGATGGTTCAC |
| 117 | CTAATCCTCTCGGCCCGATCTAATCCTCAGTC |
| | TCGCTGACAGATGGT |
| 118 | GACAGATGGTTCCCAACGACTAATCCTCATA |
| | GAACGCACTAATCCTC |
| 119 | CTAAGCCACATTCGTCTAGTACTAATCCTCTC |
| | GGCAGCCGATCTAATCCTCAGTCTAGCGCTG |
| | ACAGATGGTTCACGGGTGGATTAATTGGTGT |
| | TCCGAACA |
| 120 | GTGAGTGACAGGCGACCAGTACTATAAATA |
| | GATACGGGCCAGACAGATGGTTCCCAATACG |
| | ACTAATCCTCATAGAAGACGCACTAATCCTC |
| 121 | CTAATCCTCTCGGCCGATCTAATCCTCAGTCT |
| | GCTGACAGATGGT |
| 122 | TCTAGAGGGTATATAATGGGGGCCA |
| 123 | TTCGCATATTAAGGTGACGCGTGTGGCCTCG |
| | AACACCGAGCGACCCTGCAGCGACCCGCTTA |
| | A |
| 124 | CAGAATTAACAGTATAAATTGCATCTCTTGT |
| | TCAAGAGTTCCCTATCACTCT |
| 125 | AAAATGTGCGCATGTGCAGCCATTGCCTGGG |
| | ACGCATGCGTAGGGAGCCGCGCGACAAACT |
| | GAGCCATTGCGGCAAGACTAGCGCAGAGAG |
| | GAGAGGGAGCCGGAGATGCCAGACGCTTGG |
| | TTCTGAGGAGTGATTTGCAACGCAATGGAGC |
| | GAGGAAGG |
| 126 | AGACGCTAGCGGGGGGCTATAAAAGGGGGT |
| | GGGGGCGTTCGTCCTCACTCT |
| 127 | TAGGCGTGTACGGTGGGAGGCCTATATAAGC |
| | AGAGCTCGTTTAGTGAACCGTCAGATCGCCT |
| | GGA |
| 128 | TGCTGAGTCAGCAAGTATACGAACTAAGCCA |
| | CAGGAGTCTTGTACCATCTGTCAATCGGCTT |
| | AACCATCTGTCAAGTATCTATACCATCTGTC |
| 129 | AAGGTCATGACCTTAAAACCATGCTGACTCA |
| | GCATGCACGCATTGTGGCTTAGAATTGCGCC |
| | TATCTAATCCTCTATCAGACAGAACCATCTG |
| | TC |
| 130 | GACAGATGGTTTGTCGAGTCACTAATCCTCA |
| | TCCTTAGAGAAGAGGATTAGTTGAACAGTAT |
| | GCTGACTCAGCAAGTGTCGCAACCATCTGTC |
| 131 | GTGAGTGACAGGCGAACCAGTACTATAAAT |
| | AGATACGGGCCAGACAGATGGTTCCCAATAC |
| | GACTAATCCTCATAGAAGACGCACTAATCCT |
| | C |
| 132 | GACAGATGGTTTTGACGCCTTGTGGCTTAGA |
| | GGCACATCATGCTGAGTCAGCAACCGACGGC |
| | TGAGGATTAGTTTGCGAGTGTACCATCTGTC |
| 133 | CTAAGCCACATTCGTCTAGTACTAATCCTCTC |
| | GGCAGCCGATCTAATCCTCAGTCTAGCGCTG |
| | ACAGATGGTTCACGGGTGGATTAATTGG |
| 134 | TGCTGACTCAGCAACGACTATAAGTGGCTTA |
| | GAACATAACGTCTCTAATCCTCAGACTGCGC |
| | GTGACAGATGGTTTCGTCCCGTACCATCTGT |
| | C |
| 135 | TGCTGAGTCAGCATAGGAAGAGTCTAAGCCA |
| | CAGCACGCGTAGTCTAATCCTCAGCCTACTT |
| | AACTATAAATAGATCGAGCAATACCATCTGT |
| | C |
| 136 | ACTATAAATAGAAACTACAACTGAGGATTAG |
| | AGTACACACTTGCTGAGTCAGCAAATCTTTA |
| | GTCTAAGCCACTTAGATCGCCAACCATCTGT |
| | C |
| 137 | CTAAGCCACAGTAACATGCGACTAATCCTCT |
| | CTGTCTACAGAGTGGCTTAGTCTATCGTGTT |
| | GCTGACTCAGCATTAGATAGTGACAGATGGT |
| 279 | GTTAATCCCCTAGCTTAGATTCTATTTATAGT |
| | TATTACTATGCTGACTCAGCAATAGACGGTA |
| | GAGGATTAGAGCACTCGTTTGACAGATGGT |
| 280 | GTGGCTTAGAAAGGTCCGATGCTGAGTCAGC |
| | AAGCAAGTACTCTAATCCTCTATAGGACAAA |
| | CTATAAATAGAATACGAGGAGTTAATCCCCT |

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the invention described herein. Scope of the disclosure is thus indicated by the appended claims rather than by the foregoing description, and all changes that come within the meaning and range of equivalency of the claims are intended to be embraced therein.

What is claimed is:

1. An engineered photoreceptor-specific regulatory element comprising an enhancer region selected from the group consisting of SEQ ID NOs: 1-4, operably linked to a minimal promoter, wherein the enhancer region is heterologous to the minimal promoter, wherein the engineered photoreceptor-specific regulatory element exhibits greater activity in photoreceptor cells of a mammalian retina as compared to non-photoreceptor cells of the same mammalian retina.

2. The engineered photoreceptor-specific regulatory element of claim 1, wherein the minimal promoter is selected from the group consisting of: minP, NFkB response element, CREB response element, NFAT response element, SRF response element 1, SRF response element 2, API response element, TCF-LEF response element promoter fusion, Hypoxia responsive element, SMAD binding element, STAT3 binding site, minCMV, YB TATA, minTK, lateADE, minIL2.2, SMP, inducer molecule responsive promoters, CMV, EFS, SFFV, SV40, MND, PGK, UbC, hEFlaV1, hCAGG, hEFlaV2, hACTb, heIF4Al, hGAPDH, hGRP78, hGRP94, hHSP70, hKINb, hUBIb, and tandem repeats thereof.

3. The engineered photoreceptor-specific regulatory element of claim 2, further comprising a spacer sequence, wherein the spacer is operably linked to the enhancer region and the minimal promoter.

4. The engineered photoreceptor-specific regulatory element of claim 3, wherein the spacer is selected from the group consisting of SEQ ID NOs: 107-110.

5. An engineered photoreceptor-specific regulatory element comprising an enhancer region, wherein the enhancer region comprises an ablation of at least one nucleotide motif within a wild-type enhancer region selected from the group consisting of SEQ ID NOs: 1-4, wherein the engineered photoreceptor-specific regulatory element has greater activity than the same regulatory element without the ablation in photoreceptor cells as compared to non-photoreceptor cells.

6. The engineered photoreceptor-specific regulatory element of claim 5, wherein the engineered photoreceptor-specific regulatory element further comprises a minimal promoter, optionally wherein the engineered photoreceptor-specific regulatory element further comprises a spacer, wherein spacer is operably linked to the enhancer region and the minimal promoter.

7. The engineered photoreceptor-specific regulatory element claim 6, wherein the ablation comprises a substitution or deletion of one or more nucleotides of the at least one nucleotide motif, wherein the nucleotide substitution comprises an inert sequence.

8. The engineered photoreceptor-specific regulatory element of claim 7, wherein the ablation comprises a nucleotide substitution of the motif comprising the nucleotide sequence selected from the group consisting of SEQ ID NOs: 5, 7, 9, 11, 13, 15, and 17, wherein the wild-type enhancer region is SEQ ID NO: 1.

9. The engineered photoreceptor-specific regulatory element of claim 7, wherein the ablation comprises a nucleotide substitution of the motif comprising the nucleotide sequence selected from the group consisting of SEQ ID NOs: 19 and 20, wherein the wild-type enhancer region is SEQ ID NO: 2.

10. The engineered photoreceptor-specific regulatory element of claim 7, wherein the ablation comprises a nucleotide substitution of the motif comprising the nucleotide sequence selected from the group consisting of SEQ ID NOs: 22-24, 26, 27, and 29, wherein the wild-type enhancer region is SEQ ID NO: 3.

11. An engineered photoreceptor-specific regulatory element comprising:
a. a polynucleotide sequence having at least 75%, at least 80%, at least 85%, at least 90%, at least 95%, at least 96%, at least 97%, at least 98%, at least 99%, or 100% sequence identity to a nucleotide sequence selected from the group consisting of SEQ ID NOs: 138-163 operably linked to a minimal promoter; or
b. a polynucleotide sequence having at least 75%, at least 80%, at least 85%, at least 90%, at least 95%, at least 96%, at least 97%, at least 98%, at least 99%, or 100% sequence identity to a nucleotide sequence selected from the group consisting of SEQ ID NOs: 164-247, and 276-278.

12. A heterologous construct comprising the engineered photoreceptor-specific regulatory element according to any one of claims 1-11 operably linked to a polynucleotide, wherein the polynucleotide comprises a polynucleotide sequence encoding a polypeptide, optionally wherein the polypeptide comprises at least one effector molecule, or comprises a first effector molecule and a second effector molecule, optionally wherein the polynucleotide comprises a polynucleotide sequence encoding the first effector molecule, a linker polynucleotide sequence, and a polynucleotide sequence encoding the second effector, optionally wherein the linker polynucleotide sequence encodes one or more 2A ribosome skipping elements selected from the group consisting of: P2A, T2A, E2A, and F2A.

13. The heterologous construct of claim 12, wherein the at least one effector molecule belongs to a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme, optionally wherein each of the first effector molecule and the second effector molecule is from a separate therapeutic class, optionally wherein each of the at least one effector molecule is a human-derived effector molecule.

14. A vector comprising the heterologous construct of claim 12 or 13.

15. A dual expression vector comprising the heterologous construct according to any one of claims 12 or 13 and a second construct comprising a polynucleotide sequence encoding a second effector protein.

16. A photoreceptor cell comprising the heterologous construct of claim 12 or 13, the vector according to claim 14, or the dual expression vector according to claim 15, optionally wherein the photoreceptor cell is a rod cell or a cone cell, optionally wherein the photoreceptor cell expresses at least one effector molecule.

17. A pharmaceutical composition comprising the engineered photoreceptor-specific regulatory element according to any one of claims 1-11, the heterologous construct of claim 12 or 13, the vector of claim 14, the dual expression vector according to claim 15, or the photoreceptor cell of claim 16, and a pharmaceutically acceptable carrier, pharmaceutically acceptable excipient, or a combination thereof.

18. A method of increasing expression of a target gene, the method comprising use of the engineered photoreceptor-specific regulatory element according to any one of claims 1-11, the heterologous construct of claim 12 or 13, the vector of claim 14, the dual expression vector according to claim 15, or the photoreceptor cell of claim 16, to increase expression of the target gene.

19. A method of treating a subject in need thereof, the method comprising administering the engineered photoreceptor-specific regulatory element according to any one of claims 1-11, the heterologous construct of claim 12 or 13, the vector of claim 14, the dual expression vector according to claim 15, the photoreceptor cell of claim 16, or the pharmaceutical composition according to claim 17, optionally wherein the subject has an ocular disease or disorder.

20. A kit for treating and/or preventing a tumor, comprising the pharmaceutical composition according to claim 17, optionally wherein the kit further comprises written instructions for using the pharmaceutical composition for treating and/or preventing a tumor in a subject.


SEQUENCE LISTING (280 sequences)

SEQ ID NO: 1 - DNA, 309 bp, synthetic construct
  source 1..309: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacgg

SEQ ID NO: 2 - DNA, 417 bp, synthetic construct
  source 1..417: mol_type = other DNA
ggttacacaaccaggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctc

SEQ ID NO: 3 - DNA, 426 bp, synthetic construct
  source 1..426: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagc

SEQ ID NO: 4 - DNA, 387 bp, synthetic construct
  source 1..387: mol_type = other DNA
agcacgcgcgagctgtcaccagggcgcgagacaaccgaacacggccccgccccctgcccgtctattggcccgcccgctggagccccgcccctcaggccctgcattgcgccaacggcgcagcgctgggccgcgaccccggcaccggcacccgttccgagggttcgcgccgcaggcgcaaagtttagggtcagccacaaacgcgcggccgctttgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctc

SEQ ID NO: 5 - DNA, 30 bp, synthetic construct
  source 1..30: mol_type = other DNA
tgttgagagctcaagctctttttaacgctt

SEQ ID NO: 6 - DNA, 30 bp, synthetic construct
  source 1..30: mol_type = other DNA
ttacctactaggttaattcgtccgatagat

SEQ ID NO: 7 - DNA, 30 bp, synthetic construct
  source 1..30: mol_type = other DNA
tgccttgctgtctccaaagtattgccttca

SEQ ID NO: 8 - DNA, 30 bp, synthetic construct
  source 1..30: mol_type = other DNA
actacgaattgcgagcttctaagtccaatt

SEQ ID NO: 9 - DNA, 30 bp, synthetic construct
  source 1..30: mol_type = other DNA
tcctcatagttcaaagtgtccaccatcaca

SEQ ID NO: 10 - DNA, 30 bp, synthetic construct
  source 1..30: mol_type = other DNA
ttcggtattcgagtcgtcagactcaattat

SEQ ID NO: 11 - DNA, 30 bp, synthetic construct
  source 1..30: mol_type = other DNA
ttcacgtttcagccaataggaagaaggaaa

SEQ ID NO: 12 - DNA, 30 bp, synthetic construct
  source 1..30: mol_type = other DNA
tacacggttaagactcaatccggtaagtaa

SEQ ID NO: 13 - DNA, 30 bp, synthetic construct
  source 1..30: mol_type = other DNA
gagaaaaacaggaaaagagtttacatgcca

SEQ ID NO: 14 - DNA, 30 bp, synthetic construct
  source 1..30: mol_type = other DNA
tactgtgatcaggtgtctacaagactagta

SEQ ID NO: 15 - DNA, 30 bp, synthetic construct
  source 1..30: mol_type = other DNA
ctttatttcttttttcttttctttcttttt

SEQ ID NO: 16 - DNA, 30 bp, synthetic construct
  source 1..30: mol_type = other DNA
actatcacgtatataccagcgagttcgata

SEQ ID NO: 17-18 - skipped (2 entries)

SEQ ID NO: 19 - DNA, 30 bp, synthetic construct
  source 1..30: mol_type = other DNA
ccggcgaggggagcccggctccaggccccg

SEQ ID NO: 20 - DNA, 30 bp, synthetic construct
  source 1..30: mol_type = other DNA
ccccctggcgggctggccccgcccccgcgc

SEQ ID NO: 21 - DNA, 30 bp, synthetic construct
  source 1..30: mol_type = other DNA
tgcctaaaacgagattctgtacgataaagc

SEQ ID NO: 22 - DNA, 30 bp, synthetic construct
  source 1..30: mol_type = other DNA
agcccggctccaggccccgccccctggcgg

SEQ ID NO: 23 - DNA, 30 bp, synthetic construct
  source 1..30: mol_type = other DNA
gctggccccgcccccgcgccgcgccgcgcg

SEQ ID NO: 24 - DNA, 30 bp, synthetic construct
  source 1..30: mol_type = other DNA
ctacccgcaggccgcggcgggctgtcggcg

SEQ ID NO: 25 - DNA, 30 bp, synthetic construct
  source 1..30: mol_type = other DNA
tacggtctcgctaataggagtaagatacat

SEQ ID NO: 26 - DNA, 30 bp, synthetic construct
  source 1..30: mol_type = other DNA
gcttgccacctgccggctgcaaacggcgga

SEQ ID NO: 27 - DNA, 30 bp, synthetic construct
  source 1..30: mol_type = other DNA
cctgcccgcccacccggacaccccacccct

SEQ ID NO: 28 - DNA, 30 bp, synthetic construct
  source 1..30: mol_type = other DNA
tgttaagcatactaaactgtaaagaagcga

SEQ ID NO: 29 - DNA, 30 bp, synthetic construct
  source 1..30: mol_type = other DNA
tccccctcctttccgaagcccccctccctg

SEQ ID NO: 30 - DNA, 30 bp, synthetic construct
  source 1..30: mol_type = other DNA
gtttcgagcgacgcttaatattgctagata

SEQ ID NO: 31 - DNA, 22 bp, synthetic construct
  source 1..22: mol_type = other DNA
agcgctgacagatggttcacgg

SEQ ID NO: 32 - DNA, 25 bp, synthetic construct
  source 1..25: mol_type = other DNA
gggccagacagatggttcccaatac

SEQ ID NO: 33 - DNA, 30 bp, synthetic construct; identical to SEQ ID NO: 5
  source 1..30: mol_type = other DNA

SEQ ID NO: 34 - DNA, 30 bp, synthetic construct; identical to SEQ ID NO: 15
  source 1..30: mol_type = other DNA

SEQ ID NO: 35 - DNA, 30 bp, synthetic construct; identical to SEQ ID NO: 16
  source 1..30: mol_type = other DNA

SEQ ID NO: 36-37 - skipped (2 entries)

SEQ ID NO: 38 - DNA, 30 bp, synthetic construct; identical to SEQ ID NO: 6
  source 1..30: mol_type = other DNA

SEQ ID NO: 39 - DNA, 30 bp, synthetic construct; identical to SEQ ID NO: 7
  source 1..30: mol_type = other DNA

SEQ ID NO: 40 - DNA, 30 bp, synthetic construct; identical to SEQ ID NO: 8
  source 1..30: mol_type = other DNA

SEQ ID NO: 41 - DNA, 30 bp, synthetic construct; identical to SEQ ID NO: 9
  source 1..30: mol_type = other DNA

SEQ ID NO: 42 - DNA, 30 bp, synthetic construct; identical to SEQ ID NO: 10
  source 1..30: mol_type = other DNA

SEQ ID NO: 43 - DNA, 30 bp, synthetic construct; identical to SEQ ID NO: 11
  source 1..30: mol_type = other DNA

SEQ ID NO: 44 - DNA, 30 bp, synthetic construct; identical to SEQ ID NO: 12
  source 1..30: mol_type = other DNA

SEQ ID NO: 45 - DNA, 30 bp, synthetic construct; identical to SEQ ID NO: 13
  source 1..30: mol_type = other DNA

SEQ ID NO: 46 - DNA, 30 bp, synthetic construct; identical to SEQ ID NO: 14
  source 1..30: mol_type = other DNA

SEQ ID NO: 47 - DNA, 30 bp, synthetic construct
  source 1..30: mol_type = other DNA
ctcctgctattggtcagaatgtgtcacgtg

SEQ ID NO: 48 - DNA, 30 bp, synthetic construct
  source 1..30: mol_type = other DNA
accatactcagctgcaagggttggtgggaa

SEQ ID NO: 49 - DNA, 30 bp, synthetic construct
  source 1..30: mol_type = other DNA
atgtagtctttttttctgggaggccatgtg

SEQ ID NO: 50 - DNA, 30 bp, synthetic construct
  source 1..30: mol_type = other DNA
ttcggcttaaaattgtctttttttcttttt

SEQ ID NO: 51 - DNA, 30 bp, synthetic construct
  source 1..30: mol_type = other DNA
agtagcatggttggagcttaaccccaagtc

SEQ ID NO: 52 - DNA, 30 bp, synthetic construct
  source 1..30: mol_type = other DNA
taattttattctgcctctttatttaaaaag

SEQ ID NO: 53 - DNA, 30 bp, synthetic construct
  source 1..30: mol_type = other DNA
gcaaggaagattattttgagtcactgcacc

SEQ ID NO: 54 - DNA, 30 bp, synthetic construct
  source 1..30: mol_type = other DNA
ctttctgaacaatagctgttaccaaggtct

SEQ ID NO: 55 - DNA, 30 bp, synthetic construct
  source 1..30: mol_type = other DNA
caggagcattaggcgctcaattctatttta

SEQ ID NO: 56 - DNA, 30 bp, synthetic construct
  source 1..30: mol_type = other DNA
acgctatcacagagagcctcttagaagcac

SEQ ID NO: 57 - DNA, 30 bp, synthetic construct
  source 1..30: mol_type = other DNA
gctcgcatccccagcacgttaaattaccca

SEQ ID NO: 58 - DNA, 30 bp, synthetic construct
  source 1..30: mol_type = other DNA
agaaatgtctctggccaaacctttcaagta

SEQ ID NO: 59 - DNA, 25 bp, synthetic construct
  source 1..25: mol_type = other DNA
acgtgcagacaagagaaaatcagag

SEQ ID NO: 60 - DNA, 30 bp, synthetic construct
  source 1..30: mol_type = other DNA
gaaacaaaaatttaaaaatagctctacaag

SEQ ID NO: 61 - DNA, 30 bp, synthetic construct
  source 1..30: mol_type = other DNA
ggtaacccgattagtcaggcacctctttct

SEQ ID NO: 62 - DNA, 30 bp, synthetic construct
  source 1..30: mol_type = other DNA
cctcaaatgaccttgaggttgtaaatacga

SEQ ID NO: 63 - DNA, 30 bp, synthetic construct
  source 1..30: mol_type = other DNA
tctgtggatgccccctggtggccctaaata

SEQ ID NO: 64 - DNA, 30 bp, synthetic construct
  source 1..30: mol_type = other DNA
gaatggcttcaatggatggaaatttaacgt

SEQ ID NO: 65 - DNA, 30 bp, synthetic construct
  source 1..30: mol_type = other DNA
ggttgaagcacaggagggtaaataagaagg

SEQ ID NO: 66 - DNA, 30 bp, synthetic construct
  source 1..30: mol_type = other DNA
tttccagacaaagagactcttaaaggtaca

SEQ ID NO: 67 - DNA, 30 bp, synthetic construct
  source 1..30: mol_type = other DNA
gactcttatgtctgtccctcctccttaaag

SEQ ID NO: 68 - DNA, 27 bp, synthetic construct
  source 1..27: mol_type = other DNA
ggccagagacgctcccgagcccatctc

SEQ ID NO: 69 - DNA, 30 bp, synthetic construct
  source 1..30: mol_type = other DNA
ctcaggccctgcattgcgccaacggcgcag

SEQ ID NO: 70 - DNA, 30 bp, synthetic construct
  source 1..30: mol_type = other DNA
cgctgggccgcgaccccggcaccggcaccc

SEQ ID NO: 71 - DNA, 30 bp, synthetic construct
  source 1..30: mol_type = other DNA
gttccgagggttcgcgccgcaggcgcaaag

SEQ ID NO: 72 - DNA, 30 bp, synthetic construct
  source 1..30: mol_type = other DNA
ccacattgctatagtgctgtatagcgatta

SEQ ID NO: 73 - DNA, 30 bp, synthetic construct
  source 1..30: mol_type = other DNA
ttgagcctggcgtaaggggcggcggcgtag

SEQ ID NO: 74 - DNA, 30 bp, synthetic construct
  source 1..30: mol_type = other DNA
ggggacgttgtgaatagctggtcgaagttt

SEQ ID NO: 75 - DNA, 30 bp, synthetic construct
  source 1..30: mol_type = other DNA
ggttacacaaccaggcggggaggggccccg

SEQ ID NO: 76 - DNA, 30 bp, synthetic construct
  source 1..30: mol_type = other DNA
tagtccaagccgcttgccacctgccggctg

SEQ ID NO: 77 - DNA, 30 bp, synthetic construct
  source 1..30: mol_type = other DNA
caaacggcggagggactacgaagcccagag

SEQ ID NO: 78 - DNA, 30 bp, synthetic construct
  source 1..30: mol_type = other DNA
gtccctgcggccctgcccgcccacccggac

SEQ ID NO: 79 - DNA, 30 bp, synthetic construct
  source 1..30: mol_type = other DNA
accccaccccttccccctcctttccgaagc

SEQ ID NO: 80 - DNA, 27 bp, synthetic construct
  source 1..27: mol_type = other DNA
ccccctccctgttttttcagccccctc

SEQ ID NO: 81 - DNA, 30 bp, synthetic construct
  source 1..30: mol_type = other DNA
gggcggggagggggccggcccgcgggccgc

SEQ ID NO: 82 - DNA, 30 bp, synthetic construct
  source 1..30: mol_type = other DNA
gcagccggaagccgggaccgccaccggccc

SEQ ID NO: 83 - DNA, 30 bp, synthetic construct; identical to SEQ ID NO: 19
  source 1..30: mol_type = other DNA

SEQ ID NO: 84 - DNA, 30 bp, synthetic construct; identical to SEQ ID NO: 21
  source 1..30: mol_type = other DNA

SEQ ID NO: 85 - DNA, 30 bp, synthetic construct
  source 1..30: mol_type = other DNA
cgcgccgcgcgatcggcccgcgcccattgg

SEQ ID NO: 86 - DNA, 30 bp, synthetic construct
  source 1..30: mol_type = other DNA
ctctccggcccgccgctcaccgcccctcct

SEQ ID NO: 87 - DNA, 30 bp, synthetic construct
  source 1..30: mol_type = other DNA
ccgcaccgcccctacccgcaggccgcggcg

SEQ ID NO: 88 - DNA, 30 bp, synthetic construct
  source 1..30: mol_type = other DNA
ggctgtcggcgcggggcaccctgggacttg

SEQ ID NO: 89 - DNA, 30 bp, synthetic construct
  source 1..30: mol_type = other DNA
caggcggggaggggccccggggcggggagg

SEQ ID NO: 90 - DNA, 30 bp, synthetic construct; identical to SEQ ID NO: 26
  source 1..30: mol_type = other DNA

SEQ ID NO: 91 - DNA, 30 bp, synthetic construct
  source 1..30: mol_type = other DNA
gggactacgaagcccagaggtccctgcggc

SEQ ID NO: 92 - DNA, 30 bp, synthetic construct; identical to SEQ ID NO: 29
  source 1..30: mol_type = other DNA

SEQ ID NO: 93 - DNA, 30 bp, synthetic construct
  source 1..30: mol_type = other DNA
ttttttcagccccctcccccccatccccca

SEQ ID NO: 94 - skipped

SEQ ID NO: 95 - DNA, 30 bp, synthetic construct
  source 1..30: mol_type = other DNA
gggccggcccgcgggccgcgcagccggaag

SEQ ID NO: 96 - DNA, 30 bp, synthetic construct
  source 1..30: mol_type = other DNA
ccgggaccgccaccggcccccggcgagggg

SEQ ID NO: 97 - DNA, 30 bp, synthetic construct; identical to SEQ ID NO: 22
  source 1..30: mol_type = other DNA

SEQ ID NO: 98 - DNA, 30 bp, synthetic construct
  source 1..30: mol_type = other DNA
atcggcccgcgcccattggctctccggccc

SEQ ID NO: 99 - DNA, 30 bp, synthetic construct
  source 1..30: mol_type = other DNA
gccgctcaccgcccctcctccgcaccgccc

SEQ ID NO: 100 - DNA, 30 bp, synthetic construct
  source 1..30: mol_type = other DNA
cggggcaccctgggacttgtagtccaagcc

SEQ ID NO: 101 - DNA, 30 bp, synthetic construct
  source 1..30: mol_type = other DNA
tggggtgagtcctgctcctttgttcttccc

SEQ ID NO: 102 - DNA, 30 bp, synthetic construct
  source 1..30: mol_type = other DNA
ggtccctccctacctctgccccgcgctctg

SEQ ID NO: 103 - DNA, 30 bp, synthetic construct
  source 1..30: mol_type = other DNA
cctttgatcctctgctcggctctgagccat

SEQ ID NO: 104 - DNA, 30 bp, synthetic construct
  source 1..30: mol_type = other DNA
tcgaggcgccggcggggcggcccggcggcc

SEQ ID NO: 105 - DNA, 30 bp, synthetic construct
  source 1..30: mol_type = other DNA
ggaagtgcaccctgctcgggctttgcgccc

SEQ ID NO: 106 - DNA, 25 bp, synthetic construct
  source 1..25: mol_type = other DNA
ccggcagcttcctccgcccgcgagg

SEQ ID NO: 107 - skipped

SEQ ID NO: 108 - DNA, 10 bp, synthetic construct
  source 1..10: mol_type = other DNA
tagtaaggta

SEQ ID NO: 109 - DNA, 10 bp, synthetic construct
  source 1..10: mol_type = other DNA
tttgcgcgta

SEQ ID NO: 110 - DNA, 10 bp, synthetic construct
  source 1..10: mol_type = other DNA
agtccgggta

SEQ ID NO: 111 - DNA, 74 bp, synthetic construct
  source 1..74: mol_type = other DNA
attcgtctagtactaatcctctcggcagccgatctaatcctcagtctagcgctgacagatggttcacgggtgga

SEQ ID NO: 112 - DNA, 50 bp, synthetic construct
  source 1..50: mol_type = other DNA
accatctgtcaatcggcttaaccatctgtcaagtatctataccatctgtc

SEQ ID NO: 113 - DNA, 30 bp, synthetic construct
  source 1..30: mol_type = other DNA
accatctgtcaagtatctataccatctgtc

SEQ ID NO: 114 - DNA, 10 bp, synthetic construct
  source 1..10: mol_type = other DNA
accatctgtc

SEQ ID NO: 115 - DNA, 60 bp, synthetic construct
  source 1..60: mol_type = other DNA
tacgggccagacagatggttcccaatacgactaatcctcatagaagacgcactaatcctc

SEQ ID NO: 116 - DNA, 60 bp, synthetic construct
  source 1..60: mol_type = other DNA
tagtactaatcctctcggcagccgatctaatcctcagtctagcgctgacagatggttcac

SEQ ID NO: 117 - DNA, 47 bp, synthetic construct
  source 1..47: mol_type = other DNA
ctaatcctctcggcccgatctaatcctcagtctcgctgacagatggt

SEQ ID NO: 118 - DNA, 47 bp, synthetic construct
  source 1..47: mol_type = other DNA
gacagatggttcccaacgactaatcctcatagaacgcactaatcctc

SEQ ID NO: 119 - DNA, 102 bp, synthetic construct
  source 1..102: mol_type = other DNA
ctaagccacattcgtctagtactaatcctctcggcagccgatctaatcctcagtctagcgctgacagatggttcacgggtggattaattggtgttccgaaca

SEQ ID NO: 120 - DNA, 92 bp, synthetic construct
  source 1..92: mol_type = other DNA
gtgagtgacaggcgaccagtactataaatagatacgggccagacagatggttcccaatacgactaatcctcatagaagacgcactaatcctc

SEQ ID NO: 121 - DNA, 45 bp, synthetic construct
  source 1..45: mol_type = other DNA
ctaatcctctcggccgatctaatcctcagtctgctgacagatggt

SEQ ID NO: 122 - DNA, 25 bp, synthetic construct
  source 1..25: mol_type = other DNA
tctagagggtatataatgggggcca

SEQ ID NO: 123 - DNA, 63 bp, synthetic construct
  source 1..63: mol_type = other DNA
ttcgcatattaaggtgacgcgtgtggcctcgaacaccgagcgaccctgcagcgacccgcttaa

SEQ ID NO: 124 - DNA, 52 bp, synthetic construct
  source 1..52: mol_type = other DNA
cagaattaacagtataaattgcatctcttgttcaagagttccctatcactct

SEQ ID NO: 125 - DNA, 160 bp, synthetic construct
  source 1..160: mol_type = other DNA
aaaatgtgcgcatgtgcagccattgcctgggacgcatgcgtagggagccgcgcgacaaactgagccattgcggcaagactagcgcagagaggagagggagccggagatgccagacgcttggttctgaggagtgatttgcaacgcaatggagcgaggaagg

SEQ ID NO: 126 - DNA, 51 bp, synthetic construct
  source 1..51: mol_type = other DNA
agacgctagcggggggctataaaagggggtgggggcgttcgtcctcactct

SEQ ID NO: 127 - DNA, 65 bp, synthetic construct
  source 1..65: mol_type = other DNA
taggcgtgtacggtgggaggcctatataagcagagctcgtttagtgaaccgtcagatcgcctgga

SEQ ID NO: 128 - DNA, 93 bp, synthetic construct
  source 1..93: mol_type = other DNA
tgctgagtcagcaagtatacgaactaagccacaggagtcttgtaccatctgtcaatcggcttaaccatctgtcaagtatctataccatctgtc

SEQ ID NO: 129 - DNA, 95 bp, synthetic construct
  source 1..95: mol_type = other DNA
aaggtcatgaccttaaaaccatgctgactcagcatgcacgcattgtggcttagaattgcgcctatctaatcctctatcagacagaaccatctgtc

SEQ ID NO: 130 - DNA, 93 bp, synthetic construct
  source 1..93: mol_type = other DNA
gacagatggtttgtcgagtcactaatcctcatccttagagaagaggattagttgaacagtatgctgactcagcaagtgtcgcaaccatctgtc

SEQ ID NO: 131 - DNA, 93 bp, synthetic construct
  source 1..93: mol_type = other DNA
gtgagtgacaggcgaaccagtactataaatagatacgggccagacagatggttcccaatacgactaatcctcatagaagacgcactaatcctc

SEQ ID NO: 132 - DNA, 93 bp, synthetic construct
  source 1..93: mol_type = other DNA
gacagatggttttgacgccttgtggcttagaggcacatcatgctgagtcagcaaccgacggctgaggattagtttgcgagtgtaccatctgtc

SEQ ID NO: 133 - DNA, 91 bp, synthetic construct
  source 1..91: mol_type = other DNA
ctaagccacattcgtctagtactaatcctctcggcagccgatctaatcctcagtctagcgctgacagatggttcacgggtggattaattgg

SEQ ID NO: 134 - DNA, 94 bp, synthetic construct
  source 1..94: mol_type = other DNA
tgctgactcagcaacgactataagtggcttagaacataacgtctctaatcctcagactgcgcgtgacagatggtttcgtcccgtaccatctgtc

SEQ ID NO: 135 - DNA, 94 bp, synthetic construct
  source 1..94: mol_type = other DNA
tgctgagtcagcataggaagagtctaagccacagcacgcgtagtctaatcctcagcctacttaactataaatagatcgagcaataccatctgtc

SEQ ID NO: 136 - DNA, 94 bp, synthetic construct
  source 1..94: mol_type = other DNA
actataaatagaaactacaactgaggattagagtacacacttgctgagtcagcaaatctttagtctaagccacttagatcgccaaccatctgtc

SEQ ID NO: 137 - DNA, 93 bp, synthetic construct
  source 1..93: mol_type = other DNA
ctaagccacagtaacatgcgactaatcctctctgtctacagagtggcttagtctatcgtgttgctgactcagcattagatagtgacagatggt

SEQ ID NO: 138 - DNA, 342 bp, synthetic construct
  source 1..342: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 139 - DNA, 450 bp, synthetic construct
  source 1..450: mol_type = other DNA
ggttacacaaccaggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 140 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagccctgcaggtctagagggtatataatgggggcca

SEQ ID NO: 141 - DNA, 380 bp, synthetic construct
  source 1..380: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggttcgcatattaaggtgacgcgtgtggcctcgaacaccgagcgaccctgcagcgacccgcttaa

SEQ ID NO: 142 - DNA, 368 bp, synthetic construct
  source 1..368: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggagacgctagcggggggctataaaagggggtgggggcgttcgtcctcactct

SEQ ID NO: 143 - DNA, 555 bp, synthetic construct
  source 1..555: mol_type = other DNA
agcacgcgcgagctgtcaccagggcgcgagacaaccgaacacggccccgccccctgcccgtctattggcccgcccgctggagccccgcccctcaggccctgcattgcgccaacggcgcagcgctgggccgcgaccccggcaccggcacccgttccgagggttcgcgccgcaggcgcaaagtttagggtcagccacaaacgcgcggccgctttgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggaaaatgtgcgcatgtgcagccattgcctgggacgcatgcgtagggagccgcgcgacaaactgagccattgcggcaagactagcgcagagaggagagggagccggagatgccagacgcttggttctgaggagtgatttgcaacgcaatggagcgaggaagg

SEQ ID NO: 144 - DNA, 446 bp, synthetic construct
  source 1..446: mol_type = other DNA
agcacgcgcgagctgtcaccagggcgcgagacaaccgaacacggccccgccccctgcccgtctattggcccgcccgctggagccccgcccctcaggccctgcattgcgccaacggcgcagcgctgggccgcgaccccggcaccggcacccgttccgagggttcgcgccgcaggcgcaaagtttagggtcagccacaaacgcgcggccgctttgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 145 - DNA, 460 bp, synthetic construct
  source 1..460: mol_type = other DNA
agcacgcgcgagctgtcaccagggcgcgagacaaccgaacacggccccgccccctgcccgtctattggcccgcccgctggagccccgcccctcaggccctgcattgcgccaacggcgcagcgctgggccgcgaccccggcaccggcacccgttccgagggttcgcgccgcaggcgcaaagtttagggtcagccacaaacgcgcggccgctttgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggtaggcgtgtacggtgggaggcctatataagcagagctcgtttagtgaaccgtcagatcgcctgga

SEQ ID NO: 146 - DNA, 488 bp, synthetic construct
  source 1..488: mol_type = other DNA
ggttacacaaccaggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggttcgcatattaaggtgacgcgtgtggcctcgaacaccgagcgaccctgcagcgacccgcttaa

SEQ ID NO: 147 - DNA, 477 bp, synthetic construct
  source 1..477: mol_type = other DNA
ggttacacaaccaggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggcagaattaacagtataaattgcatctcttgttcaagagttccctatcactct

SEQ ID NO: 148 - DNA, 476 bp, synthetic construct
  source 1..476: mol_type = other DNA
ggttacacaaccaggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggagacgctagcggggggctataaaagggggtgggggcgttcgtcctcactct

SEQ ID NO: 149 - DNA, 342 bp, synthetic construct
  source 1..342: mol_type = other DNA
ttacctactaggttaattcgtccgatagattgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 150 - DNA, 342 bp, synthetic construct
  source 1..342: mol_type = other DNA
tgttgagagctcaagctctttttaacgcttactacgaattgcgagcttctaagtccaatttcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 151 - DNA, 342 bp, synthetic construct
  source 1..342: mol_type = other DNA
tgttgagagctcaagctctttttaacgctttgccttgctgtctccaaagtattgccttcattcggtattcgagtcgtcagactcaattatttcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 152 - DNA, 342 bp, synthetic construct
  source 1..342: mol_type = other DNA
tgttgagagctcaagctctttttaacgctttgccttgctgtctccaaagtattgccttcatcctcatagttcaaagtgtccaccatcacatacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 153 - DNA, 342 bp, synthetic construct
  source 1..342: mol_type = other DNA
tgttgagagctcaagctctttttaacgctttgccttgctgtctccaaagtattgccttcatcctcatagttcaaagtgtccaccatcacattcacgtttcagccaataggaagaaggaaatactgtgatcaggtgtctacaagactagtactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 154 - DNA, 342 bp, synthetic construct
  source 1..342: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttactatcacgtatataccagcgagttcgatatttagacggcctgcaggtctagagggtatataatgggggcca

SEQ ID NO: 155 - DNA, 342 bp, synthetic construct
  source 1..342: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttctttttatacactctcctgcaggtctagagggtatataatgggggcca

SEQ ID NO: 156 - DNA, 450 bp, synthetic construct
  source 1..450: mol_type = other DNA
ggttacacaaccaggcggggaggggccccggggcggggagggggccggcccgcgggccgcgcagccggaagccgggaccgccaccggccctacacggttaagactcaatccggtaagtaaccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 157 - DNA, 450 bp, synthetic construct
  source 1..450: mol_type = other DNA
ggttacacaaccaggcggggaggggccccggggcggggagggggccggcccgcgggccgcgcagccggaagccgggaccgccaccggcccccggcgaggggagcccggctccaggccccgtgcctaaaacgagattctgtacgataaagccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 158 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggtacacggttaagactcaatccggtaagtaagctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagccctgcaggtctagagggtatataatgggggcca

SEQ ID NO: 159 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggagcccggctccaggccccgccccctggcggtgcctaaaacgagattctgtacgataaagcatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagccctgcaggtctagagggtatataatgggggcca

SEQ ID NO: 160 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgccctacggtctcgctaataggagtaagatacatcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagccctgcaggtctagagggtatataatgggggcca

SEQ ID NO: 161 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccactatcacgtatataccagcgagttcgatagggactacgaagcccagaggtccctgcggccctgcccgcccacccggacaccccaccccttccccctcctttccgaagcccccctccctgttttttcagccccctcccccccatcccccatggagccctgcaggtctagagggtatataatgggggcca

SEQ ID NO: 162 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggctgttaagcatactaaactgtaaagaagcgatccccctcctttccgaagcccccctccctgttttttcagccccctcccccccatcccccatggagccctgcaggtctagagggtatataatgggggcca

SEQ ID NO: 163 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccacccctgtttcgagcgacgcttaatattgctagatattttttcagccccctcccccccatcccccatggagccctgcaggtctagagggtatataatgggggcca

SEQ ID NO: 164 - DNA, 126 bp, synthetic construct
  source 1..126: mol_type = other DNA
tgctgagtcagcaagtatacgaactaagccacaggagtcttgtaccatctgtcaatcggcttaaccatctgtcaagtatctataccatctgtccctgcaggtctagagggtatataatgggggcca

SEQ ID NO: 165 - DNA, 127 bp, synthetic construct
  source 1..127: mol_type = other DNA
tgctgagtcagcataggaagagtctaagccacagcacgcgtagtctaatcctcagcctacttaactataaatagatcgagcaataccatctgtccctgcaggtctagagggtatataatgggggcca

SEQ ID NO: 166 - DNA, 126 bp, synthetic construct
  source 1..126: mol_type = other DNA
gacagatggttttgacgccttgtggcttagaggcacatcatgctgagtcagcaaccgacggctgaggattagtttgcgagtgtaccatctgtccctgcaggtctagagggtatataatgggggcca

SEQ ID NO: 167 - DNA, 127 bp, synthetic construct
  source 1..127: mol_type = other DNA
tgctgactcagcaacgactataagtggcttagaacataacgtctctaatcctcagactgcgcgtgacagatggtttcgtcccgtaccatctgtccctgcaggtctagagggtatataatgggggcca

SEQ ID NO: 168 - DNA, 127 bp, synthetic construct
  source 1..127: mol_type = other DNA
actataaatagaaactacaactgaggattagagtacacacttgctgagtcagcaaatctttagtctaagccacttagatcgccaaccatctgtccctgcaggtctagagggtatataatgggggcca

SEQ ID NO: 169 - DNA, 124 bp, synthetic construct
  source 1..124: mol_type = other DNA
ctaagccacattcgtctagtactaatcctctcggcagccgatctaatcctcagtctagcgctgacagatggttcacgggtggattaattggcctgcaggtctagagggtatataatgggggcca

SEQ ID NO: 170 - DNA, 126 bp, synthetic construct
  source 1..126: mol_type = other DNA
gacagatggtttgtcgagtcactaatcctcatccttagagaagaggattagttgaacagtatgctgactcagcaagtgtcgcaaccatctgtccctgcaggtctagagggtatataatgggggcca

SEQ ID NO: 171 - DNA, 128 bp, synthetic construct
  source 1..128: mol_type = other DNA
aaggtcatgaccttaaaaccatgctgactcagcatgcacgcattgtggcttagaattgcgcctatctaatcctctatcagacagaaccatctgtccctgcaggtctagagggtatataatgggggcca

SEQ ID NO: 172 - DNA, 429 bp, synthetic construct
  source 1..429: mol_type = other DNA
ggttacacaaccaggcggggaggggccccggggcggggagggggccggcccgcgggccgcgggactacgaagcccagaggtccctgcggctggggtgagtcctgctcctttgttcttcccatcggcccgcgcccattggctctccggcccgccgctcaccgcccctcctccgcaccgcccgggactacgaagcccagaggtccctgcggccggggcaccctgggacttgtagtccaagcctggggtgagtcctgctcctttgttcttcccgggactacgaagcccagaggtccctgcggctggggtgagtcctgctcctttgttcttcccgggactacgaagcccagaggtccctgcggcttttttcagccccctcccccccatcccccatggagccctgcaggtctagagggtatataatgggggcca

SEQ ID NO: 173 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggggtccctccctacctctgccccgcgctctgcctttgatcctctgctcggctctgagccatatcggcccgcgcccattggctctccggcccgccgctcaccgcccctcctccgcaccgccctggggtgagtcctgctcctttgttcttccccggggcaccctgggacttgtagtccaagccgcttgccacctgccggctgcaaacggcggagggactacgaagcccagaggtccctgcggcctcctgctattggtcagaatgtgtcacgtgaccatactcagctgcaagggttggtgggaattttttcagccccctcccccccatcccccatggagccctgcaggtctagagggtatataatgggggcca

SEQ ID NO: 174 - DNA, 483 bp, synthetic construct
  source 1..483: mol_type = other DNA
caggcggggaggggccccggggcggggagggggccggcccgcgggccgcgcagccggaagccgggaccgccaccggcccccggcgaggggctcctgctattggtcagaatgtgtcacgtgaccatactcagctgcaagggttggtgggaaatcggcccgcgcccattggctctccggcccgccgctcaccgcccctcctccgcaccgccctggggtgagtcctgctcctttgttcttccccggggcaccctgggacttgtagtccaagcctggggtgagtcctgctcctttgttcttcccgggactacgaagcccagaggtccctgcggcctcctgctattggtcagaatgtgtcacgtgaccatactcagctgcaagggttggtgggaaatgtagtctttttttctgggaggccatgtgttcggcttaaaattgtctttttttctttttcctgcaggtctagagggtatataatgggggcca

SEQ ID NO: 175 - DNA, 459 bp, synthetic construct
  source 1..459: mol_type = other DNA
caggcggggaggggccccggggcggggagggggccggcccgcgggccgcgcagccggaagccgggaccgccaccggcccccggcgaggggcggggcaccctgggacttgtagtccaagccgggactacgaagcccagaggtccctgcggcatcggcccgcgcccattggctctccggcccgccgctcaccgcccctcctccgcaccgcccgggactacgaagcccagaggtccctgcggccggggcaccctgggacttgtagtccaagccgcttgccacctgccggctgcaaacggcggagggactacgaagcccagaggtccctgcggcatcggcccgcgcccattggctctccggcccgccgctcaccgcccctcctccgcaccgcccttttttcagccccctcccccccatcccccatggagccctgcaggtctagagggtatataatgggggcca

SEQ ID NO: 176 - DNA, 489 bp, synthetic construct
  source 1..489: mol_type = other DNA
cggggcaccctgggacttgtagtccaagccgggactacgaagcccagaggtccctgcggcatcggcccgcgcccattggctctccggcccgccgctcaccgcccctcctccgcaccgccccggggcaccctgggacttgtagtccaagccgggactacgaagcccagaggtccctgcggcatcggcccgcgcccattggctctccggcccgccgctcaccgcccctcctccgcaccgccccggggcaccctgggacttgtagtccaagccgggactacgaagcccagaggtccctgcggcatcggcccgcgcccattggctctccggcccgccgctcaccgcccctcctccgcaccgccccggggcaccctgggacttgtagtccaagccgggactacgaagcccagaggtccctgcggcttttttcagccccctcccccccatcccccatggagccctgcaggtctagagggtatataatgggggcca

SEQ ID NO: 177 - DNA, 489 bp, synthetic construct
  source 1..489: mol_type = other DNA
ggttacacaaccaggcggggaggggccccggggcggggagggggccggcccgcgggccgcatcggcccgcgcccattggctctccggcccgccgctcaccgcccctcctccgcaccgcccgggactacgaagcccagaggtccctgcggcggtccctccctacctctgccccgcgctctgtggggtgagtcctgctcctttgttcttcccggttacacaaccaggcggggaggggccccggggcggggagggggccggcccgcgggccgcatcggcccgcgcccattggctctccggcccgccgctcaccgcccctcctccgcaccgcccgggactacgaagcccagaggtccctgcggcggtccctccctacctctgccccgcgctctgtggggtgagtcctgctcctttgttcttcccttttttcagccccctcccccccatcccccatggagccctgcaggtctagagggtatataatgggggcca

SEQ ID NO: 178 - DNA, 513 bp, synthetic construct
  source 1..513: mol_type = other DNA
ctcctgctattggtcagaatgtgtcacgtgaccatactcagctgcaagggttggtgggaaatgtagtctttttttctgggaggccatgtgttcggcttaaaattgtctttttttctttttggtccctccctacctctgccccgcgctctgtggggtgagtcctgctcctttgttcttcccctcctgctattggtcagaatgtgtcacgtgaccatactcagctgcaagggttggtgggaaatgtagtctttttttctgggaggccatgtgttcggcttaaaattgtctttttttctttttggtccctccctacctctgccccgcgctctgtggggtgagtcctgctcctttgttcttcccctcctgctattggtcagaatgtgtcacgtgaccatactcagctgcaagggttggtgggaaatgtagtctttttttctgggaggccatgtgttcggcttaaaattgtctttttttctttttcctgcaggtctagagggtatataatgggggcca

SEQ ID NO: 179 - DNA, 513 bp, synthetic construct
  source 1..513: mol_type = other DNA
cggggcaccctgggacttgtagtccaagccgggactacgaagcccagaggtccctgcggcatcggcccgcgcccattggctctccggcccgccgctcaccgcccctcctccgcaccgccccggggcaccctgggacttgtagtccaagccgggactacgaagcccagaggtccctgcggcatcggcccgcgcccattggctctccggcccgccgctcaccgcccctcctccgcaccgccccggggcaccctgggacttgtagtccaagccgggactacgaagcccagaggtccctgcggcctcctgctattggtcagaatgtgtcacgtgaccatactcagctgcaagggttggtgggaaatgtagtctttttttctgggaggccatgtgttcggcttaaaattgtctttttttctttttggtccctccctacctctgccccgcgctctgtggggtgagtcctgctcctttgttcttccccctgcaggtctagagggtatataatgggggcca

SEQ ID NO: 180 - DNA, 489 bp, synthetic construct
  source 1..489: mol_type = other DNA
tggggtgagtcctgctcctttgttcttcccgggactacgaagcccagaggtccctgcggcatcggcccgcgcccattggctctccggcccgccgctcaccgcccctcctccgcaccgccctggggtgagtcctgctcctttgttcttcccgggactacgaagcccagaggtccctgcggcatcggcccgcgcccattggctctccggcccgccgctcaccgcccctcctccgcaccgccctggggtgagtcctgctcctttgttcttcccgggactacgaagcccagaggtccctgcggcctcctgctattggtcagaatgtgtcacgtgaccatactcagctgcaagggttggtgggaagggactacgaagcccagaggtccctgcggctggggtgagtcctgctcctttgttcttcccttttttcagccccctcccccccatcccccatggagccctgcaggtctagagggtatataatgggggcca

SEQ ID NO: 181 - DNA, 500 bp, synthetic construct
  source 1..500: mol_type = other DNA
caggcggggaggggccccggggcggggagggggccggcccgcgggccgcgcagccggaagccgggaccgccaccggcccccggcgaggggagcccggctccaggccccgccccctggcggatcggcccgcgcccattggctctccggcccgccgctcaccgcccctcctccgcaccgccccggggcaccctgggacttgtagtccaagccgcttgccacctgccggctgcaaacggcggagggactacgaagcccagaggtccctgcggctccccctcctttccgaagcccccctccctgttttttcagccccctcccccccatcccccatggagcgacagatggttttgacgccttgtggcttagaggcacatcatgctgagtcagcaaccgacggctgaggattagtttgcgagtgtaccatctgtccctgcaggttcgcatattaaggtgacgcgtgtggcctcgaacaccgagcgaccctgcagcgacccgcttaa

SEQ ID NO: 182 - DNA, 500 bp, synthetic construct
  source 1..500: mol_type = other DNA
caggcggggaggggccccggggcggggagggggccggcccgcgggccgcgcagccggaagccgggaccgccaccggcccccggcgaggggagcccggctccaggccccgccccctggcggatcggcccgcgcccattggctctccggcccgccgctcaccgcccctcctccgcaccgccccggggcaccctgggacttgtagtccaagccgcttgccacctgccggctgcaaacggcggagggactacgaagcccagaggtccctgcggctccccctcctttccgaagcccccctccctgttttttcagccccctcccccccatcccccatggagctgctgagtcagcaagtatacgaactaagccacaggagtcttgtaccatctgtcaatcggcttaaccatctgtcaagtatctataccatctgtccctgcaggttcgcatattaaggtgacgcgtgtggcctcgaacaccgagcgaccctgcagcgacccgcttaa

SEQ ID NO: 183 - DNA, 462 bp, synthetic construct
  source 1..462: mol_type = other DNA
caggcggggaggggccccggggcggggagggggccggcccgcgggccgcgcagccggaagccgggaccgccaccggcccccggcgaggggagcccggctccaggccccgccccctggcggatcggcccgcgcccattggctctccggcccgccgctcaccgcccctcctccgcaccgccccggggcaccctgggacttgtagtccaagccgcttgccacctgccggctgcaaacggcggagggactacgaagcccagaggtccctgcggctccccctcctttccgaagcccccctccctgttttttcagccccctcccccccatcccccatggagcctaagccacagtaacatgcgactaatcctctctgtctacagagtggcttagtctatcgtgttgctgactcagcattagatagtgacagatggtcctgcaggtctagagggtatataatgggggcca

SEQ ID NO: 184 - DNA, 491 bp, synthetic construct
  source 1..491: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcgacagatggttttgacgccttgtggcttagaggcacatcatgctgagtcagcaaccgacggctgaggattagtttgcgagtgtaccatctgtccctgcaggttcgcatattaaggtgacgcgtgtggcctcgaacaccgagcgaccctgcagcgacccgcttaa

SEQ ID NO: 185 - DNA, 491 bp, synthetic construct
  source 1..491: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctctgctgagtcagcaagtatacgaactaagccacaggagtcttgtaccatctgtcaatcggcttaaccatctgtcaagtatctataccatctgtccctgcaggttcgcatattaaggtgacgcgtgtggcctcgaacaccgagcgaccctgcagcgacccgcttaa

SEQ ID NO: 186 - DNA, 453 bp, synthetic construct
  source 1..453: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcctaagccacagtaacatgcgactaatcctctctgtctacagagtggcttagtctatcgtgttgctgactcagcattagatagtgacagatggtcctgcaggtctagagggtatataatgggggcca

SEQ ID NO: 187 - DNA, 360 bp, synthetic construct
  source 1..360: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 188 - DNA, 450 bp, synthetic construct; identical to SEQ ID NO: 157
  source 1..450: mol_type = other DNA

SEQ ID NO: 189 - DNA, 372 bp, synthetic construct
  source 1..372: mol_type = other DNA
gacagatggttttgacgccttgtggcttagaggcacatcatgctgagtcagcaaccgacggctgaggattagtttgcgagtgtaccatctgtctgctgagtcagcaagtatacgaactaagccacaggagtcttgtaccatctgtcaatcggcttaaccatctgtcaagtatctataccatctgtcctcctgctattggtcagaatgtgtcacgtgaccatactcagctgcaagggttggtgggaactaagccacagtaacatgcgactaatcctctctgtctacagagtggcttagtctatcgtgttgctgactcagcattagatagtgacagatggtcctgcaggtctagagggtatataatgggggcca

SEQ ID NO: 190 - DNA, 399 bp, synthetic construct
  source 1..399: mol_type = other DNA
gacagatggttttgacgccttgtggcttagaggcacatcatgctgagtcagcaaccgacggctgaggattagtttgcgagtgtaccatctgtctacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacagtaacatgcgactaatcctctctgtctacagagtggcttagtctatcgtgttgctgactcagcattagatagtgacagatggtcctgcaggtctagagggtatataatgggggcca

SEQ ID NO: 191 - DNA, 399 bp, synthetic construct
  source 1..399: mol_type = other DNA
tgctgagtcagcaagtatacgaactaagccacaggagtcttgtaccatctgtcaatcggcttaaccatctgtcaagtatctataccatctgtctacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacagtaacatgcgactaatcctctctgtctacagagtggcttagtctatcgtgttgctgactcagcattagatagtgacagatggtcctgcaggtctagagggtatataatgggggcca

SEQ ID NO: 192 - DNA, 435 bp, synthetic construct
  source 1..435: mol_type = other DNA
ctaagccacagtaacatgcgactaatcctctctgtctacagagtggcttagtctatcgtgttgctgactcagcattagatagtgacagatggtttacctactaggttaattcgtccgatagatactacgaattgcgagcttctaagtccaattttcggtattcgagtcgtcagactcaattattacacggttaagactcaatccggtaagtaatactgtgatcaggtgtctacaagactagtactcctgctattggtcagaatgtgtcacgtgaccatactcagctgcaagggttggtgggaaatgtagtctttttttctgggaggccatgtgttcggcttaaaattgtctttttttctttttactatcacgtatataccagcgagttcgataatacactctcctgcaggtctagagggtatataatgggggcca

SEQ ID NO: 193 - DNA, 395 bp, synthetic construct
  source 1..395: mol_type = other DNA
ctcaggccctgcattgcgccaacggcgcagcgctgggccgcgaccccggcaccggcacccggggacgttgtgaatagctggtcgaagtttggttgaagcacaggagggtaaataagaaggtttccagacaaagagactcttaaaggtacaggccagagacgctcccgagcccatctcctcctgctattggtcagaatgtgtcacgtgaccatactcagctgcaagggttggtgggaaatgtagtctttttttctgggaggccatgtgttcggcttaaaattgtctttttttctttttctttatttcttttttcttttctttctttttatacactctcctgcaggagacgctagcggggggctataaaagggggtgggggcgttcgtcctcactct

SEQ ID NO: 194 - DNA, 386 bp, synthetic construct
  source 1..386: mol_type = other DNA
ctcaggccctgcattgcgccaacggcgcagcgctgggccgcgaccccggcaccggcacccctcctgctattggtcagaatgtgtcacgtgaccatactcagctgcaagggttggtgggaaatgtagtctttttttctgggaggccatgtgttcggcttaaaattgtctttttttctttttggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 195 - DNA, 449 bp, synthetic construct
  source 1..449: mol_type = other DNA
ctaagccacagtaacatgcgactaatcctctctgtctacagagtggcttagtctatcgtgttgctgactcagcattagatagtgacagatggtctcaggccctgcattgcgccaacggcgcagcgctgggccgcgaccccggcaccggcacccgttccgagggttcgcgccgcaggcgcaaagccacattgctatagtgctgtatagcgattattgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 196 - DNA, 446 bp, synthetic construct
  source 1..446: mol_type = other DNA
agcacgcgcgagctgtcaccagggcgcgagactacgaattgcgagcttctaagtccaatttctattggcccgcccgctggagccccgcccctcaggccctgcattgcgccaacggcgcagcgctgggccgcgaccccggcaccggcacccgttccgagggttcgcgccgcaggcgcaaagtttagggtcagccacaaacgcgcggccgctttgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 197 - DNA, 500 bp, synthetic construct
  source 1..500: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacgcaaggaagattattttgagtcactgcaccctttctgaacaatagctgttaccaaggtctcaggagcattaggcgctcaattctattttaagtagcatggttggagcttaaccccaagtctaattttattctgcctctttatttaaaaaggacagatggttcccaacgactaatcctcatagaacgcactaatcctccctgcaggtctagagggtatataatgggggcca

SEQ ID NO: 198 - DNA, 500 bp, synthetic construct
  source 1..500: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacgcaaggaagattattttgagtcactgcaccctttctgaacaatagctgttaccaaggtctcaggagcattaggcgctcaattctattttaagtagcatggttggagcttaaccccaagtctaattttattctgcctctttatttaaaaagctaatcctctcggcccgatctaatcctcagtctcgctgacagatggtcctgcaggtctagagggtatataatgggggcca

SEQ ID NO: 199 - DNA, 500 bp, synthetic construct
  source 1..500: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacgacagatggttcccaacgactaatcctcatagaacgcactaatcctcgcaaggaagattattttgagtcactgcaccctttctgaacaatagctgttaccaaggtctcaggagcattaggcgctcaattctattttaagtagcatggttggagcttaaccccaagtctaattttattctgcctctttatttaaaaagcctgcaggtctagagggtatataatgggggcca

SEQ ID NO: 200 - DNA, 500 bp, synthetic construct
  source 1..500: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacctaatcctctcggcccgatctaatcctcagtctcgctgacagatggtgcaaggaagattattttgagtcactgcaccctttctgaacaatagctgttaccaaggtctcaggagcattaggcgctcaattctattttaagtagcatggttggagcttaaccccaagtctaattttattctgcctctttatttaaaaagcctgcaggtctagagggtatataatgggggcca

SEQ ID NO: 201 - DNA, 500 bp, synthetic construct
  source 1..500: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacagcgctgacagatggttcacgggcaaggaagattattttgagtcactgcaccctttctgaacaatagctgttaccaaggtctcaggagcattaggcgctcaattctattttaagtagcatggttggagcttaaccccaagtctaattttattctgcctctttatttaaaaaggggccagacagatggttcccaataccctgcaggtctagagggtatataatgggggcca

SEQ ID NO: 202 - DNA, 488 bp, synthetic construct
  source 1..488: mol_type = other DNA
ggttacacaaccaggcggggaggggccccggggcggggagggggccggcccgcgggccgcgcagccggaagccgggaccgccaccggccctacgggccagacagatggttcccaatacgactaatcctcatagaagacgcactaatcctccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggttcgcatattaaggtgacgcgtgtggcctcgaacaccgagcgaccctgcagcgacccgcttaa

SEQ ID NO: 203 - DNA, 464 bp, synthetic construct
  source 1..464: mol_type = other DNA
ggttacacaaccaggcggggaggggccccggggcggggagggggccggcccgcgggccgcgcagccggaagccgggaccgccaccggcccattcgtctagtactaatcctctcggcagccgatctaatcctcagtctagcgctgacagatggttcacgggtggacgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 204 - DNA, 498 bp, synthetic construct
  source 1..498: mol_type = other DNA
tacgggccagacagatggttcccaatacgactaatcctcatagaagacgcactaatcctcgtgagtgacaggcgaaccagtactataaatagatacgggccagacagatggttcccaatacgactaatcctcatagaagacgcactaatcctcactgtcgagttgtgagtgacaggcgaaccagtactataaatagatacgggccagacagatggttcccaatacgactaatcctcatagaagacgcactaatcctcactgtcgagttgtgagtgacaggcgaaccagtactataaatagatacgggccagacagatggttcccaatacgactaatcctcatagaagacgcactaatcctcactgtcgagttgtgagtgacaggcgaaccagtactataaatagatacgggccagacagatggttcccaatacgactaatcctcatagaagacgcactaatcctccctgcaggtctagagggtatataatgggggcca

SEQ ID NO: 205 - DNA, 490 bp, synthetic construct
  source 1..490: mol_type = other DNA
tagtactaatcctctcggcagccgatctaatcctcagtctagcgctgacagatggttcacctaagccacattcgtctagtactaatcctctcggcagccgatctaatcctcagtctagcgctgacagatggttcacgggtggattaattggtgttccgaacactaagccacattcgtctagtactaatcctctcggcagccgatctaatcctcagtctagcgctgacagatggttcacgggtggattaattggtgttccgaacactaagccacattcgtctagtactaatcctctcggcagccgatctaatcctcagtctagcgctgacagatggttcacgggtggattaattggtgttccgaacactaagccacattcgtctagtactaatcctctcggcagccgatctaatcctcagtctagcgctgacagatggttcacgggtggattaattggcctgcaggtctagagggtatataatgggggcca

SEQ ID NO: 206 - DNA, 400 bp, synthetic construct
  source 1..400: mol_type = other DNA
gtgagtgacaggcgaccagtactataaatagatacgggccagacagatggttcccaatacgactaatcctcatagaagacgcactaatcctcctaagccacattcgtctagtactaatcctctcggcagccgatctaatcctcagtctagcgctgacagatggttcacgggtggattaattgggtgagtgacaggcgaaccagtactataaatagatacgggccagacagatggttcccaatacgactaatcctcatagaagacgcactaatcctcctaagccacattcgtctagtactaatcctctcggcagccgatctaatcctcagtctagcgctgacagatggttcacgggtggattaattggcctgcaggtctagagggtatataatgggggcca

SEQ ID NO: 207 - DNA, 397 bp, synthetic construct
  source 1..397: mol_type = other DNA
ctaagccacattcgtctagtactaatcctctcggcagccgatctaatcctcagtctagcgctgacagatggttcacgggtggattaattggctaagccacattcgtctagtactaatcctctcggcagccgatctaatcctcagtctagcgctgacagatggttcacgggtggattaattggctaagccacattcgtctagtactaatcctctcggcagccgatctaatcctcagtctagcgctgacagatggttcacgggtggattaattggctaagccacattcgtctagtactaatcctctcggcagccgatctaatcctcagtctagcgctgacagatggttcacgggtggattaattggcctgcaggtctagagggtatataatgggggcca

SEQ ID NO: 208 - DNA, 400 bp, synthetic construct
  source 1..400: mol_type = other DNA
ctaagccacagtaacatgcgactaatcctctctgtctacagagtggcttagtctatcgtgttgctgactcagcattagatagtgacagatggttagtaaggtactaagccacattcgtctagtactaatcctctcggcagccgatctaatcctcagtctagcgctgacagatggttcacgggtggattaattggggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 209 - DNA, 400 bp, synthetic construct
  source 1..400: mol_type = other DNA
gtgagtgacaggcgaaccagtactataaatagatacgggccagacagatggttcccaatacgactaatcctcatagaagacgcactaatcctctagtaaggtactaagccacattcgtctagtactaatcctctcggcagccgatctaatcctcagtctagcgctgacagatggttcacgggtggattaattggggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 210 - DNA, 398 bp, synthetic construct
  source 1..398: mol_type = other DNA
ctaagccacattcgtctagtactaatcctctcggcagccgatctaatcctcagtctagcgctgacagatggttcacgggtggattaattggtagtaaggtactaagccacattcgtctagtactaatcctctcggcagccgatctaatcctcagtctagcgctgacagatggttcacgggtggattaattggggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 211 - DNA, 447 bp, synthetic construct
  source 1..447: mol_type = other DNA
ctaagccacattcgtctagtactaatcctctcggcagccgatctaatcctcagtctagcgctgacagatggttcacgggtggattaattggctcaggccctgcattgcgccaacggcgcagcgctgggccgcgaccccggcaccggcacccgttccgagggttcgcgccgcaggcgcaaagccacattgctatagtgctgtatagcgattattgagcctggcgtaaggggcggcggcgtagggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctccctgcaggagacgctagcggggggctataaaagggggtgggggcgttcgtcctcactct

SEQ ID NO: 212 - DNA, 491 bp, synthetic construct
  source 1..491: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcgtgagtgacaggcgaaccagtactataaatagatacgggccagacagatggttcccaatacgactaatcctcatagaagacgcactaatcctccctgcaggttcgcatattaaggtgacgcgtgtggcctcgaacaccgagcgaccctgcagcgacccgcttaa

SEQ ID NO: 213 - DNA, 489 bp, synthetic construct
  source 1..489: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcctaagccacattcgtctagtactaatcctctcggcagccgatctaatcctcagtctagcgctgacagatggttcacgggtggattaattggcctgcaggttcgcatattaaggtgacgcgtgtggcctcgaacaccgagcgaccctgcagcgacccgcttaa

SEQ ID NO: 214 - DNA, 483 bp, synthetic construct
  source 1..483: mol_type = other DNA
ggttacacaaccaggcggggaggggccccggggcggggagggggccggcccgcgggccgcgcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcgtgagtgacaggcgaaccagtactataaatagatacgggccagacagatggttcccaatacgactaatcctcatagaagacgcactaatcctccctgcaggtctagagggtatataatgggggcca

SEQ ID NO: 215 - DNA, 481 bp, synthetic construct
  source 1..481: mol_type = other DNA
ggttacacaaccaggcggggaggggccccggggcggggagggggccggcccgcgggccgcgcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcctaagccacattcgtctagtactaatcctctcggcagccgatctaatcctcagtctagcgctgacagatggttcacgggtggattaattggcctgcaggtctagagggtatataatgggggcca

SEQ ID NO: 216 - DNA, 489 bp, synthetic construct
  source 1..489: mol_type = other DNA
ggttacacaaccaggcggggaggggccccggcagccggaagccgggaccgccaccggcccctaagccacattcgtctagtactaatcctctcggcagccgatctaatcctcagtctagcgctgacagatggttcacgggtggattaattggcgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggttcgcatattaaggtgacgcgtgtggcctcgaacaccgagcgaccctgcagcgacccgcttaa

SEQ ID NO: 217 - DNA, 483 bp, synthetic construct
  source 1..483: mol_type = other DNA
ggttacacaaccaggcggggaggggccccggggcggggagggggccggcccgcgggccgcgcagccggaagccgggaccgccaccggcccgtgagtgacaggcgaaccagtactataaatagatacgggccagacagatggttcccaatacgactaatcctcatagaagacgcactaatcctccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 218 - DNA, 481 bp, synthetic construct
  source 1..481: mol_type = other DNA
ggttacacaaccaggcggggaggggccccggggcggggagggggccggcccgcgggccgcgcagccggaagccgggaccgccaccggcccctaagccacattcgtctagtactaatcctctcggcagccgatctaatcctcagtctagcgctgacagatggttcacgggtggattaattggcgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctccctgcaggtctagagggtatataatgggggcca

SEQ ID NO: 219 - DNA, 486 bp, synthetic construct
  source 1..486: mol_type = other DNA
ctcctgctattggtcagaatgtgtcacgtgaccatactcagctgcaagggttggtgggaactaagccacattcgtctagtactaatcctctcggcagccgatctaatcctcagtctagcgctgacagatggttcacgggtggattaattggctcctgctattggtcagaatgtgtcacgtgaccatactcagctgcaagggttggtgggaactaagccacattcgtctagtactaatcctctcggcagccgatctaatcctcagtctagcgctgacagatggttcacgggtggattaattggctcctgctattggtcagaatgtgtcacgtgaccatactcagctgcaagggttggtgggaactaagccacattcgtctagtactaatcctctcggcagccgatctaatcctcagtctagcgctgacagatggttcacgggtggattaattggcctgcaggtctagagggtatataatgggggcca

SEQ ID NO: 220 - DNA, 500 bp, synthetic construct
  source 1..500: mol_type = other DNA
ctaatcctctcggccgatctaatcctcagtctgctgacagatggtctcctgctattggtcagaatgtgtcacgtgaccatactcagctgcaagggttggtgggaaatgtagtctttttttctgggaggccatgtgttcggcttaaaattgtctttttttctttttctaagccacattcgtctagtactaatcctctcggcagccgatctaatcctcagtctagcgctgacagatggttcacgggtggattaattggctcctgctattggtcagaatgtgtcacgtgaccatactcagctgcaagggttggtgggaaatgtagtctttttttctgggaggccatgtgttcggcttaaaattgtctttttttctttttctaagccacattcgtctagtactaatcctctcggcagccgatctaatcctcagtctagcgctgacagatggttcacgggtggattaattggcctgcaggtctagagggtatataatgggggcca

SEQ ID NO: 221 - DNA, 397 bp, synthetic construct
  source 1..397: mol_type = other DNA
ctaagccacagtaacatgcgactaatcctctctgtctacagagtggcttagtctatcgtgttgctgactcagcattagatagtgacagatggttacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacattcgtctagtactaatcctctcggcagccgatctaatcctcagtctagcgctgacagatggttcacgggtggattaattggcctgcaggtctagagggtatataatgggggcca

SEQ ID NO: 222 - DNA, 397 bp, synthetic construct
  source 1..397: mol_type = other DNA
gtgagtgacaggcgaaccagtactataaatagatacgggccagacagatggttcccaatacgactaatcctcatagaagacgcactaatcctctacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacattcgtctagtactaatcctctcggcagccgatctaatcctcagtctagcgctgacagatggttcacgggtggattaattggcctgcaggtctagagggtatataatgggggcca

SEQ ID NO: 223 - DNA, 487 bp, synthetic construct
  source 1..487: mol_type = other DNA
ctaagccacagtaacatgcgactaatcctctctgtctacagagtggcttagtctatcgtgttgctgactcagcattagatagtgacagatggttgttgagagctcaagctctttttaacgctttgccttgctgtctccaaagtattgccttcatcctcatagttcaaagtgtccaccatcacatacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacattcgtctagtactaatcctctcggcagccgatctaatcctcagtctagcgctgacagatggttcacgggtggattaattggcctgcaggtctagagggtatataatgggggcca

SEQ ID NO: 224 - DNA, 433 bp, synthetic construct
  source 1..433: mol_type = other DNA
ctaagccacattcgtctagtactaatcctctcggcagccgatctaatcctcagtctagcgctgacagatggttcacgggtggattaattggttacctactaggttaattcgtccgatagatactacgaattgcgagcttctaagtccaattttcggtattcgagtcgtcagactcaattattacacggttaagactcaatccggtaagtaatactgtgatcaggtgtctacaagactagtactcctgctattggtcagaatgtgtcacgtgaccatactcagctgcaagggttggtgggaaatgtagtctttttttctgggaggccatgtgttcggcttaaaattgtctttttttctttttactatcacgtatataccagcgagttcgataatacactctcctgcaggtctagagggtatataatgggggcca

SEQ ID NO: 225 - DNA, 433 bp, synthetic construct
  source 1..433: mol_type = other DNA
ctaagccacattcgtctagtactaatcctctcggcagccgatctaatcctcagtctagcgctgacagatggttcacgggtggattaattggtgttgagagctcaagctctttttaacgctttgccttgctgtctccaaagtattgccttcatcctcatagttcaaagtgtccaccatcacatacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttctttttatacactctcctgcaggtctagagggtatataatgggggcca

SEQ ID NO: 226 - DNA, 433 bp, synthetic construct
  source 1..433: mol_type = other DNA
ctaagccacattcgtctagtactaatcctctcggcagccgatctaatcctcagtctagcgctgacagatggttcacgggtggattaattggtgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacggcctgcaggtctagagggtatataatgggggcca

SEQ ID NO: 227 - DNA, 396 bp, synthetic construct
  source 1..396: mol_type = other DNA
tagtactaatcctctcggcagccgatctaatcctcagtctagcgctgacagatggttcactagtaaggtactaagccacattcgtctagtactaatcctctcggcagccgatctaatcctcagtctagcgctgacagatggttcacgggtggattaattggtttgcgcgtactaagccacattcgtctagtactaatcctctcggcagccgatctaatcctcagtctagcgctgacagatggttcacgggtggattaattggagtccgggtactaagccacattcgtctagtactaatcctctcggcagccgatctaatcctcagtctagcgctgacagatggttcacgggtggattaattggcctgcaggtctagagggtatataatgggggcca

SEQ ID NO: 228 - DNA, 400 bp, synthetic construct
  source 1..400: mol_type = other DNA
ctaagccacattcgtctagtactaatcctctcggcagccgatctaatcctcagtctagcgctgacagatggttcacgggtggattaattgggtgagtgacaggcgaccagtactataaatagatacgggccagacagatggttcccaatacgactaatcctcatagaagacgcactaatcctcctaagccacattcgtctagtactaatcctctcggcagccgatctaatcctcagtctagcgctgacagatggttcacgggtggattaattgggtgagtgacaggcgaaccagtactataaatagatacgggccagacagatggttcccaatacgactaatcctcatagaagacgcactaatcctccctgcaggtctagagggtatataatgggggcca

SEQ ID NO: 229 - DNA, 400 bp, synthetic construct
  source 1..400: mol_type = other DNA
gtgagtgacaggcgaccagtactataaatagatacgggccagacagatggttcccaatacgactaatcctcatagaagacgcactaatcctcgtgagtgacaggcgaaccagtactataaatagatacgggccagacagatggttcccaatacgactaatcctcatagaagacgcactaatcctcctaagccacattcgtctagtactaatcctctcggcagccgatctaatcctcagtctagcgctgacagatggttcacgggtggattaattggctaagccacattcgtctagtactaatcctctcggcagccgatctaatcctcagtctagcgctgacagatggttcacgggtggattaattggcctgcaggtctagagggtatataatgggggcca

SEQ ID NO: 230 - DNA, 397 bp, synthetic construct
  source 1..397: mol_type = other DNA
ctaagccacattcgtctagtactaatcctctcggcagccgatctaatcctcagtctagcgctgacagatggttcacgggtggattaattggtgctgagtcagcataggaagagtctaagccacagcacgcgtagtctaatcctcagcctacttaactataaatagatcgagcaataccatctgtctgctgactcagcaacgactataagtggcttagaacataacgtctctaatcctcagactgcgcgtgacagatggtttcgtcccgtaccatctgtctgctgagtcagcaagtatacgaactaagccacaggagtcttgtaccatctgtcaatcggcttaaccatctgtcaagtatctataccatctgtctctagagggtatataatgggggcca

SEQ ID NO: 231 - DNA, 394 bp, synthetic construct
  source 1..394: mol_type = other DNA
accatctgtcaatcggcttaaccatctgtcaagtatctataccatctgtcagtccgggtatgctgagtcagcataggaagagtctaagccacagcacgcgtagtctaatcctcagcctacttaactataaatagatcgagcaataccatctgtctttgcgcgtatgctgactcagcaacgactataagtggcttagaacataacgtctctaatcctcagactgcgcgtgacagatggtttcgtcccgtaccatctgtctagtaaggtatgctgagtcagcaagtatacgaactaagccacaggagtcttgtaccatctgtcaatcggcttaaccatctgtcaagtatctataccatctgtccctgcaggtctagagggtatataatgggggcca

SEQ ID NO: 232 - DNA, 392 bp, synthetic construct
  source 1..392: mol_type = other DNA
accatctgtcaatcggcttaaccatctgtcaagtatctataccatctgtcagtccgggtatgctgagtcagcaagtatacgaactaagccacaggagtcttgtaccatctgtcaatcggcttaaccatctgtcaagtatctataccatctgtctttgcgcgtatgctgagtcagcaagtatacgaactaagccacaggagtcttgtaccatctgtcaatcggcttaaccatctgtcaagtatctataccatctgtctagtaaggtatgctgagtcagcaagtatacgaactaagccacaggagtcttgtaccatctgtcaatcggcttaaccatctgtcaagtatctataccatctgtccctgcaggtctagagggtatataatgggggcca

SEQ ID NO: 233 - DNA, 397 bp, synthetic construct
  source 1..397: mol_type = other DNA
tgctgagtcagcaagtatacgaactaagccacaggagtcttgtaccatctgtcaatcggcttaaccatctgtcaagtatctataccatctgtctgctgagtcagcaagtatacgaactaagccacaggagtcttgtaccatctgtcaatcggcttaaccatctgtcaagtatctataccatctgtctgctgagtcagcaagtatacgaactaagccacaggagtcttgtaccatctgtcaatcggcttaaccatctgtcaagtatctataccatctgtctgctgagtcagcaagtatacgaactaagccacaggagtcttgtaccatctgtcaatcggcttaaccatctgtcaagtatctataccatctgtctctagagggtatataatgggggcca

SEQ ID NO: 234 - DNA, 479 bp, synthetic construct
  source 1..479: mol_type = other DNA
tgctgagtcagcaagtatacgaactaagccacaggagtcttgtaccatctgtcaatcggcttaaccatctgtcaagtatctataccatctgtcctcaggccctgcattgcgccaacggcgcagcgctgggccgcgaccccggcaccggcacccggggacgttgtgaatagctggtcgaagtttggttgaagcacaggagggtaaataagaaggtttccagacaaagagactcttaaaggtacagactcttatgtctgtccctcctccttaaagggccagagacgctcccgagcccatctcctcctgctattggtcagaatgtgtcacgtgaccatactcagctgcaagggttggtgggaaatgtagtctttttttctgggaggccatgtgttcggcttaaaattgtctttttttctttttcctgcaggagacgctagcggggggctataaaagggggtgggggcgttcgtcctcactct

SEQ ID NO: 235 - DNA, 500 bp, synthetic construct
  source 1..500: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacccccctccctgttttttcagccccctctgctgagtcagcaagtatacgaactaagccacaggagtcttgtaccatctgtcaatcggcttaaccatctgtcaagtatctataccatctgtctcgaggcgccggcggggcggcccggcggccggaagtgcaccctgctcgggctttgcgcccccggcagcttcctccgcccgcgaggtctagagggtatataatgggggcca

SEQ ID NO: 236 - DNA, 500 bp, synthetic construct
  source 1..500: mol_type = other DNA
tgctgagtcagcaagtatacgaactaagccacaggagtcttgtaccatctgtcaatcggcttaaccatctgtcaagtatctataccatctgtcggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacccccctccctgttttttcagccccctctcgaggcgccggcggggcggcccggcggccggaagtgcaccctgctcgggctttgcgcccccggcagcttcctccgcccgcgaggtctagagggtatataatgggggcca

SEQ ID NO: 237 - DNA, 453 bp, synthetic construct
  source 1..453: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacccccctccctgttttttcagccccctctcgaggcgccggcggggcggcccggcggccggaagtgcaccctgctcgggctttgcgcccccggcagcttcctccgcccgcgaggcctgcaggttcgcatattaaggtgacgcgtgtggcctcgaacaccgagcgaccctgcagcgacccgcttaa

SEQ ID NO: 238 - DNA, 415 bp, synthetic construct
  source 1..415: mol_type = other DNA
ggttacacaaccaggcggggaggggccccggcagccggaagccgggaccgccaccggccccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacccccctccctgttttttcagccccctctcgaggcgccggcggggcggcccggcggccggaagtgcaccctgctcgggctttgcgcccccggcagcttcctccgcccgcgaggcctgcaggtctagagggtatataatgggggcca

SEQ ID NO: 239 - DNA, 339 bp, synthetic construct
  source 1..339: mol_type = other DNA
tgctgagtcagcaagtatacgaactaagccacaggagtcttgtaccatctgtcaatcggcttaaccatctgtcaagtatctataccatctgtcctcctgctattggtcagaatgtgtcacgtgaccatactcagctgcaagggttggtgggaaatgtagtctttttttctgggaggccatgtgttcggcttaaaattgtctttttttctttttctaagccacagtaacatgcgactaatcctctctgtctacagagtggcttagtctatcgtgttgctgactcagcattagatagtgacagatggtcctgcaggtctagagggtatataatgggggcca

SEQ ID NO: 240 - DNA, 425 bp, synthetic construct
  source 1..425: mol_type = other DNA
tgctgagtcagcaagtatacgaactaagccacaggagtcttgtaccatctgtcaatcggcttaaccatctgtcaagtatctataccatctgtctacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacagtaacatgcgactaatcctctctgtctacagagtggcttagtctatcgtgttgctgactcagcattagatagtgacagatggtcctgcaggagacgctagcggggggctataaaagggggtgggggcgttcgtcctcactct

SEQ ID NO: 241 - DNA, 146 bp, synthetic construct
  source 1..146: mol_type = other DNA
accatctgtctagtaaggtatgctgagtcagcaagtatacgaactaagccacaggagtcttgtaccatctgtcaatcggcttaaccatctgtcaagtatctataccatctgtccctgcaggtctagagggtatataatgggggcca

SEQ ID NO: 242 - DNA, 166 bp, synthetic construct
  source 1..166: mol_type = other DNA
accatctgtcaagtatctataccatctgtctagtaaggtatgctgagtcagcaagtatacgaactaagccacaggagtcttgtaccatctgtcaatcggcttaaccatctgtcaagtatctataccatctgtccctgcaggtctagagggtatataatgggggcca

SEQ ID NO: 243 - DNA, 186 bp, synthetic construct
  source 1..186: mol_type = other DNA
accatctgtcaatcggcttaaccatctgtcaagtatctataccatctgtctagtaaggtatgctgagtcagcaagtatacgaactaagccacaggagtcttgtaccatctgtcaatcggcttaaccatctgtcaagtatctataccatctgtccctgcaggtctagagggtatataatgggggcca

SEQ ID NO: 244 - DNA, 166 bp, synthetic construct
  source 1..166: mol_type = other DNA
tgctgagtcagcaagtatacgaactaagccacaggagtcttgtaccatctgtcaatcggcttaaccatctgtcaagtatctataccatctgtccctgcaggtaggcgtgtacggtgggaggcctatataagcagagctcgtttagtgaaccgtcagatcgcctgga

SEQ ID NO: 245 - DNA, 152 bp, synthetic construct
  source 1..152: mol_type = other DNA
tgctgagtcagcaagtatacgaactaagccacaggagtcttgtaccatctgtcaatcggcttaaccatctgtcaagtatctataccatctgtccctgcaggagacgctagcggggggctataaaagggggtgggggcgttcgtcctcactct

SEQ ID NO: 246 - DNA, 261 bp, synthetic construct
  source 1..261: mol_type = other DNA
tgctgagtcagcaagtatacgaactaagccacaggagtcttgtaccatctgtcaatcggcttaaccatctgtcaagtatctataccatctgtccctgcaggaaaatgtgcgcatgtgcagccattgcctgggacgcatgcgtagggagccgcgcgacaaactgagccattgcggcaagactagcgcagagaggagagggagccggagatgccagacgcttggttctgaggagtgatttgcaacgcaatggagcgaggaagg

SEQ ID NO: 247 - DNA, 153 bp, synthetic construct
  source 1..153: mol_type = other DNA
tgctgagtcagcaagtatacgaactaagccacaggagtcttgtaccatctgtcaatcggcttaaccatctgtcaagtatctataccatctgtccctgcaggcagaattaacagtataaattgcatctcttgttcaagagttccctatcactct

SEQ ID NO: 248 - DNA, 279 bp, synthetic construct
  source 1..279: mol_type = other DNA
tgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacgg

SEQ ID NO: 249 - DNA, 30 bp, synthetic construct; identical to SEQ ID NO: 5
  source 1..30: mol_type = other DNA

SEQ ID NO: 250 - DNA, 249 bp, synthetic construct
  source 1..249: mol_type = other DNA
tcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacgg

SEQ ID NO: 251 - DNA, 60 bp, synthetic construct
  source 1..60: mol_type = other DNA
tgttgagagctcaagctctttttaacgctttgccttgctgtctccaaagtattgccttca

SEQ ID NO: 252 - DNA, 219 bp, synthetic construct
  source 1..219: mol_type = other DNA
ttcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacgg

SEQ ID NO: 253 - DNA, 90 bp, synthetic construct
  source 1..90: mol_type = other DNA
tgttgagagctcaagctctttttaacgctttgccttgctgtctccaaagtattgccttcatcctcatagttcaaagtgtccaccatcaca

SEQ ID NO: 254 - DNA, 189 bp, synthetic construct
  source 1..189: mol_type = other DNA
gagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacgg

SEQ ID NO: 255 - DNA, 120 bp, synthetic construct
  source 1..120: mol_type = other DNA
tgttgagagctcaagctctttttaacgctttgccttgctgtctccaaagtattgccttcatcctcatagttcaaagtgtccaccatcacattcacgtttcagccaataggaagaaggaaa

SEQ ID NO: 256 - DNA, 159 bp, synthetic construct
  source 1..159: mol_type = other DNA
ctcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttttttagacgg

SEQ ID NO: 257 - DNA, 270 bp, synthetic construct
  source 1..270: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttcttttt

SEQ ID NO: 258 - skipped

SEQ ID NO: 259 - DNA, 300 bp, synthetic construct
  source 1..300: mol_type = other DNA
tgttgagagctcaagctctttttaacgctttgccttgctgtctccaaagtattgccttcatcctcatagttcaaagtgtccaccatcacattcacgtttcagccaataggaagaaggaaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctttatttcttttttcttttctttcttttt

SEQ ID NO: 260 - DNA, 90 bp, synthetic construct
  source 1..90: mol_type = other DNA
ggttacacaaccaggcggggaggggccccggggcggggagggggccggcccgcgggccgcgcagccggaagccgggaccgccaccggccc

SEQ ID NO: 261 - DNA, 297 bp, synthetic construct
  source 1..297: mol_type = other DNA
ccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctc

SEQ ID NO: 262 - DNA, 120 bp, synthetic construct
  source 1..120: mol_type = other DNA
ggttacacaaccaggcggggaggggccccggggcggggagggggccggcccgcgggccgcgcagccggaagccgggaccgccaccggcccccggcgaggggagcccggctccaggccccg

SEQ ID NO: 263 - DNA, 267 bp, synthetic construct
  source 1..267: mol_type = other DNA
cgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctc

SEQ ID NO: 264 - DNA, 90 bp, synthetic construct
  source 1..90: mol_type = other DNA
caggcggggaggggccccggggcggggagggggccggcccgcgggccgcgcagccggaagccgggaccgccaccggcccccggcgagggg

SEQ ID NO: 265 - DNA, 306 bp, synthetic construct
  source 1..306: mol_type = other DNA
gctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagc

SEQ ID NO: 266 - DNA, 120 bp, synthetic construct
  source 1..120: mol_type = other DNA
caggcggggaggggccccggggcggggagggggccggcccgcgggccgcgcagccggaagccgggaccgccaccggcccccggcgaggggagcccggctccaggccccgccccctggcgg

SEQ ID NO: 267 - DNA, 276 bp, synthetic construct
  source 1..276: mol_type = other DNA
atcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagc

SEQ ID NO: 268 - DNA, 210 bp, synthetic construct
  source 1..210: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgccc

SEQ ID NO: 269 - DNA, 186 bp, synthetic construct
  source 1..186: mol_type = other DNA
cggggcaccctgggacttgtagtccaagccgcttgccacctgccggctgcaaacggcggagggactacgaagcccagaggtccctgcggccctgcccgcccacccggacaccccaccccttccccctcctttccgaagcccccctccctgttttttcagccccctcccccccatcccccatggagc

SEQ ID NO: 270 - DNA, 270 bp, synthetic construct
  source 1..270: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagcc

SEQ ID NO: 271 - DNA, 126 bp, synthetic construct
  source 1..126: mol_type = other DNA
gggactacgaagcccagaggtccctgcggccctgcccgcccacccggacaccccaccccttccccctcctttccgaagcccccctccctgttttttcagccccctcccccccatcccccatggagc

SEQ ID NO: 272 - DNA, 330 bp, synthetic construct
  source 1..330: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggc

SEQ ID NO: 273 - DNA, 66 bp, synthetic construct
  source 1..66: mol_type = other DNA
tccccctcctttccgaagcccccctccctgttttttcagccccctcccccccatcccccatggagc

SEQ ID NO: 274 - DNA, 360 bp, synthetic construct
  source 1..360: mol_type = other DNA
caggcggggaggggccccggggcggggagggggccggcccgcgggccgcgcagccggaagccgggaccgccaccggcccccggcgaggggagcccggctccaggccccgccccctggcgggctggccccgcccccgcgccgcgccgcgcgatcggcccgcgcccattggctctccggcccgccgctcaccgcccctcctccgcaccgcccctacccgcaggccgcggcgggctgtcggcgcggggcaccctgggacttgtagtccaagccgcttgccacctgccggctgcaaacggcggagggactacgaagcccagaggtccctgcggccctgcccgcccacccggacaccccacccct

SEQ ID NO: 275 - DNA, 36 bp, synthetic construct
  source 1..36: mol_type = other DNA
ttttttcagccccctcccccccatcccccatggagc

SEQ ID NO: 276 - DNA, 372 bp, synthetic construct
  source 1..372: mol_type = other DNA
gtggcttagaaaggtccgatgctgagtcagcaagcaagtactctaatcctctataggacaaactataaatagaatacgaggagttaatcccctgttaatcccctagcttagattctatttatagttattactatgctgactcagcaatagacggtagaggattagagcactcgtttgacagatggtctcctgctattggtcagaatgtgtcacgtgaccatactcagctgcaagggttggtgggaactaagccacagtaacatgcgactaatcctctctgtctacagagtggcttagtctatcgtgttgctgactcagcattagatagtgacagatggtcctgcaggtctagagggtatataatgggggcca

SEQ ID NO: 277 - DNA, 399 bp, synthetic construct
  source 1..399: mol_type = other DNA
gtggcttagaaaggtccgatgctgagtcagcaagcaagtactctaatcctctataggacaaactataaatagaatacgaggagttaatccccttacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacagtaacatgcgactaatcctctctgtctacagagtggcttagtctatcgtgttgctgactcagcattagatagtgacagatggtcctgcaggtctagagggtatataatgggggcca

SEQ ID NO: 278 - DNA, 399 bp, synthetic construct
  source 1..399: mol_type = other DNA
gttaatcccctagcttagattctatttatagttattactatgctgactcagcaatagacggtagaggattagagcactcgtttgacagatggttacacggttaagactcaatccggtaagtaagagaaaaacaggaaaagagtttacatgccactcctgctattggtcagaatgtgtcacgtgaccatactcagctgcaagggttggtgggaaatgtagtctttttttctgggaggccatgtgttcggcttaaaattgtctttttttctttttctaagccacagtaacatgcgactaatcctctctgtctacagagtggcttagtctatcgtgttgctgactcagcattagatagtgacagatggtcctgcaggtctagagggtatataatgggggcca

SEQ ID NO: 279 - DNA, 93 bp, synthetic construct
  source 1..93: mol_type = other DNA
gttaatcccctagcttagattctatttatagttattactatgctgactcagcaatagacggtagaggattagagcactcgtttgacagatggt

SEQ ID NO: 280 - DNA, 93 bp, synthetic construct
  source 1..93: mol_type = other DNA
gtggcttagaaaggtccgatgctgagtcagcaagcaagtactctaatcctctataggacaaactataaatagaatacgaggagttaatcccct